(19) United States
(12) Patent Application Publication
(10) Pub. No.: US 2006/0241172 A1
(43) Pub. Date: Oct. 26, 2006

(54) Benzodioxane and benzodioxolane derivatives and uses thereof

(75) Inventors: Dahui Zhou, East Brunswick, NJ (US); Gary Paul Stack, Ambler, PA (US); Jonathan Laird Gross, Cranbury, NJ (US); Hong Gao, Belle Mead, NJ (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/409,466

(22) Filed: Apr. 21, 2006

Related U.S. Application Data
(60) Provisional application No. 60/673,884, filed on Apr. 22, 2005.

(51) Int. Cl. A61K 31/36; C07D 319/14

(52) U.S. Cl. 514/452; 514/465; 549/362

(57) ABSTRACT

Compounds of formula I or pharmaceutically acceptable salts thereof are provided: 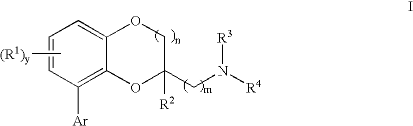
wherein each of R¹, R², R³, R⁴, y, n, m, and Ar are as defined, and described in classes and subclasses herein, which are agonists or partial agonists of the 2C subtype of brain serotonin receptors. The compounds, and compositions containing the compounds, can be used to treat a variety of central nervous system disorders such as schizophrenia.

### CROSS REFERENCE TO RELATED APPLICATIONS

This application claims priority to U.S. Provisional Patent Application Ser. No. 60/673,884, filed Apr. 22, 2005, the entirety of which is hereby incorporated herein by reference.

### FIELD OF THE INVENTION

The present invention relates to 5-HT_{2C} receptor agonists or partial agonists, processes for their preparation, and uses thereof.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

The present invention relates to 5-HT_{2C} receptor agonists or partial agonists and uses thereof. In one aspect, the invention relates to novel aryl substituted 2,3-dihydrobenzo[1,4]-dioxane and aryl substituted 2,3-dihydrobenzo[1,4]dioxolane derivatives that act as agonists or partial agonists of the 5-HT_{2C} receptor. The compounds can be used, for example, to treat schizophrenia and the concomitant mood disorders and cognitive impairments of schizophrenia and depression. In certain embodiments, compounds of the present invention are less likely to produce the body weight increases associated with current atypical antipsychotics. The compounds of the present invention can also be used for the treatment of obesity and its comorbidities. Compounds of the present invention are also useful for treating a variety of psychotic, depression and related disorders, and cognitive disorders as described in detail herein.

In certain embodiments, the present invention provides a compound of formula I: 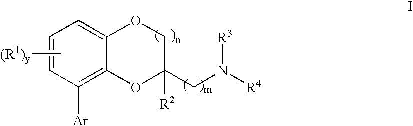
or a pharmaceutically acceptable salt thereof, wherein:
m is 1 or 2;
n is 0 or 1;
Ar is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur, wherein Ar is optionally substituted with one or more R^{x} groups;
each R^{x} is independently selected from —R, -Ph, —CN, halogen, —OR, —C(O)NH₂, —C(O)OR, —NHC(O)R, —SO₂R, or —NHSO₂R;
y is 0-3;
each R¹ is independently —R, —CN, halogen, —OR, —C(O)NH₂, —C(O)OR, —NHC(O)R, —SO₂R, or —NHSO₂R;
each R is independently hydrogen, C₁₋₆ aliphatic or fluoro-substituted C₁₋₆ aliphatic;
R² is hydrogen, C₁₋₃ alkyl, or —O(C₁₋₃ alkyl); and
each of R³ and R⁴ is independently hydrogen or C₁₋₆ aliphatic.

In certain other embodiments, the invention relates to methods for treating a patient suffering from schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, substance abuse, addiction to alcohol and various other drugs, including cocaine and nicotine, gastrointestinal disorders, obesity, or a central nervous system deficiency associated with trauma, stroke, or spinal cord injury, or other conditions or disorders as described herein, that includes administering to the patient a therapeutically effective amount of a compound of formula I, or a pharmaceutically acceptable salt thereof.

In still other embodiments, the invention relates to compositions comprising a compound of formula I or a pharmaceutically acceptable salt thereof, and one or more pharmaceutically acceptable carriers, excipients, or diluents.

### DETAILED DESCRIPTION OF THE INVENTION

1. Compounds and Definitions:

The present invention relates to novel aryl substituted 2,3-dihydrobenzo[1,4]dioxane and aryl substituted 2,3-dihydrobenzo[1,4]dioxolane derivatives that are agonists or partial agonists of the 2C subtype of brain serotonin receptors.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. In certain embodiments, aliphatic groups contain 1-4 aliphatic carbon atoms, and in yet other embodiments, aliphatic groups contain 1-3 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Such cycloaliphatic groups include cycloalkyl, cycloalkenyl, and cycloalkynyl groups. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “unsaturated,” as used herein, means that a moiety has one or more units of unsaturation.

The term “lower alkyl,” as used herein, refers to a hydrocarbon chain having up to 4 carbon atoms, preferably 1 to 3 carbon atoms, and more preferably 1 to 2 carbon atoms. The term “alkyl” includes, but is not limited to, straight and branched chains such as methyl, ethyl, n-propyl, isopropyl, n-butyl, isobutyl, sec-butyl, or t-butyl.

The term “alkoxy,” as used herein, refers to the group —OR*, wherein R* is a lower alkyl group.

The terms “halogen” or “halo,” as used herein, refer to chlorine, bromine, fluorine or iodine.

The term “haloalkyl,” as used herein, or as part of a moiety such as “haloalkoxy” refers to an alkyl group, as defined herein, that has one or more halogen substituents. In certain embodiment, every hydrogen atom on said alkyl group is replaced by a halogen atom. Such haloalkyl groups include —CF₃. Such haloalkoxy groups include —OCF₃.

The term “fluoro-substituted aliphatic,” as used herein, an aliphatic group, as defined herein, that has one or more fluorine substituents. In certain embodiment, a fluoro-substituted aliphatic group is a fluoroalkyl group.

The term “fluoroalkyl,” as used herein, or as part of a moiety such as “fluoroalkoxy” refers to an alkyl group, as defined herein, that has one or more fluorine substituents. In certain embodiment, every hydrogen atom on said alkyl group is replaced by a fluorine atom.

The term “alkenyl,” as used herein refers to an aliphatic straight or branched hydrocarbon chain having 2 to 4 carbon atoms that has one or more double bond. Examples of alkenyl groups include vinyl, prop-1-enyl, allyl, methallyl, but-1-enyl, but-2-enyl, or but-3-enyl. The term “lower alkenyl” refers to an alkenyl group having 1 to 3 carbon atoms.

The term “aryl,” as used herein refers to phenyl or an 8-10 membered bicyclic partially unsaturated or aryl ring. Exemplary aryl groups include phenyl and naphthyl. In certain embodiments, the term “aryl,” as used herein refers to an 8-10 membered bicyclic partially unsaturated the wherein at least one of the rings is aromatic.

As used herein, the term “Ph” refers to a phenyl ring.

The term “heteroaryl,” as used herein, refers to a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur. Examples of heteroaryls include, but are not limited to, thienyl, furyl, pyrrolyl, imidazolyl, pyrazolyl, oxazolyl, isoxazolyl, thiazolyl, isothiazolyl, pyridyl, pyrazinyl, pyrimidinyl, pyridazinyl, indolyl, indazolyl, benzofuranyl, isobenzofuranyl, benzothienyl, isobenzothienyl, quinolyl, isoquinolyl, quinoxalinyl, or quinazolinyl.

The terms “effective amount” and “therapeutically effective amount,” as used herein, refer to the amount of a compound of formula I that, when administered to a patient, is effective to at least partially treat a condition from which the patient is suffering from. Such conditions include, but are not limited to, schizophrenia, schizoaffective disorder, schizophreniform disorder, L-DOPA-induced psychosis, bipolar disorder, obesity, obsessive compulsive disorder, depression, panic disorder, sleep disorders, eating disorders, and epilepsy.

The term “pharmaceutically acceptable salts” or “pharmaceutically acceptable salt” includes acid addition salts, that is salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Where a compound of formula I contains a substituent with acidic properties, for instance, phenolic hydroxyl, —SO₂H or —CO₂H as R¹ or R^{x}, the term also includes salts derived from bases, for example, sodium salts.

The term “patient,” as used herein, refers to a mammal. In certain embodiments, the term “patient,” as used herein, refers to a human.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The terms “treat” or “treating,” as used herein, refers to partially or completely alleviating, inhibiting, preventing, ameliorating and/or relieving the condition.

The terms “suffer” or “suffering,” as used herein, refers to one or more conditions that a patient has been diagnosed with, or is suspected to have.

2. Description of Exemplary Compounds:

In certain embodiments, the invention relates to a compound of formula I: 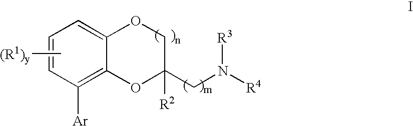
or a pharmaceutically acceptable salt thereof, wherein:
m is 1 or 2;
n is 0 or 1;
Ar is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic heteroaryl ring having 1-5 heteroatoms independently selected from nitrogen, oxygen, or sulfur, wherein Ar is optionally substituted with one or more R^{x} groups;
each R^{x} is independently selected from —R, -Ph, —CN, halogen, —OR, —C(O)NH₂, —C(O)OR, —NHC(O)R, —SO₂R, or —NHSO₂R;
y is 0-3;
each R¹ is independently —R, —CN, halogen, —OR, —C(O)NH₂, —C(O)OR, —NHC(O)R, —SO₂R, or —NHSO₂R;
each R is independently hydrogen or C₁₋₆ aliphatic or fluoro-substituted C₁₋₆ aliphatic;
R² is hydrogen, C₁₋₃ alkyl, or —O(C₁₋₃ alkyl); and
each of R³ and R⁴ is independently hydrogen or C₁₋₆ aliphatic.

As defined generally above, the n group of formula I is 0 or 1. In certain embodiments, n is 1 thus forming a compound of formula Ia having a benzodioxane ring: 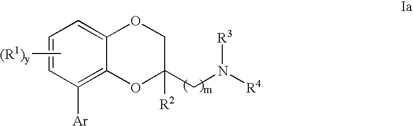
or a pharmaceutically acceptable salt thereof, wherein R¹, R², R³, R⁴, Ar, y, and m are as defined above for compounds of formula I and in classes and subclasses as described above and herein.

According to another embodiment, the n group of formula I is 0, thus forming a compound of formula Ib having a benzodioxolane ring: 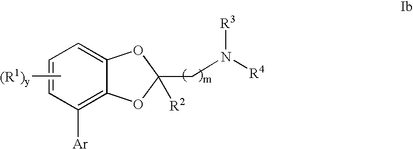
or a pharmaceutically acceptable salt thereof, wherein R¹, R², R³, R⁴, Ar, y, and m are as defined above for compounds of formula I and in classes and subclasses as described above and herein.

As defined generally above, y is 0-3 and each R¹ group of formula I is independently —R, —CN, halogen, —OR, —C(O)NH₂, —C(O)OR, —NHC(O)R, —SO₂R, or —NHSO₂R. In certain embodiments, each R¹ group of formula I is independently —R, —CN, halogen or —OR. In other embodiments, each R¹ group of formula I is independently hydrogen, C₁₋₃ aliphatic, halogen, —OH, —O(C₁₋₃ aliphatic) or —CF₃. In still other embodiments, y is 1, and R¹ is halogen.

According to one embodiment, y is 1, n is 1, and R¹ is at the 6- or 7-position of the benzodioxane ring of formula I, thus forming a compound of formula IIa or IIb: 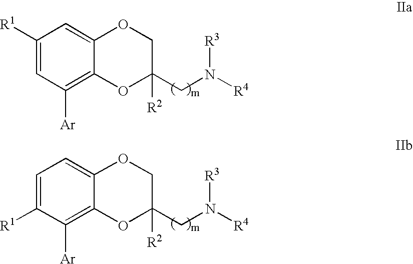
or a pharmaceutically acceptable salt thereof, wherein each R¹, R², R³, R⁴, Ar, and m are as defined above for compounds of formula I and in classes and subclasses as described above and herein.

According to another embodiment, y is 1, n is 0, and R¹ is at the 5- or 6-position of the benzodioxolane ring of formula I, thus forming a compound of formula IIc or IId: 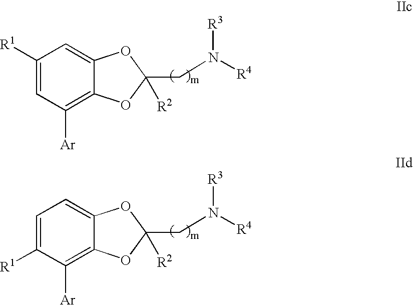
or a pharmaceutically acceptable salt thereof, wherein each R¹, R², R³, R⁴, Ar, and m are as defined above for compounds of formula I and in classes and subclasses as described above and herein.

As defined generally above, the Ar group of formula I is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur, wherein Ar is optionally substituted with one or more R^{x} groups, and wherein each R^{x} is independently selected from —R, -Ph, —CN, halogen, —OR, —C(O)NH₂, —C(O)OR, —NHC(O)R, —SO₂R, or —NHSO₂R. In certain embodiments, the Ar group of formula I is phenyl, an 8-10 membered bicyclic partially unsaturated or aryl ring, or a 5-6 membered monocyclic heteroaryl having 1-4 heteroatoms independently selected from nitrogen, oxygen, or sulfur, wherein Ar is optionally substituted with R^{x}. In other embodiments, the Ar group of formula I is pyridyl, pyrimidinyl, thienyl, or furanyl, wherein Ar is optionally substituted with R^{x}. In still other embodiments, the Ar group of formula I is phenyl, optionally substituted with one or more R^{x} groups. According to one embodiment, Ar is phenyl substituted with R^{x} in the ortho-position thus forming a compound of formula IIIa or IIIb: 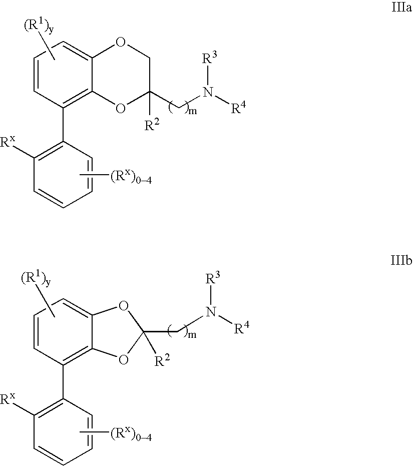
or a pharmaceutically acceptable salt thereof, wherein each R¹, R², R³, R⁴, R^{x}, y and m are as defined above for compounds of formula I and in classes and subclasses as described above and herein.

According to another embodiment, the Ar group of formula I is phenyl substituted with an R^{x} group in both ortho-positions thus forming a compound of formula IIIc or IIId: 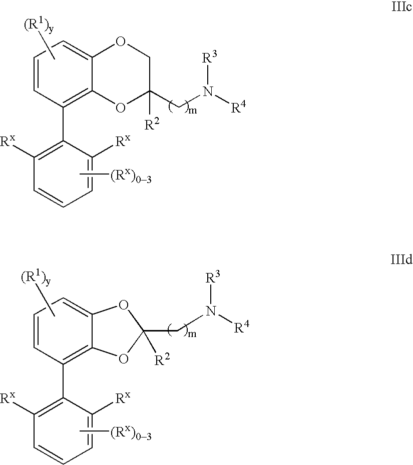
or a pharmaceutically acceptable salt thereof, wherein each R¹, R², R³, R⁴, R^{x}, y and m are as defined above for compounds of formula I and in classes and subclasses as described above and herein.

According to one aspect of the present invention, the Ar group of formula I is substituted with one or more R^{x} groups independently selected from -Ph —R, —CN, halogen or —OR. According to another aspect of the present invention, the Ar group of formula I is substituted with one or more R^{x} groups independently selected from halogen, —O(C₁₋₃ aliphatic), —C₁₋₃ aliphatic, -Ph or —CF₃. In other embodiments, each R^{x} is independently methyl, ethyl, fluoro, chloro, —CF₃, —OCH₃, —OCH₂CH₃, —OCH(CH₃)₂, —OCF₃, or CN.

In certain embodiments, the Ar group of at least one of I, Ia, Ib, IIa, IIb, IIc, IId, IIIa, IIIb, IIIc, and IIId is selected from the following:

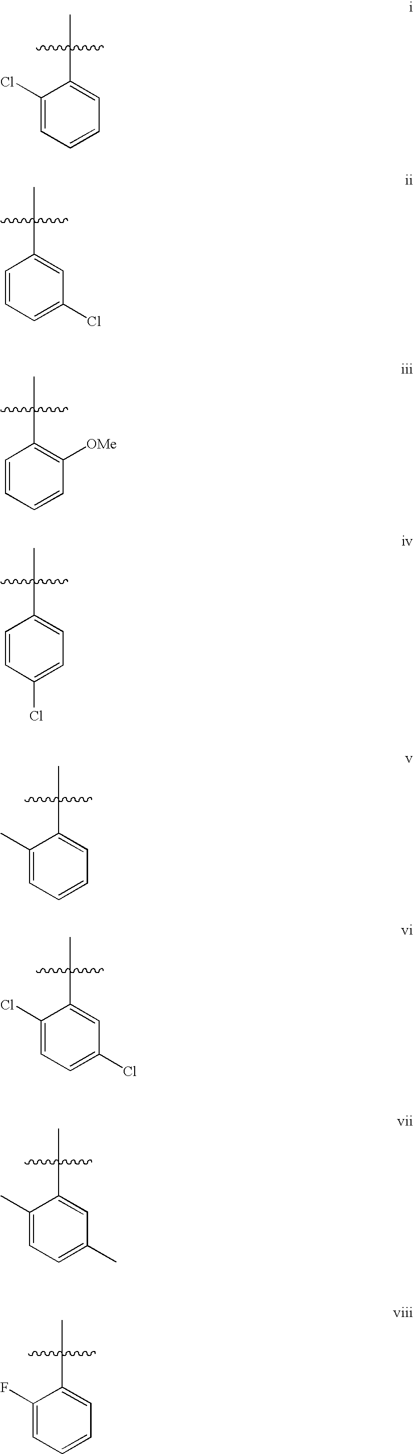

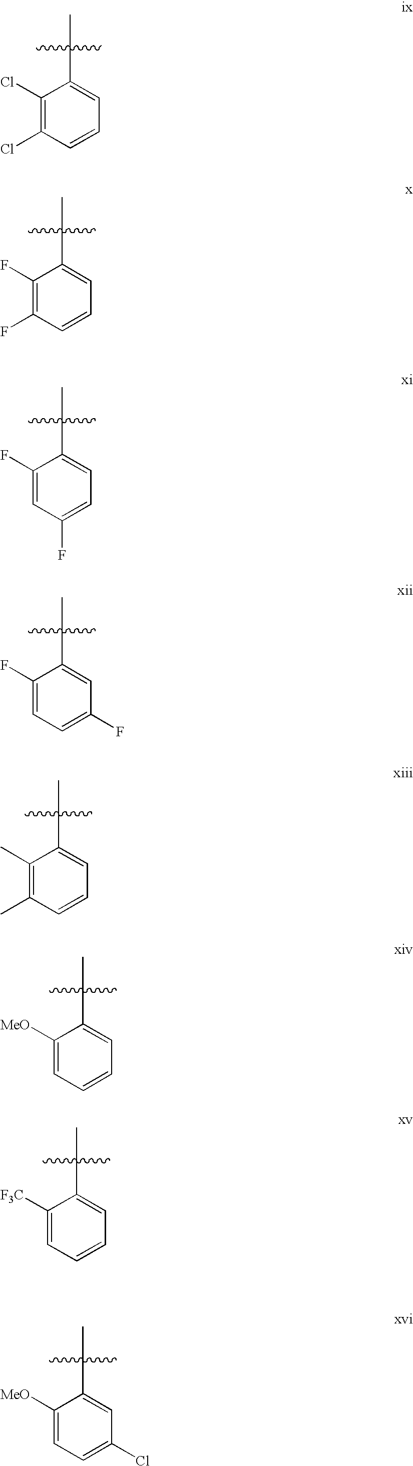

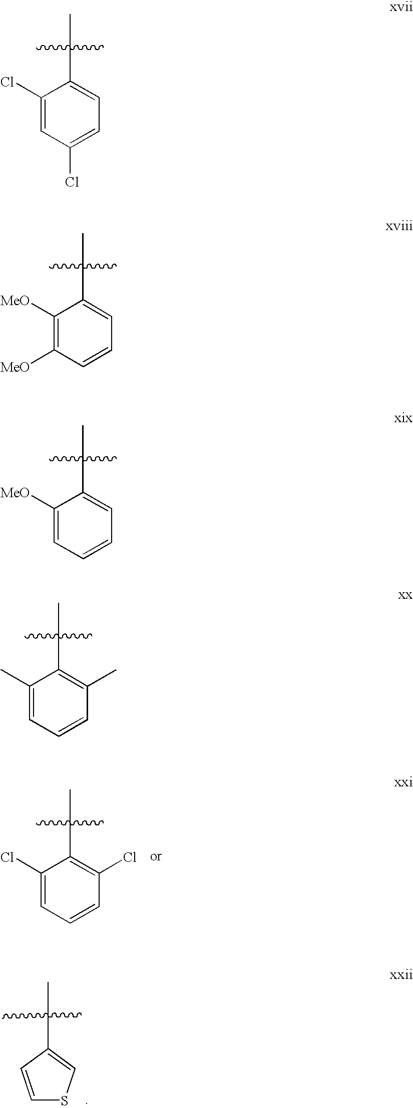

In other embodiments, the Ar group of at least one of formulae I, Ia, Ib, IIa, IIb, IIc, IId, IIIa, IIIb, IIIc, and IIId is selected from the following:

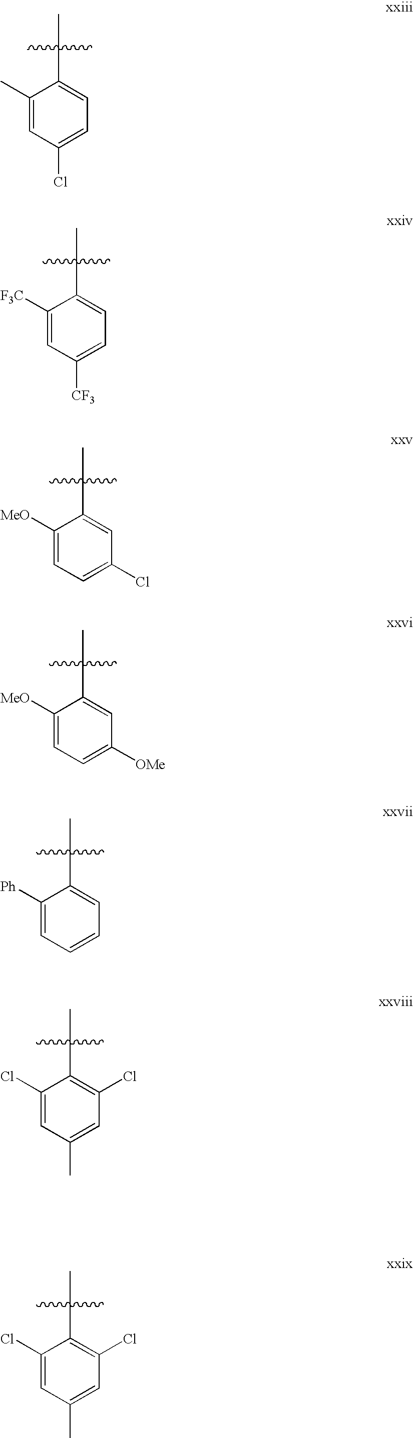

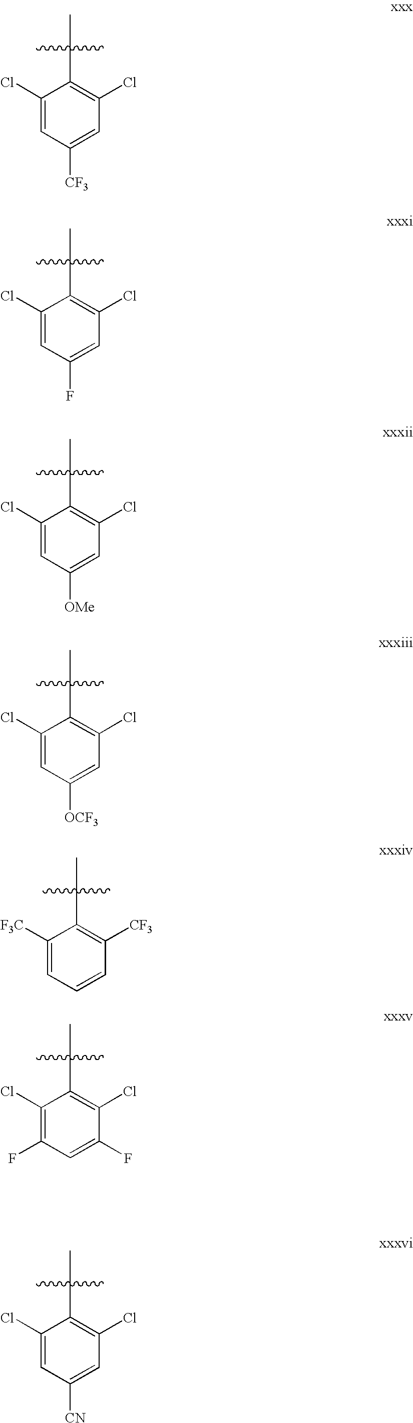

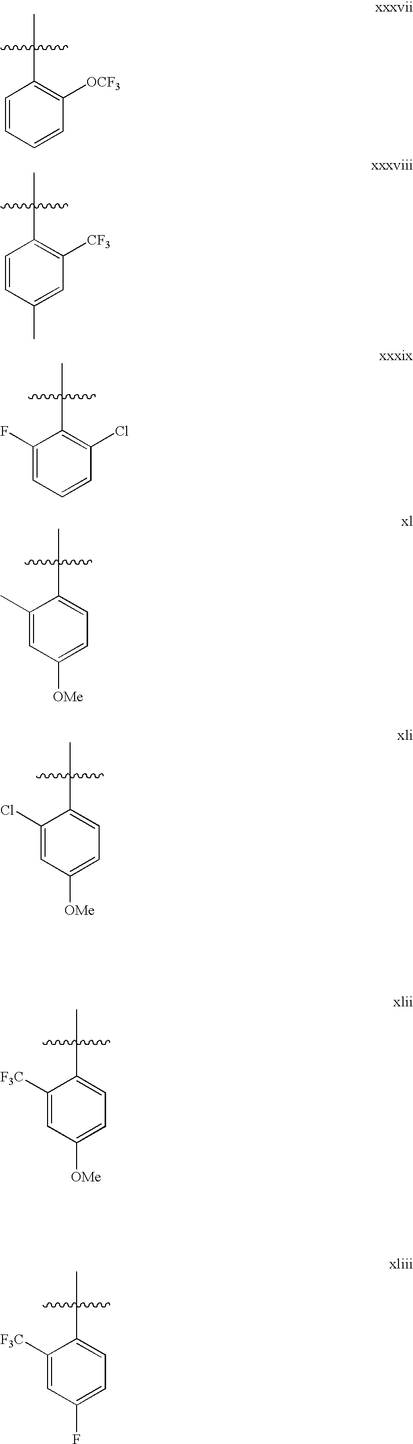

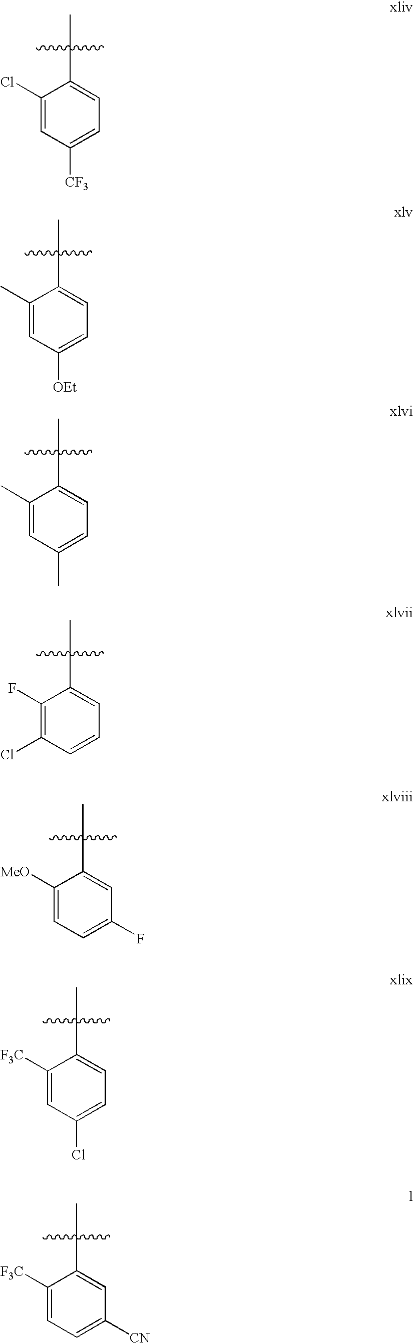

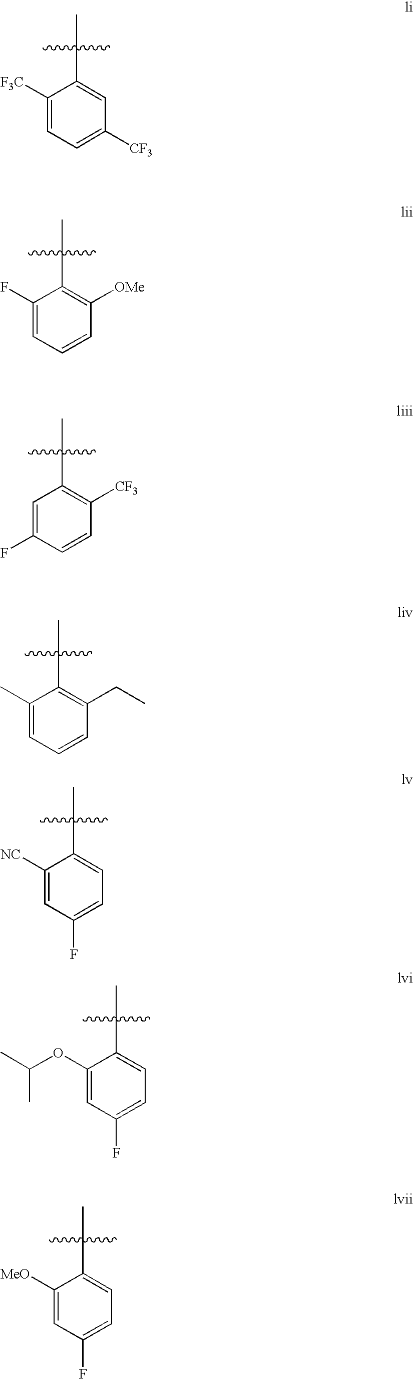

As defined generally above, the R² of formula I is hydrogen, C₁₋₃ alkyl, or —O(C₁₋₃ alkyl). In certain embodiments, the R² of formula I is hydrogen, methyl, or methoxy. In other embodiments, the R² of formula I is hydrogen or methyl.

As defined generally above, the R³ and R⁴ groups of formula I are each independently hydrogen or C₁₋₆ aliphatic. In certain embodiments, both of the R³ and R⁴ groups of formula I are hydrogen. In other embodiments, neither of the R³ or R⁴ groups of formula I is hydrogen. According to one aspect of the present invention, the R³ and R⁴ groups of formula I are independently hydrogen, methyl, ethyl, cyclopropyl, cyclopropylmethyl, n-propyl, allyl, or cyclobutyl. Yet another aspect of the present invention provides a compound of formula I wherein one of the R³ and R⁴ groups of formula I is hydrogen and the other is methyl, ethyl, cyclopropyl, cyclopropylmethyl, n-propyl, allyl, or cyclobutyl.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers.

In certain embodiments, the present invention provides a compound of formula IVa, IVb, IVc, or IVd: 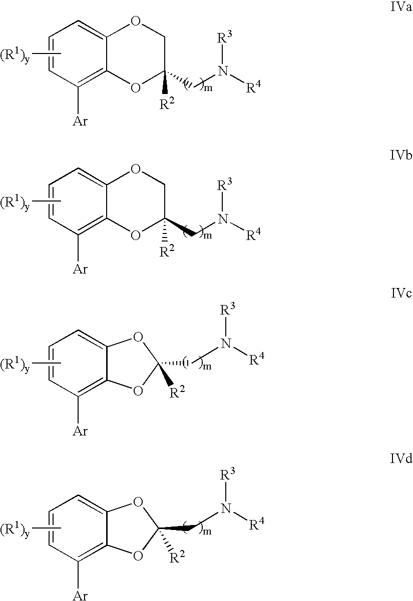
or a pharmaceutically acceptable salt thereof, wherein each R¹, R², R³, R⁴, Ar, y and m are as defined above for compounds of formula I and in classes and subclasses as described above and herein.

According to another embodiment, the present invention provides a compound of formula Va, Vb, Vc, or Vd: 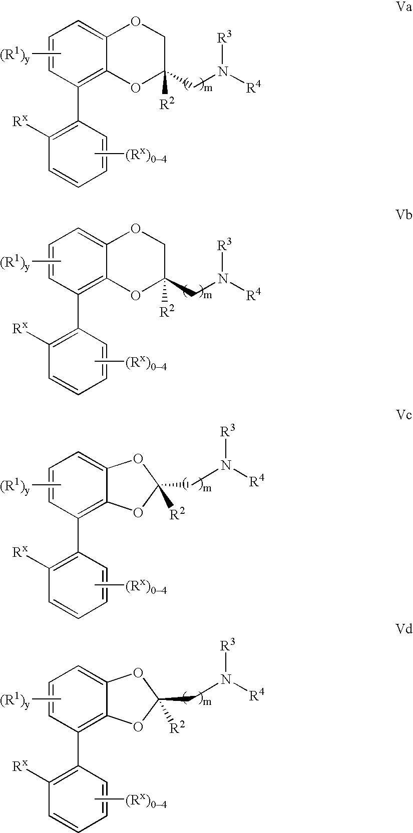
or a pharmaceutically acceptable salt thereof, wherein each R¹, R², R³, R⁴, R^{x}, y and m are as defined above for compounds of formula I and in classes and subclasses as described above and herein.

In other embodiments, the present invention provides a compound of formula VIa, VIb, VIc, or VId: 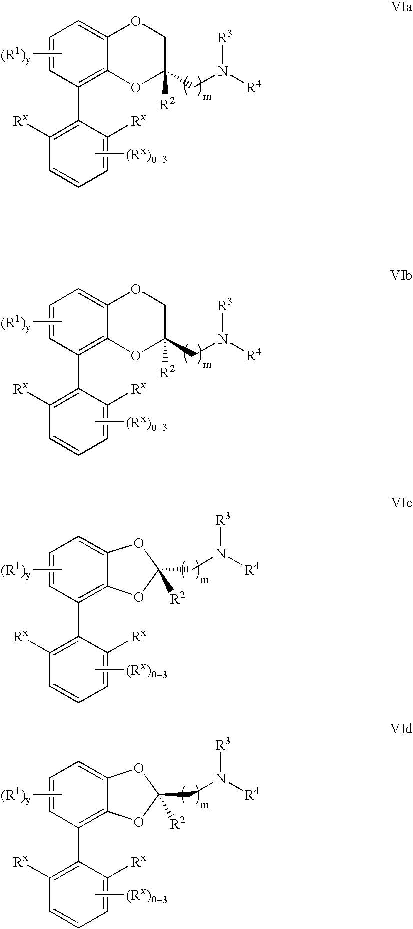
or a pharmaceutically acceptable salt thereof, wherein each R¹, R², R³, R⁴, R^{x}, y and m are as defined above for compounds of formula I and in classes and subclasses as described above and herein.

According to another embodiment, the present invention provides a compound of formula VIIa, VIIb, VIIc, or VIId: 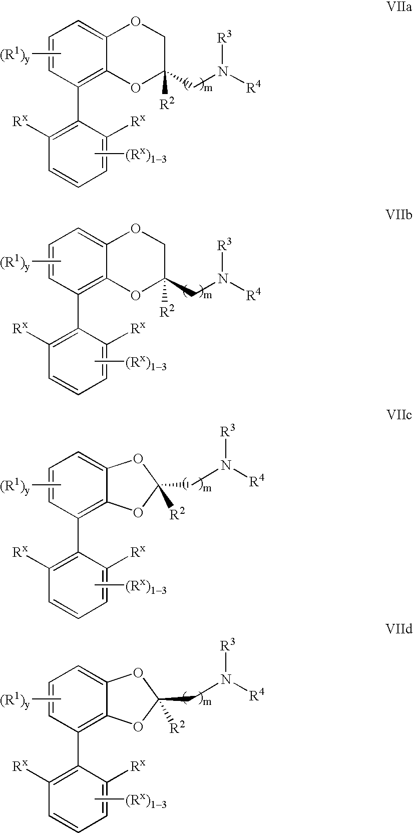
or a pharmaceutically acceptable salt thereof, wherein each R¹, R², R³, R⁴, R^{x}, y and m are as defined above for compounds of formula I and in classes and subclasses as described above and herein.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

It is further recognized that atropisomers of the present compounds may exit. The present invention thus encompasses atropisomeric forms of compounds of formula I as defined above, and in classes and subcasses described above and herein.

Exemplary compounds of formula I are set forth in Table 1, below.

TABLE 1
Exemplary Compounds of Formula I:

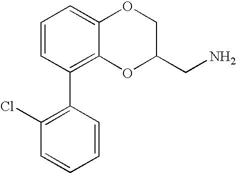

I-1

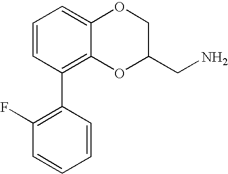

I-2

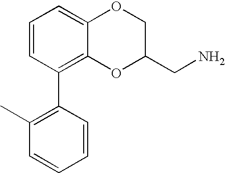

I-3

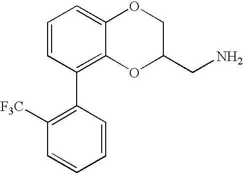

I-4

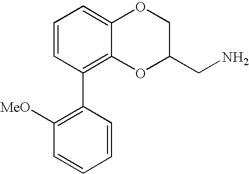

I-5

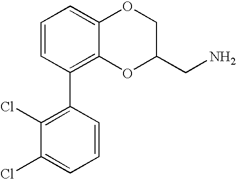

I-6

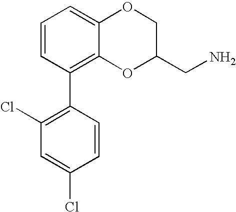

I-7

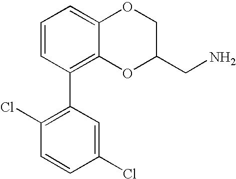

I-8

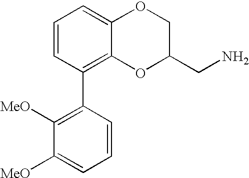

I-9

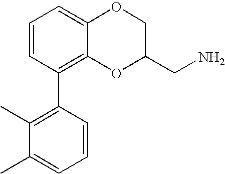

I-10

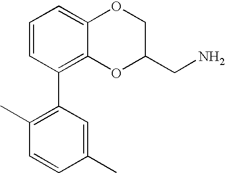

I-11

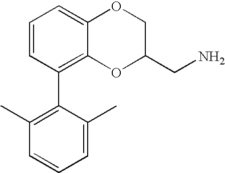

I-12

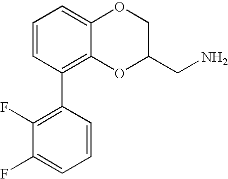

I-13

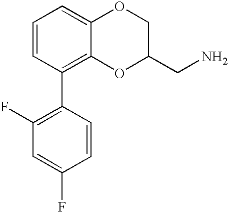

I-14

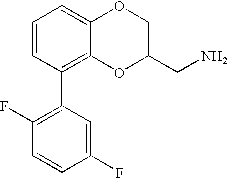

I-15

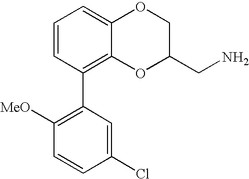

I-16

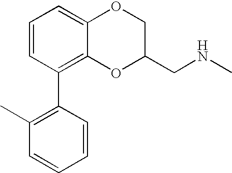

I-17

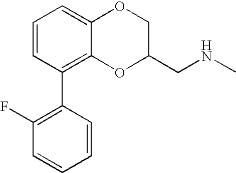

I-18

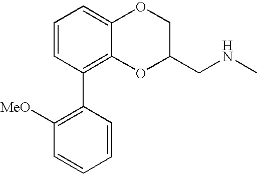

I-19

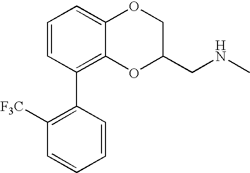

I-20

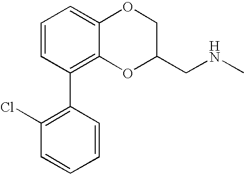

I-21

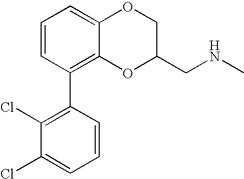

I-22

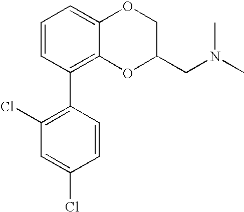

I-23

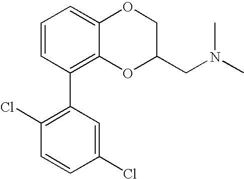

I-24

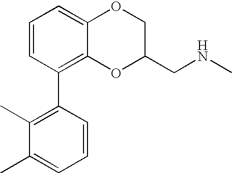

I-25

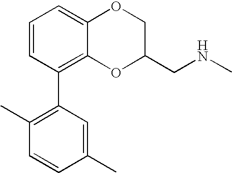

I-26

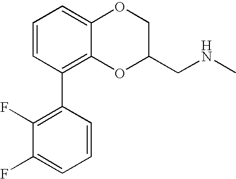

I-27

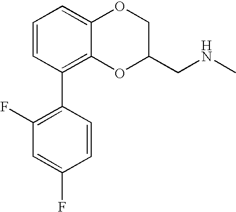

I-28

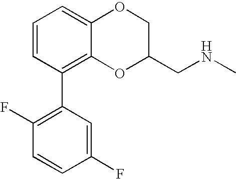

I-29

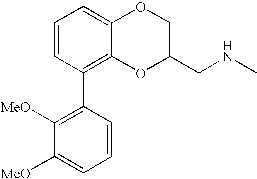

I-30

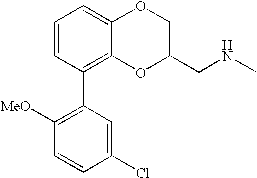

I-31

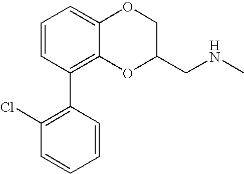

I-32

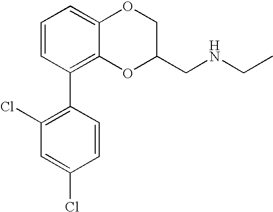

I-33

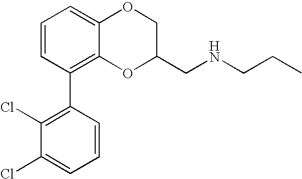

I-34

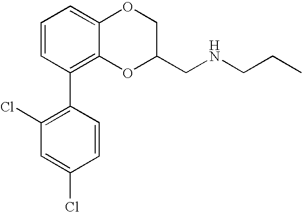

I-35

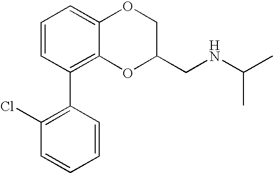

I-36

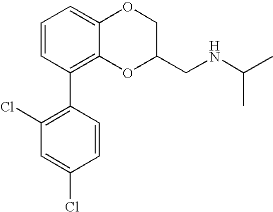

I-37

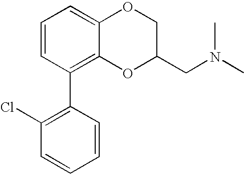

I-38

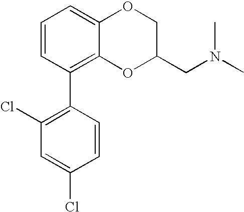

I-39

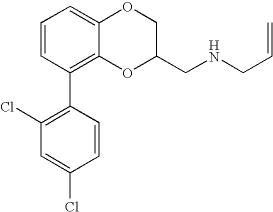

I-40

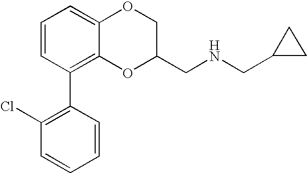

I-41

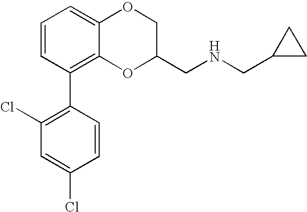

I-42

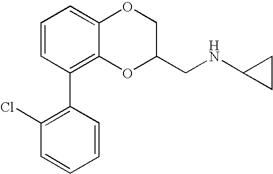

I-43

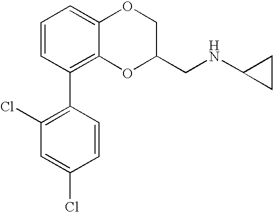

I-44

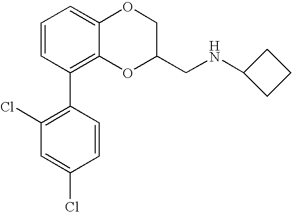

I-45

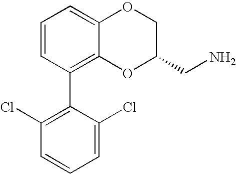

I-46

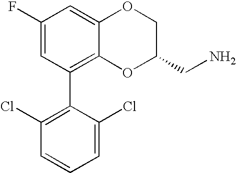

I-47

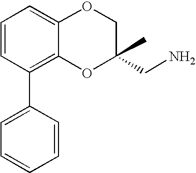

I-48

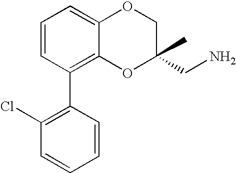

I-49

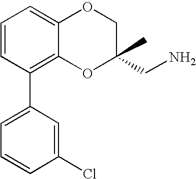

I-50

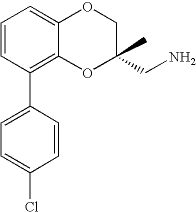

I-51

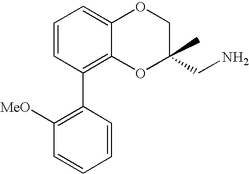

I-52

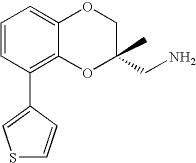

I-53

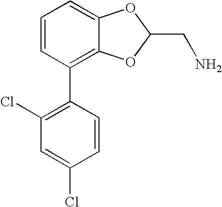

I-54

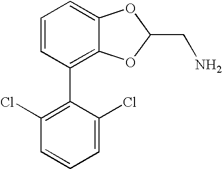

I-55

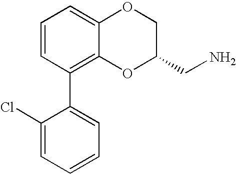

I-56

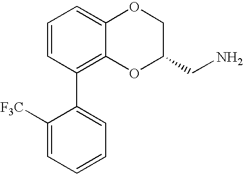

I-57

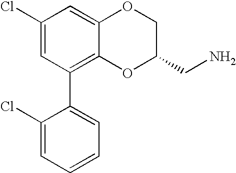

I-58

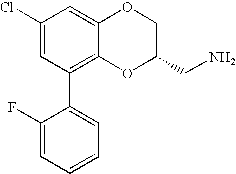

I-59

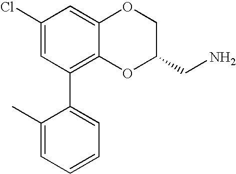

I-60

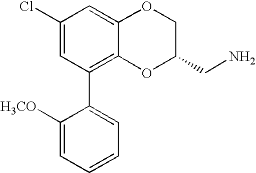

I-61

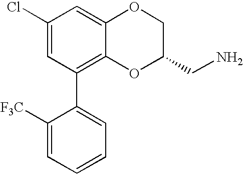

I-62

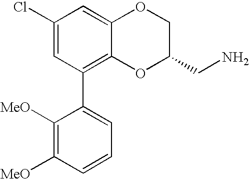

I-63

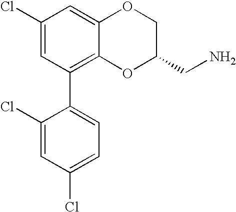

I-64

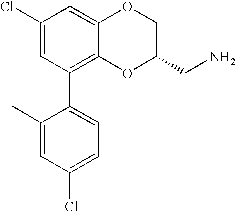

I-65

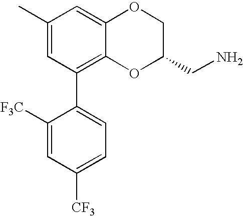

I-66

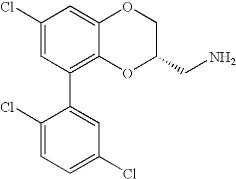

I-67

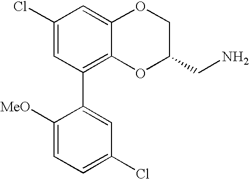

I-68

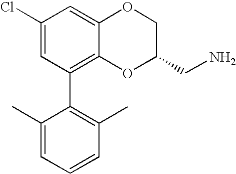

I-69

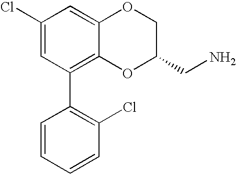

I-70

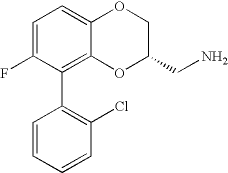

I-71

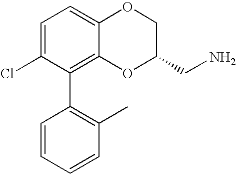

I-72

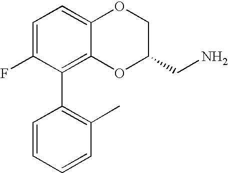

I-73

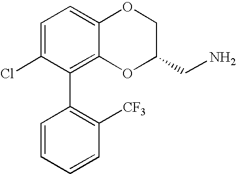

I-74

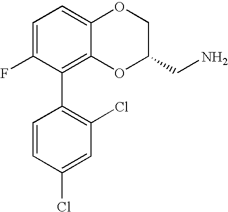

I-75

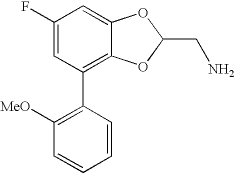

I-76

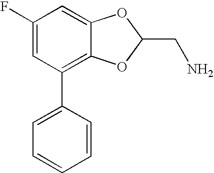

I-77

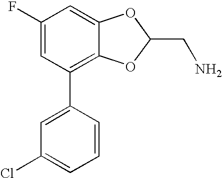

I-78

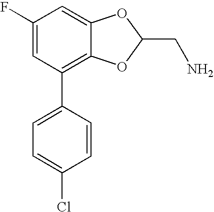

I-79

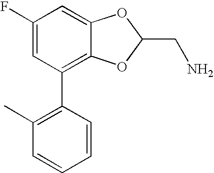

I-80

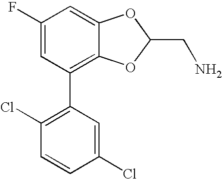

I-81

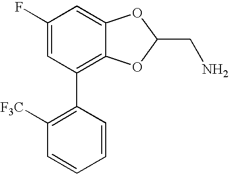

I-82

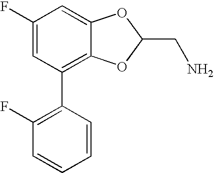

I-83

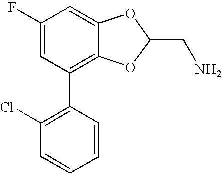

I-84

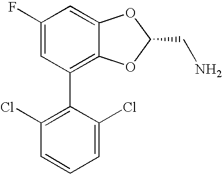

I-85

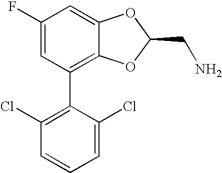

I-86

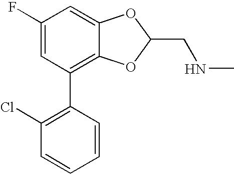

I-87

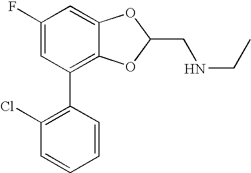

I-88

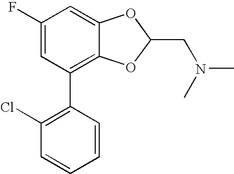

I-89

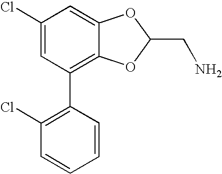

I-90

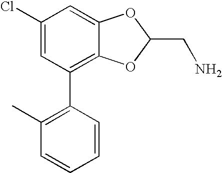

I-91

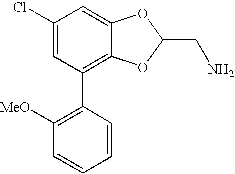

I-92

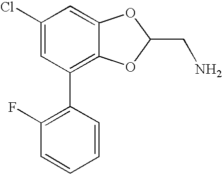

I-93

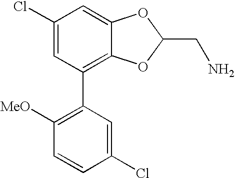

I-94

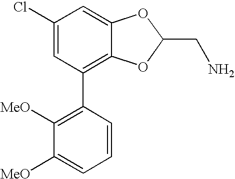

I-95

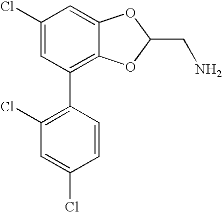

I-96

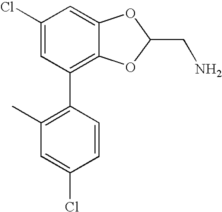

I-97

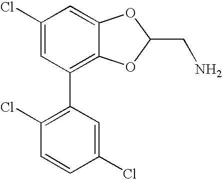

I-98

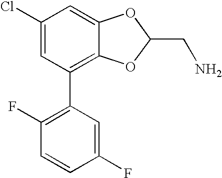

I-99

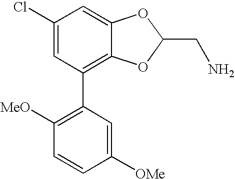

I-100

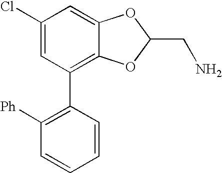

I-101

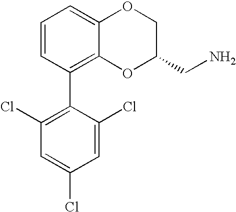

I-102

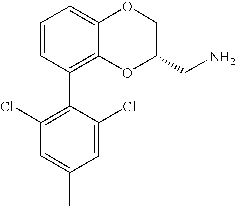

I-103

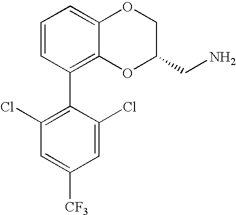

I-104

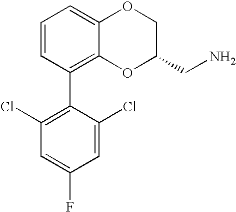

I-105

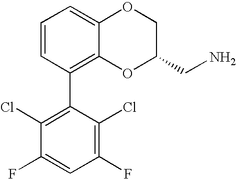

I-106

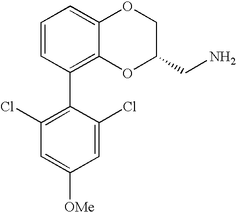

I-107

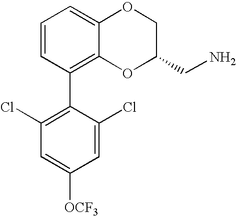

I-108

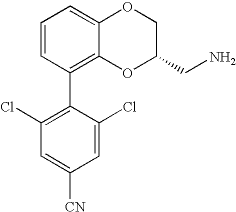

I-109

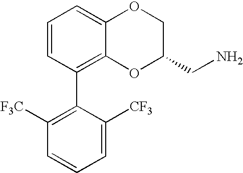

I-110

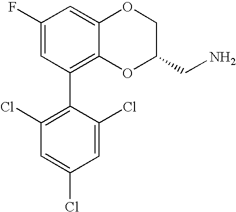

I-111

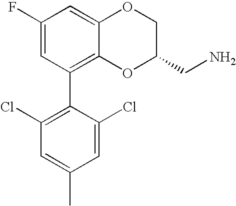

I-112

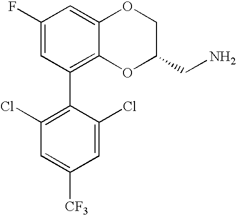

I-113

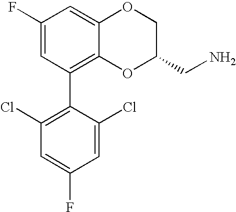

I-114

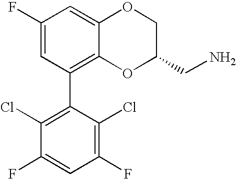

I-115

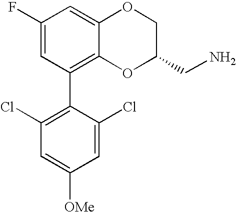

I-116

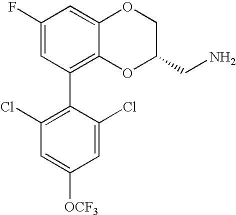

I-117

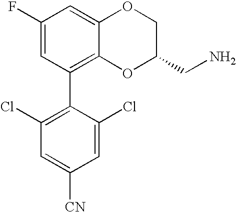

I-118

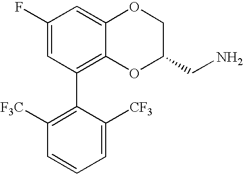

I-119

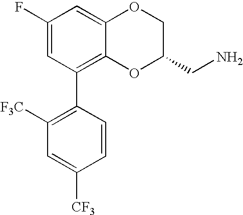

I-120

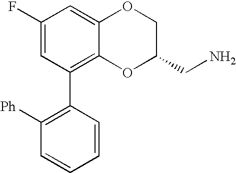

I-121

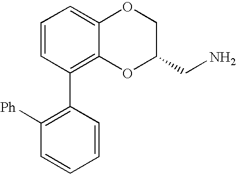

I-122

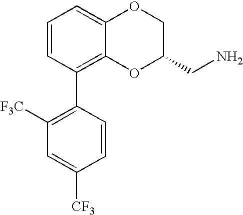

I-123

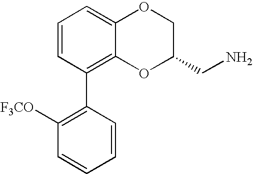

I-124

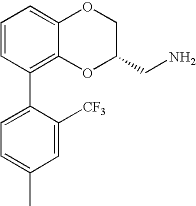

I-125

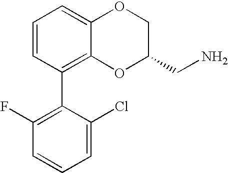

I-126

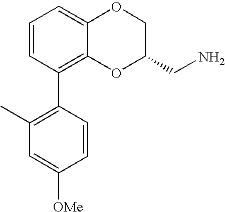

I-127

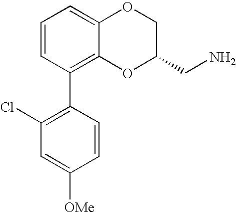

I-128

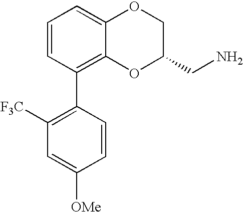

I-129

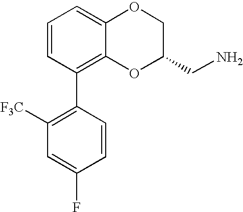

I-130

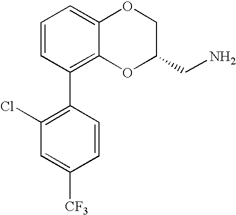

I-131

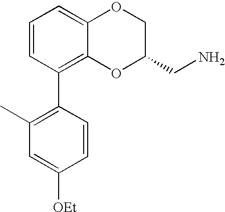

I-132

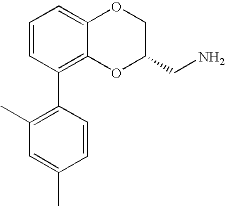

I-133

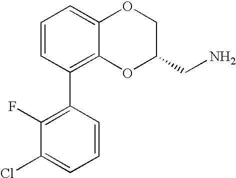

I-134

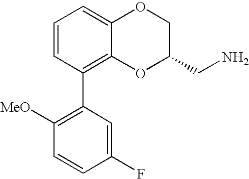

I-135

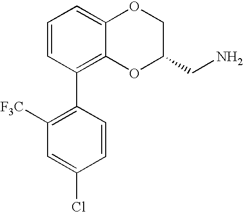

I-136

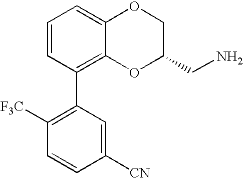

I-137

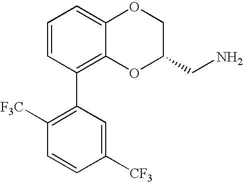

I-138

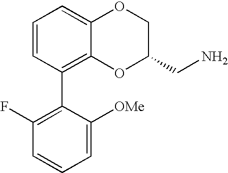

I-139

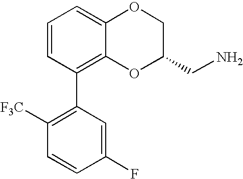

I-140

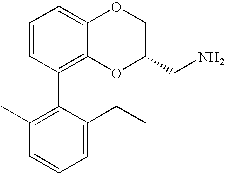

I-141

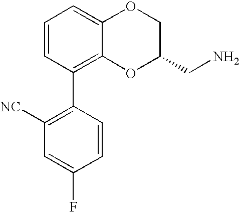

I-142

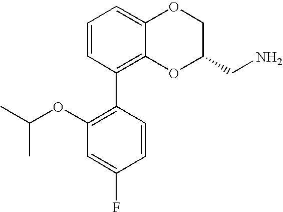

I-143

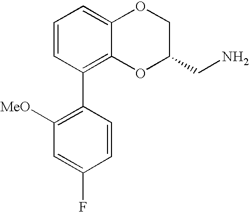

I-144

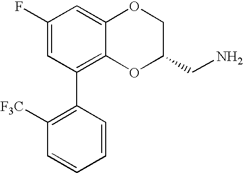

I-145

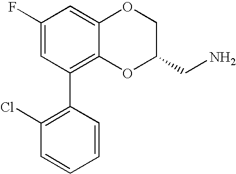

I-146

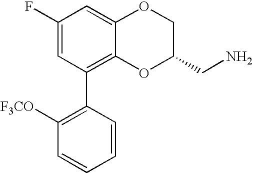

I-147

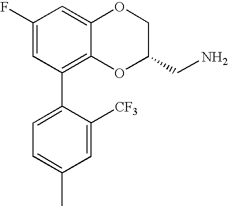

I-148

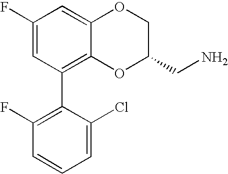

I-149

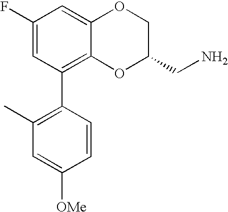

I-150

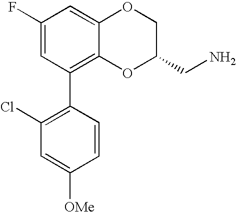

I-151

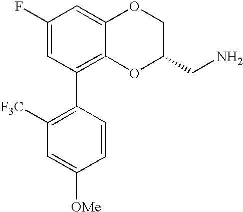

I-152

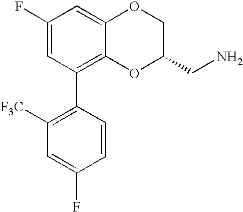

I-153

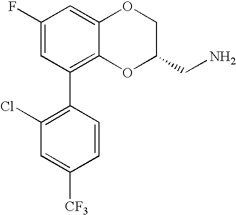

I-154

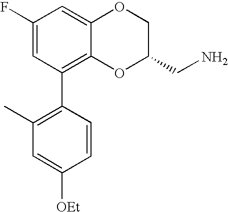

I-155

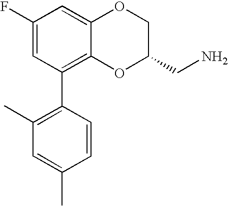

I-156

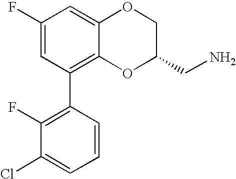

I-157

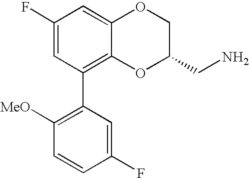

I-158

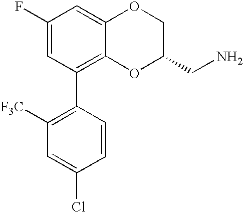

I-159

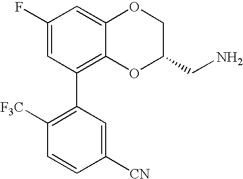

I-160

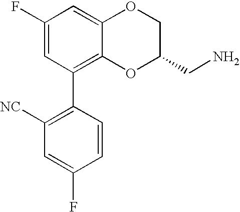

I-165

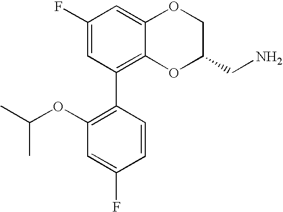

I-166

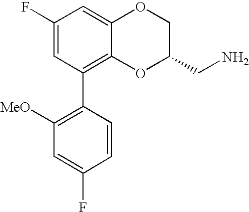

I-167

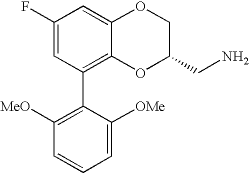

I-168

It will be appreciated that for each racemic compound disclosed in Table 1, above, both enantiomers are separately contemplated and included herein. For example, for compound I-1 depicted above as a racemate, each of its enantiomers of structures I-1a and I-1b: 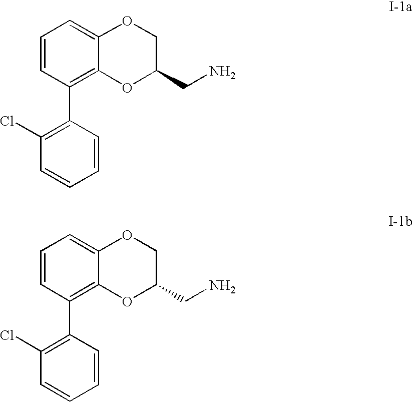
are contemplated and included herein.

It will be appreciated that for each enantiomer disclosed in Table 1, above, the opposite enantiomer is contemplated and included herein. For example, for compounds I-46 and I-47 depicted above as a single enantiomer, the opposite enantiomers of structures I-46a and I-47a: 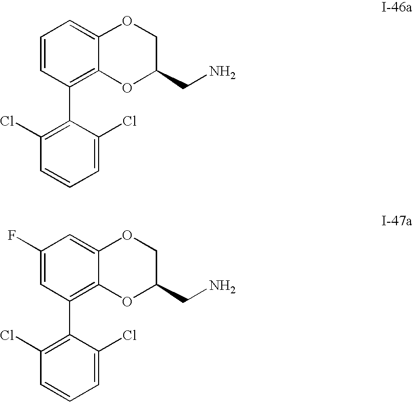
are also contemplated and included herein.

In addition, for each enantiomer disclosed in Table 1, above, the racemate of that compound is also contemplated and included herein. For example, for compounds I-46 and I-47 depicted above as a single enantiomer, their racemates of structures I-46b and I-47b: 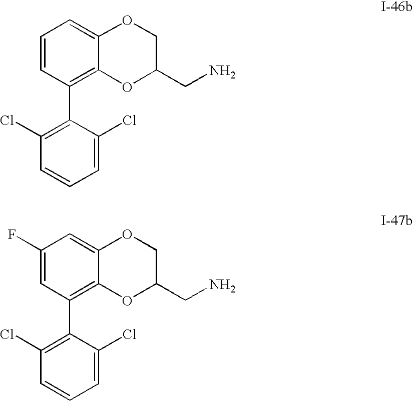
are also contemplated and included herein.
3. General Methods of Providing the Present Compounds:

Compounds of formula I of the present invention are prepared as illustrated in Scheme 1-11, below. Unless otherwise noted, all variables are as defined above and in classes and subclasses described above and herein. Specifically, the appropriately substituted toluene-4-sulfonic acid 8-formyl-2,3-dihydrobenzo[1,4]dioxin-2-ylmethyl ester (1) is converted to a phenol (2) via oxidation with meta-chloroperoxybenzoic acid (Baeyer-Villiger reaction), followed by cleavage of the resulting formate ester under basic condition, such as basic aluminum in methanol or sodium hydroxide in methanol. The phenol thus obtainable is then reacted with trifluoromethanesulfonic anhydride in presence of diisopropylethylamine in methylene chloride to generate corresponding triflate (3). Suzuki coupling of the triflate (3) with different arylboronic acid by using tetrakis(triphenylphospine)palladium (0) under basic condition produced toluene-4-sulfonic acid 8-aryl-2,3-dihydrobenzo[1,4]dioxin-2-methyl ester (4) as a key intermediate. After conversion of the tosylate to the azide (5) with sodium azide, the azide is reduced to an amine with a suitable reducing agent such as triphenylphosphine in tetrahydrofuran and water or catalytic hydrogenation with 5% Pt—S₂ on carbon in ethanol to give the title compounds of the invention of formula I. Certain aldehydes 1 are known compounds and may be obtained from commercial sources.

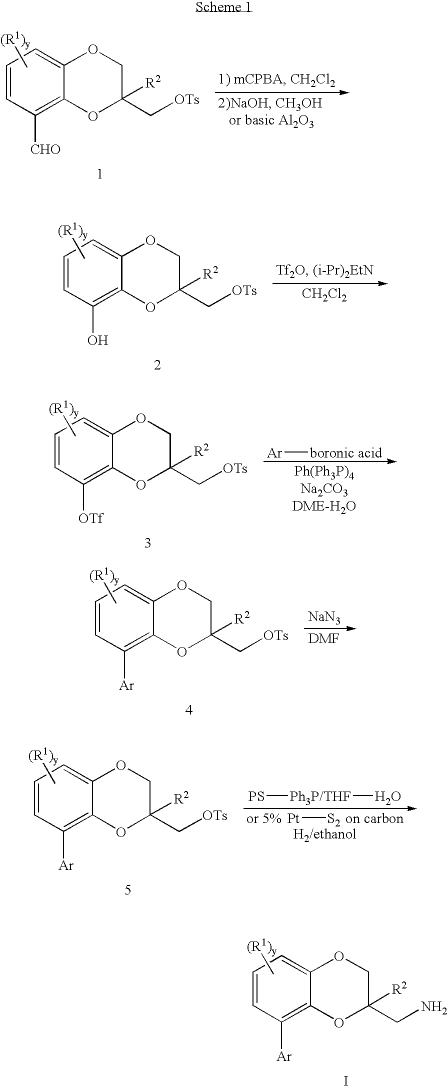

Alternatively, aldehydes of formula 1 are prepared via methods substantially similar to those described in Journal of Medicinal Chemistry (1992), 35(16), 3058-66. According to another alternative, aldehydes 1 are prepared from the 8-allyl benzodioxans described in U.S. Pat. No. 5,756,532 via isomerization of the double bond with bis(acetonitrile)palladium(II)chloride in refluxing methylene chloride, followed by cleavage with either osmium tetroxide and sodium periodate or ozonolysis as shown in Scheme 1a below.

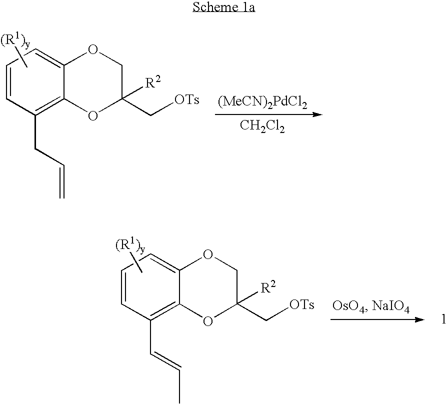

Scheme 2, below, depicts an alternate method for preparing compounds of formula I. The commercially available phenylboronic acids were coupled to different aryl bromides or aryl triflates by using Suzuki coupling reaction to obtain the intermediate 6. ortho-Halogenation of the methyl ether 6, followed by metal-halogen exchange with appropriate Grignard reagent and quenching with 1-formylpiperidine leads to the benzaldehyde derivative 8. The aldehyde moiety is then converted to a phenol 9 via Baeyer-Villiger reaction, followed by cleavage of the resulting formate ester with sodium hydroxide in methanol. The phenol thus obtained is then elaborated via alkylation with a glycidyl tosylate in the presence of a base such as sodium hydride to form an intermediate of formula 10. The methyl ether (10) is then cleaved and epoxide ring is opened by treatment with 33% HBr in acetic acid to generate 11a-b. Intermediate 11a-b is cyclized directly to benzodioxan methanol 12 under basic conditions, such as sodium hydroxide in methanol. The alcohol is converted to a tosylate of formula 13 by treatment with p-toluenesulfonyl chloride, diisopropylethylamine and catalytic amount of dimethylaminopyridine in methylene chloride. As before, displacement of the tosylate with azide, followed by azide reduction provides compounds of formula I.

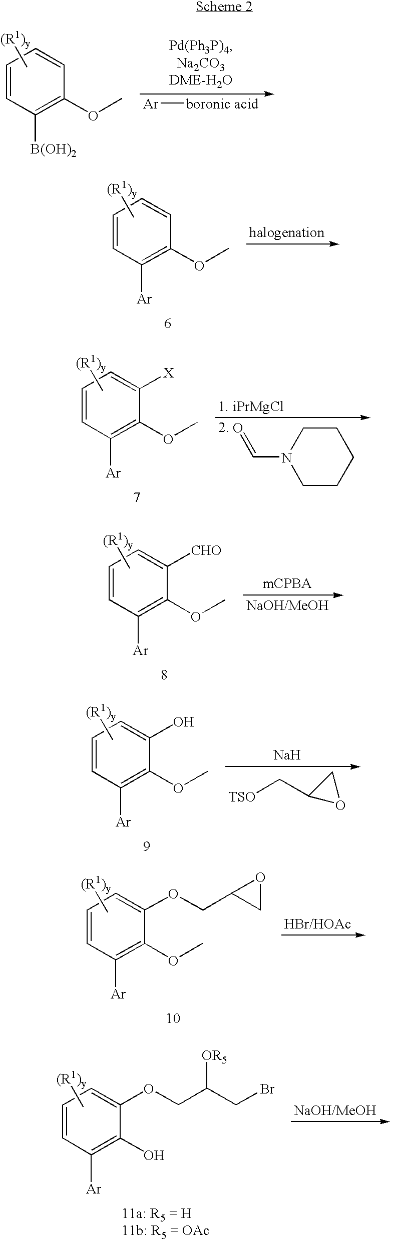

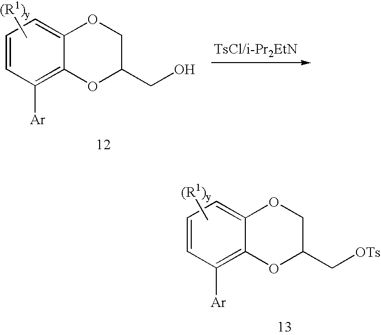

Alternatively as depicted in Scheme 3 below, the methyl ether (6) can also be cleaved by treatment with boron tribromide at −78° C. to form a phenol (14). The phenol is alkylated with allyl bromide in the presence of a suitable base such as potassium carbonate and the product (15) submitted to a Claisen rearrangement in a refluxing high-boiling solvent such as mesitylene or decahydronaphthalene. Protection of the Claisen rearrangement product (16) as the benzyl ether is effected via treatment with benzyl bromide in the presence of a base such as sodium hydride in DMF. Isomerization of the double bond of the benzyl ether into a compound (17), in which the double bond is conjugated with the aromatic ring, is carried out with bis(acetonitrile)palladium(II)chloride in refluxing methylene chloride. Cleavage of the olefin with osmium tetroxide and sodium periodate then gives the o-benzyloxybenzaldehyde (18). The aldehyde moiety is then converted to a phenol (19) via Baeyer-Villiger reaction, followed by cleavage of the resulting formate ester with sodium hydroxide in methanol. After elaboration of the phenol to the glycidyl ether (20) by treatment with a glycidyl tosylate, the benzyl ether is then cleaved and the epoxide ring is opened by treatment with 33% HBr in acetic acid. Compounds of formula I are made following the sequences shown in Scheme 2.

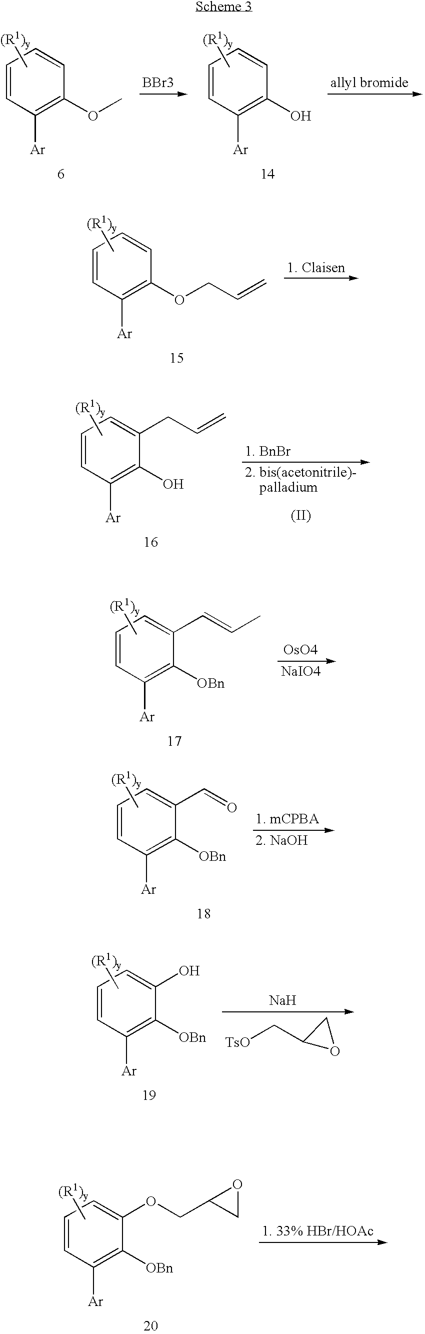

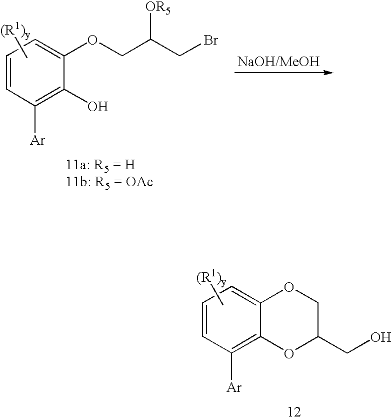

In another method (Scheme 4) the double bond of the Claisen rearrangement product (16) is isomerized to the compound (21) in which the double bond is conjugated with the aromatic ring by using bis(acetonitrile)palladium(II)chloride in refluxing methylene chloride. Cleavage of the olefin with osmium tetroxide and sodium periodate then gives the aldehyde which is then converted to the benzyl ether (18) via treatment with benzyl bromide in the presence of a base such as sodium hydride in DMF. The aldehyde moiety is then converted to a phenol (19) via Baeyer-Villiger reaction, followed by cleavage of the resulting formate ester with sodium hydroxide in methanol. After elaboration of the phenol to the glycidyl ether (20) by treatment with a glycidyl tosylate, the benzyl ether is then cleaved and the epoxide ring is opened by treatment with 10% palladium on carbon with 1,4-cyclohexadiene in the presence of a suitable base such as sodium bicarbonate or sodium carbonate. Tosylates of formula 13 are prepared by the steps shown in Scheme 2, above.

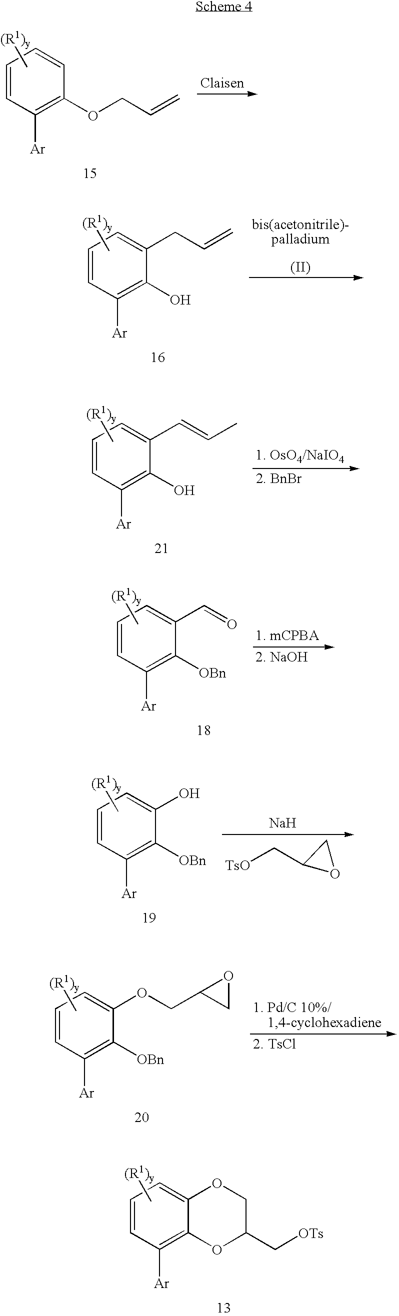

Alternatively (Scheme 5), o-halogenation of phenol (14) under different reaction conditions generates the intermediate (22). Protection of phenol (22) with R⁵ (e.g., methyl or benzyl group), followed by metal-halogen exchange with appropriate Grignard reagent and quenching with 1-formylpiperidine, the benzaldehyde derivative (18) (R⁵=Bn) or (8) (R⁵=Me) can be generated. In another method, intermediates (18) and (8) can be generated by direct o-formylation (ref. J. Chem. Soc. Perkin. Trans 1, 1994, 1823-183.) of phenol (14), followed by protection of intermediate 24 with either methyl or benzyl groups.

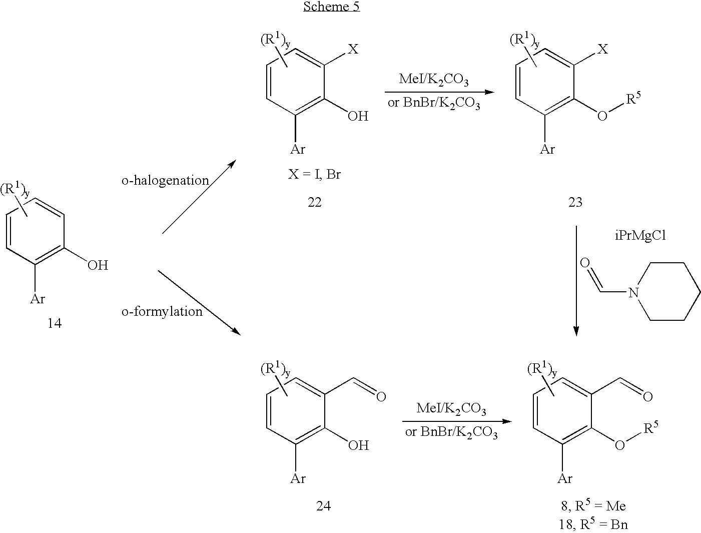

Alternatively, a benzodioxan methanol compound of formula (12) is produced by using a method depicted in Scheme 6, below. The different 2,3-dimethoxyphenyl boronic acids are coupled to aryl bromides or aryl triflates by using Suzuki coupling reaction to obtain intermediates of formula 26. The dimethyl ether (26) is cleaved by treatment with boron tribromide at room temperature to form catechol derivatives (27). After treatment of catechol (27) with a benzyl glycidyl tosylate under basic conditions, the benzodioxan methanol (12) is generated.

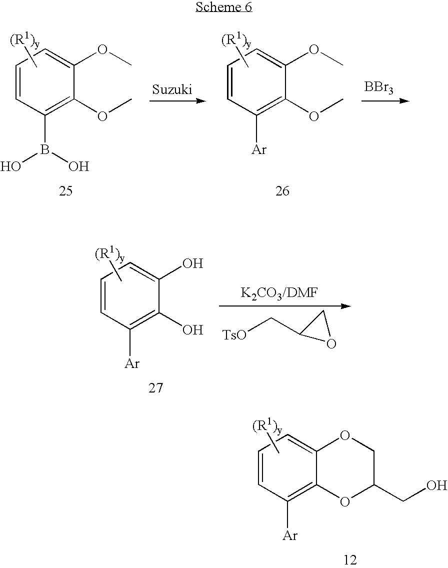

Benzodioxolane derivatives of the invention, wherein n is zero, are alternatively prepared by the sequences outlined in Scheme 7 and Scheme 8, below. Substituted salicylaldehyde (28) undergoes oxidation with meta-chloroperoxybenzoic acid, followed by cleavage of the resulting formate ester with sodium hydroxide in methanol. Catechol (29) thus obtained is condensed with diethyl dibromomalonate under basic condition such as potassium carbonate, followed by hydrolysis to generate dicarboxylic acid 31. Intermediate (31) undergoes decarboxylation in a refluxing high-boiling solvent such as mesitylene, and the resulting monoacid is converted to its methyl ester (32). The methyl ester (32) is reduced to alcohol (33) with a suitable reducing agent such as sodium borohydride. After the conversion of the alcohol to tosylate (34) with p-toluenesulfonyl chloride, different aryls and heteoaryls can be introduced by Suzuki coupling reaction. Replacement of the tosyl with azide, followed by the reduction of the resulting azide, affords the title compounds of formula I of the invention, wherein n is zero The corresponding Cbz derivatives of compound I (racemic) are injected onto a chiral Supercritical Fluid Chromatography instrument to obtain the resolved enantiomers. After removal of the Cbz group, the corresponding chiral compounds of structure I can be obtained.

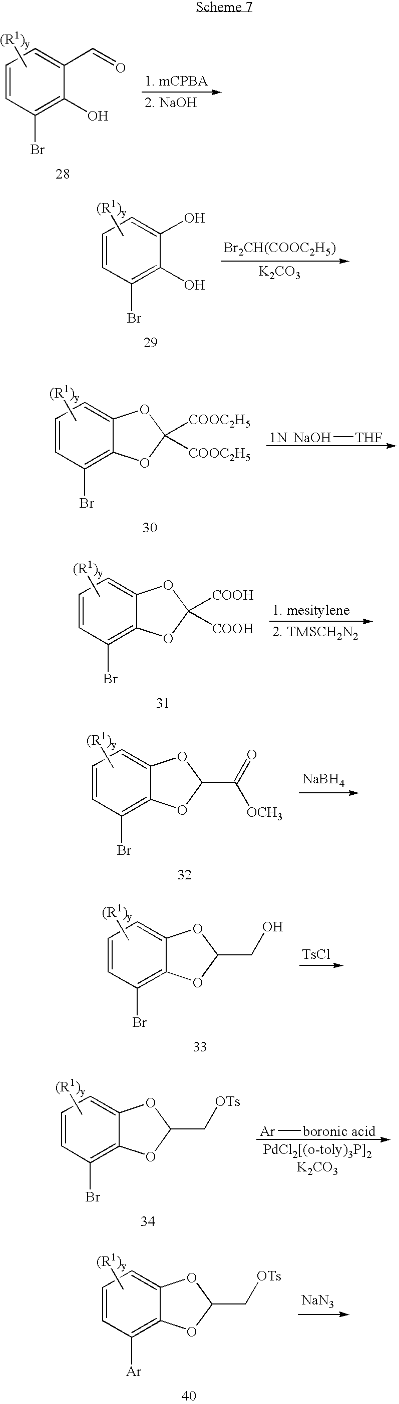

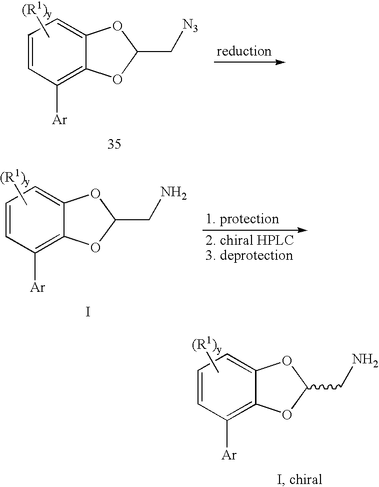

Alternatively (Scheme 8), benzodioxolane derivatives of the invention in which n is zero are made from a substituted guaiacol (9) or 2,3-dimethoxybiphenyl (26). The methyl ether of the substituted guaiacol (9) or 2,3-dimethoxybiphenyl (26) is cleaved by treatment with boron tribromide. The catechol (27) is treated sequentially with diethyl dibromomalonate and 1N sodium hydroxide solution in tetrahydrofuran to generate the dicarboxylic acid (37). After decarboxylation and esterification, the methyl ester (38) is reduced by a reducing agent such as sodium borohydride. The primary alcohol (39) is converted to the tosylate (40) by reaction with p-toluenesulfonyl chloride in the presence of diisopropylethyl amine and catalytical amount of DMAP. Replacement of tosylate (40) with azide, and subsequent reduction of azide with triphenylphosphine in tetrahydrofuran and water, affords the title compounds of formula I of the present invention wherein n is zero.

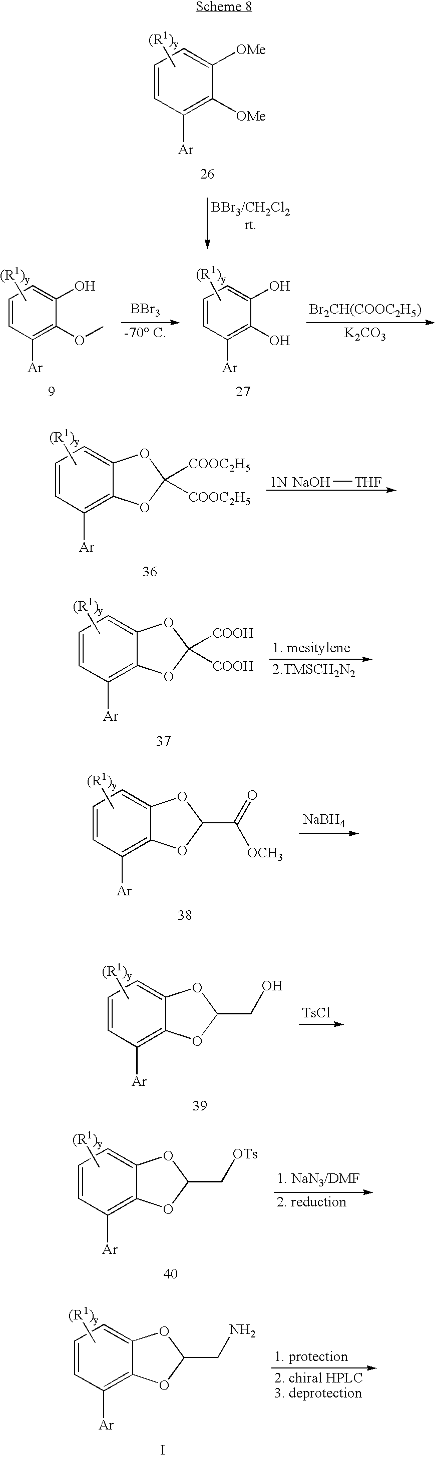

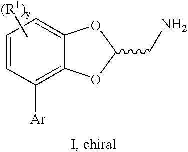

Compounds of the invention in which R³ or R⁴ are not hydrogen are prepared according to Scheme 9, below. Replacement of any of the tosylates, which are generated via the reaction sequences listed in Schemes 1-8, with the appropriately substituted amines affords compounds of formula I (Scheme 9).

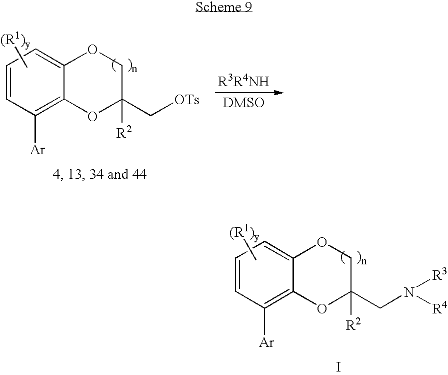

Scheme 10, below, depicts an alternative method for preparing compounds of the present invention. 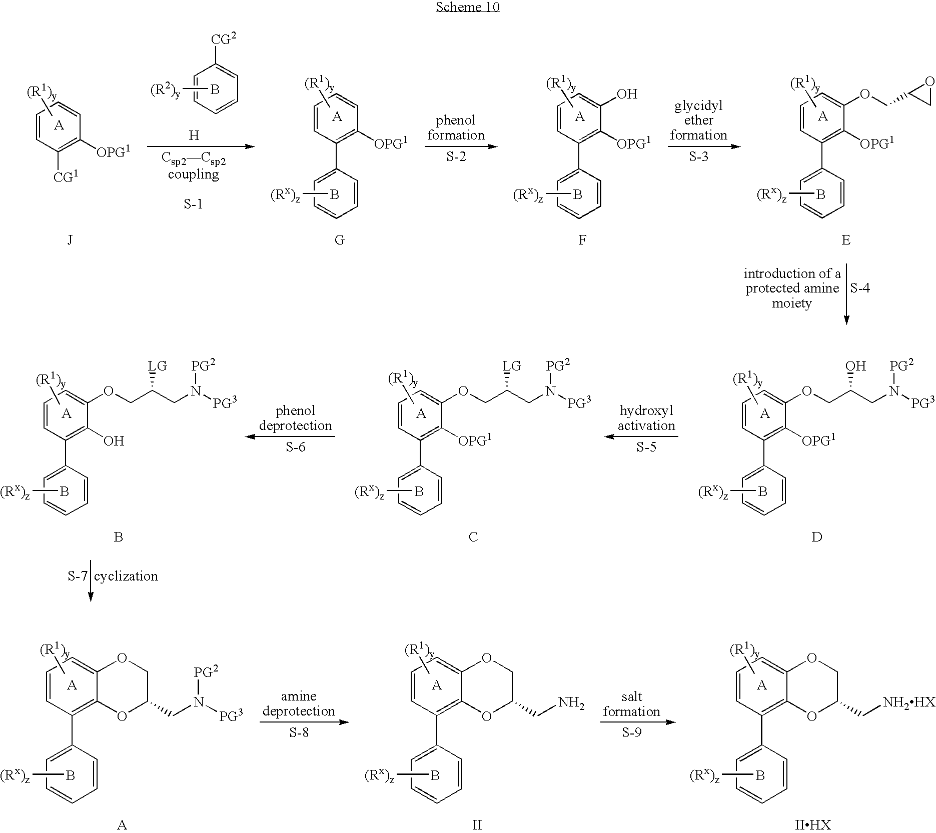
wherein each z is 0-5.

At step S-1 of Scheme 10, a compound of formula J is coupled to a compound of formula H, via a Cₛₚ₂-Cₛₚ₂ coupling reaction between the carbon centers bearing complementary coupling groups CG¹ and CG² to provide a compound of formula G. Suitable coupling reactions are well known to one of ordinary skill in the art and typically involve one of the coupling groups being an electron-withdrawing group (e.g., Cl, Br, I, OTf, etc.), such that the resulting polar carbon-CG bond is susceptible to oxidative addition by an electron-rich metal (e.g., a low-valent palladium or nickel species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-Iv} species or a Ni^{II-IV} species). Exemplary reactions and coupling groups include those described in Metal-Catalyzed Cross-Coupling Reactions, A. de Meijere and F. Diederich, Eds., 2^{nd} Edition, John Wiley & Sons, 2004. In certain embodiments, CG¹ in compounds of formula J is a boronic acid, a boronic ester, or a borane. In other embodiments, CG¹ in compounds of formula J is a boronic ester. According to one aspect of the present invention, CG¹ in compounds of formula J is a boronic acid. In certain embodiments, CG² in compounds of formula H is Br, I, or OTf. According to one aspect of the present invention, CG² in compounds of formula H is Br. In certain embodiments, the transformation is catalyzed by a palladium species. According to one aspect of the invention, the transformation is catalyzed by palladium tetrakis triphenylphosphine.

At step S-2, a hydroxyl group is introduced at the open ortho position relative to the OPG¹ group of formula G. One of ordinary skill in the art will recognize that there are a wide variety of reactions and reaction sequences that can be employed to accomplish this transformation; see generally, March's Advanced Organic Chemistry: Reactions, Mechanisms, and Structure, M. B. Smith and J. March, 5^{th} Edition, John Wiley & Sons, 2001 and Comprehensive Organic Transformaions, R. C. Larock, 2^{nd} Edition, John Wiley & Sons, 1999. Exemplary sequences include initial directed orthometallation followed by either (a) direct treatment with an electrophilic oxygen source; (b) treatment with a borate ester followed by oxidative workup of the resulting boronic ester or acid; or (c) treatment with a reagent that will allow the introduction of a formyl group (e.g., methyl formate, dimethylformamide) followed by subsequent Baeyer-Villiger reaction; for the above methods, see, e.g., Snieckus, V. Chem. Rev. 1990, 90, 879 and Schlosser, M. Angew. Chem. Int. Ed. 2005, 44, 376. Alternatively, direct orthoformylation may be utilized, followed by a Baeyer-Villiger reaction; see, e.g., Laird, T. in Comprehensive Organic Chemistry, Stoddart, J. F., Ed., Pergamon, Oxford 1979, Vol. 1, p 1105 and Hofsløkken, N. U.; Skattebøl, L. Acta Chem. Scand. 1999, 53, 258. Another exemplary sequence involves halogenation followed by a metallation/transmetallation sequence to afford a boronic acid, boronic ester, or borane, followed by peroxide oxidation; see, generally, de Meijere (2004) and Snieckus (1990).

According to one aspect of the present invention, a compound of formula G is first brominated, then is subjected to halogen-metal exchange to afford an intermediate arylmetal compound that is allowed to react with a borate ester to afford, following aqueous workup, a boronic acid, which is subsequently oxidized to provide a phenol of formula F, as depicted in Scheme II. According to another aspect of the invention, the brominating agent is N-bromosuccinimide. In certain embodiments, the bromination is conducted in the presence of para-toluenesulfonic acid and acetic acid. According to yet another aspect of the invention, the metallation/transmetallation sequence involves initial magnesium-halogen exchange, followed by treatment with a trialkyl borate. In certain embodiments, the magnesium-halogen exchange is accomplished by treating the intermediate aryl bromide with isopropylmagnesium bromide. According to one aspect of the invention, the magnesium-halogen exchange is conducted in tetrahydrofuran (THF). In other embodiments, the trialkyl borate is triisopropylborate [B(OiPr)₃]. In certain embodiments, the metallation/transmetallation step is conducted at a temperature that is between −20° C. and 20° C. In certain embodiments, the boronic acid is oxidized with hydrogen peroxide (H₂O₂) to afford compounds of formula F. In other embodiments, the boronic acid is oxidized with peroxyacetic acid (also called peracetic acid) or meta-chloroperoxybenzoic acid (mCPBA). One of ordinary skill in the art will recognize that standard procedures for magnesium-halogen exchange followed by transmetallation to a boron-containing entity, followed by oxidation to the phenol can be performed without isolation of the respective intermediate species.

At step S-3, a compound of formula F is glycidated on the phenol oxygen of compounds of formula F. Exemplary reagents that may be used to promote glycidation include epichlorohydrin, epibromohydrin, oxiranylmethyl p-toluenesulfonate (also called: oxiranylmethyl tosylate or glycidyl tosylate), oxiranylmethyl methanesulfonate (oxiranylmethyl mesylate or glycidyl mesylate), and oxiranylmethyl trifluoromethanesulfonate (oxiranylmethyl triflate or glycidyl triflate). According to one aspect of the present invention, the activated glycidol equivalent is gycidyl tosylate. In certain embodiments, at step S-3, a compound of formula F is treated with a base to form the corresponding metal phenoxide salt, which is then allowed to react with an activated glycidol equivalent to afford a compound of formula E. In certain embodiments, the base employed is selected from sodium hydroxide (NaOH), potassium carbonate (K₂CO₃), potassium tert-butoxide (KOtBu), lithium diisopropylamide (LDA), lithium hexamethyldisilazide (LHMDS), or sodium hydride (NaOH). According to one aspect of the present invention, the base is potassium tert-butoxide. In certain embodiments, the reaction is conducted using dimethylformamide (DMF), N-methylpyrrolidine (NMP), or dimethylamine (DMA) as solvent. In other embodiments, DMF is employed as solvent. In certain embodiments, the reaction is heated. In other embodiments, the reaction is conducted at a temperature that is between 20° C. and 100° C. One of ordinary skill in the art will recognize that the activated glycidol equivalents contain a stereogenic carbon, and accordingly, compounds of formula E contain a steregenic carbon corresponding thereto.

In certain embodiments, the glycidol equivalent employed at step S-3 is enantiomerically enriched, and accordingly, the mixture of enantiomers of formula E that are generated in this step is enriched in one of the enantiomers. While a single stereochemical isomer is depicted for formulae E, D, C, B, A, II, and II.HX in Scheme 10, it will be appreciated that mixtures of enantiomers of these formulae are accessible enriched in either enantiomer via methods of the present invention and those known to one of ordinary skill in the art.

As used herein, the terms “enantiomerically enriched” and “enantioenriched” denote that one enantiomer makes up at least 75% of the preparation. In certain embodiments, the terms denote that one enantiomer makes up at least 80% of the preparation. In other embodiments, the terms denote that at least 90% of the preparation is one of the enantiomers. In other embodiments, the terms denote that at least 95% of the preparation is one of the enantiomers. In still other embodiments, the terms denote that at least 97.5% of the preparation is one of the enantiomers. In yet another embodiment, the terms denote that the preparation consists of a single enantiomer to the limits of detection (also referred to as “enantiopure”). As used herein, when “enantioenriched” or “enantiomerically enriched” are used to describe a singular noun (e.g., “an enantioenriched compound of formula II” or “an enantioenriched chiral acid”), it should be understood that the “compound” or “acid” may be enantiopure, or may in fact be an enantioenriched mixture of enantiomers. Similarly, when “racemic” is used to describe a singular noun (e.g., “a racemic compound of formula E”), it should be understood that the term is in fact describing a 1:1 mixture of enantiomers.

At step S-4, a protected amine moiety is introduced via epoxide-opening to afford compounds of formula D. In compounds of formulae D, C, B, and A, PG² and PG³ are amino protecting groups. Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, and 2,2,5,5-tetramethyl-[1,2,5]azadisilolidine and the like. Notwithstanding the definition above, one of either PG² or PG³ in compounds of formulae D, C, B, and A may be hydrogen. Also notwithstanding the definitions above, the —N(PG²)(PG³) moiety of formulae D, C, B, and A may be azido. According to one aspect of the invention, the —N(PG²)(PG³) moiety of formulae D, C, B, and A, is phthalimido. According to another aspect of the invention, at step S-4, a compound of formula E is treated with potassium phthalimide to generate compounds of formula D in which the —N(PG²)(PG³) moiety is phthalimido. In other embodiments, step S-4 is performed with heating. In certain embodiments, the reaction is conducted in dimethylformamide (DMF), N-methylpyrrolidine (NMP), or dimethylamine (DMA). In other embodiments, the reaction is conducted in DMF. In certain embodiments, the reaction is conducted at a temperature that is between 40° C. and 110° C. In other embodiments, the reaction is run at 80° C.

In certain embodiments, steps S-3 and S-4 may be conducted without isolating compounds of formula E. Accordingly, one aspect of the present invention is a procedure of glycidation followed by epoxide-opening to introduce a protected amine moiety without isolation of the intermediate glycidated species. In certain embodiments, the phthalimide is directly added to the reaction mixture in which the glycidated species was formed.

At step S-5, the hydroxyl group of compounds of formula D is activated such that it becomes leaving group LG that is subject to nucleophilic displacement. A suitable “leaving group” that is “subject to nucleophilic displacement” is a chemical group that is readily displaced by a desired incoming nucleophilic chemical entity. Suitable leaving groups are well known in the art, e.g., see, Smith (2001). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. Examples of suitable leaving groups include chloro, iodo, bromo, fluoro, methanesulfonyloxy(mesyloxy), tosyloxy, triflyloxy, nitro-phenylsulfonyloxy (nosyloxy), and bromo-phenylsulfonyloxy(brosyloxy). According to one aspect of the present invention, LG in compounds of formula C is methanesulfonyloxy(mesyloxy). According to another aspect of the invention, a compound of formula D is allowed to react with methanesulfonyl chloride (mesyl chloride) to afford a compound of formula C in which LG is methanesulfonyloxy(mesyloxy). In certain embodiments this reaction is run in tetrahydrofuran (THF), dichloromethane, acetonitrile, or isopropyl acetate. In other embodiments the reaction is run in THF. According to one aspect of the present invention, the reaction is run in the presence of triethylamine (TEA). In certain embodiments, the reaction is run at a temperature that is between −20° C. and 40° C. In other embodiments, the reaction is conducted at a temperature of 0° C.

At step S-6, removal of the PG¹ protecting group in compounds of formula C affords the free phenol-containing compounds of formula B. Procedures for the removal of suitable hydroxyl protecting groups are well known in the art; see Green (1999). In certain embodiments, where PG¹ is methyl, PG¹ is removed by treatment of a compound of formula C with BBr₃, iodotrimethylsilane, or a combination of BCl₃ and LiI. According to one aspect of the present invention, where PG¹ is methyl, PG¹ is removed by treatment of a compound of formula C with BBr₃. In certain embodiments this step is conducted using toluene, dichloromethane, or isopropyl acetate as solvent. In other embodiments, this step is conducted using toluene as solvent. In certain embodiments, the reaction is conducted at a temperature between −20° C. and 40° C.

At step S-7, a compound of formula B is allowed to cyclize to afford a compound of formula A. One of ordinary skill in the art would recognize that a wide variety of reaction conditions are useful for promoting this reaction, therefore a wide variety of reaction conditions are contemplated. For example, the reaction may be conducted with or without thermal excitation, with or without base catalysis, and in protic or aprotic media. According to one aspect of the invention, the reaction is promoted by the addition of potassium carbonate, lithium diisopropylamide, or lithium hexamethyldisilazide to a compound of formula B. According to another aspect of the invention, the reaction is promoted by the addition of potassium carbonate. In certain embodiments, the reaction is conducted with dimethylformamide, N-methylpyrollidone, or dimethylamine as solvent. In other embodiments, the reaction is conducted with dimethylformamide as solvent. In certain embodiments, the reaction is conducted at a temperature between 10° C. and 60° C.

At step S-8, removal of the PG² and PG³ protecting groups in compounds of formula A affords the free amine-containing compounds of formula II. Procedures for the removal of suitable amino protecting groups are well known in the art; see Green (1999). In certain embodiments, where the —N(PG²)(PG³) moiety of formulae A is phthalimido, PG² and PG³ are removed by treatment with hydrazine or methylamine. In other embodiments, where the —N(PG²)(PG³) moiety of formulae A is phthalimido, PG² and PG³ are removed by treatment with hydrazine. In certain embodiments, this transformation is conducted with ethanol, methanol, isopropanol, or tetrahydrofuran as solvent, or with mixtures of the aforementioned solvents and/or water. In other embodiments, this transformation is conducted with ethanol as solvent. In certain embodiments, the reaction is conducted at a temperature between 40° C. and 90° C. In other embodiments the reaction is conducted with an ethanol-water mix as solvent at reflux.

One of ordinary skill in the art will appreciate that a compound of formula II, as prepared by the methods of the present invention, may be treated with a suitable Brønsted acid, HX, as depicted in step S-9, to form a salt thereof (represented by formula II.HX). Exemplary acids include hydrogen halides, carboxylic acids, sulfonic acids, sulfuric acid, and phosphoric acid. According to one aspect of the present invention, a compound of formula II is treated with HCl to form a compound of formula II.HX wherein X is Cl. In certain embodiments, where the acid is HCl, it is introduced into the medium containing the compound of formula H in gaseous form. In other embodiments, the acid is introduced into the medium containing the compound of formula II as a solution in methanol, ethanol, isopropanol, or water. In yet other embodiments, the acid is introduced into the medium containing the compound of formula II as a solution in isopropanol. In certain embodiments, the medium containing the compound of formula H is isopropanol.

Using compound I-47 to exemplify, Scheme II depicts an alternate method for preparing compounds of the present invention.

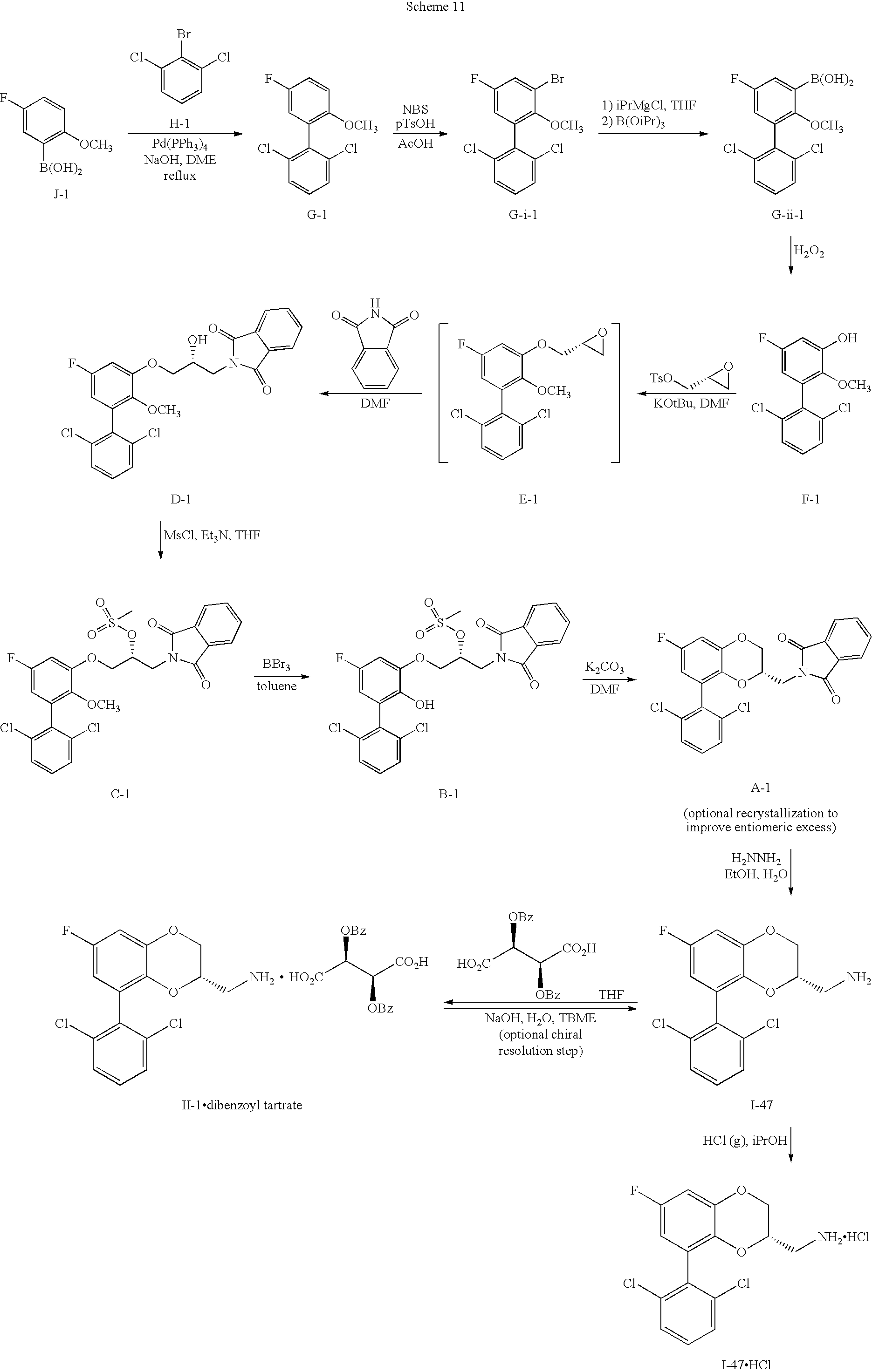

Although certain exemplary embodiments are depicted and described above and herein, it will be appreciated that compounds of the invention can be prepared according to the methods described generally above using appropriate starting materials by methods generally available to one of ordinary skill in the art. Additional embodiments are exemplified in more detail herein.

4. Uses, Formulation and Administration

Compounds of the present invention have affinity for and agonist or partial agonist activity at the 2C subtype of brain serotonin receptors and are thus of interest for the treatment of a variety of disorders and/or the alleviation of one or more associated symptoms. Such disorders associated with modulations of the 2C subtype of brain serotonin receptors are described in detail below. The present invention contemplates that compounds of formula I are associated with a rapid onset of action. In addition, compounds of formula I lack the side-effect of sexual dysfunction.

Compounds of the present invention are useful for treating one or more psychotic disorders, as described herein, without causing diabetogenesis. Diabetogenesis is a side-effect associated with atypical antipsychotic agents. Without wishing to be bound by any particular theory, it is believed that the diabetogenesis associated with atypical antipsychotic agents results from the fact that those agents are 5-HT_{2C} antagonists. As described herein, the present compounds are 5-HT_{2C} agonists, or partial agonists, and therefore are not associated with diabetogenesis.

Compounds of the present invention are useful for treating one or more psychotic disorders such as schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; and psychosis associated with Lewy body disease.

Compounds of the present invention are also useful for treating symptoms related to psychotic disorders of the schizophrenic types, including the so called “positive” and “negative” symptoms of schizophrenia. These symptoms include for example hallucinations, delusions, paranoia, anxiety, agitation, excessive aggression, tension, thought disorder, blunted affect, and social or emotional withdrawal in psychotic patients. Other symptoms often associated with psychotic disorders include cognition disorders or deficits such as poor attention and impaired function, depression, suicide, metabolic syndrome, and substance abuse. Thus, another embodiment of the present invention provides a method for treating one or more symptoms associated with a psychotic disorder.

In other embodiments, the present compounds are useful for treating anxiety disorders such as panic attack, agoraphobia, panic disorder, specific phobia, social phobia, social anxiety disorder, obsessive compulsive disorder, posttraumatic stress disorder, acute stress disorder, generalized anxiety disorder, separation anxiety disorder, substance-induced anxiety disorder, and anxiety disorder not otherwise specified.

According to another embodiment, the present compounds are useful for treating bipolar disorders. Such bipolar disorders include bipolar I disorder, bipolar II disorder, and cyclothymic disorder; bipolar mania, dementia, and depression with psychotic features. The present compounds are also useful for treating (including the preventing) of cycling that may occur between bipolar depression and bipolar mania.

A more complete description of the aforementioned mental disorders can be found in the Diagnostic and Statistical Manual of Mental Disorders, 4^{th} edition, Washington, D.C., American Psychiatric Association (1994), incorporated herein by reference in its entirety.

In certain embodiments, compounds of the present invention are administered in combination with one or more anti-psychotic agents. Such anti-psychotic agents are well known in the art and include clozapine (e.g., Clozaril®), risperidone (e.g., Risperidal®), olanzapine (e.g., Zyprexa®), quetiapine (e.g., Seroquel®), ziprasidone (e.g., Geodon®), aripiprazole, amisulpiride, chlorpromazine, fluphenazine, haloperidol (e.g., Haldol®), loxapine, mesoridazine, molindone, perphenazine, pimozide, seroquel, sulpiride, thioridazine, thiothixene, trifluoperazine, and bifeprunox to name a few.

The combination of a compound of the present invention with one or more anti-psychotic agents is useful for treating schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; psychosis associated with Lewy body disease; bipolar disorders such as bipolar I disorder, bipolar II disorder, and cyclothymic disorder; bipolar mania, dementia, and depression with psychotic features. In some embodiments, these combinations are useful in the treatment of bipolar disorder, including for example treating the cycling between bipolar depression and bipolar mania.

In other embodiments, administration of a compound of the present invention with an anti-psychotic agent provide anti-psychotic benefits while eliminating or minimizing certain side affects (e.g., akathisia, dystonia, Parkinsonism dyskinesia and late dyskinesia and the like) typically observed when the anti-psychotic agent(s) is/are taken alone.

In other embodiments, compounds of the present invention are useful for treating one or more depressive disorders such as major depressive disorder, seasonal affective disorder, dysthymic disorder, substance-induced mood disorder, depressive disorder not otherwise specified, and treatment resistant depression.

Another aspect of the present invention provides a method for treating one or more mood episodes such as major depressive episode, manic episode, mixed episode, and hypomanic episode; and adjustment disorders such as adjustment disorders with anxiety and/or depressed mood.

Compounds of the present invention are also useful for treating symptoms related to depressive disorders including somatic symptoms such as neuropathic pain and sexual dysfunction. Other somatic symptoms include hopelessness, helplessness, anxiety and worries, memory complaints with or without objective signs of cognitive impairment, loss of feeling of pleasure (anhedonia), slowed movement, irritability, and lack of interest in personal care, such as poor adherence to medical or dietary regimens.

In certain embodiments, the present invention provides a method of treating sexual dysfunction related to depression. In other embodiments, the present invention provides a method of treating sexual dysfunction associated with administering a serotonin reuptake inhibitor (SRI) for treating a depressive or other disorder. Such methods of treating sexual dysfunction are described in detail below.

In certain embodiments, compounds of the present invention are administered in combination with one or more antidepressive agents. Suitable antidepressant agents include, for example, serotonin reuptake inhibitors (SRIs), norepinephrine reuptake inhibitors (NRIs), combined serotonin-norepinephrine reuptake inhibitors (SNRIs), monoamine oxidase inhibitors (MAOIs), reversible inhibitors of monoamine oxidase (RIMAs), phosphodiesterase-4 (PDE4) inhibitors, corticotropin releasing factor (CRF) antagonists, alpha-adrenoreceptor antagonists or other compounds including atypical antidepressants. Additional antidepressants for administering in combination with compounds of the present invention include triple uptake inhibitors such as DOV 216303 and DOV 21947; melatonin agonists such as agomelotine, super neurotransmitter uptake blockers (SNUBs; e.g., NS-2389 from GlaxoSmithKline and Neurosearch; (R)-DDMA from Sepracor), and/or substance P/neurokinin receptor antagonists (e.g., aprepitant/MK-869 from Merck; NKP-608 from Novartis; CPI-122721 from Pfizer; R673 from Roche; TAK637 from Takeda; and GW-97599 from GlaxoSmithKline).

Another class of antidepressant agents for administering in combination with compounds of the present invention are noradrenergic and specific serotonergic antidepressants (NaSSAs). A suitable example of a NaSSA is mirtazepine.

Suitable NRIs for administering in combination with compounds of the present invention include tertiary amine tricyclics and secondary amine tricyclics. Suitable examples of tertiary amine tricyclics include: amitriptyline, clomipramine, doxepin, imipramine (See U.S. Pat. No. 2,554,736, incorporated herein by reference in its entirety) and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Another NRI for administering in combination with compounds of the present invention is reboxetine (Edronax™; 2-[.alpha.-(2-ethoxy)phenoxy-benzyl]morpholine, usually administered as the racemate; See U.S. Pat. No. 4,229,449, incorporated herein by reference in its entirety).

Suitable SSRIs for administering in combination with compounds of the present invention include: citalopram (1-[3-(dimethylamino)propyl]-(4-fluorophenyl)-1,3-dihydr-o-5-isobenzofurancarbonitrile; See U.S. Pat. No. 4,136,193; Christensen et al., Eur. J. Pharmacol. 41:153, 1977; Dufour et al., Int. Clin. Psychopharmacol. 2:225, 1987; Timmerman et al., ibid., 239, each of which is incorporated herein by reference in its entirety); fluoxetine (N-methyl-3-(p-trifluoromethylphenoxy)-3-phenylpropylamine, marketed in the hydrochloride salt form and as the racemic mixture of its two isoforms; see, for example, U.S. Pat. No. 4,314,081; Robertson et al., J. Med. Chem. 31:1412, 1988, each of which is incorporated herein by reference); fluoxetine/olanzapine in combination; fluvoxamine(5-methoxy-1-[4-(trifluoromethyl)phenyl]-1-pentanone O-(2-aminoethyl)oxime; See U.S. Pat. No. 4,085,225; Claassen et al., Brit. J. Pharmacol. 60:505, 1977; De Wilde et al., J. Affective Disord. 4:249, 1982; Benfield et al., Drugs 32:313, 1986, each of which is incorporated herein by reference in its entirety); paroxetine (trans-(−)-3-[(1,3-benzodioxol-5-yloxy)methyl]-4-(4-fluo-rophenyl)piperidine; See U.S. Pat. No. 3,912,743; U.S. Pat. No. 4,007,196; Lassen, Eur. J. Pharmacol. 47:351, 1978; Hassan et al., Brit. J. Clin. Pharmacol. 19:705, 1985; Laursen et al., Acta Psychiat. Scand. 71:249, 1985; Battegay et al., Neuropsychobiology 13:31, 1985, each of which is incorporated herein by reference in its entirety); sertraline, (1S-cis)-4-(3,4-dichlorophenyl)-1,2,3,4-tetrahydro-N-methyl-1-naphthylamine hydrochloride; See U.S. Pat. No. 4,536,518, incorporated herein by reference in its entirety); escitalopram (see U.S. Pat. RE34,712); and pharmaceutically acceptable salts thereof.

Suitable MAOIs for administering in combination with compounds of the present invention include: isocarboxazid, phenelzine, selegiline and tranylcypromine, and pharmaceutically acceptable salts thereof.

Suitable reversible MAOIs for administering in combination with compounds of the present invention include: moclobemide (4-chloro-N-[2-(4-morpholinyl)-ethyl]benzamide; See U.S. Pat. No. 4,210,754, incorporated herein by reference in its entirety), selegiline, and pharmaceutically acceptable salts thereof.

Suitable SNRIs for administering in combination with compounds of the present invention include venlafaxine (see U.S. Pat. No. 4,535,186, incorporated herein by reference in its entirety; see also U.S. Pat. Nos. 5,916,923, 6,274,171, 6,403,120, 6,419,958, 6,444,708, each of which is incorporated herein by reference in its entirety), and pharmaceutically acceptable salts and analogs, including the O-desmethylvenlafaxine succinate salt; milnacipran (N,N-diethyl-2-aminomethyl-1-phenylcyclopropanecarboxamide; see U.S. Pat. No. 4,478,836; Moret et al., Neuropharmacology 24:1211-19, 1985, each of which is incorporated herein by reference in its entirety); mirtazapine (see, for example, U.S. Pat. No. 5,178,878, the entire contents of which are incorporated herein by reference); nefazodone (available from Bristol Myers Squibb and Dr. Reddy Labs Inc.); duloxetine; and pharmaceutically acceptable salts thereof.

Suitable CRF antagonists for administering in combination with compounds of the present invention include those compounds described in International Patent Specification Nos. WO 94/13643, WO 94/13644, WO 94/13661, WO 94/13676 and WO 94/13677.

Suitable atypical antidepressants for administering in combination with compounds of the present invention include: bupropion (Wellbutrin™; (.+-.)-1-(3-chlorophenyl)-2-[(1,1-dim-ethylethyl)amino]-1-propanone), lithium, nefazodone, trazodone and viloxazine, and pharmaceutically acceptable salts thereof. Another suitable atypical antidepressant is sibutramine.

Particular antidepressants for administering in combination with compounds of the present invention include, but are not limited to, adinazolam, alaproclate, alnespirone, amineptine, amitriptyline, amitriptyline/chlordiazepoxide combination, amoxapine, aprepitant, atipamezole, azamianserin, bazinaprine, befuraline, bifemelane, binodaline, bipenamol, brofaromine, buproprion, caroxazone, cericlamine, cianopramine, cimoxatone, citalopram, clemeprol, clomipramine, clovoxamine, dazepinil, deanol, demexiptiline, desipramine, O-desmethylvenlafaxine, dibenzepin, dothiepin, doxepin, droxidopa, duloxetine, elzasonan, enefexine, eptapirone, escitalopram, estazolam, etoperidone, femoxetine, fengabine, fezolamine, fluotracen, fluoxetine, fluvoxamine, gepirone, idazoxan, imipramine, indalpine, indeloxazine, iprindole, isocarboxazid, levoprotiline, litoxetine, lofepramine, maprotiline, medifoxamine, metapramine, metralindole, mianserin, milnacipran, minaprine, mirtazapine, moclobemide, montirelin, nebracetam, nefopam, nefozodine, nemititide, nialamide, nomifensine, norfluoxetine, nortriptyline, orotirelin, oxaflozane, paroxetine, pheneizine, pinazepam, pirlindone, pizotyline, protryptiline, reboxetine, ritanserin, robalzotan, rolipram, selegiline, sercloremine, sertraline, setiptiline, sibutramine, sulbutiamine, sulpiride, sunepitron, teniloxazine, thozalinone, thymoliberin, tianeptine, tiflucarbine, tofenacin, tofisopam, toloxatone, tomoxetine, tranylcypromine, trazodone, trimiprimine, venlafaxine, veralipride, vilazodone, viloxazine, viqualine, zimelidine and zometrapine, and pharmaceutically acceptable salts thereof, and St. John's wort herb, or Hypencuin perforatum, or extracts thereof.

Suitable classes of anti-anxiety agents for administering in combination with compounds of the present invention include 5-HT_{1A} agonists or antagonists, especially 5-HT_{1A} partial agonists, neurokinin recepter (NK) antagonists (e.g., saredutant and osanetant) and corticotropin releasing factor (CRF) antagonists. Suitable 5-HT_{1A} receptor agonists or antagonists that may be used in the present invention include, in particular, the 5-HT_{1A} receptor partial agonists buspirone, flesinoxan, gepirone and ipsapirone, and pharmaceutically acceptable salts thereof. An example of a compound with 5-HT_{1A} receptor antagonist/partial agonist activity is pindolol. New 5HT_{1A} agonists variza, alnespirone, gepirone, sunepitron, MKC242, vilazodone, eptapirone, and ORG12962 from Organon; new 5HT_{1A} antagonists such as robalzotan; new 5-HT_{1B} agonists such as elzasonan; new 5HT₂ antagonists such as YM-992 (from Yamanouchi Pharmaceuticals) and nemifitide.

According to the present invention, the inventive combinations may be administered in conjunction with one or more other agents that is useful in treating depression or other mood disorders. Alternatively or additionally, inventive combinations may be administered with one or more other pharmaceutical agents active in treating any other symptom or medical condition present in the mammal that is related or unrelated to the depression or mood disorder being experienced by the mammal. Examples of such pharmaceutical agents include, for example, anti-angiogenic agents, anti-neoplastic agents, anti-diabetic agents, anti-infective agents, pain-relieving agents, anti-psychotic agents, gastrointestinal agents, etc., or combinations thereof. Other pharmaceutical agents useful in the practice of the present invention include, for example, adjunctive therapies typically used to enhance the effects of an antidepressant. Such adjunctive agents may include, for instance, mood stabilizers (e.g., lithium, valproic acid, carbamazepine, etc.); pindolol, stimulants (e.g., methylphenidate, dextroamphetamine, etc.); or thyroid augmenting agents (e.g., T₃); anti-psychotics, anti-anxiety agents (e.g., benzodiazepines), and/or agents that relieve sexual dysfunction (e.g., buspirone, which also has anti-anxiety effects; dopaminergic agents such as amantadine, pramipexole, bupropion, etc.).

As 5-HT_{2C} modulators, compounds of the present invention are useful for treating a variety of disorders. Such disorders include premenstrual syndrome (PMS), premenstrual dysphoric disorder (PMDD), motion or motor disorders such as Parkinson's disease; chronic fatigue syndrome, anorexia nervosa, disorders of sleep (e.g., sleep apnea), and mutism.

Premenstrual dysphoric disorder, or PMDD, is a severe form of PMS. Like PMS, PMDD typically occurs the week before the onset of menstruation and disappears a few days after. PMDD is characterized by severe monthly mood swings and physical symptoms that interfere with everyday life, especially a woman's relationships with her family and friends. PMDD symptoms go far beyond what are considered manageable or normal premenstrual symptoms.

PMDD is a combination of symptoms that may include irritability, depressed mood, anxiety, sleep disturbance, difficulty concentrating, angry outbursts, breast tenderness and bloating. The diagnostic criteria emphasize symptoms of depressed mood, anxiety, mood swings or irritability. The condition affects up to one in 20 American women who have regular menstrual periods. According to another embodiment, the present invention provides a method for treating one or more symptoms associated with PMDD.

Selective serotonin reuptake inhibitors (SSRIs) are the current preferred method for treating symptoms associated with PMDD. According to another aspect, the present invention provides a method for treating PMDD, or one or more symptoms associated with PMDD, by administering a compound of formula I in combination with an SSRI. In certain embodiments, the SSRI is fluoxetine, venlafaxine, paroxetine, duloxetine, or sertraline.

According to another embodiment, compounds of the present invention are useful for treating a variety of eating disorders. In certain embodiments, the eating disorder is hyperphagia, bulimia or anorexia nervosa. In certain embodiments, compounds of the present invention are useful for treating gastrointestinal disorders, such as malfunction of gastrointestinal motility or intestinal propulsion. Compounds of the present invention are also useful in connection with weight loss or control (e.g., reduction in calorie or food intake, and/or appetite suppression). Such methods are particularly useful for treating obesity with its consequent comorbidities including diabetes insipidus, Type II diabetes, cardiovascular disease, hypertension, hyperlipidemia, stroke, osteoarthritis, sleep apnea, gall bladder disease, gout, some cancers, some infertility, and early mortality.

In certain embodiments, compounds of the present invention are administered in combination with one or more anti-obesity agents. Such anti-obesity agents are known in the art and include apolipoprotein-B secretion/microsomal triglyceride transfer protein (apo-B/MTP) inhibitors, 11β-hydroxy steroid dehydrogenase-1 (11(β-HSD type 1) inhibitors, PYY_{3.36} and analogs thereof, MCR4 agonists, cholecystokinin-A (CCK-A) agonists, monoamine reuptake inhibitors (such as sibutramine), sympathomimetic agents, R3 adrenergic receptor agonists, dopamine agonists (such as bromocriptine), melanocyte-stimulating hormone receptor analogs, cannabinoid 1 receptor antagonists (e.g., rimonabant), melanin concentrating hormone antagonists, leptins (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists, thyromimetic agents, dehydroepiandrosterone or an analog thereof, glucocorticoid receptor agonists or antagonists, orexin receptor antagonists, urocortin binding protein antagonists, glucagon-like peptide-1 receptor agonists, ciliary neurotrophic factors (such as Axokine^{TA}), human agouti-related proteins (AGRP), ghrelin receptor antagonists, histamine 3 receptor antagonists or inverse agonists, and neuromedin U receptor agonists.

In other embodiments, a compound of the present invention is administered in combination with an anti-obesity agent selected from orlistat, sibutramine, bromocriptine, ephedrine, leptin, rimonabant, pseudoephedrine, PYY3.36 or an analog thereof, and 2-oxo-N-(5-phenylpyrazinyl)spiro-[isobenzofuran-1(3H),4′-piperidine]-1′-carboxamide. According to another aspect of the invention, a compound of the present invention is administered in combination with an anti-obesity agent in conjunction with typical treatments for obesity such as exercise and a sensible diet.

According to another embodiment, a compound of the present invention is administered in combination with one or more agents for treating diabetes and associated conditions. In certain embodiments, a compound of the present invention is administered in combination with one or more such agents including insulin and insulin analogs (e.g., LysPro Insulin); GLP-1 (7-37) (insulinotropin) and GLP-1 (7-36)-NH₂; sulfonylureas and analogs thereof: chlorpropamide, glibenclamide, tolbutamide, tolazamide, acetohexamide, Glypizide®, glimepiride, repaglinide, meglitinide; biguanides: metformin, phenform in, buformin; “2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; glucosidase inhibitors: acarbose, miglitol, emiglitate, voglibose, MDL-25,637, camiglibose, MDL-73,945; 13-agonists: BRL 35135, BRL 37344, RO 16-8714, ICI D7114, CL 316,243; or phosphodiesterase inhibitors: L-386,398.

In other embodiments, a compound of the present invention is administered in combination with one or more lipid-lowering agents: benfluorex: vanadate and vanadium complexes (e.g., Nagiivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin”), AC 2993, nateglinide, aldose reductase inhibitors (e.g., zopolrestat), glycogen phosphorylase inhibitors, sorbitol dehydrogenase inhibitors, sodium-hydrogen exchanger type 1 (NNE-1) inhibitors and/or cholesterol biosynthesis inhibitors or cholesterol absorption inhibitors, especially a HMG-CoA reductase inhibitor, or a HMG-CoA synthase inhibitor, or a HMG-CoA reductase or synthase gene expression inhibitor, a CETP inhibitor, a bile acid sequesterant, a fibrate, an ACAT inhibitor, a squalene synthetase inhibitor, or an anti-oxidant. In other embodiments, a compound of the present invention is administered in combination with one or more naturally occurring compounds that acts to lower plasma cholesterol levels. Such naturally occurring compounds are commonly referred to as nutraceuticals and include, for example, garlic extract, Hoodia plant extracts, and niacin.

In certain embodiments, compounds of the present invention are useful for inducing, assisting or maintaining desirable bladder control in a mammal. The methods are particularly useful for treating a mammal that is experiencing or susceptible to bladder instability or urinary incontinence. Inventive methods include prevention, treatment or inhibition of bladder-related urinary conditions and bladder instability, including idiopathic bladder instability, nocturnal enuresis, nocturia, voiding dysfunction and urinary incontinence (including, for example, stress incontinence, urge incontinence, and/or mixed incontinence). Also treatable or preventable by administration of a compound of this invention is bladder instability secondary to prostate hypertrophy, as is a method for enhancing urethral tone and reducing undesirable urine leakage even in an otherwise healthy person. For example, the inventive methods are applicable to alleviating urine leakage often occurring in women during the first year after childbirth.

In other embodiments, the present compounds are useful for treating urine retention or detrusor sphinctor dyssynergia. Patients suffering from urine retention include those suffering from spinal cord injuries or male patients with benign prostatic hyperplasia.

According to the present invention, a compounds of the present invention is also useful in promoting the temporary delay of urination whenever desirable. Such compounds may be utilized in accordance with the present invention to stabilize the bladder in any applicable context. Inventive methods therefore may be utilized to allow a recipient to control the urgency and frequency of urination.

In some embodiments of the invention, compounds of the present invention are administered to a mammal in need thereof for the treatment, prevention, inhibition and/or amelioration of urge urinary incontinence (also known as bladder instability, neurogenic bladder, voiding dysfunction, hyperactive bladder, detrusor overactivity, detrusor hyper-reflexia or uninhibited bladder) or mixed urinary incontinence. Inventive uses include, but are not limited to, those for bladder activities and instabilities in which the urinary urgency is associated with prostatitis, prostatic hypertrophy, interstitial cystitis, urinary tract infections or vaginitis. The methods of this invention may also be used to assist in inhibition or correction of the conditions of Frequency-Urgency Syndrome, and lazy bladder, also known as infrequent voiding syndrome.

Compounds of the present invention may also be used to treat, prevent, inhibit, or limit the urinary incontinence, urinary instability or urinary urgency associated with or resulting from administrations of other medications, including diuretics, vasopressin antagonists, anticholinergic agents, sedatives or hypnotic agents, narcotics, alpha-adrenergic agonists, alpha-adrenergic antagonists, or calcium channel blockers.

Compounds of the present invention are useful for inducing or assisting in urinary bladder control or preventing or treating the maladies described herein in humans in need of such relief, including adult and pediatric uses. They may also be utilized for veterinary applications, particularly including canine and feline bladder control methods. If desired, the methods herein may also be used with farm animals, such as ovine, bovine, porcine and equine breeds.

According to the present invention, compounds of the present invention may be administered alone to modulate bladder activity, or alternatively may be administered in combination with (whether simultaneously or sequentially) one or more other pharmaceutical agents useful in the modulation of bladder activity. Alternatively or additionally, the compounds of the present invention may be administered in combination with one or more other pharmaceutical agents useful in the treatment or prevention of one or more other symptoms, disorders, or diseases suffered by the individual in need of bladder activity modulation.

Other pharmaceutical agents useful in the modulation of bladder activity, and particularly for treatment, prevention, inhibition, and/or amelioration of urinary incontinence, include, for example, desmopressin acetate (available as DDAVP® (Nasal Spray and DDAVP® tablets from Aventis Pharmaceuticals), as well as a desmopressin acetate rhinal tube (available from Ferring Pharmaceuticals Inc.). Other products include, for example, tolterodine tartrate (available as Detroltm tablets from Pharmacia & Upjohn), oxybutinin chloride (available in the form of Ditropan® tablets and syrup and Ditropan XL® extended release tablets from ALZA Pharmaceuticals), propanthaline bromide (available in tablet form from Roxane Laboratories, Inc.), hyoscyamine and hyoscyamine sulfate (available, respectively, as Cystopaz® tablets and Cystopaz-M® timed release capsules from PolyMedica Pharmaceuticals (U.S.A.), Inc.), hyoscyamine hydrobromide, flavoxate HCl (available in Urispas® 100 mg tablets from ALZA Pharmaceuticals), imipramine HCl (available in 10 mg, 25 mg and 50 mg tablets from Geneva Pharmaceuticals, Inc.), phenylpropanolamine, midodrine HCl (available in 2.5 mg and 5 mg Proamatine® tablets from Shire US Inc.), phenoxybenzamine HCl (available as Dibenzyline®V capsules from WellSpring Pharmaceuticals Corporation), and prazosin HCl (available in Minipress® capsules from Pfizer Inc.). Each of these medicaments may be administered in the pharmaceutically effective amounts and regimens known in the art, including those listed in the Physicians' Desk Reference, 55 Edition, 2001, published by Medical Economics Company, Inc. at Monvale, N.J. 07645-1742, the relevant portions of which are incorporated herein by reference.

Yet other pharmaceutical agents that can act to modulate bladder activity include, for example, other regulators of the 5HT_{2C} receptor. For example, United States Patent Application 2004/0235856 (previously incorporated herein by reference in its entirety) describes a variety of 5HT_{2C} receptor modulators that are useful in accordance with the practice of the present invention. Additional 5HT_{2C} agonists are exemplified in Bishop et al., Expert Opin. Ther. Patent 13:1691-1705, 2003, the entire contents of which are incorporated herein by reference.

Still other pharmaceutical agents that can act to modulate bladder activity include, for example, modulators of one or more KCNQ potassium channels. In some embodiments of the present invention, compounds of the present invention are administered in conjunction with one or more agonists of KCNQ 2/3 or KCNQ3/5. Such KCNQ modulators include, for example, compounds described in U.S. Pat. No. 5,384,330 and those described in U.S. Pat. No. 5,565,483, as well as those described in United States Patent Application No. 2002/0183395; and United States Patent Application No. 2004/0029949. The entire contents of each of these patents and patent applications is incorporated herein by reference. In some embodiments of the present invention, compounds of the present invention are administered with retigabine.

In some embodiments of the present invention, compounds of the present invention are administered in conjunction with one or more compounds which act as vasopressin agonists including, but not limited to those described in U.S. Pat. No. 6,194,407 (Failli et al.), U.S. Pat. No. 6,090,803 (Failli et al.), U.S. Pat. No. 6,096,736 (Ogawa et al.), and U.S. Pat. No. 6,096,735 (Ogawa et al.).

In general, it will often be desirable in accordance with the present invention to administer one or more compounds of the present invention in conjunction with one or more alpha-adrenergic receptor agonists and/or one or more other sympathomimetic drugs.

According to the present invention, compounds of formula I may be used to treat, prevent, or alleviate dependence, withdrawal, or symptoms thereof for any of a variety of substances including, for example, recreational substances (e.g., alcohol, tobacco [for example, nicotine]), pharmacologic agents (e.g., pain relievers [for example, Vicodin®, Lortab®, Lorcet®, Percocet®, Percodan®, Tylox®, Hydrocodone, OxyContin®, methadone, Tramadol, etc], tranquilizers, stimulants, or sedatives), and illicit drugs (e.g., marijuana, heroine, cocaine, ecstasy, LSD, PCP, methamphetamine, etc.).

The term “substance abuse”, as used herein, may be defined with reference to criteria set form in the Diagnostic and Statistical Manual of Mental Disorders, 4^{th} Ed. (1994) (“DSM-IV”), which was prepared by the Task Force on Nomenclature and Statistics of the American Psychiatric Association. A feature of substance abuse is a maladaptive pattern of substance use manifested by recurrent and significant adverse consequences related to the repeated use of substances. As recited in the DSM-IV, substance abuse is defined as maladaptive pattern of substance abuse leading to clinically significant impairment or distress, as manifested by one(or more) of the following, occurring within a 12-month period: (1) recurrent substance use resulting in a failure to fulfill major role obligations at work, school, or home; (2) recurrent substance use in situations in which it is physically hazardous; (3) recurrent substance-related legal problems; and (4) continued substance use despite having persistent or recurrent social or interpersonal problems cause or exacerbated by the effects of the substance. In addition, the DSM-IV requires that the symptoms of substance abuse do not meet the criteria for substance dependence.

The term “substance dependence”, as used herein, may be defined with reference to criteria set form in the Diagnostic and Statistical Manual of Mental Disorders, 4^{th} Ed. (1994) (“DSM-IV”), which was prepared by the Task Force on Nomenclature and Statistics of the American Psychiatric Association. The criteria for substance dependence set forth in DSM-IV is a pattern of substance use, leading to clinically significant impairment or distress as manifested by at least three selected from the following group, occurring at any time within the same twelve month period: (1) tolerance as defined by either (a) a need for substantially increased amounts of the substance to achieve the desired effect; or (b) substantially diminished effect with continued use of the same amount of the substance; (2) withdrawal, as demonstrated by either (a) the characteristic withdrawal syndrome for the specific substance; or (b) the same, or a closely related substance is taken to relieve or avoid withdrawal symptoms; (3) the substance is often taken in larger amounts or over a longer period then was intended; (4) there is a persistent desire or unsuccessful efforts to cut down or control substance use; (5) a great deal of time is spent in activities to obtain the substance, use the substance, or recover from its effects; (6) important social, occupational or recreational activities are given up or reduced because of substance use; and (7) the substance use is continued despite knowledge of having a persistent or recurrent physical or psychological problem that is likely to have been caused or exacerbated by the substance. Substance dependence can be with physiological dependence; that is evidence of tolerance or withdrawal is present, or without physiological dependence, where no evidence of tolerance or withdrawal is present. Four of the conditions set forth in DSM-IV include remission. These types of remission are based on the interval of time that has elapsed since the cessation of dependencies and whether there is continued presence of one or more of the symptoms included in the criteria for dependencies.

In certain embodiments, compounds of the present invention are useful for treating alcoholism (e.g., alcohol abuse, addiction and/or dependence including treatment for abstinence, craving reduction and relapse prevention of alcohol intake) and/or tobacco abuse (e.g., smoking addiction, cessation and/or dependence including treatment for craving reduction and relapse prevention of tobacco smoking).

In evaluating substance abuse in accordance with the present invention, reference may be made, for example, to the National Survey on Drug Use and Health (NSDUH), which obtains information on nine different categories of illicit drug use: marijuana, cocaine, heroin, hallucinogens, inhalants, and nonmedical use of prescription-type pain relievers, tranquilizers, stimulants, and sedatives. In these categories, hashish is included with marijuana, and crack is considered a form of cocaine. Several drugs are grouped under the hallucinogens category, including LSD, PCP, peyote, mescaline, mushrooms, and “Ecstasy” (MDMA). Inhalants include a variety of substances, such as amyl nitrite, cleaning fluids, gasoline, paint, and glue. The four categories of prescription-type drugs (pain relievers, tranquilizers, stimulants, and sedatives) cover numerous drugs available through prescriptions and sometimes illegally “on the street.” Methamphetamine is considered a type of stimulant. Respondents are asked to report only uses of drugs that were not prescribed for them or drugs they took only for the experience or feeling they caused. Over-the-counter drugs and legitimate uses of prescription drugs are not included. NSDUH reports combine the four prescription-type drug groups into a category referred to as “any psychotherapeutics.”

The NSDUH categorizes alcohol abuse through use of questions about the frequency of the consumption of alcoholic beverages, such as beer, wine, whiskey, brandy, and mixed drinks. An extensive list of examples of the kinds of beverages covered is given to respondents prior to the question administration. A “drink” is defined as a can or bottle of beer, a glass of wine or a wine cooler, a shot of liquor, or a mixed drink with liquor in it. Times when the respondent only had a sip or two from a drink are not considered as consumption. For this report, estimates for the prevalence of alcohol use are reported primarily at three levels defined for both males and females and for all ages as follows:

Current use—At least one drink in the past 30 days (includes binge and heavy use).

Binge use—Five or more drinks on the same occasion at least once in the past 30 days (includes heavy use).

Heavy use—Five or more drinks on the same occasion on at least 5 different days in the past 30 days

The NSDUH also characterizes the use of tobacco products, including cigarettes, chewing tobacco, snuff, cigars, and pipe tobacco. For analytic purposes, data for chewing tobacco and snuff are combined as “smokeless tobacco.” Cigarette use is defined as smoking “part or all of a cigarette.” Questions to determine nicotine dependence among current cigarette smokers also are included in NSDUH. Nicotine dependence is based on criteria from the Nicotine Dependence Syndrome Scale (NDSS) or the Fagerstrom Test of Nicotine Dependence (FTND).

In other embodiments, compounds of the present invention are useful for treating withdrawal from drug addiction including addiction to nicotine, alcohol, and other substances of abuse. Individuals often suffer the symptoms of nicotine withdrawal as a consequence of the discontinued use of tobacco in any form, including, but not limited to smoking of cigarette, cigar, or pipe tobacco, or the oral or intranasal ingestion of tobacco or chewing tobacco. Such oral or intranasal tobacco includes, but is not limited to snuff and chewing tobacco. The cessation of nicotine use or reduction in the amount of nicotine use, is often followed within 24 hours by symptoms including dysphoric, depressed mood; light-headedness; insomnia; irritability, frustration or anger; anxiety; nervous tremor; difficulty concentrating; restlessness; decreased heart rate; increased appetite or weight gain; and the craving for tobacco or nicotine. These symptoms often cause clinically significant distress or impairment in social, occupational, or other important areas of functioning.

The discontinued or reduction in administration of an opioid, typically self-administration, through injection or orally, through smoking or intranasal ingestion, often results in the presence of a characteristic opioid withdrawal condition. This withdrawal condition can also be precipitated by administration of an opioid antagonist such as naloxone or naltrexone after opioid use. Opioid withdrawal is characterized by symptoms that are generally opposite to the opioid agonist effects. These withdrawal symptoms may include anxiety; restlessness; muscle aches, often in the back and legs; craving for opioids; irritability and increased sensitivity to pain; dysphoric mood; nausea or vomiting; lacrimation; rhinorrhoea; papillary dilation; piloerection; sweating; diarrhea; yawning; fever; and insomnia. When dependence is on short-acting opioids, such as heroin, withdrawal symptoms usually occur within 6-24 hours after the last dose, while with longer-acting opioids, such as methadone, symptoms may take 2-4 days to emerge. These symptoms often cause clinically significant distress or impairment in social, occupational or other important areas of functioning. The present invention is most preferably used to alleviate one or more symptoms attributed to opioid withdrawal when such symptoms are not due to a general medical condition and are not better accounted for by another medical disorder.

The discontinued or reduction in use of ethanol (ethanol containing beverages) results in the onset of ethanol withdrawal conditions. Ethanol withdrawal conditions are characterized by symptoms that begin when blood concentrations of ethanol decline sharply, within 4 to 12 hours after ethanol use has been stopped or reduced. These ethanol withdrawal symptoms include craving for ethanol; autonomic hyperactivity (such as sweating or pulse rate greater than 100); hand tremor; insomnia; nausea; vomiting; transient visual, tactile, or auditory hallucinations or illusions; psychomotor agitation; anxiety; and grand mal seizures. These symptoms often cause clinically significant distress or impairment in social, occupational, or other important areas of functioning. The present invention is most preferably used to alleviate one or more symptoms attributed to ethanol withdrawal when such symptoms are not due to a general medical condition and are not better accounted for by another medical disorder.

According to another embodiment, a compound of the present invention is administered in combination with one or more agents useful for treating substance abuse. In certain embodiments, a compound of the present invention is administered in combination with one or more agents to treat tobacco abuse. Such agents include nicotine receptor partial agonists bupropion hypochloride (Zyban™) and nicotine replacement therapies.

According to yet another embodiment, a compound of the present invention is administered in combination with one or more agents to treat alcoholism, such as opioid antagonists (e.g., naltrexone, ReVia™), nalmefene, disulfiram (Antabuse™), and acamprosate (Campral™).

In certain embodiments, a compound is administered in combination with one or more agents for reducing alcohol withdrawal symptoms such as benzodiazepines, beta-blockers, clonidine, carbamazepine, pregabalin, and gabapentin (Neurontin™). In other embodiments of the invention, therapy utilizing compounds of the present invention is administered concomitantly with, in connection with, and/or subsequent to an educational and/or behavioral modification program to enhance continued abstinence from substance dependence or abuse. The method of the present invention may be particularly useful in treating symptoms of withdrawal often observed in rehabilitation or other treatment programs. Therefore, the programs can be more effective by focusing on educational and behavioral modification goals, further reducing the incidence of program non-completion.

In certain embodiments, compounds of the present invention are useful for treating one or more intellectual deficit disorders comprising administering a compound of the present invention. In other embodiments, such intellectual deficit disorders include dementia, such as dementia of aging, vascular dementia, mild cognitive impairment, age-related cognitive decline, and mild neurocognitive disorder; Alzheimer's disease, and memory deficit, attention deficit disorders (ADD, also known as Attention Deficit Hyperactivity Disorder or ADHD) in both children and adults. In certain embodiments, the present invention provides a method of treating ADD and/or ADHD in a pediatric patient comprising administering to said patient a compound of formula I or pharmaceutical composition thereof.

In other embodiments, the present invention provides a method of treating one or more cognition disorders. According to another aspect, the cognition disorder is a learning disorder. Such learning disorders are known in the art and include autism, dyslexia, Asperger's syndrome, a neurobiological disorder similar to autism and characterized by serious deficits in social and communication skills; specific learning disability, a disorder in one or more of the basic psychological processes involved in understanding or in using spoken or written language, which may manifest itself in an imperfect ability to listen, think, speak, read, write, spell or to do mathematical calculations; dysgraphia, a disorder that causes difficulty with forming letters or writing within a defined space; dyscalculia, a disorder that causes people to have problems doing arithmetic and grasping mathematical concepts; dyspraxia, a problem with the body's system of motion that interferes with a person's ability to make a controlled or coordinated physical response in a given situation; visual perceptual deficit, difficulty receiving and/or processing accurate information from the sense of sight, although there is nothing wrong with vision; and auditory perceptual deficit, difficulty receiving accurate information through auditory means, even though there is no problem with hearing.

In certain embodiments, the present invention provides a method for treating one or more impulsivity disorders (e.g. borderline personality disorder), disruptive behavior disorders, or impulse control disorders. In certain embodiments, the present invention provides a method for treating Tourette's syndrome (TS), an inherited, neurological disorder characterized by repeated and involuntary body movements (tics) and/or uncontrollable vocal sounds.

According to another aspect, the present invention provides a method for treating one or more behavioral addictions and addictive disorders. Behavioral addictions and addictive disorders result from the intoxication one senses from the release of brain chemicals (e.g., serotonin, adrenaline, epinepherine, etc.) during certain activities. Such disorders are known in the art and include gambling, sex addiction, eating disorders, spending addiction, rage/anger, workaholism, exercise addiction, risk taking addictions, and perfectionism to name a few.

In certain embodiments, a compound of the present invention is administered in combination with one or more cognitive improvement agents. Such agents are well known in the art and include donepezil hydrochloride (Aircept™) and other acetylcholinesterase inhibitors; galantamine, neuroprotective agents (e.g., memantine); ADD/ADHD agents (e.g., methylphenidate (Ritalin^{Tn,,}), atomoxetine (Strattera™), methylphenidate, sustained release (Concerta™) and amphetamine/dextroamphetamine (Adderall™).

According to another aspect, the present invention provides a method for treating sexual dysfunction comprising administering a compound of the present invention. In certain embodiments, the sexual dysfunction is associated with a depressive disorder. In other embodiments, the sexual dysfunction is associated with treatment of a disorder by administration of a serotonin reuptake inhibitor. Compounds of the present invention are useful for treating sexual dysfunction in the male and in the female. Such disorders include male erectile dysfunction (MED) and female sexual dysfunction (FSD), e.g. female sexual arousal disorder (FSAD).

In other embodiments, the present invention provides a method for treating one or more disorders associated with sexual dysfunction including: HSDD, characterized by a deficiency, or absence of, sexual fantasies and desire for sexual activity; FSAD, characterized by a persistent or recurrent inability to attain, or to maintain until completion of the sexual activity, an adequate lubrication-swelling response of sexual excitement; FOD characterized by persistent or recurrent delay in, or absence of, orgasm following a normal sexual excitement phase; Sexual Pain Disorders such as dyspareunia and vaginismus; and/or HSDD characterized by a woman who has no or little desire to be sexual, and has no or few sexual thoughts or fantasies.

According to another embodiment, a compound of the present invention is administered in combination with one or more agents for treating male sexual dysfunction (e.g., male erectile dysfunction). Such agents are known in the art and include a dopaminergic agent (e.g. D2, D3 or D4 agonists and apomorphine); an NPY (neuropeptide Y) (preferably an NPY-1 and/or NPY-5 inhibitor); a melanocortin receptor agonist or modulator or melanocortin enhancer; an NEP inhibitor; a PDE inhibitor (preferably, a cGMP PDE-5 inhibitor); a bombesin receptor antagonist or modulator, and a soluble secreted endopeptidase inhibitor (SEPi). In certain embodiments, a compound of the present invention is administered in combination with one or more agents for treating male sexual dysfunction such as alprostadil or sildenafil.

According to yet another embodiment, a compound of the present invention is administered in combination with one or more agents for treating female sexual dysfunction. Such agents are known in the art and include estrogen receptor modulators (e.g., estrogen agonists and/or estrogen antagonists); testosterone replacement agents, testosternone (Tostrelle), dihydrotestosterone, dehydroepiandrosterone (DHEA), a testosterone implant; eg dehydroandrostendione, estrogen, estrogen, medroxyprogesterone, medroxyprogesterone acetate (MPA), a combination of estrogen and a methyl testosterone hormone replacement therapy agent; Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone, a dopaminergic agent; eg apomorphine or a selective D2, D3 or D2/D₃agonist such as, pramipexole and ropirinol, a NPY (neuropeptide Y) inhibito; eg a NPY (neuropeptide Y) inhibitor such as a NPY1 or NPY5 inhibitor, preferably NPY1 inhibitor, a melanocortin receptor modulator or a melanocortin enhancer; eg melanotan II, PT-14, PT-141, a NEP (neutral endopeptidase) inhibitor; a PDE (phosphodiesterase) inhibitor; eg sildenafil, and/or a bombesin receptor modulator.

According to the present invention, compounds of the present invention are useful for treating any of a variety of different types of pain experienced by mammals, such as humans. For example, the compounds of the present invention may be used to treat acute pain (short duration) or chronic pain (regularly reoccurring or persistent), whether centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain, or headaches such as migraines or tension headaches, or combinations of these pains. One skilled in the art will recognize that these pains may overlap one another. For example, a pain caused by inflammation may also be visceral or musculoskeletal in nature.

In one embodiment of the present invention, one or more compounds of the present invention is/are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromylagia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In some embodiments, the compounds of the present invention are used to treat chronic pain that is neuropathic pain, visceral pain, musculoskeletal pain, bony pain, headache, cancer pain or inflammatory pain or combinations thereof, in accordance with the methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. Inventive treatment methods further include treatments in which the neuropathic pain is a condition secondary to metastatic infiltration, adiposis dolorosa, burns or central pain conditions related to thalamic conditions.

Neuropathic pains described above may also be, in some circumstances, classified as “painful small fiber neuropathies” such as idiopathic small-fiber painful sensory neuropathy, or “painful large fiber neuropathies” such as demylinating neuropathy or axonal neuropathy, or combinations thereof. Such neuropathies are described in more detail, for example, in the J. Mendell et al., N. Engl. J. Med. 2003, 348:1243-1255, which is hereby incorporated by reference in its entirety.

In another embodiment, the compounds useful in the present invention may be administered to totally or partially inhibit a neuropathic pain condition from developing. For example, compounds of the present invention may be administered to a mammal who is at risk for developing a neuropathic pain condition such as a mammal who has contracted shingles or a mammal who is being treated for cancer.

In one embodiment, the compounds useful in the present invention may be administered prior to or during a surgical procedure to partially or totally inhibit development of pain associated with the surgical procedure.

As mentioned previously, the methods of the present invention may be used to treat pain that is somatic and/or visceral in nature. For example, somatic pain that can be treated in accordance with the methods of the present invention includes pain associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, chronic pain to be treated in accordance with the present invention may be with or without peripheral or central sensitization.

The present invention also provides use of the compounds of the present invention to treat acute and/or chronic pains associated with female conditions, which may also be referred to as female-specific pain. Such types of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

In certain embodiments, a compound of the present invention is administered in combination with a pain relieving agent. Examples of pain relieving agents that may be administered with compounds of the present invention include, but are not limited to, analgesics such as non-narcotic analgesics or narcotic analgesics; anti-inflammatory agents such as non-steroidal anti-inflammatory agents (NSAIDs), steroids or anti-rheumatic agents; migraine preparations such as beta adrenergic blocking agents, ergot derivatives, or isometheptene; tricyclic antidepressants such as amitryptyline, desipramine, or imipramine; anti-epileptics such as gabapentin, carbamazepine, topiramate, sodium valproate or phenyloin; α₂ agonists; or selective serotonin reuptake inhibitors/selective norepinepherine uptake inhibitors, or combinations thereof.

One skilled in the art will recognize that some agents described herein act to relieve multiple conditions such as pain and inflammation, while other agents may just relieve one symptom such as pain. A specific example of an agent having multiple properties is aspirin, where aspirin is anti-inflammatory when given in high doses, but at lower doses is just an analgesic. The pain relieving agent may include any combination of the aforementioned agents, for example, the pain relieving agent may be a non-narcotic analgesic in combination with a narcotic analgesic.

Non-narcotic analgesics useful in the practice of the present invention include, for example, salicylates such as aspirin, ibuprofen (Motrin®, Advil®), ketoprofen (Orudis®), naproxen (Naprosyn®), acetaminophen, indomethacin or combinations thereof. Examples of narcotic analgesic agents that may be used in combination with compounds of the present invention include opioid analgesics such as fentenyl, sufentanil, morphine, hydromorphone, codeine, oxycodone, buprenorphine or pharmaceutically acceptable salts thereof or combinations thereof. Examples of anti-inflammatory agents that may be used in combination with compounds of the present invention include but are not limited to aspirin; ibuprofen; ketoprofen; naproxen; etodolac (Lodine®); COX-2 inhibitors such as celecoxib (Celebrex®), rofecoxib (Vioxx®), valdecoxib (Bextra®), parecoxib, etoricoxib (MK663), deracoxib, 2-(4-ethoxy-phenyl)-3-(4-methanesulfonyl-phenyl)-pyrazolo[1,5-b]pyridazine, 4-(2-oxo-3-phenyl-2,3-dihydrooxazol-4-yl)benzenesulfonamide, darbufelone, flosulide, 4-(4-cyclohexyl-2-methyl-5-oxazolyl)-2-fluorobenzenesulfonamide), meloxicam, nimesulide, 1-Methylsulfonyl-4-(1,1-dimethyl-4-(4-fluorophenyl)cyclopenta-2,4-dien-3-yl)benzene, 4-(1,5-Dihydro-6-fluoro-7-methoxy-3-(trifluoromethyl)-(2)-benzothiopyrano(4,3-c)pyrazol-1-yl)benzenesulfonamide, 4,4-dimethyl-2-phenyl-3-(4-methylsulfonyl)phenyl)cyclo-butenone, 4-Amino-N-(4-(2-fluoro-5-trifluoromethyl)-thiazol-2-yl)-benzene sulfonamide, 1-(7-tert-butyl-2,3-dihydro-3,3-dimethyl-5-benzo-furanyl)-4-cyclopropyl butan-1-one, or their physiologically acceptable salts, esters or solvates; sulindac (Clinoril®); diclofenac (Voltaren®); piroxicam (Feldene®); diflunisal (Dolobid®), nabumetone (Relefen®), oxaprozin (Daypro®), indomethacin (Indocin®); or steroids such as Pediaped® prednisolone sodium phosphate oral solution, Solu-Medrol® methylprednisolone sodium succinate for injection, Prelone® brand prednisolone syrup.

Further examples of anti-inflammatory agents that may be used for treating pain, for example associated with rheumatoid arthritis, in accordance with the present invention include naproxen, which is commercially available in the form of EC-Naprosyn® delayed release tablets, Naprosyn®, Anaprox® and Anaprox® DS tablets and Naprosyn® suspension from Roche Labs, Celebrex® brand of celecoxib tablets, Vioxx® brand of rofecoxib, Celestone® brand of betamethasone, Cupramine® brand penicillamine capsules, Depen® brand titratable penicillamine tablets, Depo-Medrol® brand of methylprednisolone acetate injectable suspension, Arava™ leflunomide tablets, Azulfidine EN-tabs® brand of sulfasalazine delayed release tablets, Feldene® brand piroxicam capsules, Cataflam® diclofenac potassium tablets, Voltaren® diclofenac sodium delayed release tablets, Voltaren5-XR diclofenac sodium extended release tablets, or Enbrel® etanerecept products.

Examples of yet other agents used to treat inflammations, especially rheumatoid arthritis, include immunosuppressants such as Gengraf™ brand cyclosporine capsules, Neoral® brand cyclosporine capsules or oral solution, or Imuran® brand azathioprine tablets or IV injection; Indocin® brand indomethacin capsules, oral suspension or suppositories; Plaquenil® brand hydroxychloroquine sulfate; or Remicade® infliximab recombinant for IV injection; or gold compounds such as auranofin or Myochrisyine® gold sodium thiomalate injection.

In other embodiments, compounds of the present invention are useful for treating one or more central nervous system deficiencies associated, for example, with trauma, stroke, and spinal cord injuries, neurodegenerative diseases or toxic or infective CNS diseases (e.g., encephalitis or meningitis), or Parkinson's disease. The compounds of the present invention can therefore be used to improve or inhibit further degradation of central nervous system activity during or following the malady or trauma in question. Included in these improvements are maintenance or improvement in motor and motility skills, control, coordination and strength.

5. Pharmaceutically Acceptable Compositions

In other embodiments, the invention relates to compositions comprising at least one compound of formula I, or a pharmaceutically acceptable salt thereof, and one or more pharmaceutically acceptable carriers, excipients, or diluents. Such compositions include pharmaceutical compositions for treating or controlling disease states or conditions of the central nervous system. In certain embodiments, the compositions comprise mixtures of one or more compounds of formula I.

In certain embodiments, the invention relates to compositions comprising at least one compound of formula I, or a pharmaceutically acceptable salt thereof, and one or more pharmaceutically acceptable carriers, excipients, or diluents. Such compositions are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

The compounds of formula I can be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of formula I can be administered rectally or vaginally in the form of a conventional suppository. For administration by intranasal or intrabronchial inhalation or insufflation, the compounds of formula I can be formulated into an aqueous or partially aqueous solution, which can then be utilized in the form of an aerosol. The compounds of Formula 1 can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The amount of compound of formula I provided to a patient will vary depending upon what is being administered, the purpose of the administration, such as prophylaxis or therapy, the state of the patient, the manner of administration, and the like. In therapeutic applications, compounds of formula I are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case must be subjectively determined by the attending physician. The variables involved include the specific condition and the size, age, and response pattern of the patient. The treatment of substance abuse follows the same method of subjective drug administration under the guidance of the attending physician. Generally, a starting dose is about 5 mg per day with gradual increase in the daily dose to about 1000 mg per day, to provide the desired dosage level in the patient.

6. Combination with Other Agents

Compounds of formula I may be administered alone in order to treat various disorders in accordance with the present invention, or may be combined with one or more other pharmaceutical agents as described herein. Where the present invention involves administration of two or more pharmaceutical agents, the two or more agents may be administered simultaneously (such as individually at the same time, or together in a pharmaceutical composition), and/or successively with one another. In general, a compound of formula I and the other pharmaceutical agent(s) are administered in a manner so that both are present in the mammal body for a certain period of time to treat the disorder.

Also, the two or more pharmaceutical agents may be delivered via the same route of administration or by different routes. Desirable routes of administration may well depend upon the particular agent(s) chosen, many of which have recommended administration route(s) known to those skilled in the art. For example, opioids are generally administered by oral, intravenous, or intramuscular administration routes. Similarly, as is known in the art, doses of pharmaceutical agents in a composition may be affected by administration route. In general, pharmaceutical agents may be dosed and administered according to practices known to those skilled in the art such as those disclosed in references such as the Physicians' Desk Reference, 55 Edition, 2001, published by Medical Economics Co., Inc., Montvale, N.J.

A more complete list of pharmaceutically active agents, including pain relieving agents, can be found in the Physicians' Desk Reference, 55 Edition, 2001, published by Medical Economics Co., Inc., Montvale, N.J. Each of these agents may be administered in conjunction with one or more comopunds of formula I according to the present invention. For most or all of these agents, recommended effective dosages and regimes are known in the art; many can be found in the above-referenced Physicians' Desk Reference, 55 Edition, 2001, published by Medical Economics Co., Inc., Montvale, N.J.

In certain embodiments, the present invention is directed to prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

### EXAMPLES

As depicted in the Examples below, in certain exemplary embodiments, compounds are prepared according to the following general procedures. It will be appreciated that although the general methods depict the synthesis of certain compounds of the present invention, the following general methods, in addition to the Schemes set forth above and other methods known to one of ordinary skill in the art, can be applied to all compounds and subclasses and species of each of these compounds, as described herein.

The following examples illustrate the production of representative compounds of this invention.

### Intermediate 1

Toluene-4-sulfonic acid 8-hydroxy-2,3-dihydrobenzo-[1,4]dioxin-2-ylmethyl ester: To a solution of toluene-4-sulfonic acid 8-formyl-2,3-dihydrobenzo[1,4]dioxin-2-ylmethyl ester (15.4 g, 44 mmol) in methylene chloride (500 mL) was added m-CPBA (77% max, 17.2 g) at room temperature. The mixture was stirred at room temperature overnight. Then the mixture was extracted with methylene chloride and saturated sodium bicarbonate. The organic layer was washed with saturated sodium chloride and dried over anhydrous sodium sulfate and filtered. The solvent was removed under vacuum afforded a crude oil. To the solution of the crude oil in methanol (500 mL) was added basic aluminum oxide (50 g) at room temperature. The mixture was stirred at room temperature overnight. Then the mixture was filtered through a pad of celite and concentrated under the vacuum. Chromatography with methylene chloride afforded 14.2 g (96%) of the title compound as a colorless oil.

### Intermediate 2

Toluene-4-sulfonic acid-8-(trifluoromethyanesulfonyloxy)-2,3-dihydrobenzo-[1,4]dioxin-2-ylmethyl ester: To a solution of toluene-4-sulfonic acid 8-hydroxy-2,3-dihydrobenzo-[1,4]dioxin-2-ylmethyl ester (14.2 g, 42 mmol) in methylene chloride (500 mL) was added diisopropylethylamine (13.0 g, 100 mmol) at 0° C., followed by trifluoromethanesulfonic anhydride (14.1 g, 50 mmol). The reaction mixture was stirred for 2 h at room temperature. The reaction was quenched with water, and extracted with methylene chloride. The organic layer was washed sequentially with 2N HCl, saturated sodium bicarbonate and brine. The organic layer was dried over anhydrous sodium sulfate and filtered. The solvent was evaporated under vacuum. Chromatography with 50% methylene chloride in hexanes afforded the title compound 18.1 g (92%) as a white solid.

### Intermediate 3

(R)-Toluene-4-sulfonic acid 8-hydroxy-2-methyl-2,3-dihydrobenzo-[1,4]dioxin-2-ylmethyl ester: To a solution of (R)-toluene-4-sulfonic acid 8-formyl-2-methyl-2,3-dihydrobenzo[1,4]dioxin-2-ylmethyl ester (3.80 g, 10 mmol) in methylene chloride was added m-CPBA (77% max, 6.0 g) at room temperature. The mixture was stirred at room temperature overnight. Then the reaction was quenched with 10% sodium sulfite and 10% sodium bicarbonate. The mixture was extracted with methylene chloride and washed with water. The solvent was removed under vacuum afforded a crude oil. To the solution of the crude oil in methanol was added sodium hydroxide (1.6 g, 40 mmol) at 0° C. The mixture was stirred at room temperature for 2 h. Then the mixture was neutralized with concentrated hydrochloric acid and extracted with methylene chloride. The organic layer was washed with water and dried over anhydrous sodium sulfate and filtered. The solvent was removed under vacuum. Chromatography with 20% ethyl acetate in hexanes afforded 3.32 g (90%) of the title compound as a white solid: mp 81.4-82.6° C.

Elemental Anal. for C₁₇H₁₈O₆S

Theory: C, 58.27; H, 5.18.

Found: C, 58.42; H, 4.78.

### Intermediate 4

(R)-Toluene-4-sulfonic acid 2-methyl-8-trifluoromethyanesulfonyloxy-2,3-dihydrobenzo-[1,4]dioxin-2-ylmethyl ester: To a solution of (R)-toluene-4-sulfonic acid 8-hydroxy-2-methyl-2,3-dihydrobenzo-[1,4]dioxin-2-ylmethyl ester (3.32 g, 9.5 mmol) in methylene chloride (60 mL) was added trifluoromethanesulfonic anhydride (1.91 mL, 11.3 mmol) and diisopropylethylamine (2.71 mL, 14.0 mmol) at 0° C. The reaction mixture was stirred for 1 h at 0° C. and 2 h at room temperature. The reaction was quenched with water, and extracted with methylene chloride. The organic layer was washed with water, dried over anhydrous sodium sulfate and filtered. The solvent was evaporated under vacuum. Chromatography with 20% ethyl acetate in hexanes afforded the title compound (4.30 g, 94%) as a colorless oil.

Elemental Anal. for C₁₈H₁₇F₃O₈S₂

Theory: C, 44.81; H, 3.55.

Found: C, 45.15; H, 3.43.

### Intermediate 5

(R)-Toluene-4-sulfonic acid 6-chloro-8-propenyl-2,3-dihydro-benzo[1,4]dioxin-2-ylmethyl ester: To a solution of toluene-4-sulfonic acid 8-allyl-6-chloro-2,3-dihydro-benzo[1,4]dioxin-2-ylmethyl ester (3.95 g, 10 mmol) in methylene chloride (150 mL) was added dichlorobis(acetonitrile)palladium(II) (0.52 g, 2 mmol). The resulting mixture was refluxed overnight. The solvent was removed under vacuum. Chromatography with 5-20% ethyl acetate in hexanes afforded 2.4 g (61%) of the title compound as a light yellow oil.

### Intermediate 6

(R)-Toluene-4-sulfonic acid 6-chloro-8-hydroxy-2,3-dihydro-benzo[1,4]dioxin-2-ylmethyl ester: To a solution of toluene-4-sulfonic acid 6-chloro-8-propenyl-2,3-dihydro-benzo[1,4]dioxin-2-ylmethyl ester (9.4 g, 23.8 mmol) in THF (180 mL) and water (25 mL) was added osmium tetroxide solution (4% in water, 5.0 mL) and sodium periodate (15.3 g, 71.4 mmol) at 0° C. The resulting mixture was stirred at 0° C. for 2 h and poured in ice water. The mixture was extracted with ethyl acetate and washed with water. The organic layer was dried over anhydrous sodium sulfate and filtered. The solution was concentrated to provided toluene-4-sulfonic acid 6-chloro-8-formyl-2,3-dihydro-benzo[1,4]dioxin-2-ylmethyl ester. This was further treated with m-CPBA (77% max, 20.0 g) in methylene chloride (200 mL). The resulting mixture was stirred at room temperature overnight and quenched with 1:1 10% sodium sulfite in saturated sodium bicarbonate. The mixture was extracted with methylene chloride and washed with water. The solvent was removed under vacuum afforded a crude material as a light yellow oil. To a solution of the crude oil in methanol was added sodium bicarbonate (5.0 g, 59.5 mmol) at room temperature. The mixture was stirred at room temperature for 2 h and the solvent was removed under vacuum. The mixture was extracted with ethyl acetate and washed with water. The organic solvent was removed under vacuum. Chromatography with 10-40% ethyl acetate in hexanes afforded 5.9 g (67% for 3 steps) of the title compound as a colorless oil.

### Intermediate 7

(R)-Toluene-4-sulfonic acid 6-chloro-8-trifluoromethanesulfonyloxy-2,3-dihydro-benzo[1,4]dioxin-2-ylmethyl ester: To a solution of (R)-toluene-4-sulfonic acid 6-chloro-8-hydroxy-2,3-dihydro-benzo[1,4]dioxin-2-ylmethyl esther (5.9 g, 15.9 mmol) in methylene chloride (150 mL) was added trifluoromethanesulfonic anhydride (3.5 mL, 20.7 mmol), diisopropylethylamine (5.5 mL, 31.8 mmol) at 0° C. The resulting mixture was stirred at room temperature for 24 h. Then the reaction was quenched with ice water and extracted with methylene chloride. The organic layer was washed with water and dried over anhydrous sodium sulfate and filtered. The solvent was removed under vacuum. Chromatography with 5-30% ethyl acetate in hexanes afforded 7.6 g (95%) of the title compound as a white solid.

General procedure to generate biaryl derivatives from toluene-4-sulfonic acid 8-trifluoromethanesulfonyloxy-2,3-dihydrobenzo[1,4]dioxin-2-ylmethyl ester:

To a solution of toluene-4-sulfonic acid 8-trifluoromethanesulfonyloxy-2,3-dihydrobenzo[1,4]dioxin-2-ylmethyl ester (1.0 eq) and substituted benzene boronic acid (2 eq) in DME-water (4/1) was added tetrakis(triphenylphospine)palladium (0) (0.03 eq) and sodium carbonate (2.5 eq). The reaction mixture was heated at 80° C. until starting material was gone. The mixture was filtered through the pad of celite and concentrated under vacuum. Chromatography with 10% ethyl acetate in hexanes afforded product as an oil.

Using the general procedures outlined above, Intermediates 8-41 can be prepared.

### Intermediate 8

Toluene-4-sulfonic acid 8-(2-chlorophenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.235 g, 0.5 mmol) and 2-chlorobenzene boronic acid, 142 mg (66%) of the title compound was obtained as a colorless oil.

### Intermediate 9

Toluene-4-sulfonic acid 8-(2-fluorophenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2-fluorobenzene boronic acid, 410 mg (99%) of the title compound was obtained as a colorless oil.

### Intermediate 10

Toluene-4-sulfonic acid 8-(2-methyl-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2-methylbenzene boronic acid, 350 mg (85%) of the title compound was obtained as a colorless oil.

### Intermediate 11

Toluene-4-sulfonic acid 8-(2-trifluoromethyl-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2-trifluoromethylbenzene boronic acid, 440 mg (94%) of the title compound was obtained as a colorless oil.

### Intermediate 12

Toluene-4-sulfonic acid 8-(2-methoxy-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2-methoxybenzene boronic acid, 350 mg (82%) of the title compound was obtained as a colorless oil.

### Intermediate 13

Toluene-4-sulfonic acid 8-(2,3-dichloro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,3-dichlorobenzene boronic acid, 350 mg (75%) of the title compound was obtained as a colorless oil.

### Intermediate 14

Toluene-4-sulfonic acid 8-(2,4-dichloro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (235 mg, 0.5 mmol) and 2,4-dichlorobenzene boronic acid, 180 mg (77%) of the title compound was obtained as a colorless oil.

### Intermediate 15

Toluene-4-sulfonic acid 8-(2,5-dichloro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,5-dichlorobenzene boronic acid, 390 mg (83%) of the title compound was obtained as a colorless oil.

### Intermediate 16

Toluene-4-sulfonic acid 8-(2,3-dimethoxy-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,3-dimethoxybenzene boronic acid, 310 mg (68%) of the title compound was obtained as a colorless oil.

### Intermediate 17

Toluene-4-sulfonic acid 8-(2,3-dimethyl-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,3-dimethylbenzene boronic acid, 370 mg (87%) of the title compound was obtained as a colorless oil.

### Intermediate 18

Toluene-4-sulfonic acid 8-(2,5-dimethyl-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,5-dimethylbenzene boronic acid, 430 mg (100%) of the title compound was obtained as a colorless oil.

### Intermediate 19

Toluene-4-sulfonic acid 8-(2,6-dimethyl-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,6-dimethylbenzene boronic acid, 230 mg (54%) of the title compound was obtained as a colorless oil.

### Intermediate 20

Toluene-4-sulfonic acid 8-(2,3-difluoro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,3-difluorobenzene boronic acid, 400 mg (92%) of the title compound was obtained as a colorless oil.

### Intermediate 21

Toluene-4-sulfonic acid 8-(2,4-difluoro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,4-difluorobenzene boronic acid, 400 mg (92%) of the title compound was obtained as a colorless oil.

### Intermediate 22

Toluene-4-sulfonic acid 8-(2,5-difluoro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2,5-difluorobenzene boronic acid, 370 mg (85%) of the title compound was obtained as a colorless oil.

### Intermediate 23

Toluene-4-sulfonic acid 8-(2-methoxy-5-chloro-phenyl)-2,3-dihydro-benzo[1,4]dioxin-2-yl-methyl ester: Starting from toluene-4-sulfonic acid 8-trifluoromethanesulfonyloxy-2,3-dihydrobenzo-[1,4]dioxin-2-ylmethyl ester (0.47 g, 1 mmol) and 2-methoxy-5-chlorobenzene boronic acid, 460 mg (100%) of the product was obtained as a colorless oil.

### Intermediate 24

(R)-Toluene-4-sulfonic acid 2-methyl-8-phenyl-2,3-dihydro-benzo[1,4]dioxin-2-yl-methyl ester: Starting from (R)-toluene-4-sulfonic acid 2-methyl-8-trifluoromethanesulfonyloxy-2,3-dihydro-benzo[1,4]dioxin-2-yl-methyl ester (0.3 g, 0.62 mmol) and phenyl boronic acid (0.23 g, 1.9 mmol), 0.26 g (100%) of the title compound was obtained as a colorless oil. MS ESI m/e 411.1 [M+H]⁺

### Intermediate 25

(R)-Toluene-4-sulfonic acid 8-(2-chloro-phenyl)-2-methyl-2,3-dihydro-benzo[1,4]dioxin-2-yl-methyl ester: Starting from (R)-toluene-4-sulfonic acid 2-methyl-8-trifluoromethanesulfonyloxy-2,3-dihydro-benzo[1,4]dioxin-2-yl-methyl ester (0.3 g, 0.62 mmol) and 2-chlorobenzene boronic acid (0.29 g, 1.9 mmol), 0.27 g (97%) of the title compound was obtained as a colorless oil. MS ESI m/e 462.1 [M+NH₄]⁺

### Intermediate 26

(R)-Toluene-4-sulfonic acid 8-(3-chloro-phenyl)-2-methyl-2,3-dihydro-benzo[1,4]dioxin-2-yl-methyl ester: Starting from (R)-toluene-4-sulfonic acid 2-methyl-8-trifluoromethanesulfonyloxy-2,3-dihydro-benzo[1,4]dioxin-2-yl-methyl ester (0.3 g, 0.62 mmol) and 3-chlorobenzene boronic acid (0.29 g, 1.9 mmol), 0.27 g (100%) of the title compound was obtained as a colorless oil. MS ESI m/e 445.1 [M+H]⁺

### Intermediate 27

(R)-Toluene-4-sulfonic acid 8-(4-chloro-phenyl)-2-methyl-2,3-dihydro-benzo[1,4]dioxin-2-yl-methyl ester: Starting from (R)-toluene-4-sulfonic acid 2-methyl-8-trifluoromethanesulfonyloxy-2,3-dihydro-benzo[1,4]dioxin-2-yl-methyl ester (0.3 g, 0.62 mmol) and 4-chlorobenzene boronic acid (0.29 g, 1.9 mmol), 0.27 g (100%) of the title compound was obtained as a colorless oil. MS ESI m/e 462.1 [M+NH₄]⁺.

### Intermediate 28

(R)-Toluene-4-sulfonic acid 8-(2-methoxy-phenyl)-2-methyl-2,3-dihydro-benzo[1,4]dioxin-2-yl-methyl ester: Starting from (R)-toluene-4-sulfonic acid 2-methyl-8-trifluoromethanesulfonyloxy-2,3-dihydro-benzo[1,4]dioxin-2-yl-methyl ester (0.3 g, 0.62 mmol) and 2-methoxybenzene boronic acid (0.28 g, 1.9 mmol), 0.28 g (100%) of the title compound was obtained as a colorless oil. MS ESI m/e 441.1 [M+H]⁺.

### Intermediate 29

(R)-Toluene-4-sulfonic acid-2-methyl-8-thiophen-3-yl-2,3-dihydro-benzo[1,4]-dioxin-2-ylmethylamine: Starting from (R)-toluene-4-sulfonic acid 2-methyl-8-trifluoromethanesulfonyloxy-2,3-dihydro-benzo[1,4]dioxin-2-yl-methyl ester (0.3 g, 0.62 mmol) and 3-thiophenylboronic acid (0.24 g, 1.9 mmol) 0.22 g (85%) of the title compound was obtained as a colorless oil. MS ESI m/e 417.1 [M+H]⁺.

### Intermediate 30

(R)-Toluene-4-sulfonic acid 8-(2-chloro-phenyl)-6-chloro-2,3-dihydro-benzo[1,4]dioxin-2-ylmethyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2-chlorobenzene boronic acid (0.39 g, 2.5 mmol), the general procedure described above gave the title compound (0.42 g, 91%) as a colorless oil.

### Intermediate 31

(R)-Toluene-4-sulfonic acid 8-(2-fluoro-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2-fluorobenzene boronic acid (0.35 g, 2.5 mmol), the general procedure described above gave the title compound (0.23 g, 52%) as a colorless oil.

### Intermediate 32

(R)-Toluene-4-sulfonic acid 8-(2-methyl-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2-methylbenzene boronic acid (0.34 g, 2.5 mmol), the general procedure described above gave the title compound (0.38 g, 85%) as a colorless oil.

### Intermediate 33

(R)-Toluene-4-sulfonic acid 8-(2-methoxy-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2-methoxybenzene boronic acid (0.38 g, 2.5 mmol), the general procedure described above gave the title compound (0.44 g, 96%) as a colorless oil.

### Intermediate 34

(R)-Toluene-4-sulfonic acid 8-(2-trifluoromethyl-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2-trifluoromethylbenzene boronic acid (0.47 g, 2.5 mmol), the general procedure described above gave the title compound (0.41 g, 82%) as a colorless oil.

### Intermediate 35

(R)-Toluene-4-sulfonic acid 8-(2,3-dimethoxy-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2,3-dimethoxybenzene boronic acid (0.45 g, 2.5 mmol), the general procedure described above gave the title compound (0.40 g, 82%) as a colorless oil.

### Intermediate 36

(R)-Toluene-4-sulfonic acid 8-(2,4-dichloro-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2,4-dichlorobenzene boronic acid (0.47 g, 2.5 mmol), the general procedure described above gave the title compound (0.36 g, 72%) as a colorless oil.

### Intermediate 37

(R)-Toluene-4-sulfonic acid 8-(4-chloro-2-methyl-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 4-chloro-2-methyl-benzene boronic acid (0.43 g, 2.5 mmol), the general procedure described above gave the title compound (0.40 g, 83%) as a colorless oil.

### Intermediate 38

(R)-Toluene-4-sulfonic acid 8-(2,4-di-trifluoromethyl-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2,4-di-trifluoromethylbenzene boronic acid (0.64 g, 2.5 mmol), the general procedure described above gave the title compound (0.42 g, 75%) as a colorless oil.

### Intermediate 39

(R)-Toluene-4-sulfonic acid 8-(2,5-dichloro-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2,5-dichlorobenzene boronic acid (0.47 g, 2.5 mmol), the general procedure described above gave the title compound (0.39 g, 78%) as a colorless oil.

### Intermediate 40

(R)-Toluene-4-sulfonic acid 8-(5-chloro-2-methoxy-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 5-chloro-2-methoxybenzene boronic acid (0.47 g, 2.5 mmol), the general procedure described above gave the title compound (0.39 g, 81%) as a colorless oil.

### Intermediate 41

(R)-Toluene-4-sulfonic acid 8-(2,6-dimethyl-phenyl)-6-chloro-2,3-dihydro-benzo[1,4]dioxin-2-yl-methyl ester: Starting from (R)-toluene-4-sulfonic acid 6-chloro-8-trifluoromethanesulfonyloxy-2,3-dihydro-benzo[1,4]dioxin-2-yl-methyl ester (0.50 g, 1.0 mmol) and 2,6-dimethylbenzene boronic acid (0.38 g, 2.5 mmol), the general procedure described above gave the title compound (0.27 g, 59%) as a colorless oil.

General Procedure to Generate Azide Derivatives:

To a solution of tosylate (INTERMEDIATES 8-41) (1.0 eq) in DMF was added sodium azide (5 eq). The reaction mixture was heated at 70-90° C. overnight. The reaction was quenched with water. The mixture was extracted with methylene chloride. The organic layer was washed with water and dried over anhydrous sodium sulfate. The organic solvent was removed under vacuum. Chromatography with 10-20% ethyl acetate in hexanes afforded the product as an oil.

Using the general procedures outlined above, INTERMEDIATES 42-75 can be prepared.

### Intermediate 42

2-Azidomethyl-8-(2-chloro-phenyl)-2,3-dihydro-benzo[1,4]dioxine: Starting from toluene-4-sulfonic acid 8-(2-chloro-phenyl)-2,3-dihydrobenzo-[1,4]dioxin-2-ylmethyl ester (142 mg, 0.33 mmol), 0.1 g (100%) of the title compound was obtained as a colorless oil.

### Intermediate 43

2-Azidomethyl-8-(2-fluoro-phenyl)-2,3-dihydro-benzo[1,4]dioxine: Starting from toluene-4-sulfonic acid 8-(2-fluoro-phenyl)-2,3-dihydrobenzo-[1,4]dioxin-2-ylmethyl ester (205 mg, 0.5 mmol), 0.14 g (99%) of the title compound was obtained as a colorless oil.

### Intermediate 44

2-Azidomethyl-8-(2-mehyl-phenyl)-2,3-dihydro-benzo[1,4]dioxine: Starting from toluene-4-sulfonic acid 8-(2-methyl-phenyl)-2,3-dihydrobenzo-[1,4]dioxin-2-ylmethyl ester (175 mg, 0.42 mmol), 0.11 g (92%) of the title compound was obtained as a colorless oil.

### Intermediate 45

2-Azidomethyl-8-(2-trifluoromethyl-phenyl)-2,3-dihydro-benzo[1,4]dioxine: Starting from toluene-4-sulfonic acid 8-(2-trifluoromethyl-phenyl)-2,3-dihydrobenzo-[1,4]dioxin-2-ylmethyl ester (220 mg, 0.47 mmol), 0.15 g (94%) of the title compound was obtained as a colorless oil.

### Intermediate 46

2-Azidomethyl-8-(2-methoxy-phenyl)-2,3-dihydro-benzo[1,4]dioxine: Starting from toluene-4-sulfonic acid 8-(2-methoxy-phenyl)-2,3-dihydrobenzo-[1,4]dioxin-2-ylmethyl ester (175 mg, 0.41 mmol), 0.13 g (100%) of the title compound was obtained as a colorless oil.

### Intermediate 47

2-Azidomethyl-8-(2,3-dichloro-phenyl)-2,3-dihydro-benzo[1,4]dioxine: Starting from toluene-4-sulfonic acid 8-(2,3-dichloro-phenyl)-2,3-dihydrobenzo-[1,4]dioxin-2-ylmethyl ester (175 mg, 0.38 mmol), 0.14 g of the title compound was obtained as a colorless oil.

### Intermediate 48

2-Azidomethyl-8-(2,4-dichloro-phenyl)-2,3-dihydro-benzo[1,4]dioxine: Starting from toluene-4-sulfonic acid 8-(2,4-dichloro-phenyl)-2,3-dihydrobenzo-[1,4]dioxin-2-ylmethyl ester (180 mg, 0.39 mmol), 0.13 g (100%) of the title compound was obtained as a colorless oil.

### Intermediate 49

2-Azidomethyl-8-(2,5-dichloro-phenyl)-2,3-dihydro-benzo[1,4]dioxine: Starting from toluene-4-sulfonic acid 8-(2,5-dichloro-phenyl)-2,3-dihydrobenzo-[1,4]dioxin-2-ylmethyl ester (185 mg, 0.4 mmol), 0.14 g of the title compound was obtained as a colorless oil.

### Intermediate 50

2-Azidomethyl-8-(2,3-dimethoxy-phenyl)-2,3-dihydro-benzo[1,4]dioxine: Starting from toluene-4-sulfonic acid 8-(2,3-dimethoxy-phenyl)-2,3-dihydrobenzo-[1,4]dioxin-2-ylmethyl ester (155 mg, 0.34 mmol), 0.1 g (90%) of the title compound was obtained as a colorless oil.

### Intermediate 51

2-Azidomethyl-8-(2,3-dimethyl-phenyl)-2,3-dihydro-benzo[1,4]dioxine: Starting from toluene-4-sulfonic acid 8-(2,3-dimethyl-phenyl)-2,3-dihydrobenzo-[1,4]dioxin-2-ylmethyl ester (185 mg, 0.43 mmol), 0.13 g (100%) of the title compound was obtained as a colorless oil.

### Intermediate 52

2-Azidomethyl-8-(2,5-dimethyl-phenyl)-2,3-dihydro-benzo[1,4]dioxine: Starting from toluene-4-sulfonic acid 8-(2,5-dimethyl-phenyl)-2,3-dihydrobenzo-[1,4]dioxin-2-ylmethyl ester (215 mg, 0.5 mmol), 0.14 g (93%) of the title compound was obtained as a colorless oil.

### Intermediate 53

2-Azidomethyl-8-(2,6-dimethyl-phenyl)-2,3-dihydro-benzo[1,4]dioxine: Starting from toluene-4-sulfonic acid 8-(2,6-dimethyl-phenyl)-2,3-dihydrobenzo-[1,4]dioxin-2-ylmethyl ester (230 mg, 0.54 mmol), the crude title compound was obtained as a colorless oil.

### Intermediate 54

2-Azidomethyl-8-(2,3-difluoro-phenyl)-2,3-dihydro-benzo[1,4]dioxine: Starting from toluene-4-sulfonic acid 8-(2,3-difluoro-phenyl)-2,3-dihydrobenzo-[1,4]dioxin-2-ylmethyl ester (200 mg, 0.46 mmol), 0.15 g (100%) of the title compound was obtained as a colorless oil.

### Intermediate 55

2-Azidomethyl-8-(2,4-difluoro-phenyl)-2,3-dihydro-benzo[1,4]dioxine: Starting from toluene-4-sulfonic acid 8-(2,4-difluoro-phenyl)-2,3-dihydrobenzo-[1,4]dioxin-2-ylmethyl ester (200 mg, 0.46 mmol), 0.12 g (85%) of the title compound was obtained as a colorless oil.

### Intermediate 56

2-Azidomethyl-8-(2,5-difluoro-phenyl)-2,3-dihydro-benzo[1,4]dioxine: Starting from toluene-4-sulfonic acid 8-(2,5-difluoro-phenyl)-2,3-dihydrobenzo-[1,4]dioxin-2-ylmethyl ester (185 mg, 0.42 mmol), 0.12 g (92%) of the title compound was obtained as a colorless oil.

### Intermediate 57

2-Azidomethyl-8-(2-methoxy-5-chloro-phenyl)-2,3-dihydro-benzo[1,4]dioxine: Starting from toluene-4-sulfonic acid 8-(2-methoxy-5-chloro-phenyl)-2,3-dihydrobenzo-[1,4]dioxin-2-ylmethyl ester (265 mg, 0.57 mmol), 0.14 g (73%) of the title compound was obtained as a colorless oil.

### Intermediate 58

(S)-2-Azidomethyl-2-methyl-8-phenyl-2,3-dihydro-benzo[1,4]dioxine: Starting from (R)-toluene-4-sulfonic acid 8-phenyl-2-methyl-2,3-dihydro-benzo[1,4]-dioxin-2-yl-methyl ester (0.26 g, 0.63 mmol) and sodium azide (0.20 g, 3.2 mmol), 0.17 g (100%) of the title compound was obtained as a colorless oil. MS EI m/e 281 M⁺.

### Intermediate 59

(S)-2-Azidomethyl-8-(2-chloro-phenyl)-2-methyl-2,3-dihydro-benzo[1,4]dioxine: Starting from (R)-toluene-4-sulfonic acid 8-(2-chloro-phenyl)-2-methyl-2,3-dihydro-benzo[1,4]-dioxin-2-yl-methyl ester (0.27 g, 0.61 mmol) and sodium azide (0.20 g, 3.0 mmol), 0.16 g (84%) of the title compound was obtained as a colorless oil. MS EI m/e 315 M⁺.

### Intermediate 60

(S)-2-Azidomethyl-8-(3-chloro-phenyl)-2-methyl-2,3-dihydro-benzo[1,4]dioxine: Starting from (R)-toluene-4-sulfonic acid 8-(3-chloro-phenyl)-2-methyl-2,3-dihydro-benzo[1,4]-dioxin-2-yl-methyl ester (0.27 g, 0.61 mmol) and sodium azide (0.20 g, 3.0 mmol), gave the desired product 0.17 g (89%) of the title compound was obtained as a colorless oil. MS EI m/e 315 M⁺.

### Intermediate 61

(S)-2-Azidomethyl-8-(4-chloro-phenyl)-2-methyl-2,3-dihydro-benzo[1,4]dioxine: Starting from (R)-toluene-4-sulfonic acid 8-(4-chloro-phenyl)-2-methyl-2,3-dihydro-benzo[1,4]-dioxin-2-yl-methyl ester (0.27 g, 0.61 mmol) and sodium azide (0.20 g, 3.0 mmol), 0.18 g (94%) of the title compound was obtained as a colorless oil. MS EI m/e 315 M⁺.

### Intermediate 62

(S)-2-Azidomethyl-8-(2-methoxy-phenyl)-2-methyl-2,3-dihydro-benzo[1,4]dioxine: Starting from (R)-toluene-4-sulfonic acid 8-(2-methoxy-phenyl)-2-methyl-2,3-dihydro-benzo[1,4]-dioxin-2-yl-methyl ester (0.28 g, 0.63 mmol) and sodium azide (0.21 g, 3.2 mmol), 0.13 g (66%) of the title compound was obtained as a colorless oil. MS EI m/e 311 M⁺.

### Intermediate 63

(S)-2-Azidomethyl-2-methyl-8-thiophen-3-yl-2,3-dihydro-benzo[1,4]dioxine: Starting from (R)-toluene-4-sulfonic acid 8-thiophen-3-yl-2,3-dihydro-benzo[1,4]-dioxin-2-yl-methyl ester (0.22 g, 0.53 mmol) and sodium azide (0.17 g, 2.6 mmol), 0.13 g (86%) of the title compound was obtained as a colorless oil. MS EI m/e 287 M⁺.

### Intermediate 64

(S)-2-Azidomethyl-8-(2-chloro-phenyl)-6-chloro-2,3-dihydro-benzo[1,4]dioxine: Starting from (R)-toluene-4-sulfonic acid 8-(2-chloro-phenyl)-6-chloro-2,3-dihydro-benzo[1,4]dioxin-2-yl methyl ester (420 mg, 0.90 mmol), 0.29 g (96%) of the title compound was obtained as a colorless oil.

### Intermediate 65

(S)-2-Azidomethyl-8-(2-fluoro-phenyl)-6-chloro-2,3-dihydro-benzo[1,4]dioxine: Starting from (R)-toluene-4-sulfonic acid 8-(2-fluoro-phenyl)-6-chloro-2,3-dihydro-benzo[1,4]dioxin-2-yl methyl ester (350 mg, 0.72 mmol), 0.23 g (100%) of the title compound was obtained as a colorless oil.

### Intermediate 66

(S)-2-Azidomethyl-8-(2-methyl-phenyl)-6-chloro-2,3-dihydro-benzo[1,4]dioxine: Starting from (R)-toluene-4-sulfonic acid 8-(2-methyl-phenyl)-6-chloro-2,3-dihydro-benzo[1,4]dioxin-2-yl methyl ester (380 mg, 0.85 mmol), 0.26 g (96%) of the title compound was obtained as a colorless oil.

### Intermediate 67

(S)-2-Azidomethyl-8-(2-methoxy-phenyl)-6-chloro-2,3-dihydro-benzo[1,4]dioxine: Starting from (R)-toluene-4-sulfonic acid 8-(2-methoxy-phenyl)-6-chloro-2,3-dihydro-benzo[1,4]dioxin-2-yl methyl ester (440 mg, 0.95 mmol), 0.28 g (88%) of the title compound was obtained as a colorless oil.

### Intermediate 68

(S)-2-Azidomethyl-8-(2-trifluoromethyl-phenyl)-6-chloro-2,3-dihydro-benzo[1,4]dioxine: Starting from (R)-toluene-4-sulfonic acid 8-(2-trifluoromethyl-phenyl)-6-chloro-2,3-dihydro-benzo[1,4]dioxin-2-yl methyl ester (410 mg, 0.82 mmol), 0.23 g (76%) of the title compound was obtained as a colorless oil.

### Intermediate 69

(S)-2-Azidomethyl-8-(2,3-dimethoxy-phenyl)-6-chloro-2,3-dihydro-benzo[1,4]dioxine: Starting from (R)-toluene-4-sulfonic acid 8-(2,3-dimethoxy-phenyl)-6-chloro-2,3-dihydro-benzo[1,4]dioxin-2-yl methyl ester (400 mg, 0.81 mmol), 0.28 g (95%) of the title compound was obtained as a colorless oil.

### Intermediate 70

(S)-2-Azidomethyl-8-(2,4-dichloro-phenyl)-6-chloro-2,3-dihydro-benzo[1,4]dioxine: Starting from (R)-toluene-4-sulfonic acid 8-(2,4-dichloro-phenyl)-6-chloro-2,3-dihydro-benzo[1,4]dioxin-2-yl methyl ester (360 mg, 0.72 mmol), 0.29 g (92%) of the title compound was obtained as a colorless oil.

### Intermediate 71

(S)-2-Azidomethyl-8-(4-chloro-2-methyl-phenyl)-6-chloro-2,3-dihydro-benzo[1,4]dioxine: Starting from (R)-toluene-4-sulfonic acid 8-(4-chloro-2-methyl-phenyl)-6-chloro-2,3-dihydro-benzo[1,4]dioxin-2-yl methyl ester (400 mg, 0.83 mmol), 0.29 g (100%) of the title compound was obtained as a colorless oil.

### Intermediate 72

(S)-2-Azidomethyl-8-(2,4-di-trifluoromethyl-phenyl)-6-chloro-2,3-dihydro-benzo[1,4]dioxine: Starting from (R)-toluene-4-sulfonic acid 8-(2,4-di-trifluoromethyl-phenyl)-6-chloro-2,3-dihydro-benzo[1,4]dioxin-2-yl methyl ester (420 mg, 0.74 mmol), 0.33 g (98%) of the title compound was obtained as a colorless oil.

### Intermediate 73

(S)-2-Azidomethyl-8-(2,5-dichloro-phenyl)-6-chloro-2,3-dihydro-benzo[1,4]dioxine: Starting from (R)-toluene-4-sulfonic acid 8-(2,5-dichloro-phenyl)-6-chloro-2,3-dihydro-benzo[1,4]dioxin-2-yl methyl ester (390 mg, 0.78 mmol), 0.28 g (97%) of the title compound was obtained as a colorless oil.

### Intermediate 74

(S)-2-Azidomethyl-8-(5-chloro-2-methoxy-phenyl)-6-chloro-2,3-dihydro-benzo[1,4]dioxine: Starting from (R)-toluene-4-sulfonic acid 8-(5-chloro-2-methoxy-phenyl)-6-chloro-2,3-dihydro-benzo[1,4]dioxin-2-yl methyl ester (400 mg, 0.81 mmol), 0.29 g (98%) of the title compound was obtained as a colorless oil.

### Intermediate 75

(S)-2-Azidomethyl-8-(2,6-di-methyl-phenyl)-6-chloro-2,3-dihydro-benzo[1,4]dioxine: Starting from (R)-toluene-4-sulfonic acid 8-(2,6-dimethyl-phenyl)-6-chloro-2,3-dihydro-benzo[1,4]dioxin-2-yl methyl ester (530 mg, 1.15 mmol), 0.38 g (100%) of the title compound was obtained as a colorless oil.

### Intermediate 76

2′,6′-Dichloro-5-fluoro-2methoxy-biphenyl: To a solution of 2,6-dichlorobromobenzene (3.5 g, 15.7 mmol) and sodium hydroxide (3.14 g, 78.5 mmol) in DME-water (2:1) was added 5-fluoro-2-methoxybenzene boronic acid (4.0 g, 23.5 mmol) at 90° C., followed by tetrakis(triphenylphosphine)palladium (0) (0.9 g, 0.78 mmol). The reaction mixture was heated at 90° C. overnight and cooled to room temperature. The mixture was extracted with methylene chloride and washed with water. The organic solvent was removed under vacuum. Chromatography with 5% ethyl acetate in hexanes afforded 2.62 g (87%) of the title compound as a colorless oil. MS EI m/e 270 M⁺.

### Intermediate 77

3-Bromo-2′,6′-dichloro-5-fluoro-2-methoxy-biphenyl: To a solution of 2′,6′-dichloro-5-fluoro-2-methoxy-biphenyl (5.73 g, 21 mmol) in acetic acid (100 mL) was added iron powder (cat. amount) and bromine (3.3 mL, 63 mmol) slowly at room temperature. The reaction mixture was stirred at 60° C. overnight. The acetic acid was removed under vacuum. The residue was washed with methylene chloride and saturated sodium sulfite. The organic layer was combined and washed with more sodium sulfite solution and dried over anhydrous sodium sulfate. The solvent was removed under vacuum. Chromatography with 5% ethyl acetate in hexanes afforded 6.28 g (85%) of the title compound as a light yellow oil.

### Intermediate 78

2′,6′-Dichloro-5-fluoro-2-methoxy-biphenyl-3-carbaldehyde: To a solution of 3-bromo-2′,6′-dichloro-5-fluoro-2-methoxy-biphenyl (5.5 g, 16 mmol) in anhydrous tetrahydrofuran was added i-PrMgCl (2.0 M in hexane, 12 mL, 24 mmol) at 0° C. The resulting mixture was stirred at 0° C. for hours until no more starting material was present. Then 1-formylpiperidine (2.3 mL, 20.8 mmol) was introduced at −30° C. The reaction mixture was stirred at −30° C. to 10° C. overnight. The reaction was quenched with 2N HCl and extracted with methylene chloride. The solvent was removed under vacuum. Chromatography with 30% ethyl acetate in hexane afforded the title compound, (4.69 g, 99%) as a colorless oil. MS EI m/e 298 M⁺;

Elemental Anal. for C₁₄H₉FO₂Cl₂

Theory: C, 56.21; H, 3.03.

Found: C, 55.90; H, 3.03.

### Intermediate 79

2′,6′-Dichloro-5-fluoro-2-methoxy-biphenyl-3-ol: To a solution of 2′,6′-dichloro-5-fluoro-2-methoxy-biphenyl-3-carbaldehyde (2.35 g, 7.8 mmol) in methylene chloride (100 mL) was added m-CPBA (77% max, 4.2 g) slowly at room temperature. The reaction mixture was stirred at room temperature overnight. The white solid was filtered off. The reaction mixture was quenched with 10% sodium sulfite and 10% sodium bicarbonate at 0° C. and extracted with methylene chloride. The organic layer was washed with brine and dried over anhydrous sodium sulfate. The solvent was removed under vacuum to afford the crude material as a light yellow oil. To a solution of the crude material in methanol was added sodium hydroxide (1.25 g, 31.2 mmol) at 0° C. The reaction mixture was stirred at room temperature for 2 h, then poured into ice-water. The mixture was neutralized with concentrated hydrochloric acid, and the mixture was extracted with methylene chloride and washed with water. The solvent was removed under vacuum. Chromatography with 30% ethyl acetate in hexanes afforded 1.38 g (79%) of the title compound as a white solid; mp 65-67° C. MS ESI m/e 285.0 [M−H]⁻

Elemental Anal. for C₁₃H₉FO₂Cl₂

Theory: C, 54.38; H, 3.16.

Found: C, 54.15; H, 3.03.

### Intermediate 80

(R)-2-(2′,6′-Dichloro-5-fluoro-2-methoxy-biphenyl-3-yloxymethyl)-oxirane: To a suspension of sodium hydride (60%, 0.62 g, 15.4 mmol) in DMF was added 2′,6′-dichloro-5-fluoro-2-methoxy-biphenyl-3-ol (2.95 g, 10.2 mmol) at 0° C. The mixture was stirred at room temperature for 30 min. Then a solution of the (R)-(−)-glydicyl tosylate (4.7 g, 20.4 mmol) in DMF was introduced at room temperature. The resulting mixture was heated at 100° C. overnight and poured into ice-water. The mixture was extracted with methylene chloride. The organic layer was washed with water and dried over anhydrous sodium sulfate and filtered. The solvent was removed under vacuum. Chromatography with 20% ethyl acetate in hexanes afforded 2.73 g (77%) of title compound as a colorless oil. [α]=−13.2° (c 1% solution, MeOH); HRMS ESI m/e 360.0573 (M+NH₄)⁺

### Intermediate 81

(S)-3-(3-Bromo-2-hydroxy-propoxy)-2′,6′-dichloro-5-fluoro-biphenyl-2-ol (81-1); (S)-Acetic acid 1-bromomethyl-2-(2′,6′-dichloro-5-fluoro-2-hydroxy-biphenyl-3-yloxy)-ethyl ester (81-2): A solution of (R)-2-(2′,6′-dichloro-5-fluoro-2-methoxy-biphenyl-3-yloxymethyl)-oxirane (0.42 g 1.2 mmol) in 33% HBr in acetic acid (15 mL) was heated at 65° C. for 1 h. The mixture was poured into ice-water and extracted with methylene chloride. The organic layer was washed with water and dried over sodium sulfate and filtered. The organic solvent was removed under vacuum. Chromatography with 20-60% ethyl acetate in hexanes afforded 0.3 g (60%) of the title compound (54-1), HRMS ESI m/e 425.9678 [M+NH₄]⁺; and 0.22 (39%) of the product (54-2), HRMS ESI m/e 467.9789 [M+NH₄]⁺ as colorless oils.

### Intermediate 82

(S)-[8-(2,6-Dichlorophenyl)-6-fluoro-2,3-dihydro-benzo[1,4]dioxin-2-yl]-methanol: To a solution mixture of (S)-3-(3-bromo-2-hydroxy-propoxy)-2′,6′-dichloro-5-fluoro-biphenyl-2-ol (0.3 g) and (S)-acetic acid 1-bromomethyl-2-(2′,6′-dichloro-5-fluoro-2-hydroxy-biphenyl-3-yloxy)-ethyl ester (0.22 g) in methanol (30 mL) at 0° C. was added 2.5 N NaOH (10 mL). The resulting mixture was stirred at 0° C. for 1 h. Then the mixture was extracted with methylene chloride and washed with water. The solvent was removed under vacuum. Chromatography with 20-60% ethyl acetate in hexanes afforded 0.36 g of the title compound as a colorless oil. HRMS EI m/e 328.0056 M⁺; [α]=+31.60 (c 1% solution in Methanol).

### Intermediate 83

(R)-Toluene-4-sulfonic acid 8-(2,6-dichlorophenyl)-6-fluoro-2,3-dihydro-benzo[1,4]dioxin-2-yl-methyl ester: To a solution of (S)-[8-(2,6-dichlorophenyl)-6-fluoro-2,3-dihydro-benzo[1,4]dioxin-2-yl]-methanol (1.38 g, 4.2 mmol) in methylene chloride (60 mL) was added p-toluenesulfonyl chloride (1.2 g, 6.3 mmol), diisopropylethylamine (2.2 mL, 12.6 mmol) and DMAP (catalytical amount) at room temperature. The resulting mixture was stirred at room temperature for 24 h. Then the reaction was quenched with ice-water and extracted with methylene chloride. The organic layer was washed with water and dried over anhydrous sodium sulfate and filtered. The solvent was removed under vacuum. Chromatography with 10-40% ethyl acetate in hexanes afforded 1.78 g (88%) of the title compound as a thick colorless oil. HRMS ESI m/e 500.0505 [M+NH₄]⁺; [α]=+33.26 (c 6.4 mg in 0.7 mL methanol).

### Intermediate 84

(S)-2-Azidomethyl-8-(2,6-dichlorophenyl)-6-fluoro-2,3-dihydro-benzo[1,4]dioxin: To a solution of (R)-toluene-4-sulfonic acid 8-(2,6-dichlorophenyl)-6-fluoro-2,3-dihydro-benzo[1,4]dioxin-2-yl-methyl ester (0.40 g, 0.83 mmol) in DMF was added sodium azide (0.27 g, 4.1 mmol) at room temperature. The resulting mixture was heated at 90° C. overnight. The reaction mixture was poured in water and extracted with methylene chloride. The organic layer was washed with water and dried over anhydrous sodium sulfate and filtered. The solvent was removed. Chromatography with 0-30% ethyl acetate in hexanes afforded 0.26 g (89%) of the title compound as a colorless oil. HRMS EI m/e 353.0125 M⁺; [α]=+39.4° (c 1% solution in methanol).

### Intermediate 85

2′,6′-Dichloro-2-methoxy-biphenyl: To a solution of 2,6-dichlorobenzene bromide (22.9 g, 87 mmol) and sodium hydroxide (10.1 g, 0.22 mol) in DME-water (2:1) was added 2-methoxy benzene boronic acid (20 g, 0.13 mol) at 90° C., followed by tetrakis(triphenylphosphine)-palladium (0) (5.8 g, 4.3 mmol). The reaction mixture was heated at 90° C. overnight and cooled to room temperature. The mixture was extracted with methylene chloride and washed with water. The organic solvent was removed under vacuum. Chromatography with 5% ethyl acetate in hexanes afforded 23.57 g (93%) of the title compound as a colorless oil. MS EI m/e 252 M⁺

### Intermediate 86

2,2′-Dichloro-6-methoxy-biphenyl: To a solution of 2-chlorobromobenzene (15.5 g, 80.6 mmol) and sodium carbonate (9.0 g, 84.9 mmol) in DME-water (5:1) was added 2-chloro-6-methoxybenzene boronic acid (5.0 g, 26.8 mmol) at 82° C., followed by tetrakis(triphenylphosphine)palladium (0) (1.5 g, 1.4 mmol). The reaction mixture was heated at 82° C. overnight and cooled to room temperature. The mixture was extracted with ethyl acetate and washed with water. The organic solvent was removed under vacuum. Chromatography with 5% ethyl acetate in hexanes afforded 5.0 g (73%) of the title compound as a colorless oil.

### Intermediate 87

6-Chloro-2-methoxy-2′-methyl-biphenyl: This intermediate was prepared by the same procedure as for 2,2′-dichloro-6-methoxy-biphenyl (Intermediate 86). Starting from 2-chloro-6-methoxy benzene boronic acid (5.0 g, 26.9 mmol) and 2-methylbromobenzene (13.8 g, 80.6 mmol), afforded 3.85 g (62%) of the title compound was obtained as a colorless oil.

### Intermediate 88

6-Chloro-2-methoxy-2′-trifluoromethyl-biphenyl: This intermediate was prepared by the same procedure as for 2,2′-dichloro-6-methoxy-biphenyl (Intermediate 86). Starting from 2-chloro-6-methoxy benzene boronic acid (5.0 g, 26.9 mmol) and 2-trifluoromethylbromobenzene (12.0 g, 53.8 mmol), afforded 1.6 g (21%) of the title compound as a colorless oil.

### Intermediate 89

2′-Chloro-2-fluoro-6-methoxy-biphenyl: This intermediate was prepared by the same procedure as for 2,2′-dichloro-6-methoxy-biphenyl (Intermediate 86). Starting from 2-fluoro-6-methoxy benzene boronic acid (10.0 g, 58.8 mmol) and 2-chlorobromobenzene (14.8 g, 77.6 mmol), 17.0 g of the title compound was obtained as a colorless oil.

### Intermediate 90

6-Fluoro-2-methoxy-2′-methyl-biphenyl: This intermediate was prepared by the same procedure as for 2,2′-dichloro-6-methoxy-biphenyl (Intermediate 86). Starting from 2-fluoro-6-methoxy benzene boronic acid (5.0 g, 29.4 mmol) and 2-methylbromobenzene (10.1 g, 58.8 mmol), 2.35 g (37%) of the title compound was obtained as a colorless oil.

### Intermediate 91

2,4-Dichloro-6′-fluoro-2′-methoxy-biphenyl: This intermediate was prepared by the same procedure as for 2,2′-dichloro-6-methoxy-biphenyl (Intermediate 86). Starting from 2-fluoro-6-methoxy benzene boronic acid (5.0 g, 29.4 mmol) and 2,4-dichlorobromobenzene (13.8 g, 61.2 mmol), 3.3 g (42%) of the title compound was obtained as a colorless oil.

### Intermediate 92

2′,6′-Dichloro-biphenyl-2-ol: To a solution of 2′,6′-dichloro-2-methoxybiphenyl (23.57 g, 93 mmol) in methylene chloride was added boron tribromide (13.2 mL, 0.14 mol) at −78° C. The resulting mixture was stirred at −78° C. to room temperature overnight. The reaction mixture was poured in the ice-NH₄OH and extracted with methylene chloride. The organic layer was washed with water and dried over anhydrous and filtered. The solvent was removed under vacuum. Chromatography with 10-40% ethyl acetate in hexanes afforded 21.65 g (97%) of the title compound as a colorless oil. MS ESI m/e 236.99 [M−H]⁺

### Intermediate 93

2′,6-Dichloro-biphenyl-2-ol: 2,2′-dichloro-6-methoxy-biphenyl (5.0 g, 20.9 mmol) was heated in hydrogen bromide in acetic acid (60 mL, 33%) at 65° C. overnight. The resulting mixture was cooled to room temperature. The reaction mixture was poured in water and extracted with ethyl acetate. The organic layer was washed with water and dried over anhydrous sodium sulfate and filtered. The solvent was removed under vacuum. Chromatography with 10-40% ethyl acetate in hexanes afforded 4.2 g (84%) of the title compound as a colorless oil.

### Intermediate 94

2′-Chloro-6-fluoro-biphenyl-2-ol: This intermediate was prepared by the same procedure as described for 2′,6-dichloro-biphenyl-2-ol (Intermediate 93). Starting from 2′-chloro-2-fluoro-6-methoxy-biphenyl (17.0 g), 7.5 g (57% for two steps) of the title compound was obtained as a colorless oil.

### Intermediate 95

6-Chloro-2′-methyl-biphenyl-2-ol: This intermediate was prepared by the same procedure as for 2′,6-dichloro-biphenyl-2-ol (Intermediate 93). Starting from 6-chloro-2-methoxy-2′-methyl-biphenyl (15.0 g), 10.9 g (77%) of the title compound was obtained as a colorless oil.

### Intermediate 96

6-Chloro-2′-trifluoromethyl-biphenyl-2-ol: This intermediate was prepared by the same procedure as for 2′,6-dichloro-biphenyl-2-ol (Intermediate 93). Starting from 6-chloro-2-methoxy-2′-trifluoromethyl-biphenyl (1.6 g), 1.3 g (92%) of the title compound was obtained as a colorless oil.

### Intermediate 97

6-Fluoro-2′-methyl-biphenyl-2-ol: This intermediate was prepared by the same procedure as for 2′,6-dichloro-biphenyl-2-ol (Intermediate 93). Starting from 6-fluoro-2-methoxy-2′-methyl-biphenyl (6.2 g, 28.7 mmol), 6.0 g (100%) of the title compound was obtained as a colorless oil.

### Intermediate 98

2′,4′-Dichloro-6-fluoro-biphenyl-2-ol: This intermediate was prepared by the same procedure as for 2′,6-dichloro-biphenyl-2-ol (Intermediate 93). Starting from 2,4-dichloro-6′-fluoro-2′-methoxy-biphenyl (5.0 g, 18.4 mmol), 4.2 g (89%) of the title compound was obtained as a colorless oil.

### Intermediate 99

2-Allyloxy-2′,6′-dichloro-biphenyl: To a solution of 2′,6′-dichloro-biphenyl-2-ol (21.65 g, 90 mmol) in DMF was added allyl bromide (11.75 mL, 0.135 mol) and potassium carbonate (31.23 g, 0.225 mol) at room temperature. The resulting mixture was stirred at room temperature overnight and poured into water. The mixture was extracted with methylene chloride and washed with water. The solvent was removed under vacuum. Chromatography with 0-30% ethyl acetate in hexanes afforded 24.8 g (98%) of the title compound as a light yellow oil. MS EI m/e 278 M⁺

### Intermediate 100

6-Allyloxy-2,2′-dichloro-biphenyl: To a solution of 2′,6-dichloro-biphenyl-2-ol (10.0 g, 41.8 mmol) in DMF was added sodium hydride (60% in mineral oil, 2.5 g, 62.7 mmol) and allyl bromide (5.4 mL, 62.7 mmol) at room temperature. The resulting mixture was stirred at room temperature overnight. The mixture was extracted with ethyl acetate and washed with water. The solvent was removed under vacuum. Chromatography with 0-30% ethyl acetate in hexanes afforded 11.6 g (100%) of the title compound as a light yellow oil.

### Intermediate 101

6-Allyloxy-2′-chloro-2-fluoro-biphenyl: This intermediate was prepared by the same procedure as described for 6-allyloxy-2,2′-dichloro-biphenyl (Intermediate 100). Starting from 2′-chloro-6-fluoro-biphenyl-2-ol (7.5 g, 33.7 mmol), 9.0 g (100%) of the title compound was obtained as a colorless oil.

### Intermediate 102

3-Allyl-2′,6′-dichloro-biphenyl-2-ol: A solution of 2-allyloxy-2′,6′-dichloro-biphenyl (24.8 g, 88.7 mmol) in decahydronaphthalene (100 mL) was refluxed for 24 h. The solvent was removed under vacuum. Chromatography with 0-20% ethyl acetate in hexanes afforded 23 g (93%) of the title compound as a light yellow oil. MS ESI m/e 278.9 [M+H]⁺

### Intermediate 103

3-Allyl-2′,6-dichloro-biphenyl-2-ol: A solution of 6-allyloxy-2,2′-dichloro-biphenyl (11.6 g, 41.8 mmol) in mesitylene (100 mL) was refluxed for 24 h. The solvent was removed under vacuum. Chromatography with 0-20% ethyl acetate in hexanes afforded 9.0 g (77%) of the title compound as a light yellow oil.

### Intermediate 104

3-Allyl-2′-chloro-6-fluoro-biphenyl-2-ol: This intermediate was prepared by the same procedure as described for 3-allyl-2′ 6-dichloro-biphenyl-2-ol (Intermediate 103). Starting from 6-allyloxy-2′-chloro-2-fluoro-biphenyl (9.0 g, 33.7 mmol), 7.0 g (81%) of the title compound was obtained as a colorless oil.

### Intermediate 105

3-Allyl-2-benzyloxy-2′,6′-dichloro-biphenyl: To a suspension of sodium hydride (60%, 2.5 g, 61.5 mmol) in DMF at 0° C. was added a solution of 3-allyl-2′,6′-dichloro-biphenyl-2-ol (11.47 g, 41 mmol) in DMF. The resulting mixture was stirred at room temperature for 1 h, then benzyl bromide (7.33 mL, 61.5 mmol) was introduced at room temperature. The resulting mixture was stirred at 60° C. overnight and poured into ice water. The mixture was extracted with methylene chloride and washed with water. The solvent was removed under vacuum. Chromatography with 0-30% ethyl acetate in hexanes afforded 15.08 g (99%) of the title compound as a light yellow oil. MS ESI m/e 386.1 [M+NH⁴]⁺

### Intermediate 106

2-Benzyloxy-2′,6′-dichloro-3-propenyl-biphenyl: A solution of 3-allyl-2-benzyloxy-2′,6′-dichloro-biphenyl (15.08 g, 41 mmol) and bis(acetonitrile)dichloropalladium (II) (0.53 g, 2.1 mmol) in methylene chloride was refluxed for 24 h. The solvent was removed under vacuum. Chromatography with 0-30% ethyl acetate in hexanes afforded 14.63 g (97%) of the title compound as a colorless oil. MS ESI m/e 369.1 [M+H]⁺

### Intermediate 107

2′,6-Dichloro-3-propenyl-biphenyl-2-ol: To a solution of 3-allyl-2′,6-dichloro-biphenyl-2-ol (6.2 g, 22.2 mmol) in methylene chloride (100 mL) was added dichlorobis(acetonitrile)-palladium(II) (0.86 g, 3.3 mmol). The resulting mixture was refluxed overnight. The solvent was removed under vacuum. Chromatography with 5-20% ethyl acetate in hexanes afforded 3.0 g (48%) of the title compound as a light yellow oil.

### Intermediate 108

2′-Chloro-6-fluoro-3-propenyl-biphenyl-2-ol: This intermediate was prepared by the same procedure as described for 2′,6-dichloro-3-propenyl-biphenyl-2-ol (Intermediate 107). Starting from 3-allyl-2′-chloro-6-fluoro-biphenyl-2-ol (3.8 g, 14.5 mmol), 1.5 g (39%) of the title compound was obtained as a colorless oil.

### Intermediate 109

2-Benzyloxy-2′,6′-dichloro-biphenyl-3-carbaldehyde: To a solution of 2-benzyloxy-2′,6′-dichloro-3-propenyl-biphenyl (5.0 g, 13.5 mmol) in methanol (50 mL) and water (7.5 mL) was added osmium tetroxide solution (4% in water, 1.7 mL) and sodium periodate (8.7 g, 40.5 mmol) at 0° C. The resulting mixture was stirred at 0° C. for 2 h and poured in ice water. The mixture was extracted with methylene chloride and washed with water. The organic layer was dried over anhydrous sodium sulfate and filtered. Chromatography with 0-40% ethyl acetate in hexanes afforded 3.71 g (77%) of the title compound as a colorless oil. MS ESI m/e 357.0 [M+H]⁺

### Intermediate 110

2-Benzyloxy-2′,6-dichloro-biphenyl-3-carbaldehyde: To a solution of 2′,6-dichloro-3-propenyl-biphenyl-2-ol (3.0 g, 10.7 mmol) in THF (80 mL) and water (10 mL) was added osmium tetroxide solution (4% in water, 2.5 mL) and sodium periodate (7.2 g, 33.6 mmol) at 0° C. The resulting mixture was stirred at 0° C. for 2 h and poured in ice water. The mixture was extracted with ethyl acetate and washed with water. The organic layer was dried over anhydrous sodium sulfate and filtered. The solution was concentrated to provided 2′,6-dichloro-2-hydroxy-biphenyl-3-carbaldehyde. This was further treated with sodium hydride (0.54 g, 13.5 mmol) and benzyl bromide (1.5 mL, 13.5 mmol) in DMF at room temperature overnight. The mixture was extracted with ethyl acetate and washed with water. The solvent was removed under vacuum. Chromatography with 0-25% ethyl acetate in hexanes afforded 2.0 g (52% for 2 steps) of the title compound as a light yellow oil, which hardened into an off-white solid.

### Intermediate 111

2-Benzyloxy-2′-chloro-6-fluoro-biphenyl-3-carbaldehyde: This intermediate was prepared by the same procedure as described for 2-benzyloxy-2′,6-dichloro-biphenyl-3-carbaldehyde (Intermediate 110). Starting from 2′-chloro-6-fluoro-3-propenyl-biphenyl-2-ol (4.0 g, 15.2 mmol), 2.5 g (48% for two steps) of the title compound was obtained.

### Intermediate 112

6-Chloro-2-hydroxy-2′-methylbiphenyl-3-carbaldehyde: To a solution of 6-chloro-2′-methylbiphenyl-2-ol (2.18 g, 10.0 mmol) in chloroform (10 mL) and water (0.36 mL) was added sodium hydroxide (2.0 g, 50.0 mmol). The reaction mixture was heated at 55° C. for 4 h. The resulting mixture was cooled to room temperature and neutralized with 1N HCl. The mixture was extracted with ethyl acetate and washed with water. The solvent was removed under vacuum. Chromatography with 0-25% ethyl acetate in hexanes afforded 0.65 g (26%) of the title compound.

### Intermediate 113

6-Chloro-2-hydroxy-2′-(trifluoromethyl)biphenyl-3-carbaldehyde: To a solution of 6-chloro-2′-trifluoromethylbiphenyl-2-ol (1.4 g, 5.13 mmol) in methanol (5 mL) was added magnesium methoxide (6-10% in methanol, 6.0 mL, ˜6 mmol), the mixture was heated at 85° C. and solvent distilled off. Toluene (10 mL) was added and the resulting mixture heated at 85° C. for 2 h. while distilling off the low boiling point by-product and this was followed by adding paraformaldehyde (0.48 g, 15.4 mmol). The resulting mixture was heated with concurrent removal of volatile materials under reduced pressure for 1.5 h. The mixture was cooled to room temperature and treated with 10% sulfuric acid with care until slightly acidic. The mixture was extracted with ethyl acetate and washed with water. The solvent was removed under vacuum. Chromatography with 0-25% ethyl acetate in hexanes afforded 0.72 g (47%) of the title compound. MS ES m/z 299.0 [M−H]⁻.

### Intermediate 114

6-Fluoro-2-hydroxy-2′-methylbiphenyl-3-carbaldehyde: This intermediate was prepared by the same procedure as for 6-chloro-2-hydroxy-2′-(trifluoromethyl)biphenyl-3-carbaldehyde (Intermediate 113). Starting from 6-fluoro-2′-methylbiphenyl-2-ol (2.0 g, 10.0 mmol), 1.21 g (53%) of the title compound was obtained. MS ES m/z 231.1 [M+H]⁺ m/z 229.1 [M−H]⁻.

### Intermediate 115

2′,4′-Dichloro-6-fluoro-2-hydroxybiphenyl-3-carbaldehyde: This intermediate was prepared by the same procedure as for 6-chloro-2-hydroxy-2′-(trifluoromethyl)biphenyl-3-carbaldehyde (Intermediate 113). Starting from 2′,4′-dichloro-6-fluorobiphenyl-2-ol (2.0 g, 7.78 mmol), 1.35 g (61%) of the title compound was obtained.

### Intermediate 116

2-(Benzyloxy)-6-chloro-2′-methylbiphenyl-3-carbaldehyde: To a solution of 6-chloro-2-hydroxy-2′-methylbiphenyl-3-carbaldehyde (1.54 g, 6.24 mmol) in DMF (10 mL) was added sodium hydride (60% in mineral oil, 0.37 g, 9.36 mmol) followed by benzyl bromide (0.96 mL, 8.15 mmol). The resulting mixture was stirred at room temperature overnight. The mixture was extracted with ethyl acetate and washed with water. The solvent was removed under vacuum. Chromatography with 0-25% ethyl acetate in hexanes afforded 2.0 g (95%) of the title compound.

### Intermediate 117

2-(Benzyloxy)-6-fluoro-2′-methylbiphenyl-3-carbaldehyde: This intermediate was prepared by the same procedure as for 2-(benzyloxy)-6-chloro-2′-methylbiphenyl-3-carbaldehyde (Intermediate 117). Starting from 6-fluoro-2-hydroxy-2′-methylbiphenyl-3-carbaldehyde (1.2 g, 5.21 mmol), 60.88 g (53%) of the title compound was obtained as a colorless oil.

### Intermediate 118

2-(Benzyloxy)-6-chloro-2′-(trifluoromethyl)biphenyl-3-carbaldehyde: This intermediate was prepared by the same procedure as for 2-(benzyloxy)-6-chloro-2′-methylbiphenyl-3-carbaldehyde (Intermediate 116). Starting from 6-chloro-2-hydroxy-2′-(trifluoromethyl)biphenyl-3-carbaldehyde (0.73 g, 2.42 mmol), 0.7 g (74%) of the title compound was obtained as a white solid.

### Intermediate 119

2-(Benzyloxy)-2′,4′-dichloro-6-fluorobiphenyl-3-carbaldehyde: This intermediate was prepared by the same procedure as for 2-(benzyloxy)-6-chloro-2′-methylbiphenyl-3-carbaldehyde (Intermediate 116). Starting from 2′,4′-dichloro-6-fluoro-2-hydroxybiphenyl-3-carbaldehyde (1.35 g, 4.73 mmol), 1.4 g (79%) of the title compound was obtained.

### Intermediate 120

2-Benzyloxy-2′,6′-dichloro-biphenyl-3-ol: To a solution of 2-benzyloxy-2′,6′-dichloro-biphenyl-3-carbaldehyde (3.71 g, 10 mmol) was added m-CPBA (77% max, 5.8 g) in methylene chloride (100 mL). The resulting mixture was stirred at room temperature overnight and quenched with 10% sodium sulfite and 10% sodium bicarbonate. The mixture was extracted with methylene chloride and washed with water. The solvent was removed under vacuum to afford a crude material as a light yellow oil. To a solution of crude oil in methanol was added sodium hydroxide (1.66 g, 40 mmol) at room temperature. The mixture was stirred at room temperature for 2 h and neutralized with concentrated hydrochloric acid. The mixture was extracted with methylene chloride and washed with water. The organic solvent was removed under vacuum. Chromatography with 1040% ethyl acetate in hexanes afforded 2.70 g (75%) of the title compound as a colorless oil. MS ESI m/e 343.0 [M−H]⁻

### Intermediate 121

2-Benzyloxy-2′,6-dichloro-biphenyl-3-ol: To a solution of 2-benzyloxy-2′,6-dichloro-biphenyl-3-carbaldehyde (2.0 g, 5.6 mmol) was added m-CPBA (77% max, 3.5 g) in methylene chloride (100 mL). The resulting mixture was stirred at room temperature overnight and quenched with 1:1 10% sodium sulfite in saturated sodium bicarbonate. The mixture was extracted with methylene chloride and washed with water. The solvent was removed under vacuum to afford a crude material as a light yellow oil. To a solution of the crude oil in methanol was added sodium bicarbonate (1.0 g, 12 mmol) at room temperature. The mixture was stirred at room temperature for 2 h and the solvent was removed under vacuum. The mixture was extracted with ethyl acetate and washed with water. The organic solvent was removed under vacuum. Chromatography with 0-40% ethyl acetate in hexanes afforded 1.67 g (86%) of the title compound as a colorless oil.

### Intermediate 122

2-Benzyloxy-2′-chloro-6-fluoro-biphenyl-3-ol: This intermediate was prepared by the same procedure as described for 2-benzyloxy-2′,6-dichloro-biphenyl-3-ol (Intermediate 121). Starting from 2-benzyloxy-2′-chloro-6-fluoro-biphenyl-3-carbaldehyde (2.5 g, 7.3 mmol), 1.4 g (58%) of the product was obtained as a colorless oil.

### Intermediate 123

2-(Benzyloxy)-6-chloro-2′-methylbiphenyl-3-ol: This intermediate was prepared by the same procedure as for 2-benzyloxy-2′,6-dichloro-biphenyl-3-ol (Intermediate 121). Starting from 2-(benzyloxy)-6-chloro-2′-methylbiphenyl-3-carbaldehyde (2.0 g, 5.94 mmol), 1.2 g (62%) of the title compound was obtained as a colorless oil.

### Intermediate 124

2-(Benzyloxy)-6-chloro-2′-(trifluoromethyl)biphenyl-3-ol: This intermediate was prepared by the same procedure as for 2-benzyloxy-2′,6-dichloro-biphenyl-3-ol (Intermediate 121). Starting from 2-(benzyloxy)-6-chloro-2′-(trifluoromethyl)biphenyl-3-carbaldehyde (0.7 g, 1.79 mmol), 0.7 g (100%) of the title compound was obtained.

### Intermediate 125

2-Benzyloxy-6-fluoro-2′-methyl-biphenyl-3-ol: This intermediate was prepared by the same procedure as for 2-benzyloxy-2′,6-dichloro-biphenyl-3-ol (Intermediate 121). Starting from 2-benzyloxy-6-fluoro-2′-methyl-biphenyl-3-carbaldehyde (0.88 g, 2.75 mmol), 0.6 g (71%) of the title compound was obtained as a white solid. MS ES m/z 307.1 [M−H]⁻.

### Intermediate 126

2-(Benzyloxy)-2′,4′-dichloro-6-fluorobiphenyl-3-ol: This intermediate was prepared by the same procedure as for 2-benzyloxy-2′,6-dichloro-biphenyl-3-ol (Intermediate 121). Starting from 2-(benzyloxy)-2′,4′-dichloro-6-fluorobiphenyl-3-carbaldehyde (1.4 g, 3.73 mmol), 1.35 g (99%) of the title compound was obtained.

### Intermediate 127

(R)-2-(2-Benzyloxy-2′,6′-dichloro-biphenyl-3-yl oxymethyl)-oxirane: To a suspension of sodium hydride (60%, 0.47 g, 11.7 mmol) in DMF was added 2-benzyloxy-2′,6′-dichloro-biphenyl-3-ol (2.70 g, 7.8 mmol) at 0° C. The mixture was stirred at room temperature for 30 min. Then a solution of (R)-(−)-glydcidyl tosylate (3.57 g, 15.6 mmol) in DMF was introduced at room temperature. The resulting mixture was heated at 100° C. overnight and poured into the ice water. The mixture was extracted with methylene chloride. The organic layer washed with water and dried over anhydrous sodium sulfate and filtered. The solvent was removed under vacuum. Chromatography with 20% ethyl acetate in hexanes afforded 2.2 g (86%) of title compound as a colorless oil. MS ESI m/e 418.0965 [M+NH₄]⁺

### Intermediate 128

(R)-2-(2-Benzyloxy-2′,6-dichloro-biphenyl-3-yloxymethyl)-oxirane: To a suspension of sodium hydride (60%, 0.23 g, 5.8 mmol) in DMF (50 mL) was added 2-benzyloxy-2′,6-dichloro-biphenyl-3-ol (1.67 g, 4.84 mmol) at 0° C. The mixture was stirred at room temperature for 30 min. Then a solution of (R)-(−)-glycidyl tosylate (1.32 g, 5.8 mmol) in DMF was introduced at room temperature. The resulting mixture was heated at 80° C. overnight and cooled to room temperature. The mixture was extracted with ethyl acetate and washed with water and dried over anhydrous sodium sulfate and filtered. The solvent was removed under vacuum. Chromatography with 10-30% ethyl acetate in hexanes afforded 1.4 g (72%) of title compound as a colorless oil.

### Intermediate 129

(R)-2-(2-Benzyloxy-2′-chloro-6-fluoro-biphenyl-3-yloxymethyl)-oxirane: This intermediate was prepared by the same procedure as described for (R)-2-(2-benzyloxy-2′,6-dichloro-biphenyl-3-yloxymethyl)-oxirane (Intermediate 128). Starting from 2-benzyloxy-2′-chloro-6-fluoro-biphenyl-3-ol (1.4 g, 4.26 mmol), 0.44 g of the title compound as a colorless oil was obtained, together with recovered starting material.

### Intermediate 130

(2R)-2-[(2-(Benzyloxy)-6-chloro-2′-methylbiphenyl-3-yloxy)methyl]oxirane: This intermediate was prepared by the same procedure as for (2R)-2-(2-benzyloxy-2′,6-dichloro-biphenyl-3-yloxymethyl)-oxirane (Intermediate 128). Starting from 2-(benzyloxy)-6-chloro-2′-methylbiphenyl-3-ol (1.2 g, 3.69 mmol), 1.19 g (89%) of the title compound was obtained.

### Intermediate 131

(2R)-[2-(2-(Benzyloxy)-6-chloro-2′-(trifluoromethyl)biphenyl-3-yloxy)methyl]oxirane: This intermediate was prepared by the same procedure as for (2R)-2-(2-benzyloxy-2′,6-dichloro-biphenyl-3-yloxymethyl)-oxirane (Intermediate 128). Starting from 2-(benzyloxy)-6-chloro-2′-(trifluoromethyl)biphenyl-3-ol (0.7 g, 1.85 mmol), 0.6 g (75%) of the title compound was obtained.

### Intermediate 132

(2R)-2-[2-(Benzyloxy)-6-fluoro-2′-methylbiphenyl-3-yloxy)methyl]oxirane: This intermediate was prepared by the same procedure as for (2R)-2-(2-benzyloxy-2′,6-dichloro-biphenyl-3-yloxymethyl)-oxirane (Intermediate 128). Starting from 2-(benzyloxy)-6-fluoro-2′-methylbiphenyl-3-ol (0.6 g, 1.94 mmol), 0.32 g (45%) of the title compound was obtained as a colorless oil.

### Intermediate 133

(2R)-2-[2-(Benzyloxy)-2′,4′-dichloro-6-fluorobiphenyl-3-yloxy)methyl]oxirane: This intermediate was prepared by the same procedure as for (2R)-2-(2-benzyloxy-2′,6-dichloro-biphenyl-3-yloxymethyl)-oxirane (Intermediate 128). Starting from 2-(benzyloxy)-2′,4′-dichloro-6-fluorobiphenyl-3-ol (1.35 g, 3.72 mmol), 1.3 g (84%) of the title compound was obtained as a colorless oil.

### Intermediate 134

(S)-3-(3-Bromo-2-hydroxy-propoxy)-2′,6′-dichloro-biphenyl-2-ol (125-1); (S)-Acetic acid 1-bromomethyl-2-(2′,6′-dichloro-2-hydroxy-biphenyl-3-yloxy)-ethyl ester (125-2):

A solution of (R)-2-(2′,6′-dichloro-2-methoxy-biphenyl-3-yloxymethyl)-oxirane (2.2 g) in 33% HBr in acetic acid (15 mL) was heated at 65° C. for 1 h. The mixture was poured into ice water and extracted with methylene chloride. The organic layer was washed with water and dried over sodium sulfate and filtered. The organic solvent was removed under vacuum. Chromatography with 60% ethyl acetate in hexanes afforded 1.33 g (40%) of the title compound (67-1) as a colorless oil, [α]=−12.0° (1% solution in methanol), HRMS ESI m/e 407.9779 [M+NH₄]⁺; and 1.32 (43%) of the product (67-2) as a colorless oil, [α]=+4.0° (1% solution in methanol) HRMS ESI m/e 449.9886 [M+NH₄]⁺;

### Intermediate 135

(S)-[8-(2,6-Dichlorophenyl)-2,3-dihydro-benzo[1,4]dioxin-2-yl]-methanol: To a mixture of (S)-3-(3-bromo-2-hydroxy-propoxy)-2′,6′-dichloro-biphenyl-2-ol (1.33 g) and (S)-acetic acid 1-bromomethyl-2-(2′,6′-dichloro-5-fluoro-2-hydroxy-biphenyl-3-yloxy)-ethyl ester (1.32 g) in methanol (30 mL) at 0° C. was added 2.5 N NaOH (10 mL). The resulting mixture was stirred at 0° C. for 1 h. Then the mixture was extracted with methylene chloride and washed with water. The solvent was removed under vacuum. Chromatography with 20-60% ethyl acetate in hexanes afforded 1.77 g (91%) of the title compound as a colorless oil. HRMS ESI m/e 328.0514 [M+NH₄]⁺; [α]=+25.66° (c 4.8 mg/0.7 mL methanol).

### Intermediate 136

(R)-Toluene-4-sulfonic acid 8-(2,6-dichlorophenyl)-2,3-dihydro-benzo[1,4]dioxin-2-yl-methyl ester: To a solution of (S)-[8-(2,6-dichlorophenyl)-2,3-dihydro-benzo[1,4]dioxin-2-yl]-methanol (1.77 g, 5.6 mmol) in methylene chloride (60 mL) was added p-toluenesulfonyl chloride (1.63 g, 8.5 mmol), diisopropylethyl amine (2.48 mL, 14 mmol) and DMAP (catalytical amount) at room temperature. The resulting mixture was stirred at room temperature for 24 h. Then the reaction was quenched with ice water and extracted with methylene chloride. The organic layer was washed with water and dried over anhydrous sodium sulfate and filtered. The solvent was removed under vacuum. Chromatography with 10-40% ethyl acetate in hexanes afforded 1.78 g (88%) of the title compound as a clear thick oil. HRMS ESI m/e 482.0596 [M+NH₄]⁺; [α]=+23.00° (c 1% solution in methanol).

### Intermediate 137

(R)-Toluene-4-sulfonic acid 7-chloro-8-(2-chloro-phenyl)-2,3-dihydro-benzo[1,4]dioxin-2-ylmethyl ester: To a solution of (R)-2-(2-benzyloxy-6,2′-dichloro-biphenyl-3-yloxymethyl)-oxirane (0.26 g, 0.65 mmol) in ethanol (10 mL) was added catalyst palladium on carbon (10%, 95 mg) and followed by 1,4-cyclohexadiene (0.5 mL, 5.12 mmol). The resulting mixture was stirred at room temperature overnight. The mixture was filtered through a pad of celite and concentrated to provide crude [7-chloro-8-(2-chloro-phenyl)-2,3-dihydro-benzo[1,4]dioxin-2-yl]-methanol. This intermediate was further dissolved in methylene chloride (15 mL) and treated with diisopropylethylamine (0.24 mL, 1.4 mmol), p-toluenesulfonyl chloride (0.19 g, 0.97 mmol) and DMAP (catalytical amount) at room temperature. The resulting mixture was stirred at room temperature for 24 h. Then the reaction was quenched with ice water and extracted with methylene chloride. The organic layer was washed with water and dried over anhydrous sodium sulfate and filtered. The solvent was removed under vacuum. Chromatography with 5-25% ethyl acetate in hexanes afforded 0.16 g of the title compound as a colorless oil.

### Intermediate 138

(R)-Toluene-4-sulfonic acid 8-(2-chloro-phenyl)-7-fluoro-2,3-dihydro-benzo[1,4]dioxin-2-ylmethyl ester: This intermediate was prepared by the same procedure as described for toluene-4-sulfonic acid 7-chloro-8-(2-chloro-phenyl)-2,3-dihydro-benzo[1,4]dioxin-2-ylmethyl ester Intermediate 137). Starting from (R)-2-(2-Benzyloxy-2′-chloro-6-fluoro-biphenyl-3-yloxymethyl)-oxirane (0.44 g, 1.14 mmol), 0.26 g (51% for two steps) of the product was obtained as a thick oil.

### Intermediate 139

(2R)-(7-Chloro-8-o-tolyl-2,3-dihydrobenzo[b][1,4]dioxin-2-yl)methyl 4-methylbenzenesulfonate: This intermediate was prepared by the same procedure as for toluene-4-sulfonic acid 7-chloro-8-(2-chloro-phenyl)-2,3-dihydro-benzo[1,4]dioxin-2-ylmethyl ester (Intermediate 137). Starting from (2R)-2-((2-(benzyloxy)-6-chloro-2′-methylbiphenyl-3-yloxy)methyl)oxirane (1.19 g, 3.29 mmol), 0.86 g (59% for two steps) of the title compound was obtained as a thick oil.

### Intermediate 140

(2R)-{7-Chloro-8-[2-(trifluoromethyl)phenyl]-2,3-dihydrobenzo[b][1,4]dioxin-2-yl]}methyl 4-methylbenzenesulfonate: This intermediate was prepared by the same procedure as for toluene-4-sulfonic acid 7-chloro-8-(2-chloro-phenyl)-2,3-dihydro-benzo[1,4]dioxin-2-ylmethyl ester (Intermediate 137). Starting from (2R)-2-((2-(benzyloxy)-6-chloro-2′-(trifluoromethyl)biphenyl-3-yloxy)methyl)oxirane (0.6 g, 1.38 mmol), 0.5 g (73% for two steps) of the title compound was obtained as a thick oil.

### Intermediate 141

(2R)-[7-Fluoro-8-(o-tolyl-2,3-dihydrobenzo[b][1,4]dioxin-2-yl]methyl 4-methylbenzenesulfonate: This intermediate was prepared by the same procedure as for toluene-4-sulfonic acid 7-chloro-8-(2-chloro-phenyl)-2,3-dihydro-benzo[1,4]dioxin-2-ylmethyl ester (Intermediate 137). Starting from (2R)-2-((2-(benzyloxy)-6-fluoro-2′-methylbiphenyl-3-yloxy)methyl)oxirane (0.32 g, 0.88 mmol), 0.2 g (53% for two steps) of the title compound was obtained as a thick oil

### Intermediate 142

(2R)-8-(2,4-Dichlorophenyl)-7-fluoro-2,3-dihydrobenzo[b][1,4]dioxin-2-yl)methyl 4-methylbenzenesulfonate: This intermediate was prepared by the same procedure as for toluene-4-sulfonic acid 7-chloro-8-(2-chloro-phenyl)-2,3-dihydro-benzo[1,4]dioxin-2-ylmethyl ester (Intermediate 137). Starting from (2R)-2-((2-(benzyloxy)-2′,4′-dichloro-6-fluorobiphenyl-3-yloxy)methyl)oxirane (1.3 g, 3.1 mmol), 0.8 g (53% for two steps) of the title compound was obtained as a thick oil

### Intermediate 143

(S)-2-Azidomethyl-8-(2,6-dichlorophenyl)-2,3-dihydro-benzo[1,4]dioxin: To a solution of (R)-toluene-4-sulfonic acid 8-(2,6-dichlorophenyl)-2,3-dihydrobenzo-[1,4]dioxin-2-yl-methyl ester (2.33 g, 5.0 mmol) in DMF was added sodium azide (1.63 g, 25 mmol) t room temperature. The resulting mixture was heated at 90° C. overnight. The reaction mixture was poured in water and extracted with methylene chloride. The organic layer was washed with water and dried over anhydrous sodium sulfate and filtered. The solvent was removed. Chromatography with 0-30% ethyl acetate in hexanes afforded 1.33 g (79%) of the title compound as a colorless oil. MS EI m/e 335 M⁺; [α]=+46.0° (c 1% solution in methanol).

### Intermediate 144

(S)-2-Azidomethyl-7-chloro-8-(2-chloro-phenyl)-2,3-dihydro-benzo[1,4]dioxine: Starting from (R)-toluene-4-sulfonic acid 7-chloro-8-(2-chloro-phenyl)-2,3-dihydro-benzo[1,4]dioxin-2-ylmethyl ester (160 mg, 0.34 mmol), the procedure described for Intermediate 143 afforded 0.10 g (87%) of the title compound as a colorless oil.

### Intermediate 145

(S)-2-Azidomethyl-8-(2-chloro-phenyl)-7-fluoro-2,3-dihydro-benzo[1,4]dioxine: Starting from (R)-toluene-4-sulfonic acid 8-(2-chloro-phenyl)-7-fluoro-2,3-dihydro-benzo[1,4]dioxin-2-ylmethyl ester (0.26 g, 0.58 mmol), the procedure described for Intermediate 143 afforded 0.18 g (100%) of the title compound as a colorless oil.

### Intermediate 146

(2S)-2-(Azidomethyl)-7-chloro-8-o-tolyl-2,3-dihydrobenzo[b][1,4]dioxine: Starting from ((2R)-7-chloro-8-o-tolyl-2,3-dihydrobenzo[b][1,4]dioxin-2-yl)methyl 4-methylbenzenesulfonate (0.84 g, 1.89 mmol), the procedure described for Intermediate 143 afforded 0.6 g (100%) of the title compound as a colorless oil.

### Intermediate 147

(2S)-2-(Azidomethyl)-7-chloro-8-(2-(trifluoromethyl)phenyl)-2,3-dihydrobenzo[b][1,4]dioxine: Starting from ((2R)-7-chloro-8-(2-(trifluoromethyl)phenyl)-2,3-dihydrobenzo [b][1,4]dioxin-2-yl)methyl 4-methylbenzenesulfonate (0.5 g, 1.0 mmol), the procedure described for Intermediate 143 afforded 0.25 g (67%) of the title compound as a colorless oil.

### Intermediate 148

(2S)-2-(Azidomethyl)-7-fluoro-8-o-tolyl-2,3-dihydrobenzo[b][1,4]dioxine: Starting from ((2R)-7-fluoro-8-o-tolyl-2,3-dihydrobenzo[b][1,4]dioxin-2-yl)methyl-4-methylbenzene-sulfonate (0.2 g, 0.47 mmol), the procedure described for Intermediate 143 afforded 0.12 g (85%) of the title compound as a colorless oil.

### Intermediate 149

(2S)-2-(Azidomethyl)-8-(2,4-dichlorophenyl)-7-fluoro-2,3-dihydrobenzo[b][1,4]dioxine: Starting from ((2R)-8-(2,4-dichlorophenyl)-7-fluoro-2,3-dihydrobenzo[b][1,4]dioxin-2-yl)methyl 4-methylbenzenesulfonate (1.2 g, 2.48 mmol), the procedure described for Intermediate 143 afforded 0.8 g (98%) of the title compound as a colorless oil.

### Intermediate 150

3-Bromo-2′,6′-dichlorobiphenyl-2-ol: To a solution of 2′,6′-dichlorobiphenyl-2-ol (0.1 g, 0.42 mmol) in 4 mL methylene chloride at room temperature was added diisopropylamine (0.12-1.0 eq), followed by NBS (0.067 g, 0.38 mmol, dissolved in 2 mL methylene chloride) during a 30 minute period. The reaction was stirred at room temperature for an additional 30 minutes. The solvent was removed under vacuum. Chromatography with 15% ethyl acetate/hexanes afforded the desired title compound as a white solid, mp: 44-45° C. MS ES m/e 314.9 [M−H]⁻.

### Intermediate 151

3-Iodo-2′,6′-dichlorobiphenyl-2-ol: To a solution of 2′,6′-dichlorobiphenyl-2-ol (7.7 g, 0.032 mol) and copper acetate (1.0 eq, 5.8 g) in acetic acid (150 mL) was slowly added a solution of I₂ (8.2 g, 32.2 mmol) in acetic acid at 120° C. The mixture was heated at this temperature overnight and filtered through the pad of the celite. The mixture was extracted with methylene chloride and the organic layer was washed with Na₂SO₃ solution. The solvent was removed under vacuum and chromatography 5-20% ethyl acetate in hexanes afforded 5.5 g title compound as a white solid, mp: 51-53° C. MS ES m/e 362.9 [M−H]⁻.

### Intermediate 152

2′,6′-Dichloro-2,3-dimethoxybiphenyl: To a solution of 2,6-dichlorobromobenzene (5.0 g, 22 mmol) and sodium hydroxide (4.4 g, 0.11 mol) in DME-water (2:1, 180 mL) was added 2,3-dimethoxybenzene boronic acid (8.0 g, 44 mmol) at 90° C., followed by tetrakis(triphenyl-phosphine)palladium (0) (0.77 g, 0.66 mmol). The reaction mixture was heated at 90° C. overnight and cooled to room temperature. The mixture was extracted with methylene chloride and washed with water. The organic solvent was removed under vacuum. Chromatography with 5% ethyl acetate in hexanes afforded 4.57 g (72%) of the title compound as a white solid, mp: 49-50° C. MS EI m/e 282 M⁺.

### Intermediate 153

2′,6′-Dichlorobiphenyl-2,3-diol: To a solution of 2′,6′-dichloro-2,3-dimethoxybiphenyl (4.56 g, 0.016 mmol) in 100 mL methylene chloride at 0° C. was added BBr₃ (3 eq, 4.56 mL) during the 5-minute period. The reaction was stirred at room temperature overnight. The reaction was poured into the ice-water slowly and extracted with methylene chloride (3×100 mL). The organic layer was washed with water and dried over anhydrous sodium sulfate. The solvent was removed under vacuum. Column chromatography with 5-30% ethyl acetate/hexanes afforded the desired title compound (3.9 g, 95%) as a colorless oil. MS ES m/e 252.9 [M−H]⁻.

### Intermediate 154

(S)-8-(2,6-Dichlorophenyl)-2,3-dihydrobenzo[b][1,4]dioxin-2-yl)-methanol: To a solution of 2′,6′-dichloro-biphenyl-2-ol (3.9 g, 0.015 mmol) in 100 mL DMF at room temperature was added (R)-(−)-glycidyl tosylate (1.2 eq, 4.2 g) and potassium carbonate (5.3 g, 37.5 mmol). The reaction was heated at 70° C. overnight. The reaction was poured into the water and extracted with ethyl acetate (3×100 mL). The organic layer was washed with water (3×100 mL) and dried over anhydrous sodium sulfate. The solvent was removed under vacuum. Chromatography with 0-30% ethyl acetate/hexanes afforded the title compound (2.0 g, 42%) as a colorless oil. [α]_{D}²⁵=+250; MS EI m/e 310 M⁺.

### Intermediate 155

3-Bromo-5-fluoro-benzene-1,2-diol: To a solution of 3-bromo-5-fluoro-2-hydroxy-benzaldehyde (12.04 g, 55 mmol) in methylene chloride (200 mL) was added m-CPBA (77% max, 5.3 g). The resulting mixture was stirred at room temperature overnight and quenched with 10% sodium sulfite and 10% sodium bicarbonate. The mixture was extracted with methylene chloride and washed with water. The solvent was removed under vacuum to afford crude material as a light yellow oil. To a solution of the crude oil in methanol was added sodium hydroxide (8.8 g, 0.22 mol) at room temperature. The mixture was stirred at room temperature overnight and neutralized with concentrated hydrochloric acid. The mixture was extracted with methylene chloride and washed with water. The organic solvent was removed under vacuum. Chromatography with 10-40% ethyl acetate in hexanes afforded 6.8 g (60%) of the title compound as a yellow oil. MS APPI m/e 205 [M−H]⁻

### Intermediate 156

3-Bromo-5-chloro-benzene-1,2-diol: To a solution of 3-bromo-5-chloro-2-hydroxy-benzaldehyde (17.0 g, 72.2 mmol) in methylene chloride (300 mL) was added m-CPBA (77% max, 42 g). The resulting mixture was stirred at room temperature overnight and quenched with 1:1 10% sodium sulfite and saturated sodium bicarbonate. The mixture was extracted with methylene chloride and washed with water. The solvent was removed under vacuum to afford a crude material as a light yellow oil. To a solution of the crude oil in methanol was added sodium bicarbonate (12 g, 144 mmol) at room temperature. The mixture was stirred at room temperature for 3 hrs and the solvent removed under vacuum. The mixture was extracted with ethyl acetate and washed with water. The organic solvent was removed under vacuum. Chromatography with 10-40% ethyl acetate in hexanes afforded 10.5 g (65%) of the title compound as a white solid.

### Intermediate 157

2′,6′-Dichloro-5-fluoro-biphenyl-2,3-diol: To a solution of 2′,6′-dichloro-5-fluoro-2-methoxy-biphenyl-3-ol (2.65 g, 9.2 mmol) im methylene chloride (60 mL) was added boron tribromide (1.30 mL, 13.8 mmol) at −78° C. The mixture was stirred at −78° C. to room temperature overnight. The reaction mixture was poured into ice-NH₄OH, extracted with methylene chloride and the methylene chloride extract washed with water. The solvent was removed under vacuum. Chromatography with 0-40% ethyl acetate in hexanes afforded 1.45 g (58%) of the title compound as a yellow oil. MS ESI m/e 271.1 [M−H]⁻

### Intermediate 158

4-Bromo-6-chloro-benzo[1,3]dioxole-2,2-dicarboxylic acid diethyl ester: To a suspension of 3-bromo-5-chlorobenzene-1,2-diol (10.5 g, 47 mmol) and potassium carbonate (16.2 g, 117 mmol) in DMF (100 mL) was added diethyl dibromomalonate (9.78 mL, 51 mmol) at room temperature. The resulting mixture was stirred at room temperature overnight. The solvent was removed under vacuum and mixture was extracted with ethyl acetate and water. The organic layer was washed with water and dried over anhydrous sodium sulfate and filtered. The solvent was removed under vacuum. Chromatography with 0-30% ethyl acetate in hexanes afforded 8.0 g (45%) of the title compound as a white solid.

### Intermediate 159

4-Bromo-6-fluoro-benzo[1,3]dioxole-2,2-dicarboxylic acid diethyl ester: To a suspension of 3-bromo-5-fluorobenzene-1,2-diol (6.8 g, 33 mmol) and potassium carbonate (11.3 g, 82.5 mmol) in acetone (150 mL) was added diethyl dibromomalonate (6.5 mL, 34.6 mmol) at room temperature. The resulting mixture was stirred at room temperature overnight. The acetone was removed under vacuum and mixture was extracted with methylene chloride and water. The organic layer was washed with water and dried over anhydrous sodium sulfate and filtered. The solvent was removed under vacuum. Chromatography with 0-30% ethyl acetate in hexanes afforded 7.0 g (59%) of the title compound as a colorless oil. MS EI m/e 362 M⁺.

### Intermediate 160

4-(2,6-Dichloro-phenyl)-6-fluoro-benzo[1,3]dioxole-2,2-dicarboxylic acid diethyl ester: Starting from 2′,6′-dichloro-5-fluoro-biphenyl-2,3-diol (1.45 g, 5.3 mmol) and following the procedure described for Intermediate 159 above, the desired title compound 1.45 g (64%) was obtained as a colorless oil. ME ESI m/e 446.0 [M+NH₄]⁺.

### Intermediate 161

4-Bromo-6-fluoro-benzo[1,3]dioxole-2,2-dicarboxylic acid: A solution of 4-bromo-6-fluoro-benzo[1,3]dioxole-2,2-dicarboxylic acid diethyl ester (7.0 g) in THF (75 mL) and 1N sodium hydroxide (75 mL) was stirred at room temperature for 2 days. The mixture was neutralized with concentrated hydrochloric acid and extracted with methylene chloride. The solvent was removed under vacuum to afford 5.0 g (84%) of the title compound as a light yellow oil. HRMS ESI m/e [M−H]⁻.

### Intermediate 162

4-Bromo-6-chloro-benzo[1,3]dioxole-2,2-dicarboxylic acid: A solution of 4-bromo-6-chloro-benzo[1,3]dioxole-2,2-dicarboxylic acid diethyl ester (8.0 g) in THF (75 mL) and 1N sodium hydroxide (75 mL) was stirred at room temperature for 2 days. The mixture was neutralized with concentrated hydrochloric acid and extracted with methylene chloride. The solvent was removed under vacuum to afford 5.5 g (81%) of the title compound as a dark yellow oil.

### Intermediate 163

4-(2,6-Dichloro-phenyl)-6-fluoro-benzo[1,3]dioxole-2,2-dicarboxylic acid: Starting from 4-(2,6-dichloro-phenyl)-6-fluoro-benzo[1,3]dioxole-2,2-dicarboxylic acid diethyl ester (1.45 g) and following the procedure described for Intermediate 161 above, the desired title compound (1.40 g, 94%) was obtained as a colorless oil. HRMS ESI m/e 370.9532 [M+NH₄]⁺.

### Intermediate 164

4-Bromo-6-fluoro-benzo[1,3]dioxole-2-carboxylic acid: A solution of 4-bromo-6-fluoro-benzo[1,3]dioxole-2,2-dicarboxylic acid (5.0 g) in mesitylene (40 mL) was refluxed for 7 h. The solvent was removed under vacuum, to afford the title compound (2.86 g, 65%) as a yellow oil. HRMS ESI m/e 260.9210 [M−H]⁻.

### Intermediate 165

4-Bromo-6-chloro-benzo[1,3]dioxole-2-carboxylic acid: A solution of 4-bromo-6-chloro-benzo[1,3]dioxole-2,2-dicarboxylic acid (5.5 g) in mesitylene (50 mL) was refluxed overnight. The solvent was removed under vacuum to afford the title compound (4.4 g, 93%) as a pale yellow solid. MS ESI m/e 278 [M−H]⁻.

### Intermediate 166

4-(2,6-Dichlorophenyl)-6-fluoro-benzo[1,3]dioxole-2-carboxylic acid: Starting from 4-(2,6-dichloro-phenyl)-6-fluoro-benzo[1,3]dioxole-2,2-dicarboxylic acid (1.40 g) and following the procedure described for Intermediate 164 above, the title compound was obtained as a colorless oil. HRMS ESI m/e 326.9641 [M−H]⁻.

### Intermediate 167

4-Bromo-6-fluoro-benzo[1,3]dioxole-2-carboxylic acid methyl ester: To a solution of the crude 4-bromo-6-fluoro-benzo[1,3]dioxole-2-carboxylic acid (2.4 g, 9.1 mmol) in methylene chloride (30 mL) was added (trimethylsilyl)diazomethane (2.0 M in hexanes, 6.8 mL, 13.6 mmol) very slowly at 0° C. The mixture was stirred at 0° C. for 15 min and poured into ice water. The mixture was extracted with methylene chloride and washed with water. The solvent was removed under vacuum. Chromatography with 0-30% ethyl acetate and hexanes afforded 0.91 g (35%) of the title compound as a light yellow oil. MS APPI m/e 275 [M−H]⁻.

### Intermediate 168

4-Bromo-6-chloro-benzo[1,3]dioxole-2-carboxylic acid methyl ester: To a solution of the crude 4-bromo-6-chloro-benzo[1,3]dioxole-2-carboxylic acid (4.4 g, 15.7 mmol) in methylene chloride (50 mL) was added (trimethylsilyl)diazomethane (2.0 M in hexanes, 14 mL, 28 mmol) very slowly at 0° C. The mixture was stirred at 0° C. for 30 min and poured into ice water. The mixture was extracted with methylene chloride and washed with water. The solvent was removed under vacuum. Chromatography with 5-25% ethyl acetate and hexanes afforded 3.7 g (80%) of the title compound as a white solid. MS EI m/e 292 [M−H]⁻.

### Intermediate 169

4-(2,6-Dichloro-phenyl)-6-fluoro-benzo[1,3]dioxole-2-carboxylic acid methyl ester: Starting from 4-(2,6-dichloro-phenyl)-6-fluoro-benzo[1,3]dioxole-2-carboxylic acid and following the procedure described for Intermediate 168 above, the desired title compound (0.72 g, 54%) was obtained as a colorless oil. MS ESI m/e 360.0 [M+NH₄]⁺.

### Intermediate 170

(4-Bromo-6-fluoro-benzo[1,3]dioxol-2-yl)methanol: To a solution of 4-bromo-6-fluoro-benzo[1,3]dioxole-2-carboxylic acid methyl ester (0.46 g, 1.7 mmol) in THF was added sodium borohydride (0.62 g, 17 mmol) at room temperature. The mixture was stirred at room temperature overnight and quenched with methanol slowly at 0° C. The mixture was extracted with methylene chloride and washed with water. The solvent was removed under vacuum. Chromatography with 0-60% ethyl acetate in hexanes afforded 0.24 g (58%) of the title product as a colorless oil. MS ESI m/e 246.9 [M−H]⁻.

### Intermediate 171

(4-Bromo-6-chloro-benzo[1,3]dioxol-2-yl)-methanol: To a solution of 4-bromo-6-chloro-benzo[1,3]dioxole-2-carboxylic acid methyl ester (3.7 g, 12.6 mmol) in THF (100 mL) was added sodium borohydride (4.8 g, 127 mmol) at room temperature. The mixture was stirred at room temperature overnight and quenched with methanol slowly with an ice bath underneath. The mixture was extracted with ethyl acetate and the extract washed with water. The solvent was removed under vacuum. Chromatography with 0-60% ethyl acetate in hexanes afforded 2.7 g (80%) of the title compound as a white solid. MS EI m/e 264 [M−H]⁻.

### Intermediate 172

[4-(2,6-Dichloro-phenyl)-6-fluoro-benzo[1,3]dioxol-2-yl]-methanol: Starting from 4-(2,6-dichloro-phenyl)-6-fluoro-benzo[1,3]dioxole-2-carboxylic acid methyl ester (0.72 g, 2.1 mmol) and following the procedure described for Intermediate 170, the desired product 0.31 g (47%) was obtained as a colorless oil. MS ESI m/e 312.9 [M−H]⁻.

### Intermediate 173

Toluene-4-sulfonic acid 4-bromo-6-fluoro-benzo[1,3]dioxol-2-ylmethyl ester: To a solution of 4-bromo-6-fluoro-benzo[1,3]dioxol-2-yl)methanol (0.24 g, 0.96 mmol) in methylene chloride (30 mL) was added p-toluenesulfonyl chloride (0.27 g, 1.15 mmol), diisopropylethylamine (0.42 mL, 2.4 mmol) and DMAP (catalytical amount) at room temperature. The resulting mixture was stirred at room temperature for 24 h. Then the reaction was quenched with ice water and extracted with methylene chloride. The organic layer was washed with water and dried over anhydrous sodium sulfate and filtered. The solvent was removed under vacuum. Chromatography with 10-40% ethyl acetate in hexanes afforded 0.34 g (88%) of the title compound as a colorless oil. HRMS ESI m/e 419.9919 [M+NH₄]⁺

### Intermediate 174

Toluene-4-sulfonic acid 4-bromo-6-chloro-benzo[1,3]dioxol-2-ylmethyl ester: To a solution of 4-bromo-6-chloro-benzo[1,3]dioxol-2-yl)methanol (2.7 g, 10.2 mmol) in methylene chloride (100 mL) was added p-toluenesulfonyl chloride (2.9 g, 15.3 mmol), diisopropylethylamine (3.5 mL, 20.3 mmol) and 4-DMAP (catalytical amount) at room temperature. The resulting mixture was stirred at room temperature for 24 h. Then the reaction was quenched with ice water and extracted with methylene chloride. The organic layer was washed with water and dried over anhydrous sodium sulfate and filtered. The solvent was removed under vacuum. Chromatography with 10-40% ethyl acetate in hexanes afforded 4.3 g (100%) of the title compound as a white solid. MS ES m/z 436 [M+NH₄]⁺.

### Intermediate 175

Toluene-4-sulfonic acid 4-(2,6-Dichloro-phenyl)-6-fluoro-benzo[1,3]dioxol-2-ylmethyl ester: Starting from [4-(2,6-dichloro-phenyl)-6-fluoro-benzo[1,3]dioxol-2-yl]-methanol (0.31 g, 0.98 mmol) and following the procedure described for Intermediate 173, the desired product 0.43 g (93%) was obtained as a colorless oil. HRMS ESI m/e 486.0338 [M+NH₄]⁺.

### Intermediate 176

Toluene-4-sulfonic acid 4-(2,4-dichloro-phenyl)-6-fluoro-benzo[1,3]dioxol-2-ylmethyl ester: To a solution toluene-4-sulfonic acid 4-bromo-6-fluoro-benzo[1,3]dioxol-2-ylmethyl ester of (0.34 g, 8.4 mmol) and 2,4-dichlorobenzene boronic acid (0.64 g, 2.5 mmol) in dioxane-water (4/1) was added dichlorobis(tri-o-tolyphosphine)-palladium (II) (0.02 g, 0.02 mmol) and potassium carbonate (0.29 g, 2.1 mmol). The reaction mixture was heated at 90° C. for 0.5 h. The mixture was filtered through the pad of celite and concentrated under vacuum. Chromatography with 10-30% ethyl acetate in hexanes afforded 0.32 g (81%) of the title compound as an colorless oil. HRMS ESI m/e 486.0349 [M+NH₄]⁺

### Intermediate 177

Toluene-4-sulfonic acid 4-(2-methyl-phenyl)-6-fluoro-benzo[1,3]dioxol-2-ylmethyl ester: Starting from 2-methylbenzene boronic acid (0.16 g, 0.39 mmol) and following the procedure described for Intermediate 176, the title compound (0.14 g, 88%) was obtained as a colorless oil. MS ES m/e 432.1 [M+NH₄]⁺.

### Intermediate 178

Toluene-4-sulfonic acid 4-(2-methoxy-phenyl)-6-fluoro-benzo[1,3]dioxol-2-ylmethyl ester: Starting from 2-methoxybenzene boronic acid (0.16 g, 0.39 mmol) and following the procedure described for Intermediate 176, the title compound (0.13 g, 80%) was obtained as a colorless oil. MS ESI m/e 448.1 [M+NH₄]⁺.

### Intermediate 179

Toluene-4-sulfonic acid 4-(2-fluoro-phenyl)-6-fluoro-benzo[1,3]dioxol-2-ylmethyl ester: Starting from 2-fluorobenzene boronic acid (0.16 g, 0.39 mmol) and following the procedure described for Intermediate 176, the title compound (0.16 g, 94%) was obtained as a colorless oil. MS ES m/e 436.1 [M+NH₄]⁺.

### Intermediate 180

Toluene-4-sulfonic acid 4-(2-chloro-phenyl)-6-fluoro-benzo[1,3]dioxol-2-ylmethyl ester: Starting from 2-chlorobenzene boronic acid (0.16, 0.39 mmol) and following the procedure described for Intermediate 176, the title compound (0.15 g, 85%) was obtained as a colorless oil. MS ES m/e 452.0 [M+NH₄]⁺.

### Intermediate 181

Toluene-4-sulfonic acid 4-phenyl-6-fluoro-benzo[1,3]dioxol-2-ylmethyl ester: Starting from phenyl boronic acid (0.13 g, 0.37 mmol) and following the procedure described for Intermediate 176, the title compound (0.13 g, 90%) was obtained as a colorless oil. MS ES m/e 418.1 [M+NH₄]⁺.

### Intermediate 182

Toluene-4-sulfonic acid 4-(3-chloro-phenyl)-6-fluoro-benzo[1,3]dioxol-2-ylmethyl ester: Starting from 3-chlorobenzene boronic acid (0.17 g, mmol) and following the procedure described Intermediate 176, the title compound (0.15 g, 85%) was obtained as a colorless oil. MS ES m/e 452.0 [M+NH₄]⁺.

### Intermediate 183

Toluene-4-sulfonic acid 4-(4-chloro-phenyl)-6-fluoro-benzo[1,3]dioxol-2-ylmethyl ester: Starting from 4-chlorobenzene boronic acid (0.15 g, 0.37 mmol) and following the procedure described Intermediate 176, the title compound (0.13 g, 78%) was obtained as a colorless oil. MS ES m/e 452.0 [M+NH₄]⁺.

### Intermediate 184

Toluene-4-sulfonic acid 4-(2-trifluoromethyl-phenyl)-6-fluoro-benzo[1,3]dioxol-2-ylmethyl ester: Starting from 2-trifluormethylbenzene boronic acid (0.16 g, 0.39 mmol) and following the procedure described for Intermediate 176, the title compound (0.15 g, 80%) was obtained as a colorless oil. MS ES m/e 486.0 [M+NH₄]⁺.

### Intermediate 185

Toluene-4-sulfonic acid 4-(2,5-dichloro-phenyl)-6-fluoro-benzo[1,3]dioxol-2-ylmethyl ester: Starting from 2,5-dichlorobenzene bornic acid (0.16 g, 0.39 mmol) and following the procedure described for Intermediate 176, the title compound (0.14 g, 85%) was obtained as a colorless oil. MS ES m/e 486.0 [M+NH₄]⁺.

General procedure to generate biaryl derivatives from toluene-4-sulfonic acid 4-bromo-6-chloro-benzo[1,3]dioxol-2-yl-methyl ester

To a solution of toluene-4-sulfonic acid 4-bromo-6-chloro-benzo[1,3]dioxol-2-yl-methyl ester (1.0 eq) and substituted benzene boronic acid (3 eq) in DME-water (4/1) was added tetrakis(triphenylphospine)palladium (0) (0.1 eq) and sodium carbonate (3 eq). The reaction mixture was heated at 80° C. until starting material was gone. The mixture was filtered through the pad of celite and concentrated under vacuum. Chromatography with 10% ethyl acetate in hexanes afforded the title compounds.

Using the general procedure outlined above, INTERMEDIATES 186-197 can be prepared.

### Intermediate 186

Toluene-4-sulfonic acid 4-(2-chloro-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2-chlorobenzene boronic acid (0.33 g, 2.1 mmol), the general procedure described above gave the title compound (0.28 g, 81%) as a colorless oil.

### Intermediate 187

Toluene-4-sulfonic acid 4-(2-methyl-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2-methylbenzene boronic acid (0.28 g, 2.1 mmol), the general procedure described above gave the title compound (0.30 g, 98%) as a colorless oil.

### Intermediate 188

Toluene-4-sulfonic acid 4-(2-methoxy-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2-methoxybenzene boronic acid (0.28 g, 2.1 mmol), the general procedure described above gave the title compound (0.30 g, 98%) as a colorless oil.

### Intermediate 189

Toluene-4-sulfonic acid 4-(2-fluoro-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2-fluorobenzene boronic acid (0.29 g, 2.1 mmol), the general procedure described above gave the title compound (0.27 g, 87%) as a colorless oil.

### Intermediate 190

Toluene-4-sulfonic acid 4-(5-chloro-2-methoxy-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 5-chloro-2-methoxybenzene boronic acid (0.39 g, 2.1 mmol), the general procedure described above gave the title compound (0.27 g, 79%) as a colorless oil.

### Intermediate 191

Toluene-4-sulfonic acid 4-(2,3-dimethoxy-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2,3-dimethoxybenzene boronic acid (0.38 g, 2.1 mmol), the general procedure described above gave the title compound (0.30 g, 88%) as a colorless oil.

### Intermediate 192

Toluene-4-sulfonic acid 4-(2,4-dichloro-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2,4-dichlorobenzene boronic acid (0.40 g, 2.1 mmol), the general procedure described above gave the title compound (0.20 g, 58%) as a colorless oil.

### Intermediate 193

Toluene-4-sulfonic acid 4-(4-chloro-2-methyl-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 4-chloro-2-methylbenzene boronic acid (0.36 g, 2.1 mmol), the general procedure described above gave the title compound (0.34 g, 100%) as a colorless oil.

### Intermediate 194

Toluene-4-sulfonic acid 4-(2,5-dichloro-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2,5-dichlorobenzene boronic acid (0.40 g, 2.1 mmol), the general procedure described above gave the title compound (0.24 g, 69%) as a colorless oil.

### Intermediate 195

Toluene-4-sulfonic acid 4-(2,5-difluoro-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2,5-difluorobenzene boronic acid (0.33 g, 2.1 mmol), the general procedure described above gave the title compound (0.30 g, 93%) as a colorless oil.

### Intermediate 196

Toluene-4-sulfonic acid 4-(2,5-dimethoxy-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.30 g, 0.71 mmol) and 2,5-dimethoxybenzene boronic acid (0.38 g, 2.1 mmol), the general procedure described above gave the title compound (0.20 g, 59%) as a colorless oil.

### Intermediate 197

Toluene-4-sulfonic acid 4-(2-phenyl-phenyl)-6-chloro-benzo[1,3]dioxol-2-yl-methyl ester: Starting from toluene-4-sulfonic acid 4-bromo-6-chloro-benzo[1,3]dioxol-2-yl-methyl ester (0.40 g, 0.95 mmol) and 2-phenylbenzene boronic acid (0.40 g, 2.0 mmol) according to the general procedure described above gave the desired product (0.48 g, 100%) as a colorless oil.

### Intermediate 198

2-Azidomethyl-4-(2,4-dichloro-phenyl)-6-fluoro-benzo[1,3]dioxole: A mixture of toluene-4-sulfonic acid 4-(2,4-dichloro-phenyl)-6-fluoro-benzo[1,3]dioxol-2-ylmethyl ester (0.32 g, 0.79 mmol) and sodium azide (0.26 g, 3.95 mmol) in DMF was heated at 90° C. overnight. The reaction was quenched with water. The mixture was extracted with methylene chloride. The organic layer was washed with water and dried over sodium sulfate. The organic solvent was removed under vacuum. Chromatography with 10-20% ethyl acetate in hexanes afforded 0.24 g (89%) of the title compound as a colorless oil. MS EI m/e 339 M⁺.

### Intermediate 199

2-Azidomethyl-4-(2-methoxy-phenyl)-6-fluoro-benzo[1,3]dioxole: Starting from toluene-4-sulfonic acid 4-(2-methoxy-phenyl)-6-fluoro-benzo[1,3]dioxol-2-ylmethyl ester (0.13 g, 0.30 mmol) and following the procedure described for Intermediate 198, gave the title compound (77 mg, 85%) was obtained as a colorless oil.

### Intermediate 200

2-Azidomethyl-4-phenyl-6-fluoro-benzo[1,3]dioxole: Starting from toluene-4-sulfonic acid 4-phenyl-6-fluoro-benzo[1,3]dioxol-2-ylmethyl ester (0.13 g, 0.32 mmol) and following the procedure described for Intermediate 198, the title compound (76 mg, 85%) was obtained as a colorless oil. MS EI m/e 271 M⁺.

### Intermediate 201

2-Azidomethyl-4-(3-chloro-phenyl)-6-fluoro-benzo[1,3]dioxole: Starting from toluene-4-sulfonic acid 4-(3-chloro-phenyl)-6-fluoro-benzo[1,3]dioxol-2-ylmethyl ester (0.15 g, 0.34 mmol) and following the procedure described for Intermediate 198, the title compound (90 mg, 86%) was obtained as a colorless oil. MS EI m/e 305 M⁺.

### Intermediate 202

2-Azidomethyl-4-(4-chloro-phenyl)-6-fluoro-benzo[1,3]dioxole: Starting from toluene-4-sulfonic acid 4-(4-chloro-phenyl)-6-fluoro-benzo[1,3]dioxol-2-ylmethyl ester (0.13 g, 0.30 mmol) and following the procedure described for Intermediate 198, the title compound (73 mg, 80%) was obtained as a colorless oil. MS EI m/e 305 M⁺.

### Intermediate 203

2-Azidomethyl-4-(2,5-dichloro-phenyl)-6-fluoro-benzo[1,3]dioxole: Starting from toluene-4-sulfonic acid 4-(2,5-dichloro-phenyl)-6-fluoro-benzo[1,3]dioxol-2-ylmethyl ester (0.14 g, 0.30 mmol) and following the procedure described for Intermediate 198, the title compound (99 mg, 88%) was obtained as a colorless oil. MS EI m/e 339 M⁺.

### Intermediate 204

2-Azidomethyl-4-(2-trifluoromethyl-phenyl)-6-fluoro-benzo[1,3]dioxole: Starting from toluene-4-sulfonic acid 4-(2-trifluoromethyl-phenyl)-6-fluoro-benzo[1,3]dioxol-2-ylmethyl ester (0.15 g, 0.32 mmol) and following the procedure described for Intermediate 198, the title compound (85 mg, 79%) was obtained as a colorless oil. MS EI m/e 339 M⁺.

### Intermediate 205

2-Azidomethyl-4-(2-methyl-phenyl)-6-fluoro-benzo[1,3]dioxole: Starting from toluene-4-sulfonic acid 4-(2-methyl-phenyl)-6-fluoro-benzo[1,3]dioxol-2-ylmethyl ester (0.14 g, 0.34 mmol) and following the procedure described for Intermediate 198, the title compound (78 mg, 90%) was obtained as a colorless oil. MS EI m/e 285 M⁺.

### Intermediate 206

2-Azidomethyl-4-(2-chloro-phenyl)-6-fluoro-benzo[1,3]dioxole: Starting from toluene-4-sulfonic acid 4-(2-chloro-phenyl)-6-fluoro-benzo[1,3]dioxol-2-ylmethyl ester (0.15 g, 0.34 mmol) and following the procedure described for Intermediate 198, the desired product (93 mg, 88%) was obtained as a colorless oil. MS EI m/e 305 M⁺.

### Intermediate 207

2-Azidomethyl-4-(2-fluoro-phenyl)-6-fluoro-benzo[1,3]dioxole: Starting from toluene-4-sulfonic acid 4-(2-fluoro-phenyl)-6-fluoro-benzo[1,3]dioxol-2-ylmethyl ester (0.16 g, 0.38 mmol) and following the procedure described for Intermediate 198, the desired product (97 mg, 88%) was obtained as a colorless oil. MS EI m/e 289 M⁺.

### Intermediate 208

2-Azidomethyl-6-chloro-4-(2-chloro-phenyl)-benzo[1,3]dioxole: Starting from toluene-4-sulfonic acid 4-(2-chloro-phenyl)-6-chloro-benzo[1,3]dioxol-2-yl-methyl ester (280 mg, 0.62 mmol) and following the procedure described for Intermediate 198, 0.18 g (90%) of the title compound was obtained as a colorless oil.

### Intermediate 209

2-Azidomethyl-6-chloro-4-(2-methyl-phenyl)-benzo[1,3]dioxole: Starting from toluene-4-sulfonic acid 4-(2-methyl-phenyl)-6-chloro-benzo[1,3]dioxol-2-yl-methyl ester (0.30 g, 0.69 mmol) and following the procedure described Intermediate 198, 0.20 g (95%) of the title compound was obtained as a colorless oil.

### Intermediate 210

2-Azidomethyl-6-chloro-4-(2-methoxy-phenyl)-benzo[1,3]dioxole: Starting from toluene-4-sulfonic acid 4-(2-methoxy-phenyl)-6-chloro-benzo[1,3]dioxol-2-yl-methyl ester (0.26 g, 0.69 mmol) and following the procedure described Intermediate 198, 0.18 g (100%) of the title compound was obtained as a colorless oil.

### Intermediate 211

2-Azidomethyl-6-chloro-4-(2-fluoro-phenyl)-benzo[1,3]dioxole: Starting from toluene-4-sulfonic acid 4-(2-fluoro-phenyl)-6-chloro-benzo[1,3]dioxol-2-yl-methyl ester (0.27 g, 0.62 mmol) and following the procedure described Intermediate 198, 0.18 g (95%) of the title compound was obtained as a colorless oil.

### Intermediate 212

2-Azidomethyl-6-chloro-4-(5-chloro-2-methoxy-phenyl)-benzo[1,3]dioxole: Starting from toluene-4-sulfonic acid 4-(5-chloro-2-methoxy-phenyl)-6-chloro-benzo[1,3]dioxol-2-yl-methyl ester (0.27 g, 0.56 mmol) and following the procedure described Intermediate 198, 0.19 g (95%) of the tile product was obtained as a colorless oil.

### Intermediate 213

2-Azidomethyl-6-chloro-4-(2,3-dimethoxy-phenyl)-benzo[1,3]dioxole: Starting from toluene-4-sulfonic acid 4-(2,3-dimethoxy-phenyl)-6-chloro-benzo[1,3]dioxol-2-yl-methyl ester (0.30 g, 0.63 mmol) and following the procedure described Intermediate 198, 0.20 g (91%) of the title compound was obtained as a colorless oil.

### Intermediate 214

2-Azidomethyl-6-chloro-4-(2,4-dichloro-phenyl)-benzo[1,3]dioxole: Starting from toluene-4-sulfonic acid 4-(2,4-dichloro-phenyl)-6-chloro-benzo[1,3]dioxol-2-yl-methyl ester (0.20 g, 0.41 mmol) and following the procedure described Intermediate 198, 0.12 g (82%) of the title compound was obtained as a colorless oil.

### Intermediate 215

2-Azidomethyl-6-chloro-4-(4-chloro-2-methyl-phenyl)-benzo[1,3]dioxole: Starting from toluene-4-sulfonic acid 4-(4-chloro-2-methyl-phenyl)-6-chloro-benzo[1,3]dioxol-2-yl-methyl ester (0.34 g, 0.73 mmol) and following the procedure described Intermediate 198, 0.20 g (83%) of the title compound was obtained as a colorless oil.

### Intermediate 216

2-Azidomethyl-6-chloro-4-(2,5-dichloro-phenyl)-benzo[1,3]dioxole: Starting from toluene-4-sulfonic acid 4-(2,5-dichloro-phenyl)-6-chloro-benzo[1,3]dioxol-2-yl-methyl ester (0.24 g, 0.49 mmol) and following the procedure described Intermediate 198, 0.16 g (90%) of the title compound was obtained as a colorless oil.

### Intermediate 217

2-Azidomethyl-6-chloro-4-(2,5-difluoro-phenyl)-benzo[1,3]dioxole: Starting from toluene-4-sulfonic acid 4-(2,5-difluoro-phenyl)-6-chloro-benzo[1,3]dioxol-2-yl-methyl ester (0.30 g, 0.66 mmol) and following the procedure described Intermediate 198, 0.20 g (95%) of the title compound was obtained as a colorless oil.

### Intermediate 218

2-Azidomethyl-6-chloro-4-(2,5-dimethoxy-phenyl)-benzo[1,3]dioxole: Starting from toluene-4-sulfonic acid 4-(2,5-dimethoxy-phenyl)-6-chloro-benzo[1,3]dioxol-2-yl-methyl ester (0.20 g, 0.42 mmol) and following the procedure described Intermediate 198, 0.14 g (96%) of the title compound was obtained as a colorless oil.

### Intermediate 219

2-Azidomethyl-4-biphenyl-2-yl-6-chloro-benzo[1,3]dioxole: Starting from toluene-4-sulfonic acid 4-biphenyl-6-chloro-benzo[1,3]dioxol-2-yl-methyl ester (0.47 g, 0.95 mmol) and following the procedure described Intermediate 198, 0.31 g (90%) of the title compound was obtained as a colorless oil.

### Intermediate 220

2-Azidomethyl-4-(2,6-dichloro-phenyl)-6-fluoro-benzo[1,3]dioxole: Starting from toluene-4-sulfonic acid 4-(2,6-dichloro-phenyl)-6-fluoro-benzo[1,3]dioxol-2-ylmethyl ester (0.24 g, 0.51 mmol) and following the procedure described for Intermediate 198 above, the title compound (0.16 g, 92%) was obtained as a colorless oil. MS EI m/e 339 M⁺.
General Procedure to Prepare Examples 102-108:

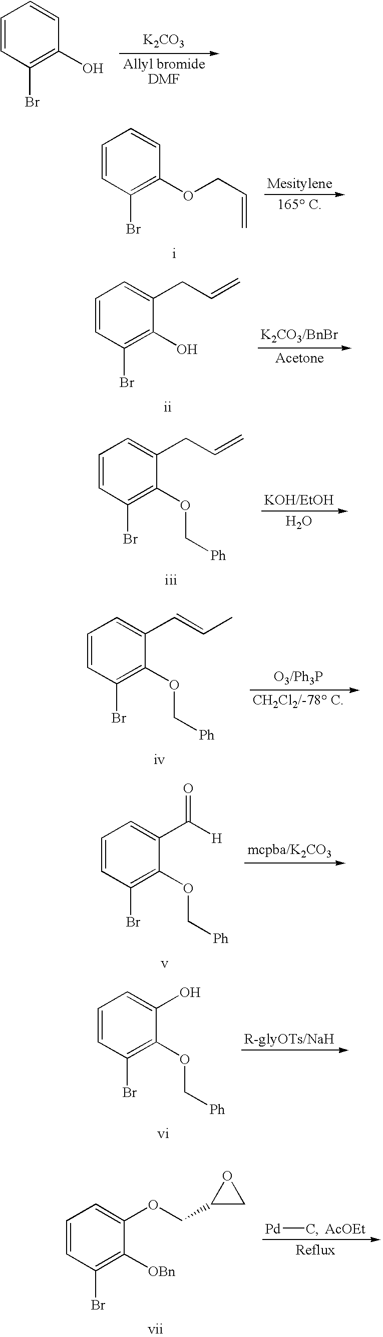

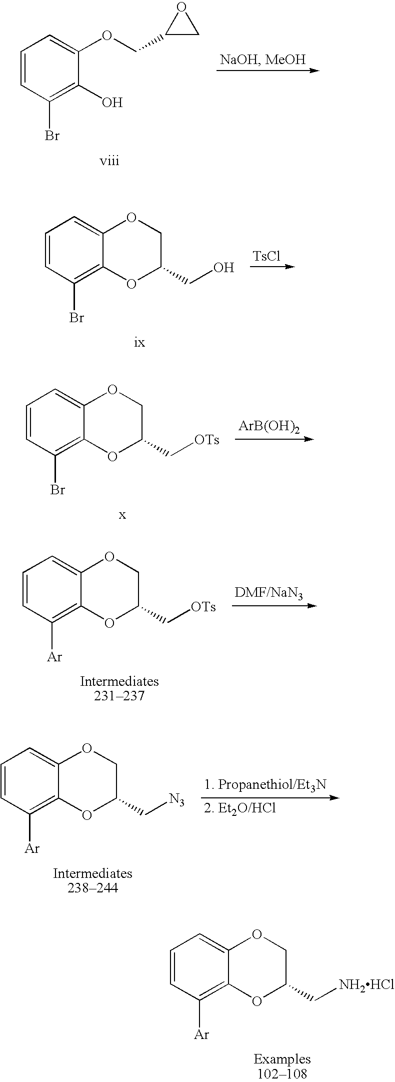

### Intermediate 221

1-allyloxy-2-bromo-benzene: A solution of 2-bromo-phenol (100 g, 578 mmol) and K₂CO₃ (159 g, 1.15 mol) in DMF (300 mL) was heated at 50° C. for 30 min. The resulting mixture was cooled to RT and allyl bromide (71 mL, 694 mmol) was added dropwise. After completion of addition the reaction mixture was allowed to stir at 50° C. for 12 h. The reaction mixture was filtered and the filtrate diluted with ethyl acetate (1000 mL) and washed with water (5×100 mL). The organic layer was dried (sodium sulfate) and concentrated under vacuum. Chromatography with 5% ethyl acetate in hexanes afforded 120 g (98%) of the title compound as a colorless oil.

### Intermediate 222

2-allyl-6-bromo-phenol: A solution of 1-allyloxy-2-bromo-benzene (100 g) in mesitylene (140 mL) was refluxed for 72 h. The mixture was diluted with hexane and basified with concentrated NaOH solution. The aqueous layer was washed with hexane (3×100 ml), acidified with concentrated HCl and extracted with ethylacetate (5×200 mL). Combined organic layers were dried (sodium sulfate) and concentrated under vacuum to afford 80 g of the crude title compound, which was used as such in the next step.

### Intermediate 223

1-allyl-2-benzyloxy-3-bromo-benzene: To a solution of 2-allyl-6-bromo-phenol (25 g, 0.12 mol) in acetone (1 L) was added potassium carbonate (33.1 g, 0.24 mol) followed by benzylbromide (25.6 g, 0.15 mol). The resulting mixture was allowed to stir at 50° C. for 12 h. The solvent was removed under vacuum and the residue diluted with water (500 ml) and extracted with ethyl acetate (2×300 ml). The combined organic layers were washed with water (500 ml), brine (500 ml), dried (sodium sulphate), and evaporated under vacuum. Chromatography with 10% ethyl acetate in hexanes afforded 30.0 g (85%) of the title compound as a pale yellow oil.

### Intermediate 224

2-benzyloxy-1-bromo-3-((E)-propenyl)-benzene: 1-allyl-2-benzyloxy-3-bromo-benzene (6.7 g, 0.02 mol) was added to a solution of KOH (7.6 g, 0.13 mol) in H₂O (3 mL) and EtOH (20 mL). The reaction mixture was heated at 90° C. for 5 h. The solvent was removed under vacuum and the residue diluted with water (20 ml) and extracted with DCM (2×20 ml). The combined organic layers were washed with water (20 ml) and brine (20 ml), dried (sodium sulphate), and the solvent removed under vacuum. Chromatography with 10% ethyl acetate in hexanes afforded 6.0 g (90%) of the title compound.

### Intermediate 225

2-benzyloxy-3-bromo-benzaldehyde: A solution of 2-benzyloxy-1-bromo-3-((E)-propenyl)-benzene (20 g, 65 mmol) in dry CH₂Cl₂ (200 mL) was cooled at −78° C. and gaseous O₃ (generated by passing O₂ through ozonolysis instrument) was bubbled for 2 hours. Triphenyl phosphine (18.16 g, 69 mmol) was added and the reaction mixture was allowed to stir for 12 h at r.t. The mixture was concentrated under vacuum. Chromatography with 10-20% ethyl acetate in hexanes afforded 16.0 g (83%) of the title compound.

### Intermediate 226

2-benzyloxy-3-bromo-phenol: To a solution of 2-benzyloxy-3-bromo-benzaldehyde (30 g, 103 mmol) in dry CH₂Cl₂ (250 mL) was added m-chloroperbenzoic acid (40.2 g, 233 mmol). The resulting mixture was stirred at room temperature for 12 h. The reaction mixture was diluted with CH₂Cl₂ (200 mL), washed with 10% NaHCO₃ solution and water. The organic layer was dried (sodium sulfate) and concentrated under vacuum. The crude oil thus obtained was dissolved in methanol (300 mL) and basic aluminum oxide (115 g) was added. The reaction mixture was stirred at RT for 12 h. Reaction mixture was filtered and the filtrate concentrated under vacuum. Chromatography with 10-20% ethyl acetate in hexanes afforded 18.0 g (62.6%) of the title compound.

### Intermediate 227

(R)-2-(3-bromo-2-benzyloxy-phenoxymethyl)-oxirane: To a solution of 2-benzyloxy-3-bromo-phenol (5.0 g, 17.9 mmole) in dry DMF (30 mL) was added 60% sodium hydride-mineral oil dispersion (17.9 mmole) and the mixture thus obtained was stirred at room temperature under nitrogen for 30 min. R-glycidyl tosylate (4.8, 21.5 mmole) was added to the reaction in one portion. The mixture was heated at 70° C. for 16 hours. The solvent was removed under vacuum and the crude was dissolved in methylene chloride (30 mL). The solution was washed with water (2×15 mL) and the aqueous back-extracted with methylene chloride (15 mL). The combined organics were washed with brine, dried (sodium sulphate), and concentrated under vacuum. Chromatography with 0-20% ethyl acetate in hexanes afforded 4.3 g (71%) of the title compound.

### Intermediate 228

2-bromo-6-((R)-1-oxiranylmethoxy)-phenol: A heterogeneous solution of (R)-2-(3-bromo-2-benzyloxy-phenoxymethyl)-oxirane (2.9 g, 8.6 mmol) and 5% Pd—C (1.35 g) in a cyclohexene:AcOEt (2.7 mL:25 mL) mixture, was refluxed under nitrogen atmosphere for 30 min. The reaction mixture was filtered on celite and concentrated under vacuum to afford 2.3 g of crude title compound, which was used as such in the following synthetic steps.

### Intermediate 229

((S)-8-Bromo-2,3-dihydrobenzo[1,4]dioxin-2-yl)-methanol: To a solution of 2-bromo-6-((R)-1-oxiranylmethoxy)-phenol (2.3 g crude) in methanol (100 mL) was added K₂CO₃ (1.5 g) and the reaction mixture was stirred for 4 h at RT. Reaction mixture was filtered and concentrated under vacuum. The residue was dissolved in CH₂Cl₂ (200 mL), washed with brine, dried (sodium sulfate) and evaporated under vacuum to afford 1.2 g of crude title compound, which was used as such in the following synthetic step.

### Intermediate 230

Synthesis of toluene-4-sulfonic acid (R)-8-bromo-2,3-dihydro-benzo[1,4]dioxin-2-ylmethyl ester: To a solution of ((S)-8-Bromo-2,3-dihydro-benzo[1,4]dioxin-2-yl)-methanol (1.2 g crude) and Et₃N (1.7 mL, 12.2 mmol) in CH₂Cl₂ (30 mL) was added p-toluensulfonyl chloride (900 mg, 4.9 mmol). The reaction mixture was stirred at RT for 12 h and then diluted with CH₂Cl₂ (200 mL), washed with 1N HCl solution and brine. The organic layer was dried (sodium sulfate) and evaporated under vacuum. Chromatography with 10-20% ethyl acetate in hexanes afforded 820 mg (24%, three steps) of the title compound.

General procedure to generate biaryl derivatives from toluene-4-sulfonic acid (R)-8-bromo-2,3-dihydro-benzo[1,4]dioxin-2-ylmethyl ester:

To a solution of toluene-4-sulfonic acid (R)-8-bromo-2,3-dihydro-benzo[1,4]dioxin-2-ylmethyl ester (1 eq) and substituted benzene boronic acid (1.5 eq) in DME-water (4/1) was added, under N₂ atmosphere, tetrakis(triphenylphospine)palladium (0) (0.1 eq) and sodium carbonate (3 eq). The reaction mixture was heated at 85° C. until starting material disappeared. [Reaction monitored by TLC]. After reaction completion the cooled reaction mixture was diluted with water and extracted with ethyl acetate. Combined organic layers were washed with brine, dried (sodium sulfate) and concentrated under vacuum. Chromatography with 10% ethyl acetate in hexanes afforded product as an oil.

Using the general procedures outlined above, Intermediates 231-237 were prepared

### Intermediate 231

Toluene-4-sulfonic acid (R)-8-(2,4-dimethyl-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (820 mg, 2 mmol) and 2,4-dimethylbenzene boronic acid, 620 mg (73%) was obtained as a colorless oil.

### Intermediate 232

Toluene-4-sulfonic acid (R)-8-(4-methoxy-2-methyl-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (400 mg, 1 mmol) and 2-methyl-4-methoxybenzene boronic acid, 400 mg (90%) was obtained as a colorless oil.

### Intermediate 233

Toluene-4-sulfonic acid (R)-8-(4-ethoxy-2-methyl-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (500 mg, 1.3 mmol) and 2-methyl-4-ethoxybenzene boronic acid, 460 mg (78%) was obtained as a colorless oil.

### Intermediate 234

Toluene-4-sulfonic acid (R)-8-(2,6-dimethoxy-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (500 mg, 1.3 mmol) and 2,6-dimethoxybenzene boronic acid, 260 mg (44%) was obtained as a colorless oil.

### Intermediate 235

Toluene-4-sulfonic acid (R)-8-(4-fluoro-2-isopropoxy-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (500 mg, 1.3 mmol) and 4-fluoro-2-isopropoxybenzene boronic acid, 500 mg (81%) was obtained as a colorless oil.

### Intermediate 236

Toluene-4-sulfonic acid (R)-8-(4-fluoro-2-methoxy-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (750 mg, 1.88 mmol) and 4-fluoro-2-methoxybenzene boronic acid, 350 mg (42%) was obtained as a colorless oil.

### Intermediate 237

Toluene-4-sulfonic acid (R)-8-(2-chloro-4-methoxy-phenyl)-2,3-dihydro-benzo[1,4]dioxin-2-ylmethyl ester: Starting from toluene-4-sulfonic acid (R)-8-bromo-2,3-dihydro-benzo[1,4]dioxin-2-ylmethyl ester (500 mg, 1.25 mmol) and 2-chloro-4-methoxybenzene boronic acid, 525 mg (90%) was obtained as a colorless oil.

General procedure to generate azido derivatives from toluene-4-sulfonic acid (R)-8-aryl-2,3-dihydro-benzo[1,4]dioxin-2-ylmethyl ester:

To a solution of toluene-4-sulfonic acid (R)-8-aryl-2,3-dihydro-benzo[1,4]dioxin-2-ylmethyl ester (1 eq) in DMSO, NaN₃ (8 eq) was added under inert atmosphere and reaction mixture was allowed to stir at 70° C. until starting material disappeared. Reaction mixture was diluted with water and extracted with ethyl acetate. Combined organic layers were washed with brine, dried over anhydrous Na₂SO₄ and concentrated under vacuum. The products obtained were used as such in the next step.

Using the general procedures outlined above, Intermediates 238-244 were prepared.

### Intermediate 238

(S)-2-Azidomethyl-8-(2,4-dimethyl-phenyl)-2,3-dihydro-benzo[1,4]dioxine: Starting from toluene-4-sulfonic acid (R)-8-(2,4-dimethyl-phenyl)-2,3-dihydro-benzo[1,4]dioxin-2-ylmethyl ester (600 mg, 1.4 mmol), 450 mg was obtained as an oil.

### Intermediate 239

(S)-2-Azidomethyl-8-(4-methoxy-2-methyl-phenyl)-2,3-dihydro-benzo[1,4]dioxine: Starting from toluene-4-sulfonic acid (R)-8-(4-methoxy-2-methyl-phenyl)-2,3-dihydro-benzo[1,4]dioxin-2-ylmethyl ester (450 mg, 1.0 mmol), 320 mg was obtained as an oil.

### Intermediate 240

(S)-2-Azidomethyl-8-(4-ethoxy-2-methyl-phenyl)-2,3-dihydro-benzo[1,4]dioxine: Starting from toluene-4-sulfonic acid (R)-8-(4-ethoxy-2-methyl-phenyl)-2,3-dihydro-benzo[1,4]dioxin-2-ylmethyl ester 460 mg, 1.1 mmol), 320 mg was obtained as an oil.

### Intermediate 241

(S)-2-Azidomethyl-8-(2,6-dimethoxy-phenyl)-2,3-dihydro-benzo[1,4]dioxine: Starting from toluene-4-sulfonic acid (R)-8-(2,6-dimethoxy-phenyl)-2,3-dihydro-benzo[1,4]dioxin-2-ylmethyl ester (260 mg, 0.6 mmol), 140 mg was obtained as an oil.

### Intermediate 242

(S)-2-Azidomethyl-8-(4-fluoro-2-isopropoxy-phenyl)-2,3-dihydro-benzo[1,4]dioxine: Starting from toluene-4-sulfonic acid (R)-8-(4-fluoro-2-isopropoxy-phenyl)-2,3-dihydro-benzo[1,4]dioxin-2-ylmethyl ester (500 mg, 1.2 mmol), 240 mg was obtained as an oil.

### Intermediate 243

(S)-2-Azidomethyl-8-(4-fluoro-2-methoxy-phenyl)-2,3-dihydro-benzo[1,4]dioxine: Starting from toluene-4-sulfonic acid (R)-8-(4-fluoro-2-methoxy-phenyl)-2,3-dihydro-benzo[1,4]dioxin-2-ylmethyl ester (350 mg, 0.8 mmol), 250 mg was obtained as an oil.

### Intermediate 244

(S)-2-Azidomethyl-8-(2-chloro-4-methoxy-phenyl)-2,3-dihydro-benzo[1,4]dioxine: Starting from toluene-4-sulfonic acid (R)-8-(2-chloro-4-methoxy-phenyl)-2,3-dihydro-benzo[1,4]dioxin-2-ylmethyl ester (500 mg, 1.2 mmol), 370 mg was obtained as an oil.

General procedure to generate amino derivatives from (S)-2-Azidomethyl-8-(aryl)-2,3-dihydro-benzo[1,4]dioxine:

To a solution of (S)-2-Azidomethyl-8-(aryl)-2,3-dihydro-benzo[1,4]dioxine (1 eq) and Et₃N (3 eq) in dry methanol cooled at 0° C., 1,3-propanedithiol (3 eq) was added under inert atmosphere. The solution was allowed to stir at RT for 12 h. The mixture was filtered through the pad of celite and concentrated under vacuum. Chromatography with 1% methanol in methylene chloride afforded the product as an oil. The oil was dissolved in methylene chloride and converted to a white solid hydrochloric salt.

### Example 1

{[8-(2-Chlorophenyl)-2,3-dihydro-1,4-benzodioxin-2-yl]methyl}amine: A solution of 2-azidomethyl-8-(2-chloro-phenyl)-2,3-dihydro-benzo[1,4]dioxine (100 mg, 0.33 mmol) and 5% Pt—S₂ on carbon (0.25 g) in methanol (50 mL) was hydrogenated under 55-60 Psi in a Parr apparatus overnight. The mixture was filtered through the pad of celite. The solvent was removed under vacuum to form a colorless oil. The oil was dissolved in ethanol and converted to a white solid fumaratefumarate salt (37 mg); mp 210-211° C.;

MS (ES) m/z 276 [M+H]⁺

Elemental Anal. for C₁₅H₁₄ClNO2.C₄H₄O4:

Theory: C, 58.25; H, 4.63; N, 3.57.

Found: C, 57.81; H, 4.58; N, 5.67.

General procedure to generate I from azide derivatives:

To a solution of intermediate azide (1.0 mmol) in tetrahydrofuran was added polymer-supported triphenylphosphine (˜3 mmol/g, 2.0 mmol) and water. The mixture was stirred at room temperature for 24 hours, and filtered through the pad of celite. The solvent was removed under vacuum to form a colorless oil. The oil was dissolved in ethanol and fumarate converted to the fumarate salt as above.

Using the general procedures outlined above, Examples 2-6, 8-16, 56-77, 78-84 and 90-101 were prepared.

### Example 2

{[8-(2-Fluorophenyl)-2,3-dihydro-1,4-benzodioxin-2-yl]methyl}amine: Starting from 2-azidomethyl-8-(2-fluoro-phenyl)-2,3-dihydro-benzo[1,4]dioxine (140 mg, 0.5 mmol), 87 mg (47%) of the title compound was obtained as a fumarate salt; mp 188-190° C.;

MS (ESI) m/z 260 [M+H]⁺

Elemental Anal. for C₁₅H₁₄FNO₂.C₄H₄O₄:

Theory: C, 60.80; H, 4.83; N, 3.73.

Found: C, 61.14; H, 4.42; N, 3.74.

### Example 3

{[8-(2-Methylphenyl)-2,3-dihydro-1,4-benzodioxin-2-yl]methyl}amine: Starting from 2-azidomethyl-8-(2-methyl-phenyl)-2,3-dihydro-benzo[1,4]dioxine (110 mg, 0.39 mmol), 42 mg (29%) of the title compound was obtained as a fumarate salt, mp 201-202° C.; MS (ESI)

m/z 256 [M+H]⁺.

Elemental anal. for C₁₆H₁₇NO₂.C₄H₄O₄:

Theory: C, 64.68; H, 5.70; N, 3.77.

Found: C, 64.70; H, 5.46; N, 3.71.

### Example 4

({8-[2-(Trifluoromethyl)phenyl]-2,3-dihydro-1,4-benzodioxin-2-yl}methyl)amine: Starting from 2-azidomethyl-8-(2-trifluoromethyl-phenyl)-2,3-dihydro-benzo[1,4]dioxine (150 mg, 0.45 mmol), 109 mg (57%) of the title compound ws obtained as a fumarate salt; mp 208-209° C.; MS (ESI) m/z 310 [M+H]⁺

Elemental anal. for C₁₆H₁₄F₃NO₂.C₄H₄O₄:

Theory: C, 56.47; H, 4.27; N, 3.29.

Found: C, 56.56; H, 4.15; N, 3.17.

### Example 5

{[8-(2-Methoxyphenyl)-2,3-dihydro-1,4-benzodioxin-2-yl]methyl}amine: Starting from 2-azidomethyl-8-(2-methoxy-phenyl)-2,3-dihydro-benzo[1,4]dioxine (130 mg, 0.39 mmol), 99 mg (58%) of the title compound was obtained as a fumarate salt, mp 186-187° C.; MS (ESI) m/z 272 [M+H]⁺; MS (ESI) m/z 313.

Elemental Anal. for C₁₆H₁₇NO₃.C₄H₄O₄:

Theory: C, 62.01; H, 5.46; N, 3.62.

Found: C, 61.91; H, 5.50; N, 3.26.

### Example 6

{[8-(2,3-Dichlorophenyl)-2,3-dihydro-1,4-benzodioxin-2-yl]methyl}amine: Starting from 2-azidomethyl-8-(2,3-dichloro-phenyl)-2,3-dihydro-benzo[1,4]dioxine (175 mg, 0.52 mmol), 76 mg (34%) of the title compound was obtained as a fumarate salt; mp 211-212° C.; MS (ESI) m/z 310 [M+H]⁺.

Elemental Anal. for C₁₅H₁₃C₁₂NO₂.C₄H₄O₄:

Theory: C, 53.54; H, 4.02; N, 3.29.

Found: C, 52.67; H, 3.34; N, 3.05.

### Example 7

{[8-(2,4-Dichlorophenyl)-2,3-dihydro-1,4-benzodioxin-2-yl]methyl}amine: A solution of 2-azidomethyl-8-(2,4-dichloro-phenyl)-2,3-dihydro-benzo[1,4]dioxine (130 mg, 0.39 mmol) and 5% Pt—S₂ on carbon (0.25 g) in methanol (50 mL) was hydrogenated under 55-60 psi overnight. The mixture was filtered through the pad of celite. The solvent was removed under vacuum to form a colorless oil. The oil was dissolved in ethanol and converted to a white solid fumarate salt (30 mg); mp 192-193° C.; MS (ES) m/z 310.0 [M+H]⁺.

Elemental Anal. for C₁₅H₁₃C₁₂NO₂.C₄H₄O₄:

Theory: C, 53.54; H, 4.02; N, 3.29.

Found: C, 53.56; H, 3.93; N, 3.09.

### Example 8

{[8-(2,5-Dichlorophenyl)-2,3-dihydro-1,4-benzodioxin-2-yl]methyl}amine: Starting from 2-azidomethyl-8-(2,5-dichloro-phenyl)-2,3-dihydro-benzo[1,4]dioxine (140 mg), 87 mg (49%) of the title compound was obtained as a fumarate salt; mp 206-207° C.; MS (ESI) m/z 310 [M+H]⁺.

Elemental Anal. for C₁₅H₁₃Cl₂NO₂.C₄H₄O₄:

Theory: C, 53.54; H, 4.02; N, 3.29.

Found: C, 53.64; H, 3.49; N, 3.13.

### Example 9

{[8-(2,3-Dimethoxyphenyl)-2,3-dihydro-1,4-benzodioxin-2-yl]methyl}amine: Starting from 2-azidomethyl-8-(2,3-dimethoxy-phenyl)-2,3-dihydro-benzo[1,4]dioxine (100 mg, 0.30 mmol), 59 mg (46%) of the title compound was obtained as a fumarate salt; mp 198-199° C.;

MS (ESI) m/z 302 [M+H]⁺.

Elemental Anal. for C₁₇H₁₉NO₄.C₄H₄O₄:

Theory: C, 60.43; H, 5.55; N, 3.36.

Found: C, 60.05; H, 5.44; N, 3.18.

### Example 10

{[8-(2,3-Dimethylphenyl)-2,3-dihydro-1,4-benzodioxin-2-yl]methyl}amine: Starting from 2-azidomethyl-8-(2,3-dimethyl-phenyl)-2,3-dihydro-benzo[1,4]dioxine (130 mg, 0.44 mmol), 104 mg (61%) of the title compound was obtained as a fumarate salt; mp 204-205° C.; MS (ESI) m/z 270; MS (ESI) m/z 311 [M+H]⁺.

Elemental Anal. for C₁₇H₁₉NO₂.C₄H₄O₄:

Theory: C, 65.44; H, 6.02; N, 3.63.

Found: C, 65.39; H, 5.99; N, 3.27.

### Example 11

{[8-(2,5-Dimethylphenyl)-2,3-dihydro-1,4-benzodioxin-2-yl]methyl}amine: Starting from 2-azidomethyl-8-(2,5-dimethyl-phenyl)-2,3-dihydro-benzo[1,4]dioxine (140 mg, 0.47 mmol), 103 mg (56%) of the title compound was obtained as a fumarate salt; mp 199-200° C.; MS (ES) m/z 270.2 [M+H]⁺.

Elemental Anal. for C₁₇H₁₉NO₂.C₄H₄O₄:

Theory C, 65.44; H, 6.02; N, 3.63.

Found: C, 65.24; H, 5.45; N, 3.44.

### Example 12

{[8-(2,6-Dimethylphenyl)-2,3-dihydro-1,4-benzodioxin-2-yl]methyl}amine: Starting from 2-azidomethyl-8-(2,6-dimethyl-phenyl)-2,3-dihydro-benzo[1,4]dioxine, 30 mg of the title compound was obtained as a fumarate salt; mp 205-206° C.; MS (ES) m/z 270.1 [M+H]+

Elemental Anal. for C₁₇H₁₉NO₂.C₄H₄O₄:

Theory: C, 65.44; H, 6.02; N, 3.63.

Found: C, 65.27; H, 6.12; N, 3.48.

### Example 13

{[8-(2,3-Difluorophenyl)-2,3-dihydro-1,4-benzodioxin-2-yl]methyl}amine: Starting from 2-azidomethyl-8-(2,3-difluoro-phenyl)-2,3-dihydro-benzo[1,4]dioxine (150 mg, 0.49 mmol), 73 mg (38%) of the title compound was obtained as a fumarate salt; mp 189-190° C.; MS (ESI) m/z 278 [M+H]⁺.

Elemental Anal. for C₁₅H₁₃F₂NO₂.C₄H₄O₄:

Theory: C, 58.02; H, 4.36; N, 3.56.

Found: C, 57.82; H, 4.30; N, 2.78.

### Example 14

{[8-(2,4-Difluorophenyl)-2,3-dihydro-1,4-benzodioxin-2-yl]methyl}amine: Starting from 2-azidomethyl-8-(2,4-difluoro-phenyl)-2,3-dihydro-benzo[1,4]dioxine (120 mg, 0.39 mmol), 62 mg (40%) of the title compound was obtained as a fumarate salt; mp 183-184° C.; MS (ES) m/z 278.1 [M+H]⁺.

Elemental Anal. for C₁₅H₁₃F₂NO₂.C₄H₄O₄:

Theory: C, 58.02; H, 4.36; N, 3.56.

Found: C, 58.03; H, 4.44; N, 3.32.

### Example 15

{[8-(2,5-Difluorophenyl)-2,3-dihydro-1,4-benzodioxin-2-yl]methyl}amine: Starting from 2-azidomethyl-8-(2,5-didifluoro-phenyl)-2,3-dihydro-benzo[1,4]dioxine (120 mg, 0.40 mmol), 46 mg (30%) of the title compound was obtained as a fumarate salt; mp 189-190° C.;

MS (ESI) m/z 278 [M+H]⁺.

Elemental Anal. for C₁₅H₁₃F₂NO₂.C₄H₄O₄:

Theory: C, 58.02; H, 4.36; N, 3.56.

Found: C, 57.86; H, 4.50; N, 3.21.

### Example 16

{[8-(5-Chloro-2-methoxyphenyl)-2,3-dihydro-1,4-benzodioxin-2-yl]methyl}amine: Starting from 2-azidomethyl-8-(5-chloro-2-methoxy-phenyl)-2,3-dihydro-benzo[1,4]-dioxine (140 mg, 0.42 mmol), 104 mg (58%) of the title compound was obtained as a fumarate salt; mp 192-193° C.; MS (ESI) m/z 306 [M+H]⁺.

Elemental Anal. for C₁₆H₁₆ClNO₃.0.50 C₄H₄O₄:

Theory: C, 59.43; H, 4.99; N, 3.85.

Found: C, 59.20; H, 4.87; N, 3.55.

General procedure to generate I from corresponding tosylate:

A solution of tosylate (1 eq) and corresponding amines (10 eq) in DMSO was heated at 70° C. overnight. The reaction was quenched with saturated sodium bicarbonate and extracted with methylene chloride. The organic layer was washed with water and dried over anhydrous sodium sulfate. The solvent was removed under vacuum and afforded a crude material. The crude oil was dissolved in ethanol and converted into its fumaric salt by addition of one equivalent of fumaric acid.

Using the general procedures outlined above, Examples 17-45 were prepared.

### Example 17

N-Methyl-N-{[8-(2-methylphenyl)-2,3-dihydro-1,4-benzodioxin-2-yl]methyl}amine: Starting from toluene-4-sulfonic acid 8-(2-methyl-phenyl)-2,3-dihydro-benzo[1,4]dioxin-2-yl-methyl ester (175 mg, 0.43 mmol), 116 mg (70%) of the title compound was obtained as a fumarate salt; mp 177-178° C.; MS (ES) m/z 270.2 [M+H]⁺.

Elemental anal. for C₁₇H₁₉NO₂.C₄H₄O₄

Theory: C, 65.44; H, 6.02; N, 3.63.

Found: C, 65.25; H, 6.02; N, 3.35.

### Example 18

{[8-(2-Fluorophenyl)-2,3-dihydro-1,4-benzodioxin-2-yl]methyl}methylamine: Starting from toluene-4-sulfonic acid 8-(2-fluoro-phenyl)-2,3-dihydro-benzo[1,4]dioxin-2-yl-methyl ester (205 mg, 0.49 mmol), 148 mg (77%) of the title compound was obtained as a fumarate salt, mp 191-192° C.; MS (ES) m/z 274.1 [M+H]⁺.

Elemental Anal. for C₁₆H₁₆FNO₂.C₄H₄O₄:

Theory C, 61.69; H, 5.18; N, 3.60.

Found: C, 61.36; H, 4.92; N, 3.23.

### Example 19

{[8-(2-Methoxyphenyl)-2,3-dihydro-1,4-benzodioxin-2-yl]methyl}methylamine: Starting from toluene-4-sulfonic acid 8-(2-methoxy-phenyl)-2,3-dihydro-benzo[1,4]-dioxin-2-yl-methyl ester (175 mg, 0.41 mmol,) 123 mg (74%) of the title compound was obtained as a fumarate salt; mp 168-169° C.; MS (ES) m/z 286.1 [M+H]⁺.

Elemental Anal. for C₁₇H₁₉NO₃.C₄H₄O₄:

Theory: C, 62.84; H, 5.78; N, 3.49.

Found: C, 62.43; H, 5.94; N, 3.31.

### Example 20

N-Methyl-1-{8-[2-(trifluoromethyl)phenyl]-2,3-dihydro-1,4-benzodioxin-2-yl}methanamine: Starting from toluene-4-sulfonic acid 8-(2-trifluoromethyl-phenyl)-2,3-dihydro-benzo[1,4]-dioxin-2-yl-methyl ester (220 mg, 0.47 mmol), 116 mg (72%) of the title compound was obtained as a fumarate salt; mp 184-185° C.; MS (ES) m/z 324.1 [M+H]⁺.

Elemental Anal. for C₁₇H₁₆F₃NO₂.C₄H₄O₄:

Theory: C, 57.41; H, 4.59; N, 3.19.

Found: C, 57.45; H, 4.40; N, 2.99.

### Example 21

{[8-(2-Chlorophenyl)-2,3-dihydro-1,4-benzodioxin-2-yl]methyl}methylamine: Starting from toluene-4-sulfonic acid 8-(2-chloro-phenyl)-2,3-dihydro-benzo[1,4]-dioxin-2-yl-methyl ester (143 mg, 0.33 mmol), 74 mg (55%) of the title compound was obtained as a fumarate salt; mp 174-175° C.; MS (ES) m/z 290.1 [M+H]⁺.

### Example 22

{[8-(2,3-Dichlorophenyl)-2,3-dihydro-1,4-benzodioxin-2-yl]methyl}methylamine: Starting from toluene-4-sulfonic acid 8-(2,3-dichloro-phenyl)-2,3-dihydro-benzo[1,4]-dioxin-2-yl-methyl ester (175 mg, 0.38 mmol), 117 mg (71%) of the title compound was obtained as a fumarate salt; mp 184-185° C.; MS (ES) m/z 324.1 [M+H]⁺.

Elemental Anal. for C₁₆H₁₅C₁₂NO₂.C₄H₄O₄

Theory: C, 54.56; H, 4.35; N, 3.18.

Found: C, 54.49; H, 3.99; N, 2.78.

### Example 23

{[8-(2,4-Dichlorophenyl)-2,3-dihydro-1,4-benzodioxin-2-yl]methyl}methylamine: Starting from toluene-4-sulfonic acid 8-(2,4-dichloro-phenyl)-2,3-dihydro-benzo[1,4]-dioxin-2-yl-methyl ester (150 mg, 0.32 mmol), 87 mg (61%) of the title compound was obtained as a fumarate salt, mp 180-181° C.; MS (ES) m/z 324.1 [M+H]⁺.

### Example 24

{[8-(2,5-Dichlorophenyl)-2,3-dihydro-1,4-benzodioxin-2-yl]methyl}methylamine: Starting from toluene-4-sulfonic acid 8-(2,5-dichloro-phenyl)-2,3-dihydro-benzo[1,4]-dioxin-2-yl-methyl ester (185 mg, 0.4 mmol), 141 mg (80%) of the title compound was obtained as a fumarate salt; mp 166-167° C.; MS (ES) m/z 324.1 [M+H]⁺.

Elemental Anal. for C₁₆H₁₅C₁₂NO₂.C₄H₄O₄:

Theory: C, 54.56; H, 4.35; N, 3.18.

Found: C, 54.46; H, 4.38; N, 2.88.

### Example 25

{[8-(2,3-Dimethylphenyl)-2,3-dihydro-1,4-benzodioxin-2-yl]methyl}methylamine: Starting from toluene-4-sulfonic acid 8-(2,3-dimethyl-phenyl)-2,3-dihydro-benzo[1,4]-dioxin-2-yl-methyl ester (185 mg, 0.43 mmol), 115 mg (66%) of the title compound was obtained as a fumarate salt; mp 194-195° C.; MS (ES) m/z 284.1 [M+H]⁺.

Elemental Anal. for C₁₈H₂₁NO₂.C₄H₄O₄:

Theory: C, 66.15; H, 6.31; N, 3.51.

Found: C, 66.10; H, 6.12; N, 3.42.

### Example 26

{[8-(2,5-Dimethylphenyl)-2,3-dihydro-1,4-benzodioxin-2-yl]methyl}methylamine: Starting from toluene-4-sulfonic acid 8-(2,5-methyl-phenyl)-2,3-dihydro-benzo[1,4]-dioxin-2-yl-methyl ester (215 mg, 0.50 mmol), 131 mg (67%) of the title compound was obtained as a fumarate salt; mp 174-175° C.; MS (ES) m/z 284.2 [M+H]⁺.

Elemental Anal. for C₁₈H₂₁NO₂.C₄H₄O₄:

Theory: C, 66.15; H, 6.31; N, 3.51.

Found: C, 65.89; H, 6.27; N, 3.17.

### Example 27

{[8-(2,3-Difluorophenyl)-2,3-dihydro-1,4-benzodioxin-2-yl]methyl}methylamine: Starting from toluene-4-sulfonic acid 8-(2,3-difluoro-phenyl)-2,3-dihydro-benzo[1,4]-dioxin-2-yl-methyl ester (200 mg, 0.46 mmol), 125 mg (66%) of the title compound was obtained as a fumarate salt; mp 181-182° C.; MS (ES) m/z 292.1 [M+H]⁺.

Elemental Anal. for C₁₆H₁₅F₂NO₂.C₄H₄O₄:

Theory: C, 58.97; H, 4.70; N, 3.44.

Found: C, 58.80; H, 4.69; N, 3.13.

### Example 28

{[8-(2,4-Difluorophenyl)-2,3-dihydro-1,4-benzodioxin-2-yl]methyl}methylamine: Starting from toluene-4-sulfonic acid 8-(2,4-difluoro-phenyl)-2,3-dihydro-benzo[1,4]-dioxin-2-yl-methyl ester (200 mg, 0.46 mmol), 135 mg (71%) of the title compound was obtained as a fumarate salt; mp 174-175° C.; MS (ES) m/z 292.1 [M+H]⁺.

Elemental Anal. for C₁₆H₁₅F₂NO₂.C₄H₄O₄:

Theory: C, 58.97; H, 4.70; N, 3.44.

Found: C, 58.86; H, 4.77; N, 3.37.

### Example 29

{[8-(2,5-Difluorophenyl)-2,3-dihydro-1,4-benzodioxin-2-yl]methyl}methylamine: Starting from toluene-4-sulfonic acid 8-(2,5-difluoro-phenyl)-2,3-dihydro-benzo[1,4]-dioxin-2-yl-methyl ester (185 mg, 0.42 mmol), 135 mg (77%) of the title compound was obtained as a fumarate salt; mp 193-194° C.; MS (ES) m/z 292.1 [M+H]⁺.

Elemental Anal. for C₁₆H₁₅F₂NO₂.C₄H₄O₄:

Theory: C, 58.97; H, 4.70; N, 3.44.

Found: C, 58.64; H, 4.51; N, 3.25.

### Example 30

{[8-(2,3-Dimethoxyphenyl)-2,3-dihydro-1,4-benzodioxin-2-yl]methyl}methylamine: Starting from toluene-4-sulfonic acid 8-(2,3-dimethoxy-phenyl)-2,3-dihydro-benzo[1,4]-dioxin-2-yl-methyl ester (155 mg, 0.34 mmol), 37 mg (25%) of the title compound was obtained as a fumarate salt; mp 159-160° C.; MS (ES) m/z 316.1.

Elemental Anal. for C₁₈H₂₁NO₄.C₄H₄O₄:

Theory: C, 61.25; H, 5.84; N, 3.25.

Found: C, 61.04; H, 5.79; N, 3.19.

### Example 31

{[8-(5-Chloro-2-methoxyphenyl)-2,3-dihydro-1,4-benzodioxin-2-yl]methyl}methylamine: Starting from toluene-4-sulfonic acid 8-(5-chloro-2-methoxy-phenyl)-2,3-dihydro-benzo[1,4]-dioxin-2-yl-methyl ester (230 mg, 0.50 mmol), 51 mg (24%) of the title compound was obtained as a fumarate salt; mp 172-173° C.; MS (ES) m/z 320.1.

Elemental Anal. for C₁₇H₁₈ClNO₃.C₄H₄O₄:

Theory: C, 57.87; H, 5.09; N, 3.21.

Found: C, 57.82; H, 4.42; N, 3.16.

### Example 32

N-{[8-(2-Chlorophenyl)-2,3-dihydro-1,4-benzodioxin-2-yl]methyl}ethanamine: Starting from toluene-4-sulfonic acid 8-(2-chloro-phenyl)-2,3-dihydro-benzo[1,4]-dioxin-2-yl-methyl ester (142 mg, 0.33 mmol), 107 mg (77%) of the title compound was obtained as a fumarate salt, mp 196-197° C.; MS (ES) m/z 304.1.

### Example 33

N-{[8-(2,4-Dichlorophenyl)-2,3-dihydro-1,4-benzodioxin-2-yl]methyl}ethanamine: Starting from toluene-4-sulfonic acid 8-(2,4-dichloro-phenyl)-2,3-dihydro-benzo[1,4]-dioxin-2-yl-methyl ester (150 mg, 0.32 mmol), 92 mg (62%) of the title compound was obtained as a fumarate salt; mp 179-180° C.; MS (ES) m/z 338.1 [M+H]⁺.

### Example 34

N-{[8-(2-Chlorophenyl)-2,3-dihydro-1,4-benzodioxin-2-yl]methyl}propan-1-amine: Starting from toluene-4-sulfonic acid 8-(2-chloro-phenyl)-2,3-dihydro-benzo[1,4]-dioxin-2-yl-methyl ester (140 mg, 0.32 mmol), 88 mg (62%) of the title compound was obtained as a fumarate salt, mp 166-167° C.; MS (ES) m/z 318.1 [M+H]⁺.

### Example 35

N-{[8-(2,4-Dichlorophenyl)-2,3-dihydro-1,4-benzodioxin-2-yl]methyl}propan-1-amine: Starting from toluene-4-sulfonic acid 8-(2,4-dichloro-phenyl)-2,3-dihydro-benzo[1,4]-dioxin-2-yl-methyl ester (150 mg, 0.32 mmol), 77 mg (51%) of the title compound was obtained as a fumarate salt, mp 178-179° C.; MS (ES) m/z 352.1 [M+H]⁺.

### Example 36

N-{[8-(2-Chlorophenyl)-2,3-dihydro-1,4-benzodioxin-2-yl]methyl}propan-2-amine: Starting from toluene-4-sulfonic acid 8-(2-chloro-phenyl)-2,3-dihydro-benzo[1,4]-dioxin-2-yl-methyl ester (140 mg, 0.33 mmol), 58 mg (41%) of the title compound was obtained as a fumarate salt; mp 199-200° C.; MS (ES) m/z 318.1 [M+H]⁺.

### Example 37

N-{[8-(2,4-Dichlorophenyl)-2,3-dihydro-1,4-benzodioxin-2-yl]methyl}propan-2-amine: Starting from toluene-4-sulfonic acid 8-(2,4-dichloro-phenyl)-2,3-dihydro-benzo[1,4]-dioxin-2-yl-methyl ester (150 mg, 0.32 mmol), 70 mg (51%) of the title compound was obtained as a fumarate salt, mp 222-223° C.; MS (ES) m/z 352.1 [M+H]⁺.

### Example 38

{[8-(2-Chlorophenyl)-2,3-dihydro-1,4-benzodioxin-2-yl]methyl}dimethylamine: Starting from toluene-4-sulfonic acid 8-(2-chloro-phenyl)-2,3-dihydro-benzo[1,4]-dioxin-2-yl-methyl ester (140 mg, 0.33 mmol), 69 mg (51%) of the title compound was obtained as a fumarate salt; mp 196-197° C.; MS (ES) m/z 304.1 [M+H]⁺.

### Example 39

{[8-(2,4-Dichlorophenyl)-2,3-dihydro-1,4-benzodioxin-2-yl]methyl}dimethylamine: Starting from toluene-4-sulfonic acid 8-(2,4-dichloro-phenyl)-2,3-dihydro-benzo[1,4]-dioxin-2-yl-methyl ester (150 mg, 0.32 mmol), 84 mg (51%) of the title compound was obtained as a fumarate salt, mp 232-233° C.; MS (ES) m/z 338.1 [M+H]⁺.

### Example 40

N-{[8-(2,4-Dichlorophenyl)-2,3-dihydro-1,4-benzodioxin-2-yl]methyl}prop-2-en-1-amine: Starting from toluene-4-sulfonic acid 8-(2,4-dichloro-phenyl)-2,3-dihydro-benzo[1,4]-dioxin-2-yl-methyl ester (150 mg, 0.32 mmol), 58 mg (39%) of the title compound was obtained as a fumarate salt, mp 167-168° C.; MS (ES) m/z 350.1 [M+H]⁺.

### Example 41

{[8-(2-Chlorophenyl)-2,3-dihydro-1,4-benzodioxin-2-yl]methyl}(cyclopropylmethyl)-amine: Starting from toluene-4-sulfonic acid 8-(2-chloro-phenyl)-2,3-dihydro-benzo[1,4]-dioxin-2-yl-methyl ester (142 mg, 0.33 mmol), 112 mg (76%) of the title compound was obtained as a fumarate salt; mp 138-140° C.; MS (ES) m/z 330.1 [M+H]⁺.

### Example 42

(Cyclopropylmethyl) {[8-(2,4-dichlorophenyl)-2,3-dihydro-1,4-benzodioxin-2-yl]methyl}amine: Starting from toluene-4-sulfonic acid 8-(2,4-dichloro-phenyl)-2,3-dihydro-benzo[1,4]-dioxin-2-yl-methyl ester (150 mg, 0.32 mmol), 61 mg (39%) of the title compound was obtained as a fumarate salt, mp 155-156° C.; MS (ES) m/z 364.1 [M+H]⁺.

### Example 43

N-{[8-(2-Chlorophenyl)-2,3-dihydro-1,4-benzodioxin-2yl]methyl}cyclopropanamine: Starting from toluene-4-sulfonic acid 8-(2-chloro-phenyl)-2,3-dihydro-benzo[1,4]-dioxin-2-yl-methyl ester (142 mg, 0.33 mmol), 79 mg (55%) of the title compound was obtained as a fumarate salt; mp 148-149° C.; MS (ES) m/z 316.1 [M+H]⁺.

### Example 44

N-{[8-(2,4-Dichlorophenyl)-2,3-dihydro-1,4-benzodioxin-2-yl]methyl}cyclopropanamine: Starting from toluene-4-sulfonic acid 8-(2,4-dichloro-phenyl)-2,3-dihydro-benzo[1,4]-dioxin-2-yl-methyl ester (150 mg, 0.32 mmol), 66 mg (44%) of the title compound was obtained as a fumarate salt, mp 181-182° C.; MS (ES) m/z 350.1 [M+H]⁺.

### Example 45

N-{[8-(2,4-Dichlorophenyl)-2,3-dihydro-1,4-benzodioxin-2-yl]methyl}cyclobutanamine: Starting from toluene-4-sulfonic acid 8-(2,4-dichloro-phenyl)-2,3-dihydro-benzo[1,4]-dioxin-2-yl-methyl ester (150 mg, 0.32 mmol), 109 mg (70%) of the title compound was obtained as a fumarate salt, mp 204-205° C.; MS (ES) m/z 364.1 [M+H]⁺.

### Example 46

{[(2S)-8-(2,6-Dichlorophenyl)-2,3-dihydro-1,4-benzodioxin-2-yl]methyl}amine: To a solution of (S)-2-azidomethyl-8-(2,6-dichlorophenyl)-2,3-dihydro-benzo-[1,4]dioxin (1.33 g, 3.95 mmol) in tetrahydrofuran was added triphenylphosphine (1.5 g, 5.9 mmol) and water. The mixture was stirred at room temperature for 24 hours. The solvent was removed under vacuum. Chromatography with 0-10% methanol in ethyl acetate plus 1% NH₄OH afforded a colorless oil. The oil was dissolved in ethyl acetate and made into its hydrochloric salt (0.95 g, 69%) using excess ethereal hydrochloric acid to give a white solid, mp 165-167° C.; [α]^{D}₂₅=+20.00° (c 1% solution in MeOH).

Elemental Anal. for C₁₅H₁₃Cl₂NO₂.HCl:

Theory: C, 51.97; H, 4.07; N, 4.04.

Found: C, 51.60; H, 4.39; N, 3.64.

### Example 47

{[(2S)-8-(2,6-Dichlorophenyl)-6-fluoro-2,3-dihydro-1,4-benzodioxin-2-yl]methyl}amine:

To a solution of (S)-2-azidomethyl-8-(2,6-dichlorophenyl)-6-fluoro-2,3-dihydro-benzo-[1,4]dioxin (0.26 g, 0.73 mmol) in tetrahydrofuran was added polymer-supported triphenylphosphine (˜3 mmol/g, 2.0 mmol) and water. The mixture was stirred at room temperature for 24 hours, and filtered through the pad of celite. The solvent was removed under vacuum to form a colorless oil. The oil was dissolved in ethyl acetate and made into its hydrochloric salt (0.17 g, 65%) using excess ethereal hydrochloric acid to give a white solid, mp 165-167° C.; [α]^{D}₂₅=+16.81° (c=5.4 MG/0.7 ML, MeOH).

Elemental Anal. for C₁₅H₁₂Cl₂FNO₂.HCl:

Theory C, 49.41; H, 3.59; N, 3.84.

Found: C, 49.02; H, 3.54; N, 3.64.

### Example 48

{[(2S)-2-Methyl-8-phenyl-2,3-dihydro-1,4-benzodioxin-2-yl]methyl}amine: To a solution of (S)-2-azidomethyl-2-methyl-8-phenyl-2,3-dihydro-benzo[1,4]dioxine (0.17 g, 0.60 mmol) in tetrahydrofuran was added polymer-supported triphenylphosphine (˜3 mmol/g, 2.0 mmol) and water. The mixture was stirred at room temperature for 24 hours, and filtered through the pad of celite. The solvent was removed under vacuum to form a colorless oil. The oil was dissolved in ethyl acetate and converted to a white solid fumarate salt (29 mg, 16%) by crystallization from ethanol with addition of one equivalent of fumaric acid. mp 130-133° C.;

MS (ES) m/z 256.1 [M+H]⁺.

Elemental Anal. for C₁₆H₁₇NO₂.C₄H₄O₄:

Theory: C, 64.68; H, 5.70; N, 3.77.

Found: C, 64.42; H, 5.73; N, 3.46.

### Example 49

{[(2S)-8-(2-Chlorophenyl)-2-methyl-2,3-dihydro-1,4-benzodioxin-2-yl]methyl}amine: Starting from (S)-2-azidomethyl-8-(2-chloro-phenyl)-2-methyl-2,3-dihydro-benzo[1,4]-dioxine (0.16 g, 0.51 mmol) and polymer-supported triphenylphosphine (˜3 mmol/g, 1.5 mmol), the procedure described for Example 48 gave the title compound (0.10 g, 91%) as a colorless oil. The oil was dissolved in isopropyl alcohol and one equivalent of fumaric acid added to give the fumarate as a white solid, mp 149-152° C.; MS (ES) m/z 290.1 [M+H]⁺.

Elemental Anal. for C₁₆H₁₆ClNO₂.C₄H₄O₄.0.85H₂O:

Theory: C, 57.04; H, 5.19; N, 3.33.

Found: C, 56.59; H, 4.74; N, 3.06.

### Example 50

{[(2S)-8-(3-Chlorophenyl)-2-methyl-2,3-dihydro-1,4-benzodioxin-2-yl]methyl}amine: Starting from (S)-2-azidomethyl-8-(3-chloro-phenyl)-2-methyl-2,3-dihydro-benzo[1,4]-dioxine (0.17 g, 0.54 mmol) and polymer-supported triphenylphosphine (˜3 mmol/g, 0.27 g,) the procedure described for Example 48, gave the title compound (0.13 g, 83%) as a colorless oil. The oil was dissolved in isopropyl alcohol and one equivalent of fumaric acid added to give the fumarate as a white solid, mp 110-113° C.; MS (ES) m/z 290.1 [M+H]⁺.

Elemental Anal. for C₁₆H₁₆ClNO₂.C₄H₄O₄.0.50H₂O:

Theory: C, 57.91; H, 5.10; N, 3.38.

Found: C, 57.84; H, 5.12; N, 3.38.

### Example 51

{[(2S)-8-(4-Chlorophenyl)-2-methyl-2,3-dihydro-1,4-benzodioxin-2-yl]methyl}amine: Starting from (S)-2-azidomethyl-8-(4-chloro-phenyl)-2-methyl-2,3-dihydro-benzo[1,4]-dioxine (0.23 g, 0.73 mmol) and polymer-supported triphenylphosphine (˜3 mmol/g, 0.24 g), the procedure described for Example 48, gave the title compound (0.18 g, 85%) as a colorless oil. The oil was dissolved in isopropyl alcohol and one equivalent of fumaric acid added to give the fumarate as a white solid, mp 155-158° C.; MS (ES) m/z 290.1; MS (ES) m/z 331.1 [M+H]⁺.

Elemental Anal. for C₁₆H₁₆ClNO₂.C₄H₄O₄.H₂O:

Theory: C, 56.68; H, 5.23; N, 3.30.

Found: C, 56.75; H, 4.87; N, 3.07.

### Example 52

{[(2S)-8-(2-Methoxyphenyl)-2-methyl-2,3-dihydro-1,4-benzodioxin-2-yl]methyl}amine: Starting from (S)-2-azidomethyl-8-(2-methoxyphenyl)-2-methyl-2,3-dihydro-benzo[1,4]-dioxine (0.13 g, 0.42 mmol) and polymer-supported triphenylphosphine (˜3 mmol/g, 0.14 g), the procedure described for Example 48 gave the title compound (0.11 g, 90%) as a colorless oil. The oil was dissolved in isopropyl alcohol and one equivalent of fumaric acid added to give the fumarate as a white solid, mp 128-131° C.; MS (ES) m/z 286.1 [M+H]⁺.

Elemental Anal. for C₁₇H₁₉NO₃.C₄H₄O₄.1.75H₂O:

Theory: C, 58.26; H, 6.17; N, 3.24.

Found: C, 58.00; H, 5.87; N, 3.19.

### Example 53

{[(2S)-2-Methyl-8-thien-3-yl-2,3-dihydro-1,4-benzodioxin-2-yl]methyl}amine: Starting from (S)-2-azidomethyl-8-thien-3-yl-2,3-dihydro-benzo[1,4]-dioxine (0.13 g, 0.45 mmol) and polymer-supported triphenylphosphine (˜3 mmol/g, 0.15 g), the procedure described for Example 48 gave the desired product (0.11 g, 93%) as a colorless oil. The oil was dissolved in isopropyl alcohol and one equivalent of fumaric acid added to give the fumarate as a white solid, mp 158-161° C.; MS (ES) m/z 262.1; MS (ES) m/z 303.1 [M+H]⁺

Elemental Anal. for C₁₄H₁₅NO₂S.C₄H₄O₄.0.25H₂O:

Theory: C, 56.61; H, 5.15; N, 3.67.

Found: C, 56.85; H, 5.02; N, 3.47.

### Example 54

1-[4-(2,4-Dichlorophenyl)-6-fluoro-1,3-benzodioxol-2-yl]methanamine: To a solution of 2-azidomethyl-4-(2,4-dichloro-phenyl)-6-fluoro-benzo[1,3]dioxole (0.24 g, 0.7 mmol) in tetrahydrofuran was added triphenylphosphine (0.22 g, 0.84 mmol) and water. The mixture was stirred at room temperature for 24 hours, and filtered through the pad of celite. The solvent was removed under vacuum to form a colorless oil. The oil was dissolved in ethyl acetate and made into its hydrochloride salt (115 mg, 46%) using excess ethereal hydrochloric acid to give a white solid, mp 218-220° C.; MS (ES) m/z 314.0 [M+H]⁺.

Elemental Anal. for C₁₄H₁₀C₁₂FNO₂.HCl.0.25H₂O:

Theory: C, 47.35; H, 3.26; N, 3.94.

Found: C, 47.24; H, 2.99; N, 3.89.

### Example 55

1-[4-(2,6-Dichlorophenyl)-6-fluoro-1,3-benzodioxol-2-yl]methanamine: To a solution of 2-azidomethyl-4-(2,6-dichloro-phenyl)-6-fluoro-benzo[1,3]dioxole (0.16 g, 0.47 mmol) in tetrahydrofuran was added triphenylphosphine (0.18 g, 0.71 mmol) and water. The mixture was stirred at room temperature for 24 hours, and filtered through the pad of celite. The solvent was removed under vacuum to form a colorless oil. The oil was dissolved in ethyl acetate and made into its hydrochloric salt (76 mg, 46%) using excess ethereal hydrochloric acid to give a white solid, mp 224-226° C.; MS (ES) m/z 314.0 [M+H]⁺.

Elemental anal. for C₁₄H₁₀C₁₂FNO₂.HCl:

Theory: C, 47.96; H, 3.16; N, 3.99.

Found: C, 48.20; H, 2.96; N, 3.86.

### Example 56

1-[(2S)-8-(2-CHLOROPHENYL)-2,3-DIHYDRO-1,4-BENZODIOXIN-2-YL]METHANAMINE: To a solution of (R)-toluene-4-sulfonic acid 8-trifluoromethanesulfonyloxy-2,3-dihydrobenzo[1,4]dioxin-2-ylmethyl ester (0.90 g, 1.9 mmol), prepared from (R)-toluene-4-sulfonic acid 8-formyl-2,3-dihydrobenzo[1,4]dioxin-2-ylmethyl ester by the procedure described for Intermediate 2, and 2-chlorobenzene boronic acid (1.2 g, 7.6 mmol) in 50 mL of DME, was added 10 mL of water and 0.50 g (4.7 mmol) of sodium carbonate. The mixture was brought to reflux under Argon and 112 mg of tetrakis(triphenylphosphine)palladium (0) was added. Reflux was continued overnight. The solvent was removed in vacuum and replaced with 400 mL of methylene chloride. The solution was washed with 250 mL portions of water and saturated brine, dried over sodium sulfate, filtered and concentrated in vacuum. Column chromatography on silica gel with 10% ethyl acetate in hexane gave 730 mg of (R)-toluene-4-sulfonic acid 8-(2-chlorophenyl)-2,3-dihydrobenzo[1,4]dioxin-2-yl methyl ester. This was dissolved in 25 mL of DMF, 650 mg (10 mmol) of sodium azide added and the the mixture heated at 70-80° C. overnight. The solvent was removed in vacuum and 250 mL of methylene chloride added. The mixture was washed with 200 mL portions of wate and saturated brine, dried over sodium sulfate, filtered and concentrated in vacuum to 510 mg of (S)-2-azidomethyl-8-(2-chlorophenyl)-2,3-dihydrobenzo[1,4]dioxine as a yellow oil. A solution of this oil in 50 mL of THF was treated at room temperature with 2.4 g of polymer-supported triphenylphosphine and 10 mL of water for 4 days. The mixture was filtered through Celite and concentrated to an oil in vacuum. Column chromatography on silica gel with 0-5% methanol in methylene chloride, followed by recrystallization from ethanol with addition of 150 mg of fumaric acid gave 0.40 g of the title compound as a white solid, mp 200-1° C. [α]_{D}²⁵=+12.000 (c=1% SOLUTION, MeOH); MS (ES) m/z 276.0.

Elemental anal. for C₁₅H₁₄ClNO₂.C₄H₄O₄:

Theory: C, 58.25; H, 4.63; N, 3.57.

Found: C, 58.15; H, 4.59; N, 3.42.

### Example 57

1-{(2S)-8-[2-(trifluoromethyl)phenyl]-2,3-dihydro-1,4-benzodioxin-2-yl}methanamine: To a solution of (R)-toluene-4-sulfonic acid 8-trifluoromethanesulfonyloxy-2,3-dihydrobenzo[1,4]dioxin-2-ylmethyl ester (0.90 g, 1.9 mmol), prepared from (R)-toluene-4-sulfonic acid 8-formyl-2,3-dihydrobenzo[1,4]dioxin-2-ylmethyl ester by the procedure described for Intermediate 2, and 2-trifluoromethylbenzene boronic acid (1.44 g, 7.6 mmol) in 50 mL of DME, was added 10 mL of water and 0.50 g (4.7 mmol) of sodium carbonate. The mixture was brought to reflux under Argon and 112 mg of tetrakis(triphenylphosphine)palladium (0) was added. Reflux was continued overnight. The solvent was removed in vacuum and replaced with 400 mL of methylene chloride. The solution was washed with 250 mL portions of water and saturated brine, dried over sodium sulfate, filtered and concentrate in vacuum. Column chromatography on silica gel with 10% ethyl acetate in hexane gave 750 mg of (R)-toluene-4-sulfonic acid 8-(2-trifluoromethylphenyl)-2,3-dihydrobenzo[1,4]dioxin-2-yl methyl ester. This was dissolved in 25 mL of DMF, 650 mg (10 mmol) of sodium azide added and the the mixture heated at 70-80° C. for 24 h. The solvent was removed in vacuum and 250 mL of methylene chloride added. The mixture was washed with 250 mL portions of wate and saturated brine, dried over sodium sulfate, filtered and concentrated in vacuum to 540 mg of (S)-2-azidomethyl-8-(2-trifluoromethylphenyl)-2,3-dihydrobenzo[1,4]dioxine as a yellow oil. A solution of this oil in 50 mL of THF was treated at room temperature with 2.4 g of polymer-supported triphenylphosphine and 10 mL of water for 4 days. The mixture was filtered through Celite and concentrated to an oil in vacuum. Column chromatography on silica gel with 0-5% methanol in methylene chloride, followed by recrystallization from ethanol with addition of 150 mg of fumaric acid gave 0.44 g of the title compound as a white solid, mp 205-6° C. [α]_{D}²⁵=−13.2° (c=1% SOLUTION, MeOH); MS (ES) m/z 310.1.

Elemental anal. for C₁₆H₁₄F₃NO₂.C₄H₄O₄:

Theory: C, 56.47; H, 4.27; N, 3.29.

Found: C, 56.38; H, 4.03; N, 3.22. PASS

### Example 58

(2S)-[6-Chloro-8-(2-chloro-phenyl)-2,3-dihydro-benzo[1,4]dioxin-2-yl]-methylamine: Starting from (S)-2-azidomethyl-8-(2-chloro-phenyl)-6-chloro-2,3-dihydro-benzo[1,4]dioxine (0.29 g, 0.86 mmol), the procedure described in Example 54 afforded 0.18 g (58%) of the title compound as a hydrochloride salt, mp 228-230° C.; HRMS ESI m/z 310.0399 [M+H]⁺.

Anal. for C₁₅H₁₃Cl₂NO₂.HCl:

Theory: C, 51.97; H, 4.07; N, 4.04.

Found: C, 51.94; H, 3.59; N, 3.84.

### Example 59

(2S)-[6-Chloro-8-(2-fluoro-phenyl)-2,3-dihydro-benzo[1,4]dioxin-2-yl]-methylamine: Starting from (S)-2-azidomethyl-8-(2-fluoro-phenyl)-6-chloro-2,3-dihydro-benzo[1,4]dioxine (0.23 g, 0.72 mmol), the procedure described in Example 54 afforded 0.21 g (88%) of the title compound as a hydrochloride salt, mp 176-178° C.; HRMS ESI m/z 294.0710 [M+H]⁺.

Elemental Anal. for C₁₅H₁₃ClFNO₂.HCl:

Theory: C, 54.56; H, 4.27; N, 4.24.

Found: C, 55.41; H, 4.41; N, 3.87.

### Example 60

(2S)-[6-Chloro-8-(2-methyl-phenyl)-2,3-dihydro-benzo[1,4]dioxin-2-yl]-methylamine: Starting from (S)-2-azidomethyl-8-(2-methyl-phenyl)-6-chloro-2,3-dihydro-benzo[1,4]dioxine (0.26 g, 0.82 mmol), the procedure described in Example 54 afforded 0.20 g (75%) of the title compound as a hydrochloride salt, mp>245° C.; HRMS ESI m/z 290.0956 [M+H]⁺.

Elemental Anal. for C₁₆H₁₆ClNO₂.HCl:

Theory: C, 58.91; H, 5.25; N, 4.29.

Found: C, 59.03; H, 4.92; N, 4.20.

### Example 61

(2S)-[6-Chloro-8-(2-methoxy-phenyl)-2,3-dihydro-benzo[1,4]dioxin-2-yl]-methylamine: Starting from (S)-2-azidomethyl-8-(2-methoxy-phenyl)-6-chloro-2,3-dihydro-benzo[1,4]dioxine (0.28 g, 0.84 mmol), the procedure described in Example 54 afforded 0.21 g (74%) of the title compound as a hydrochloride salt, mp 188-189° C.; HRMS ESI m/z 306.0905 [M+H]⁺.

Elemental Anal. for C₁₆H₁₆ClNO₃.HCl:

Theory: C, 56.16; H, 5.01; N, 4.09.

Found: C, 56.84; H, 4.82; N, 3.52.

### Example 62

(2S)-[6-Chloro-8-(2-trifluoromethyl-phenyl)-2,3-dihydro-benzo[1,4]dioxin-2-yl]-methylamine: Starting from (S)-2-azidomethyl-8-(2-trifluoromethyl-phenyl)-6-chloro-2,3-dihydro-benzo[1,4]dioxine (0.23 g, 0.62 mmol), the procedure described in Example 54 afforded 0.22 g (92%) of the title compound as a hydrochloride salt, mp 231-233° C.; HRMS ESI m/z 344.0667 [M+H]⁺.

Elemental Anal. for C₁₆H₁₃ClF₃NO₂.HCl:

Theory: C, 50.55; H, 3.71; N, 3.68.

Found: C, 50.68; H, 3.41; N, 3.62.

### Example 63

(2S)-[6-Chloro-8-(2,3-dimethoxy-phenyl)-2,3-dihydro-benzo[1,4]dioxin-2-yl]-methyl amine: Starting from (S)-2-azidomethyl-8-(2,3-dimethoxy-phenyl)-6-chloro-2,3-dihydro-benzo[1,4]dioxine (0.28 g, 0.77 mmol), the procedure described in Example 54 afforded 0.21 g (72%) of the title compound as a hydrochloride salt, mp 197-199° C.; HRMS ESI m/z 336.1009 [M+H]⁺.

Elemental Anal. for C₁₇H₁₈ClNO₄.HCl:

Theory: C, 54.85; H, 5.14; N, 3.76.

Found: C, 54.64; H, 5.09; N, 3.65.

### Example 64

(2S)-[6-Chloro-8-(2,4-dichloro-phenyl)-2,3-dihydro-benzo[1,4]dioxin-2-yl]-methyl amine: Starting from (S)-2-azidomethyl-8-(2,4-dichloro-phenyl)-6-chloro-2,3-dihydro-benzo[1,4]dioxine (0.29 g, 0.78 mmol), the procedure described in Example 54 afforded 0.14 g (47%) of the title compound as a hydrochloride salt, mp 140° C.; HRMS ESI m/z 344.0009 [M+H]⁺.

Elemental Anal. for C₁₅H₁₂Cl₃NO₂.HCl:

Theory: C, 47.28; H, 3.44; N, 3.68.

Found: C, 47.30; H, 3.76; N, 3.35.

### Example 65

(2S)-[6-Chloro-8-(4-chloro-2-methyl-phenyl)-2,3-dihydro-benzo[1,4]dioxin-2-yl]-methylamine: Starting from (S)-2-azidomethyl-8-(4-chloro-2-methyl-phenyl)-6-chloro-2,3-dihydro-benzo[1,4]dioxine (0.29 g, 0.83 mmol), the procedure described in Example 54 afforded 0.17 g (55%) of the title compound as a hydrochloride salt, mp 110° C.; HRMS ESI m/z 324.0555 [M+H]⁺.

Elemental Anal. for C₁₆H₁₅Cl₂NO₂.HCl:

Theory: C, 53.28; H, 4.47; N, 3.88.

Found: C, 54.00; H, 4.76; N, 3.36.

### Example 66

(2S)-[6-Chloro-8-(2,4-di-trifluoromethyl-phenyl)-2,3-dihydro-benzo[1,4]dioxin-2-yl]-methylamine: Starting from (S)-2-azidomethyl-8-(2,4-di-trifluoromethyl-phenyl)-6-chloro-2,3-dihydro-benzo[1,4]dioxine (0.33 g, 0.75 mmol), the procedure described in Example 54 afforded 0.25 g (75%) of the title compound as a hydrochloride salt, mp 120° C.; HRMS ESI m/z 412.0540 [M+H]⁺.

Elemental Anal. for C₁₇H₁₂ClF₆NO₂.HCl:

Theory: C, 45.56H, 2.92; N, 3.13.

Found: C, 45.45; H, 2.64; N, 2.97.

### Example 67

(2S)-[6-Chloro-8-(2,5-dichloro-phenyl)-2,3-dihydro-benzo[1,4]dioxin-2-yl]-methyl amine: Starting from (S)-2-azidomethyl-8-(2,5-dichloro-phenyl)-6-chloro-2,3-dihydro-benzo[1,4]dioxine (0.28 g, 0.75 mmol), the procedure described in Example 54 afforded 0.23 g (79%) of the title compound as a hydrochloride salt, mp 205-207° C.; HRMS ESI m/z 344.0009 [M+H]⁺.

Anal. for C₁₅H₁₂Cl₃NO₂.HCl:

Theory: C, 47.28; H, 3.44; N, 3.68.

Found: C, 48.87; H, 3.62; N, 3.29.

### Example 68

(2S)-[6-Chloro-8-(5-chloro-2-methoxy-phenyl)-2,3-dihydro-benzo[1,4]dioxin-2-yl]-methylamine: Starting from (S)-2-azidomethyl-8-(5-chloro-2-methoxy-phenyl)-6-chloro-2,3-dihydro-benzo[1,4]dioxine (0.29 g, 0.79 mmol), the procedure described in Example 54 afforded 0.23 g (78%) of the title compound as a hydrochloride salt, mp 230-232° C.

HRMS ESI m/z 340.0510 [M+H]⁺.

Elemental Anal. for C₁₆H₁₅Cl₂NO₃.HCl:

Theory: C, 51.02; H, 4.28; N, 3.72.

Found: C, 50.90; H, 4.17; N, 3.63.

### Example 69

(2S)-[6-Chloro-8-(2,6-dimethyl-phenyl)-2,3-dihydro-benzo[1,4]dioxin-2-yl]-methyl amine: Starting from (S)-2-azidomethyl-8-(2,6-dimethyl-phenyl)-6-chloro-2,3-dihydro-benzo[1,4]dioxine (0.38 g, 1.15 mmol), the procedure described in Example 54 afforded 0.26 g (67%) of the title compound as a hydrochloride salt, mp 220-222° C.; HRMS ESI m/z 304.1108 [M+H]⁺.

Elemental Anal. for C₁₇H₁₈ClNO₂.HCl:

Theory: C, 60.01; H, 5.63; N, 4.12.

Found: C, 60.11; H, 5.65; N, 3.90.

### Example 70

(2S)-[7-Chloro-8-(2-chloro-phenyl)-2,3-dihydro-benzo[1,4]dioxin-2-yl]-methylamine: Starting from (S)-2-azidomethyl-7-chloro-8-(2-chloro-phenyl)-2,3-dihydro-benzo[1,4]dioxine (0.1 g, 0.29 mmol), the procedure described in Example 54 afforded 55 mg (54%) of the title compound as a hydrochloride salt, mp 105° C.; HRMS ESI m/z 310.0411 [M+H]⁺.

Elemental Anal. for C₁₅H₁₃Cl₂NO₂.HCl:

Theory: C, 51.97; H, 4.07; N, 4.04.

Found: C, 52.49; H, 4.32; N, 3.67.

### Example 71

(2S)-[8-(2-Chloro-phenyl)-7-fluoro-2,3-dihydro-benzo[1,4]dioxin-2-yl]-methylamine: Starting from (S)-2-azidomethyl-8-(2-chloro-phenyl)-7-fluoro-2,3-dihydro-benzo[1,4]dioxine (0.18 g, 0.58 mmol), the procedure described in Example 54 afforded 65 mg (34%) of the title compound as a hydrochloride salt, mp 208-210° C.; HRMS ESI m/z 294.0688 [M+H]⁺.

### Example 72

((2S)-7-Chloro-8-o-tolyl-2,3-dihydrobenzo[b][1,4]dioxin-2-yl)methanamine: Starting from (2S)-2-(azidomethyl)-7-chloro-8-o-tolyl-2,3-dihydrobenzo[b][1,4]dioxine (0.60 g, 1.9 mmol), the procedure described in Example 54 afforded 0.48 g (77%) of the title compound as a foam-like hydrochloride salt; no sharp mp was obtained; MS ES m/z 290.0 [M+H]⁺.

Elemental Anal. for C₁₆H₁₆ClNO₂.HCl:

Theory: C, 58.91; H, 5.25; N, 4.29.

Found: C, 57.47; H, 5.8; N, 3.95.

### Example 73

((2S)-7-Chloro-8-(2-(trifluoromethyl)phenyl)-2,3-dihydrobenzo[b][1,4]dioxin-2-yl)methanamine: Starting from (2S)-2-(azidomethyl)-7-chloro-8-(2-(trifluoromethyl)phenyl)-2,3-dihydrobenzo[b][1,4]dioxine (0.25 g, 0.67 mmol), the procedure described in Example 54 afforded 97 mg (38%) of the title compound as a hydrochloride salt, mp 84° C.; MS ES m/z 344.0 [M+H]⁺.

Elemental Anal. for C₁₆H₁₃ClF₃NO₂.HCl.0.3 C₁₆H₁₄:

Theory: C, 52.65; H, 4.52; N, 3.45.

Found: C, 52.54; H, 4.61; N, 3.06.

### Example 74

((2S)-7-Fluoro-8-o-tolyl-2,3-dihydrobenzo[b][1,4]dioxin-2-yl)methanamine: Starting from (2S)-2-(azidomethyl)-7-fluoro-8-o-tolyl-2,3-dihydrobenzo[b][1,4]dioxine (0.12 g, 0.40 mmol), the procedure described in Example 54 afforded 44 mg (35%) of the title compound as a hydrochloride salt, mp 183-185° C.; HRMS ESI m/z 274.1253 [M+H]⁺.

Elemental Anal. for C₁₆H₁₆FNO₂.HCl:

Theory: C, 54.56; H, 4.27; N, 4.24.

Found: C, 55.68; H, 4.35; N, 4.02.

### Example 75

((2S)-7-Fluoro-8-(2,4-di-chloro-phenyl)-2,3-dihydrobenzo[b][1,4]dioxin-2-yl) methanamine: Starting from (2S)-2-(azidomethyl)-7-fluoro-8-(2,4-di-chloro-phenyl-2,3-dihydrobenzo[b][1,4]dioxine (0.8 g, 0.40 mmol), the procedure described in Example 54 afforded 166 mg of the title compound as a hydrochloride salt, mp 65° C.; MS ES m/z 328.0 [M+H]⁺.

Elemental Anal. for C₁₅H₁₂Cl₂FNO₂.HCl.0.3 C₁₆H₁₄:

Theory: C, 51.66; H, 4.44; N, 3.59.

Found: C, 51.76; H, 4.24; N, 3.45.

### Example 76

1-[6-Fluoro-4-(2-methoxyphenyl)-1,3-benzodioxol-2-yl]methanamine: Starting from 2-azidomethyl-4-(2-methyoxy-phenyl)-6-fluoro-benzo[1,3]dioxole (77 mg, 0.26 mmol), the procedure described in Example 54 gave the title compound (25 mg, 31%) as a white solid hydrochloride salt, mp 184-186° C.; MS (ES) m/z 276.1 [M+H]⁺.

Elemental Anal. for C₁₅H₁₄FNO₃.HCl:

Theory: C, 57.79; H, 4.85; N, 4.49.

Found: C, 57.33; H, 4.60; N, 4.28.

### Example 77

1-(6-Fluoro-4-phenyl-1,3-benzodioxol-2-yl)methanamine: Starting from 2-azidomethyl-4-phenyl-6-fluoro-benzo[1,3]dioxole (76 mg, 0.86 mmol), the procedure described in Example 54 gave the title compound (44.5 mg, 56%) as a white solid hydrochloride salt, mp 227-229° C. MS (ES) m/z 246.1; HRMS: Theory for C₁₄H₁₂FNO₂+H⁺, 246.09248; found ESI, [M+H]⁺, 246.0923.

### Example 78

1-[4-(3-Chlorophenyl)-6-fluoro-1,3-benzodioxol-2-yl]methanamine: Starting from 2-azidomethyl-4-(3-chloro-phenyl)-6-fluoro-benzo[1,3]dioxole (90 mg, 0.29 mmol), the procedure described in Example 54 gave the title compound, (38.9 mg, 42%) as a white solid hydrochloride salt, mp 267-270° C. HRMS: Theory for C₁₄H₁₁ClFNO₂+H⁺, 280.05351; found (ESI, [M+H]⁺), 280.0535.

### Example 79

1-[4-(4-Chloro-phenyl)-6-fluoro-1,3-benzodioxol-2-yl]methanamine: Starting from 2-azidomethyl-4-(4-chloro-phenyl)-6-fluoro-benzo[1,3]dioxole (73 mg, 0.24 mmol), the procedure described in Example 54 gave the title compound, (46.3 mg, 61%) as a white solid hydrochloride salt, mp 262-264° C. HRMS: Theory for C₁₄H₁₁ClFNO₂+H⁺, 280.05351; found (ESI, [M+H]⁺), 280.0535.

### Example 80

1-[4-(2-Methyl-phenyl)-6-fluoro-1,3-benzodioxol-2-yl]methanamine: Starting from 2-azidomethyl-4-(2-methyl-phenyl)-6-fluoro-benzo[1,3]dioxole (78 mg, 0.27 mmol), the procedure described in Example 54 gave the title compound (52.7 mg, 59%) as a white solid hydrochloride salt, mp 197-200° C. MS (ES) m/z 260.1.

### Example 81

1-[4-(2,5-Dichloro-phenyl)-6-fluoro-1,3-benzodioxol-2-yl]methanamine: Starting from 2-azidomethyl-4-(2,5-dichloro-phenyl)-6-fluoro-benzo[1,3]dioxole (88 mg, 0.26 mmol), the procedure described in Example 54 gave the title compound (54.8 mg, 60%) as a white solid hydrochloride salt, mp 204-205° C.; MS (ES) m/z 314.0.

Elemental Anal. for C₁₄H₁₀C₁₂FNO₂.HCl.0.25H₂O:

Theory: C, 47.35; H, 3.26; N, 3.94.

Found: C, 47.24; H, 2.99; N, 3.89.

### Example 82

1-[4-(2-Trifluoromethyl-phenyl)-6-fluoro-1,3-benzodioxol-2-yl]methanamine: Starting from 2-azidomethyl-4-(2-trifluoromethyl-phenyl)-6-fluoro-benzo[1,3]dioxole (85 mg, 0.25 mmol), the procedure described in Example 54 gave the title compound (51.5 mg, 59%) as a white solid hydrochloride salt, mp 178-180° C.; MS (ES) m/z 314.0; HRMS: Theory for C₁₅H₁₁F₄NO₂+H⁺, 314.0804; found (ESI, [M+H]⁺), 314.0804.

### Example 83

1-[4-(2-Fluoro-phenyl)-6-fluoro-1,3-benzodioxol-2-yl]methanamine: Starting from 2-azidomethyl-4-(2-fluoro-phenyl)-6-fluoro-benzo[1,3]dioxole (97 mg, 0.34 mmol), the procedure described in Example 54 gave the title compound (44.4 mg, 44%) as a white solid hydrochloride salt, mp 234-235° C.; MS (ES) m/z 264.1; HRMS: Theory for C₁₄H₁₁F₂NO₂+H⁺, 264.0836; found (ESI, [M+H]⁺), 264.0821.

### Example 84

1-[4-(2-Chloro-phenyl)-6-fluoro-1,3-benzodioxol-2-yl]methanamine: Starting from 2-azidomethyl-4-(2-chloro-phenyl)-6-fluoro-benzo[1,3]dioxole (93 mg, 0.30 mmol), the procedure described in Example 54 gave the title compound (42.7 mg, 47%) as a white solid hydrochloride salt, mp 192-194° C.; MS (ES) m/z 280.1.

### Example 85

1-[(2S)-4-(2,6-dichlorophenyl)-6-fluoro-1,3-benzodioxol-2-yl]methanamine: To a solution of 1-[4-(2,6-dichlorophenyl)-6-fluoro-1,3-benzodioxol-2-yl]methanamine (2.88 g, 9.2 mmol) in tetrahydrofuran was added benzylchloroformate (1.71 mL, 12 mmol) and diisopropylethylamine (4.0 mL, 23 mmol) at 0° C. The mixture was stirred at 0° C. for 2 hours, and quenched with water. The mixture was extracted with methylene chloride. The solvent was removed under vacuum to form a colorless oil, 6.25 g. The corresponding Cbz derivative of the (2S)-enantiomer was isolated by Super Fluid Chromatography (SFC). The desired fractions were combined and the solvent was removed under the vacuum. The crude oil (1.75 g, 3.8 mmol) was dissolved in acetonitrile at 0° C. and iodotrimethylsilane (1.65 mL, 11.4 mmol) and hydrogen hydrochloride (1.0 M in diethyl ether, 4.24 mL, 4.2 mmol) were added to the solution at 0° C. The reaction mixture was stirred at 0° C. for 3 hours and then quenched with 1N HCl aquous solution. The mixture was extracted with ether (3×50 mL). The organic layer was washed with 1N HCl (3×50 mL). The aqueous layer was combined and neutralized with 10% potassium hydroxide (PH>7). The neutralized aqueous solution was extracted with methylene chloride (3×50 mL) and the organic layers were combined. The solvent was removed under vacuum and chromatography with 10% methanol in methylene chloride afforded the desired (2S)-enantiomer as a colorless oil. The oil was dissolved in ethyl acetate and converted to its hydrochloride salt (1.0 g) using excess ethereal hydrochloric acid to give a white solid, mp 224-225° C.; [α]_{D}²⁵=+53.00° (c=1% SOLUTION, DMSO); MS (ES) m/z 314.0.

Elemental anal. for C₁₄H₁₀C₁₂FNO₂.HCl:

Theory: C, 47.96; H, 3.16; N, 3.99.

Found: C, 47.92; H, 3.12; N, 3.84.

### Example 86

1-[(2R)-4-(2,6-dichlorophenyl)-6-fluoro-1,3-benzodioxol-2-yl]methanamine: The title compound was prepared according to the procedure described in Example 85. By using the SFC method the desired Cbz derivative of the (2R)-enantiomer (1.91 g) was obtained at the same time as the (2S)-enantiomer was isolated. After removal of the Cbz protecting group, the crude product was purified by column chromatography (10% methanol in methylene chloride) and afforded the title compound as a colorless oil. The oil was dissolved in ethyl acetate and converted to its hydrochloride salt (1.01 g) using excess ethereal hydrochloric acid to give a white solid, mp 206-208° C.; MS (ES) m/z 314.0 [M+H]⁺; [α]_{D}²⁵=−50.000 (c=1% SOLUTION, DMSO).

Elemental anal. for C₁₄H₁₀C₁₂FNO₂.HCl:

Theory: C, 47.96; H, 3.16; N, 3.99.

Found: C, 47.78; H, 3.05; N, 3.89.

### Example 87

1-[4-(2-Chlorophenyl)-6-fluoro-1,3-benzodioxol-2-yl]-N-methylmethanamine: A solution of toluene-4-sulfonic acid 4-(2-chloro-phenyl)-6-fluoro-benzo[1,3]dioxol-2-yl-methyl ester (0.1 g 0.23 mmol) and methylamine (1.0 M in THF, 10 eq,) in DMSO was heated at 70° C. overnight. The reaction was quenched with 1N sodium hydroxide and extracted with methylene chloride. The organic layer was washed with water and dried over anhydrous sodium sulfate. The solvent was removed under vacuum. The crude product was purified by column chromatography (10% methanol in methylene chloride) to afford the title compound as a colorless oil. The oil was dissolved in ethyl acetate and converted to its hydrochloride salt (1.01 g) using excess ethereal hydrochloric acid to give a white solid, mp 132-134° C.; MS (ES) m/z 294.0.

Elemental anal. for C₁₅H₁₃ClFNO₂.HCl:

Theory: C, 54.56; H, 4.27; N, 4.24.

Found: C, 54.36; H, 3.99; N, 4.00.

### Example 88

N-{[4-(2-Chlorophenyl)-6-fluoro-1,3-benzodioxol-2-yl]methyl}ethanamine: Starting from toluene-4-sulfonic acid 4-(2-chloro-phenyl)-6-fluoro-benzo[1,3]dioxol-2-yl-methyl ester (0.1 g 0.23 mmol) and ethylamine (1.0 M in THF, 10 eq,), the procedure described for Example 87 gave 32.3 mg (41%) of the title compound as a white solid hydrochloride salt, mp 182-183° C.; MS (ES) m/z 308.1.

Elemental anal. for C₁₆H₁₅ClFNO₂.HCl.0.25H₂O:

Theory: C, 55.11; H, 4.77; N, 4.02.

Found: C, 55.13; H, 4.46; N, 3.74.

### Example 89

1-[4-(2-chlorophenyl)-6-fluoro-1,3-benzodioxol-2-yl]-N,N-dimethylmethanamine: Starting from toluene-4-sulfonic acid 4-(2-chloro-phenyl)-6-fluoro-benzo[1,3]dioxol-2-yl-methyl ester (0.1 g 0.23 mmol) and N,N-dimethylamine (1.0 M in THF, 10 eq,), the procedure described for Example 87 gave 55.3 mg (70%) of the title compound as a white solid hydrochloride salt, mp 211-212° C.; MS (ES) m/z 308.1.

### Example 90

[6-Chloro-4-(2-chloro-phenyl)-benzo[1,3]dioxol-2-yl]-methylamine: Starting from 2-azidomethyl-6-chloro-4-(2-chloro-phenyl)-benzo[1,3]dioxole (0.18 g, 0.56 mmol), the procedure described in Example 54 afforded 73 mg (39%) of the title compound as a hydrochloride salt, mp>250° C.; MS ES m/z 296.0 [M+H]⁺.

Elemental Anal. for C₁₄H₁₁Cl₂NO₂.HCl:

Theory: C, 50.56; H, 3.64; N, 4.21.

Found: C, 50.69; H, 3.27; N, 4.17.

### Example 91

[6-Chloro-4-(2-methyl-phenyl)-benzo[1,3]dioxol-2-yl]-methylamine: Starting from 2-azidomethyl-6-chloro-4-(2-methyl-phenyl)-benzo[1,3]dioxole (0.20 g, 0.66 mmol), the procedure described in Example 54 afforded 0.12 g (59%) of the title compound as a hydrochloride salt, mp>250° C.; MS ES m/z 276.1 [M+H]⁺.

Elemental Anal. for C₁₅H₁₄ClO₂.HCl:

Theory: C, 57.71; H, 4.84; N, 4.49.

Found: C, 57.93; H, 4.99; N, 4.36.

### Example 92

[6-Chloro-4-(2-methoxy-phenyl)-benzo[1,3]dioxol-2-yl]-methylamine: Starting from 2-azidomethyl-6-chloro-4-(2-methoxy-phenyl)-benzo[1,3]dioxole (0.18 g, 0.57 mmol), the procedure described in Example 54 afforded 82 mg (44%) of the title compound as a hydrochloride salt, mp 245-246° C.; MS ES m/z 292.1 [M+H]⁺.

Elemental Anal. for C₁₅H₁₄ClNO₃.HCl:

Theory: C, 54.90; H, 4.61; N, 4.27.

Found: C, 54.91; H, 4.80; N, 4.18.

### Example 93

[6-Chloro-4-(2-fluoro-phenyl)-benzo[1,3]dioxol-2-yl]-methylamine: Starting from 2-azidomethyl-6-chloro-4-(2-fluoro-phenyl)-benzo[1,3]dioxole (0.18 g, 0.59 mmol), the procedure described in Example 54 afforded 86 mg (46%) of the title compound as a hydrochlorIe salt, mp>250° C.; MS ES m/z 280.1 [M+H]⁺.

Elemental Anal. for C₁₄H₁₁ClFNO₂.HCl:

Theory: C, 53.19; H, 3.83; N, 4.43.

Found: C, 53.34; H, 3.75; N, 4.30.

### Example 94

[6-Chloro-4-(2-methoxy-5-chloro-phenyl)-benzo[1,3]dioxol-2-yl]-methylamine: Starting from 2-azidomethyl-6-chloro-4-(2-methoxy-5-chlorophenyl)-benzo[1,3]dioxole (0.19 g, 0.54 mmol), the procedure described in Example 54 afforded 0.117 g (60%) of the title compound as a hydrochloride salt, mp 228-230° C.; MS ES m/z 326.0 [M+H]⁺.

Elemental Anal. for C₁₅H₁₃Cl₂NO₃.HCl.0.5H₂O:

Theory: C, 48.48; H, 4.07; N, 3.77.

Found: C, 48.53; H, 4.06; N, 3.68.

### Example 95

[6-Chloro-4-(2,3-dimethoxy-phenyl)-benzo[1,3]dioxol-2-yl]-methylamine: Starting from 2-azidomethyl-6-chloro-4-(2,3-dimethoxy-phenyl)-benzo[1,3]dioxole (0.20 g, 0.57 mmol), the procedure described in Example 54 afforded 0.115 g (56%) of the title compound as a hydrochloride salt, mp 228-230° C.; MS ES m/z 322.1 [M+H]⁺.

Elemental Anal. for C₁₆H₁₆ClNO₄.HCl.0.5H₂O:

Theory: C, 52.33; H, 4.94; N, 3.81.

Found: C, 52.43; H, 4.59; N, 3.67.

### Example 96

[6-Chloro-4-(2,4-dichloro-phenyl)-benzo[1,3]dioxol-2-yl]-methylamine: Starting from 2-azidomethyl-6-chloro-4-(2,4-dichloro-phenyl)-benzo[1,3]dioxole-(0.12 g, 0.34 mmol), the procedure described in Example 54 afforded 53 mg (42%) of the title compound as a hydrochloride salt, mp>250° C.; MS ES m/z 330.0 [M+H]⁺.

Elemental Anal. for C₁₄H₁₀Cl₃NO₂.HCl:

Theory: C, 45.81; H, 3.02; N, 3.82.

Found: C, 45.98; H, 3.44; N, 3.61.

### Example 97

[6-Chloro-4-(4-chloro-2-methyl-phenyl)-benzo[1,3]dioxol-2-yl]-methylamine: Starting from 2-azidomethyl-6-chloro-4-(4-chloro-2-methylphenyl)-benzo[1,3]dioxole (0.20 g, 0.59 mmol), the procedure described in Example 54 afforded 90 mg (44%) of the title compound as a hydrochloride salt, mp>250° C.; MS ES m/z 310.0 [M+H]⁺.

### Example 98

[6-Chloro-4-(2,5-dichloro-phenyl)-benzo[1,3]dioxol-2-yl]-methylamine: Starting from 2-azidomethyl-6-chloro-4-(2,5-dichloro-phenyl)-benzo[1,3]dioxole (0.16 g, 0.45 mmol), the procedure described in Example 54 afforded 28 mg (17%) of the title compound as a hydrochloride salt, mp 246-248° C.; MS ES m/z 330.0 [M+H]⁺.

### Example 99

[6-Chloro-4-(2,5-difluoro-phenyl)-benzo[1,3]dioxol-2-yl]-methylamine: Starting from 2-azidomethyl-6-chloro-4-(2,5-difluoro-phenyl)-benzo[1,3]dioxole (0.20 g, 0.62 mmol), the procedure described in Example 54 afforded 92 mg (44%) of the title compound as a hydrochloride salt, mp>250° C.; MS ES m/z 298.0 [M+H]⁺.

Elemental Anal. for C₁₄H₁₀Cl₃NO₂.HCl:

Theory: C, 50.32; H, 3.32; N, 4.19.

Found: C, 50.48; H, 3.10; N, 4.08.

### Example 100

[6-Chloro-4-(2,5-dimethoxy-phenyl)-benzo[1,3]dioxol-2-yl]-methylamine: Starting from 2-azidomethyl-6-chloro-4-(2,5-dimethoxy-phenyl)-benzo[1,3]dioxole (0.14 g, 0.40 mmol), the procedure described in Example 54 afforded 53 mg (37%) of the title compound as a hydrochloride salt, mp 225-227° C.; MS ES m/z 322.1 [M+H]⁺.

Elemental Anal. for C₁₆H₁₆ClNO₄.HCl:

Theory: C, 53.65; H, 4.78; N, 3.91.

Found: C, 53.40; H, 4.44; N, 3.81.

### Example 101

[6-Chloro-4-biphenyl-benzo[1,3]dioxol-2-yl]-methylamine: Starting from 2-azidomethyl-4-biphenyl-2-yl-6-chloro-benzo[1,3]dioxole (0.31 g, 0.85 mmol), the procedure described in Example 54 afforded 0.25 g amine. 80 mg of the amine was used to generate 38 mg of the title compound as a hydrochloride salt, mp 196-198° C.; MS ES m/Z 338.1 [M+H]⁺.

Using the general procedures outlined above, Examples 102-108 were prepared.

### Example 102

C—[(S)-8-(2,4-Dimethyl-phenyl)-2,3-dihydro-benzo[1,4]dioxin-2-yl]-methylamine (I-133): Starting from (S)-2-Azidomethyl-8-(2,4-dimethyl-phenyl)-2,3-dihydro-benzo[1,4]dioxine (450 mg, 2.25 mmol), 270 mg (40%) of the title compound was obtained as a hydrochloric salt. MS (ESI) m/z 270.2 [M+H]⁺.

### Example 103

C—[(S)-8-(4-methoxy-2-methyl-phenyl)-2,3-dihydro-benzo[1,4]dioxin-2-yl]-methylamine (I-127): Starting from (S)-2-Azidomethyl-8-(4-methoxy-2-methyl-phenyl)-2,3-dihydro-benzo[1,4]dioxine (310 mg, 1.55 mmol), 210 mg (42%) of the title compound was obtained as a hydrochloric salt. MS (ESI) m/z 286.4 [M+H]⁺.

### Example 104

C—[(S)-8-(4-ethoxy-2-methyl-phenyl)-2,3-dihydro-benzo[1,4]dioxin-2-yl]-methylamine (I-132): Starting from (S)-2-Azidomethyl-8-(4-ethoxy-2-methyl-phenyl)-2,3-dihydro-benzo[1,4]dioxine (320 mg, 1.6 mmol), 200 mg (37%) of the title compound was obtained as a hydrochloric salt. MS (ESI) m/z 300.4 [M+H]⁺.

### Example 105

C—[(S)-8-(2,6-dimethoxy-phenyl)-2,3-dihydro-benzo[1,4]dioxin-2-yl]-methylamine (I-168): Starting from (S)-2-Azidomethyl-8-(2,6-dimethoxy-phenyl)-2,3-dihydro-benzo[1,4]dioxine (140 mg, 0.7 mmol), 60 mg (25%) of the title compound was obtained as a hydrochloric salt. MS (ESI) m/z 302.2 [M+H]⁺.

### Example 106

C—[(S)-8-(4-fluoro-2-isopropoxy-phenyl)-2,3-dihydro-benzo[1,4]dioxin-2-yl]-methylamine (I-143): Starting from (S)-2-Azidomethyl-8-(4-fluoro-2-isopropoxy-phenyl)-2,3-dihydro-benzo[1,4]dioxine (240 mg, 1.2 mmol), 180 mg (43%) of the title compound was obtained as a hydrochloric salt. MS (ESI) m/z 318.3 [M+H]⁺.

### Example 107

C—[(S)-8-(4-fluoro-2-methoxy-phenyl)-2,3-dihydro-benzo[1,4]dioxin-2-yl]-methylamine (I-144): Starting from (S)-2-Azidomethyl-8-(4-fluoro-2-methoxy-phenyl)-2,3-dihydro-benzo[1,4]dioxine (250 mg, 2.25 mmol), 164 mg (22%) of the title compound was obtained as a hydrochloric salt. MS (ESI) m/z 290.3 [M+H]⁺.

### Example 108

C—[(S)-8-(2-chloro-4-methoxy-phenyl)-2,3-dihydro-benzo[1,4]dioxin-2-yl]-methylamine (I-128): Starting from (S)-2-Azidomethyl-8-(2-chloro-4-methoxy-phenyl)-2,3-dihydro-benzo[1,4]dioxine (370 mg, 1.12 mmol), 185 mg (48%) of the title compound was obtained as a hydrochloric salt. MS (ESI) m/z 306.2 [M+H]⁺.

### Biological Assays

The compounds of this invention are agonists and partial agonists at the 2C subtype of brain serotonin receptors and are thus of interest for the treatment of schizophrenia and related disorders such as schizoaffective disorder, schizophreniform disorder, L-DOPA-induced psychosis and bipolar disorder, depression, including related disorders such as obsessive compulsive disorder and panic disorder, and obesity, with its consequent comorbidities including Type II diabetes, cardiovascular disease, hypertension, hyperlipidemia, stroke, osteoarthritis, sleep apnea, gall bladder disease, gout, some cancers, some infertility, and early mortality. These, and other disorders the treatment of which the present compounds are useful, are discussed herein.

A. Assessment of Effectiveness of Compounds as 5HT_{2C} Agonists and Partial Agonists

The ability of the compounds of this invention to act as 5HT_{2C} agonists and partial agonists was established using several standard pharmacological test procedures; the procedures used and results obtained are provided below. In the test procedures, 5-HT stands for 5-hydroxytryptamine, mCPP stands for meta-chlorophenylpiperazine, and DOI stands for 1-(2,5-dimethoxy-4-iodophenyl)isopropylamine.

To evaluate the affinity of various compounds of formula I for activity at the 5-HT_{2C} receptor, a CHO (Chinese Hamster Ovary) cell line transfected with the cDNA expressing the human 5-hydroxytryptamine-2C (h5-HT_{2C}) receptor was maintained in DMEM (Dulbecco's Modified Eagle Media) supplied with fetal calf serum, glutamine, and the markers: guaninephosphoribosyl transferase (GTP) and hypoxanthinethymidine (HT). The cells were allowed to grow to confluence in large culture dishes with intermediate changes of media and splitting. Upon reaching confluence, the cells were harvested by scraping. The harvested cells were suspended in half volume of fresh physiological phosphate buffered saline (PBS) solution and centrifuged at low speed (900×g). This operation was repeated once. The collected cells were then homogenized with a polytron at setting #7 for 15 sec in ten volumes of 50 mM Tris.HCl, pH 7.4 and 0.5 mM EDTA. The homogenate was centrifuged at 900×g for 15 min to remove nuclear particles and other cell debris. The pellet was discarded and the supernatant fluid recentrifuged at 40,000×g for 30 min. The resulting pellet was resuspended in a small volume of Tris.HCl buffer and the tissue protein content was determined in aliquots of 10-25 μL volumes. Bovine Serum Albumin (BSA) was used as the standard in the protein determination by the method of Lowry et al., (J. Biol. Chem., 193:265 (1951). The volume of the suspended cell membranes was adjusted with 50 mM Tris.HCl buffer containing: 0.1% ascorbic acid, 10 mM pargyline and 4 mM CaCl₂ to give a tissue protein concentration of 1-2 mg per mL of suspension. The preparation membrane suspension (many times concentrated) was aliquoted in 1 mL volumes and stored at −70° C. until used in subsequent binding experiments.

Binding measurements were performed in a 96 well microtiter plate format, in a total volume of 200 μL. To each well was added: 60 μL of incubation buffer made in 50 mM Tris.HCl buffer, pH 7.4 and containing 4 mM CaCl₂; 20 μL of [¹²⁵I] DOI (S.A., 2200 Ci/mmol, NEN Life Science).

The dissociation constant, KD of [¹²⁵I] DOI at the human serotonin 5-HT_{2C} receptor was 0.4 nM by saturation binding with increasing concentrations of [¹²⁵I] DOI. The reaction was initiated by the final addition of 100 μL of tissue suspension containing 50 μg of receptor protein. Nonspecific binding is measured in the presence of 1 μM unlabeled DOI added in 20.0 μL volume. Test compounds were added in 20.0 μL. The mixture was incubated at room temperature for 60 min. The incubation was stopped by rapid filtration. The bound ligand-receptor complex was filtered off on a 96 well unifilter with a Packard® Filtermate 196 Harvester. The bound complex caught on the filter disk was dried in a vacuum oven heated to 60° C. and the radioactivity measured by liquid scintillation with 40 μL Microscint-20 scintillant in a Packard TopCount® equipped with six (6) photomultiplier detectors.

Specific binding is defined as the total radioactivity bound less the amount bound in the presence of 1 μM unlabeled DOI. Binding in the presence of varying concentrations of test drugs is expressed as percent of specific binding in the absence of drug. These results are then plotted as log % bound vs log concentration of test drug. Non linear regression analysis of data points yields both the IC₅₀ and the Kᵢ values of test compounds with 95% confidence limits. Alternatively, a linear regression line of decline of data points is plotted, from which the IC₅₀ value can be read off the curve and the Kᵢ value determined by solving the following equation:

$${K}_{i} = \frac{{IC}_{50}}{1 + L / {K}_{D}}$$

where L is the concentration of the radioactive ligand used and the K_{D} is the dissociation constant of the ligand for the receptor, both expressed in nM.

The following Kᵢ's (95% confidence interval) are provided for various reference compounds in Table 2, below:

TABLE 2
Kᵢ Data for Reference Compounds

| Compound | Kᵢ | |
|---|---|---|
| Ritanserin | 2.0 | (1.3-3.1) nM |
| Ketanserin | 94.8 | (70.7-127.0) nM |
| Mianserin | 2.7 | (1.9-3.8) nM |
| Clozapine | 23.2 | (16.0-34.0) nM |
| Methiothepin | 4.6 | (4.0-6.0) nM |
| Methysergide | 6.3 | (4.6-8.6) nM |
| Loxapine | 33.0 | (24.0-47.0) nM |
| mCPP | 6.5 | (4.8-9.0) nM |
| DOI | 6.2 | (4.9-8.0) nM |

The ability of the compounds of formula I to produce an agonist response at brain 5-HT_{2C} was assessed by determining their effect on calcium mobilization using the following procedure: CHO cells stably expressing the human 5-HT_{2C} receptor were cultured in Dulbecco's modified Eagle's medium (DMEM) supplemented with 10% fetal bovine serum and non-essential amino acids. Cells were plated at a density of 40K cells/well in 96-well clear-bottom black-wall plates 24 hours prior to the evaluation of 5-HT_{2C} receptor-stimulated calcium mobilization. For calcium studies, cells were loaded with the calcium indicator dye Fluo-3-AM in Hank's buffered saline (HBS) for 60 minutes at 37° C. Cells were washed with HBS at room temperature and transferred to the fluorometric imaging plate reader (FLIPR, Molecular Devices, Sunnyvale, Calif.) for acquisition of calcium images. Excitation at 488 nm was achieved with an Argon ion laser and a 510-560 nm emission filter was used. Fluorescence images and relative intensities were captured at 1 second intervals and cells were stimulated by addition of agonist after 10 baseline measurements using the internal fluidics module of the FLIPR. An increase in fluorescence counts corresponds to an increase in intracellular calcium.

For the evaluation of agonist pharmacology the calcium changes in response to different concentrations of agonist were determined using a maximum minus minimum calculation of the raw fluorescence count data. Calcium changes were then expressed as a percentage of the response observed with a maximally effective concentration of 5-HT. EC₅₀ values were estimated by non-linear regression analysis of the log-concentration % maximum 5-HT response curves using the 4-parameter logistic function. In certain embodiments, compounds of the present invention provide an EC₅₀ of ≦about 1000 nM. In other embodiments, compounds of the present invention provide an EC₅₀ of ≦about 100 nM, in yet other embodiments ≦about 20 nM, in still other embodiments ≦about 5 nM, and certain embodiments ≦about 2 nM.

The following EC₅₀'s are provided for various reference compounds in Table 3, below

TABLE 3
EC₅₀ Data for Reference Compounds:

| Compound | EC₅₀ | |
|---|---|---|
| 5-HT EC₅₀ | 0.5 | nM |
| DOI EC₅₀ | 0.5 | nM |
| mCPP EC₅₀ | 5.4 | nM |
| SB242084 | 0.01 | nM |
| SB206553 | 13 | nM |

Table 4 below shows the results of the activity of selected compounds of this invention in the assays described above. The compound numbers correspond to the compound numbers in Table 1, supra. Compounds having an activity designated as “A” provided a Kᵢ value of less than or euqal to 50 nM; compounds having an activity designated as “B” provided a Kᵢ value between 50 nM and 200 nM; and compounds having an activity designated as “C” provided a Kᵢ value greater than 200 nM. Compounds having an activity designated as “D” provided an IC₅₀ value of less than or equal to 100 nM; compounds having an activity designated as “E” provided a an IC₅₀ value between 100 nM and 500 nM; and compounds having an activity designated as “F” provided a an IC₅₀ value greater than 500 nM. An activity designated as “−”, for any compound listed in Table 4, below, means that the data was not provided for that compound.

TABLE 4
5-HT2c Activity of Selected Compounds

| | | |
|---|---|---|
| Compound | 5-HT_{2C} Binding | 5-HT_{2C} Function |

| Number | Kᵢ avg (nM) | EC₅₀ (nM) | EMax (%) |
|---|---|---|---|
| 1 | A | D | 60 |
| 2 | A | D | 70 |
| 3 | A | D | 60 |
| 4 | A | D | 70 |
| 5 | B | D | 60 |
| 6 | B | E | 70 |
| 7 | A | D | 70 |
| 8 | A | D | 60 |
| 9 | A | D | 50 |
| 10 | C | E | 50 |
| 11 | C | E | 60 |
| 12 | A | D | 70 |
| 13 | A | D | 70 |
| 14 | A | D | 70 |
| 15 | A | D | 60 |
| 16 | B | D | 60 |
| 17 | — | D | 60 |
| 18 | A | D | 50 |
| 19 | C | E | 20 |
| 20 | B | F | 40 |
| 21 | A | D | 40 |
| 22 | — | D | 40 |
| 23 | — | E | 40 |
| 24 | A | D | 70 |
| 25 | — | F | 30 |
| 26 | — | D | 50 |
| 27 | — | E | 20 |
| 28 | — | F | 30 |
| 29 | — | F | — |
| 30 | B | F | — |
| 31 | C | E | 20 |
| 32 | B | E | 30 |
| 33 | B | F | 30 |
| 34 | B | F | 20 |
| 35 | B | F | — |
| 36 | C | F | — |
| 37 | C | F | 20 |
| 38 | A | E | 30 |
| 39 | A | E | 20 |
| 40 | C | F | 30 |
| 41 | B | F | 20 |
| 42 | C | F | — |
| 43 | C | F | 30 |
| 44 | B | F | — |
| 45 | C | F | 30 |
| 46 | A | D | 50 |
| 47 | A | D | 60 |
| 48 | C | — | — |
| 49 | A | F | 70 |
| 50 | C | — | — |
| 51 | C | — | — |
| 52 | C | — | — |
| 53 | C | — | — |
| 54 | A | D | 80 |
| 55 | A | D | 95 |
| 56 | A | — | — |
| 57 | A | A | 50 |
| 58 | A | D | 50 |
| 59 | A | E | 40 |
| 60 | A | E | 50 |
| 61 | A | E | 50 |
| 62 | A | E | 60 |
| 63 | A | F | 50 |
| 64 | A | E | 60 |
| 65 | A | — | — |
| 66 | B | — | — |
| 67 | A | E | 50 |
| 68 | B | — | — |
| 69 | A | E | 60 |
| 70 | A | D | 50 |
| 71 | A | D | 40 |
| 72 | A | — | — |
| 73 | — | — | — |
| 74 | A | D | 40 |
| 75 | — | — | — |
| 76 | A | D | 70 |
| 77 | B | — | — |
| 78 | B | — | — |
| 79 | B | — | — |
| 80 | A | D | 90 |
| 81 | A | D | 80 |
| 82 | A | D | 70 |
| 83 | A | D | 70 |
| 84 | A | D | 80 |
| 85 | A | D | 90 |
| 86 | A | F | 80 |
| 87 | A | D | 80 |
| 88 | B | — | — |
| 89 | B | — | — |
| 90 | A | D | 80 |
| 91 | A | D | 80 |
| 92 | B | — | — |
| 93 | B | — | — |
| 94 | C | — | — |
| 95 | B | — | — |
| 96 | A | D | 80 |
| 97 | B | — | — |
| 98 | A | D | 80 |
| 99 | B | — | — |
| 100 | C | — | — |

The compounds of this invention thus have affinity for and agonist or partial agonist activity at brain serotonin 5HT_{2C} receptors. They are therefore of interest for the treatment of the central nervous system conditions described previously herein.

B. Assessment of Effectiveness of Compounds in Obesity Models

Obesity Model A

To evaluate acute in vivo efficacy of various compounds, 7 weeks-old male C57BL/6J mice were obtained from The Jackson Laboratory (Bar Harbor, Me.) and 6 weeks-old lean Zucker rats were purchased from Charles River Laboratories (Wilmington, Mass.). Mice and rats were single housed in a temperature-controlled (25° C.) facility with a 12-h light/dark cycle. Animals were allowed normal chow diet (Rodent chow #5001, PharmaServ, Framingham, Mass.) and water ad libitum. After one week acclimation, animals were randomized to vehicle (saline) or treatment groups. Animals were fasted overnight (16 hrs) and orally dosed with vehicle or compounds. Thirty minutes after compound administration, animals were given a weighed amount of food, and food intake was recorded 30 minutes, 1 h, 2 h, 4 h, 7 h and 24 h after refeeding. Results are summarized in Table 5, below.

TABLE 5

| Compound | Dose | % Reduction food intake (vs vehicle) | | |
|---|---|---|---|---|
| # | (mpk) | 0.5 hour | 2 hours | 4 hours |
| I-46 | 1 | 0 ± 12 | 0 ± 6 | 1 ± 8 |
| I-46 | 3 | 12 ± 8 | 11 ± 5 | 14 ± 7 |
| I-46 | 10 | 74 ± 7 | 68 ± 5 | 51 ± 4 |
| I-46 | 30 | 78 ± 14 | 88 ± 8 | 82 ± 7 |
| I-46 | 50 | 100 ± 11 | 100 ± 5 | 98 ± 4 |
| I-4 | 10 | 9 ± 6 | 5 ± 8 | 2 ± 7 |
| I-4 | 30 | 29 ± 7 | 14 ± 8 | 0 ± 16 |
| I-4 | 50 | 56 ± 10 | 27 ± 11 | 8 ± 14 |
| I-1 | 10 | 4 ± 8 | 6 ± 8 | 16 ± 8 |
| I-1 | 30 | 60 ± 12 | 40 ± 8 | 42 ± 5 |
| I-1 | 50 | 74 ± 17 | 55 ± 13 | 48 ± 13 |

Obesity Model B

To assess in vivo efficacy of various 5-HT_{2C} compounds on weight loss, 5 weeks-old male C57BL/6-DIO mice are fed a high-fat high-sucrose diet (58 kcal % fat, 16.4 kcal % protein, 25.5 kcal % carbohydrate) for 11 weeks. 6 weeks-old male Zucker fa/fa rats purchased from Charles River Laboratories are also used. Mice and rats were single housed in a temperature-controlled (25° C.) facility with a 12-h light/dark cycle. Animals are allowed food and water ad libitum. After one week acclimation, animals are randomized to vehicle (saline) or treatment groups. Animals are orally dosed once daily for 14 days. Body weight, food consumption, and/or body composition (NMR) are recorded. Epidydimal adipose tissue is collected at the end of the study.

C. Assessment of Effectiveness in Treatment of Pain

Compounds of formula I may be evaluated in accordance with the present invention to establish the extent of their effectiveness to treat pain, and may optionally be compared with other pain treatments.

A variety of methods have been established in the art to evaluate the effectiveness of compounds for relieving pain. See e.g., Bennett et al, Pain 33: 87-107, 1988; Chaplan et al, J. Neurosci. Methods 53:55-63, 1994; and Mosconi et al, Pain 64:37-57, 1996. Below is a specific description of one strategy that may be employed.

Procedure: Individually housed Spraque-Dawley rats are given free access to rat chow and water. A 12-h light/12-h dark cycle is put in effect (lights on from 6:00 am to 6:00 pm). Animal maintenance and research are conducted in accordance with the guidelines provided by the National Institutes of Health Committee on Laboratory Animal Resources. These subjects are used in the tests as set forth below.

Test Method 1: Prostaglandin E₂-Induced Thermal Hypersensitivity.

The terminal 10 cm of the tail is placed into a thermos bottle containing water warmed to 38, 42, 46, 50, 54, or 58° C. The latency in seconds for the animal to remove the tail from the water is used as a measure of nociception. If the animal does not remove the tail within 20 sec, the experimenter removes the tail from the water and a maximum latency of 20 sec is recorded.

Following the assessment of baseline thermal sensitivity, thermal hypersensitivity is produced by a 50 μL injection of 0.1 mg prostaglandin E₂ (PGE₂) into the terminal 1 cm of the tail. Temperature-effect curves are generated before (baseline) and after (15, 30, 60, 90 and 120 min) the PGE₂ injection. Previous studies in other species (e.g., monkeys; Brandt et al., J. Pharmacol. Exper. Ther. 296:939, 2001) have demonstrated that PGE₂ produces a dose- and time-dependent thermal hypersensitivity that peaks 15 min after injection and dissipates after 2 hr.

Single compound studies. The ability of drugs to reverse PGE₂-induced thermal hypersensitivity is assessed using a single dose time-course procedure. Under this procedure, a single dose of the compound to be tested is administered intraperitoneally (IP), orally (PO) or intranasally (IN) 30 min before the injection of PGE₂. Tactile sensitivity is assessed 30 min after PGE₂ injection.

Combination compound studies. Combination studies with two or more potential pain treatment agents can be conducted. A minimally effective dose of a first agent, e.g., morphine is administered alone and in combination with ineffective doses of one or more compounds of formula I in the thermal warm-water tail withdrawal assay. Compounds are administered IP at the same time 30 min before testing.

Combination studies can also be conducted in the PGE₂-induced thermal hypersensitivity assay. For example, a dose of morphine that completely reverses thermal hypersensitivity (i.e., return to baseline) can be administered alone and in combination with doses of one or more compounds of formula I in the PGE₂-induced thermal warm-water tail withdrawal assay. Compounds are administered IP at the same time as PGE₂, which is administered 30 min before testing.
Test Method 1 Data Analysis The temperature that produced a half-maximal increase in the tail-withdrawal latency (i.e., T₁₀) is calculated from each temperature-effect curve. The T₁₀ is determined by interpolation from a line drawn between the point above and the point below 10 sec on the temperature-effect curve. For these studies, thermal hypersensitivity is defined as a leftward shift in the temperature-effect curve and a decrease in the T₁₀ value. Reversal of thermal hypersensitivity is defined as a return to baseline of the temperature-effect curve and the T₁₀ value and is calculated according to the following equation:

$$\% MPE = \frac{( {T}_{10}^{drug + PGE 2} ) - ( {T}_{10}^{PGE2} )}{( {T}_{10}^{baseline} ) - ( {T}_{10}^{PGE 2} )} ⨯ 100$$

in which T₁₀^{drug+PGE2} is the T₁₀ after a drug in combination with PGE₂, T₁₀^{PGE2} is the T₁₀ after PGE₂ alone, and T₁₀^{baseline} is the T₁₀ under control conditions. A % MPE value of 100 indicates a complete return to the baseline thermal sensitivity observed without the PGE₂ injection. A value of greater than 100% indicates that the compound tested reduced thermal sensitivity more than the baseline thermal sensitivity without the PGE₂ injection.
Test Method 2: Chronic Constriction Injury

Rats are anesthetized with 3.5% halothane in O₂ at 1 L/min and maintained with 1.5% halothane in O₂ during surgery. A modified chronic sciatic nerve constriction injury (Mosconi & Kruger, 1996; Bennett & Xie, 1988) is produced by a cutaneous incision and a blunt dissection through the biceps femoris to expose the sciatic nerve. A PE 90 Polyethylene tubing (Intramedic, Clay Adams; Becton Dickinson Co.) cuff (2 mm length) is placed around the sciatic nerve at the level of the mid-thigh. The wound is closed in layers using 4-0 silk suture and wound clips. Testing is conducted 6-10 days after surgery.

Animals are placed in elevated wire cages and allowed 45-60 minutes to acclimate to the testing room. Baseline tactile sensitivity is assessed using a series of calibrated von Frey monofilaments (Stoelting; Wood Dale, Ill.) 0-3 days before surgery. Von Frey monofilaments are applied to the mid-plantar hind paw in sequential ascending or descending order, as necessary, to hover as closely as possible to the threshold of responses. The threshold is indicated by the lowest force that evoked a brisk withdrawal response to the stimuli. Thus, a withdrawal response leads to the presentation of the next lighter stimulus and the lack of a withdrawal response leads to the presentation of the next stronger stimulus. Rats with baseline thresholds <4 g force are excluded from the study. Approximately one week following CCI surgery, tactile sensitivities are reassessed and animals that exhibit motor deficiency (i.e. paw dragging) or failure to exhibit subsequent tactile hypersensitivity (threshold ≧10 g) are excluded from further testing. Under cumulative dosing conditions, compounds are administered IP every 30 minutes with the cumulative dose increasing in ½ log unit increments. Tactile hypersensitivity is assessed 20-30 minutes following each drug administration.
Test Method 2 Data Analysis. The 50% threshold values (in gm force) estimated by the Dixon non-parametric test (Chaplan et al, 1994) are calculated and fifteen-grams of force is used as the maximal force. Dose-effect curves are generated for each experimental condition for each rat. Individual tactile hypersensitivity threshold values are averaged to provide a mean (±1 SEM). Reversal of tactile hypersensitivity was defined as a return to baseline tactile sensitivity and was calculated according to the following equation:

$$\% Reversal = \frac{( 50 {\%}^{drug + CCI} ) - ( 50 {\%}^{CCI} )}{( 50 {\%}^{baseline} ) - ( 50 {\%}^{CCI} )} ⨯ 100$$

in which 50%^{drug+CCI} is the 50% value after compound in animals approximately one week after CCI surgery, 50%^{CCI} is the 50% value approximately one week after CCI surgery alone, and 50%^{baseline} is the 50% value before CCI surgery. Maximal effect of 100% reversal represents a return to the mean pre-operative threshold value for subjects in that experimental condition.
Test Method 3: Scheduled-Controlled Responding.

Rats are trained under a multiple-cycle procedure during experimental sessions conducted five days each week. Each training cycle consists of a 10-min pretreatment period followed by a 10-min response period. During the pretreatment period, stimulus lights are not illuminated, and responding has no scheduled consequences. During the response period, the left or right stimulus lights are illuminated (counterbalanced among subjects), the response lever is extended and subjects can respond under a fixed ratio 30 schedule of food presentation. Training sessions consist of 3 consecutive cycles. Testing sessions are identical to training sessions except that a single dose of drug is administered at the start of the first cycle.

Test Method 3 Data analysis. Operant response rates from individual animals are averaged for the three cycles during test sessions and are converted to percent of control response rates using the average rate from the previous training day as the control value (i.e., average of three cycles). Data are presented as the mean (±1 SEM) response rate as a percent of control. Thus, for example, a test value of 100% would indicate the response rate after administration of the compound to be tested is the same as the control response rate and there is no adverse effect of the compound tested.

Test Method 4: Assessment of Effectiveness in Tactile Allodynia Model

Compound: Test compounds are dissolved in sterile saline and gabapentin is suspended in 2% Tween 80 in 0.5% methylcellulose and sterile water. All compounds are administered intraperitoneally (i.p.).

Subjects: Male Sprague-Dawley rats (125-150 g, Harlan; Indianapolis, Ind.) are individually housed on bedding. For all studies animals are maintained in climate-controlled rooms on a 12-hour light/dark cycle (lights on at 0630) with food and water available ad libitum.

Surgery: All surgical procedures are performed under 4% isoflurane/O₂ anesthesia, delivered via nose cone and maintained at 2.5% for the duration of the surgery.

L5 Spinal Nerve Ligation (SNL): Surgery is performed as previously described (Kim and Chung) with the exception that nerve injury is produced by tight ligation of the left L5 spinal nerve.

Assessment of Tactile Allodynia (Tactile Sensitivity): Tactile thresholds are assessed using a series of calibrated von Frey monofilaments (Stoelting; Wood Dale, Ill.). The threshold that produced a 50% likelihood of a withdrawal is determined using the up-down method, as previously described (Chaplan et al., 1994). Animals are placed in elevated wire cages and allowed 45-60 minutes to acclimate to the testing room. Von Frey monofilaments are applied to the mid-plantar left hind paw in sequential ascending or descending order, as necessary, to hover as closely as possible to the threshold of responses. The lowest force that evokes a brisk withdrawal response to the stimuli determined the pain threshold. Tactile thresholds are determined on the day prior to surgery and rats with baseline thresholds <10 g force are excluded from studies. Three weeks after SNL surgery tactile thresholds are reassessed and animals that fail to exhibit subsequent tactile allodynia (threshold ≧5 g) are excluded from further testing. Subjects are pseudo-randomly divided into test groups (n=8-10) so that average baseline and post-surgery sensitivities are similar among groups. Rats are administered a test compound (3, 10 or 17.8, i.p.), gabapentin (100 mg/kg, i.p., postivie control) or vehicle and tactile thresholds are assessed up to 60, 180 and 300 minutes after dosing.
Analysis of Results: Statistical analysis is done using a repeated measures analysis of variance (ANOVA) using a customized SAS-excel application (SAS Institute, Cary, N.C.). Significant main effects are analyzed further by subsequent least significant difference analysis. The criterion for significant differences is p<0.05. Reversal of tactile allodynia is calculated according to the following equation:

$$\% Reversal = \frac{\begin{matrix}( 50 \% {threshold}^{drug + post surgury} ) - \\ ( 50 \% {threshold}^{post surgery} )\end{matrix}}{\begin{matrix}( 50 \% {threshold}^{pre surgery} ) - \\ ( 50 \% {threshold}^{post surgery} )\end{matrix}} ⨯ 100$$

In which 50% threshold^{drug+post surgery} is the 50% threshold in g force after drug in nerve injured subjects, 50% threshold^{post surgery} is the 50% threshold in g force in nerve injured subjects, and 50% threshold^{pre surgery} is the 50% threshold in g force before nerve injury. Maximal effect of 100% reversal represents a return to the mean pre-operative threshold value for subjects in that experimental condition.
Test Method 5:Assessment of Effectiveness in Chronic Inflammatory Pain Compounds:
Test compounds are dissolved in sterile saline and administered intraperitoneally (i.p.). Celecoxib was used as a positive control and is suspended in 2% Tween 80 in 0.5% methylcellulose and administered orally (p.o.).
Subjects: Male Sprague-Dawley rats (125-150 g, Harlan; Indianapolis, Ind.) are housed 3/cage on bedding and. animals are maintained in climate-controlled rooms on a 12-hour light/dark cycle (lights on at 0630) with food and water available ad libitum.
Freund's complete adjuvant (FCA) of mechanical hyperalgesia: The hind paw withdrawal thresholds (PWTs) to a noxious mechanical stimulus are determined using an analgesimeter (model 7200; Ugo Basile). Cutoff was set at 250 g, and the endpoint taken is complete paw withdrawal. PWT is determined once for each rat at each time point (n=10/group). Baseline PWT is determined, and the rats were anesthetized with isofluorane (2% in oxygen) and received an intraplantar injection of 50% FCA (50 μl, diluted in saline) to the left hind paw. Twenty-four hours after FCA injection, pre-drug PWTs were measured, and the rats are administered vehicle or compound and assessed on PWTs 1, 3, 5, and 24 hours post-drug administration.
Analysis of Results: Statistical analysis is done using a one way analysis of variance (ANOVA) using a customized SAS-excel application (SAS Institute, Cary, N.C.). Significant main effects are analyzed further by subsequent least significant difference analysis. The criterion for significant differences is p<0.05 from vehicle-treated FCA rats. Data is presented as percent reversal according to the following equation: percent reversal=[(post-dose threshold)−pre-dose threshold))/(baseline threshold-pre-dose threshold)]×100.
D. Assessment of Effectiveness in Treatment of Depression

Effectiveness of compounds of the present invention may be determined by the tail suspension test. While not a direct model of depression, the tail suspension test is an assay that can evaluate antidepressant-like effects of drugs. Clinically effective drugs such as Prozac (fluoxetine) are effective in this assay. Specifically, they decrease the amount of time the mice spend immobile after being hung upside down by their tails during the test. It is impossible to determine if a mouse is indeed depressed. However, the fact that clinically effective antidepressants reduce immobility lends predictive validity to the model.

Male Swiss Webster mice (Charles River) weighing 25-35 g are housed in groups of five per cage in an AALAC-accredited facility that is maintained on a 12-h light dark cycle (lights on at 0600 h) and have free access to food and water. Experimental groups consist of 12 mice, randomly assigned to treatment groups. Experiments are performed between 9:00 AM and noon in accordance to the Guide for the Care and Use of Laboratory Animals as adopted and promulgated by the National Institutes of Health (Pub. 85-23, 1985).

Solutions of test compounds are dissolved in distilled water. Compounds are injected i.p. at a volume of 10 ml/kg body weight. Combination treatments are cotreated, 30 minutes prior to the test.

The procedure described herein is substantially similar to that described by Steru et al. (1985). 30 minutes following treatment, the mice are suspended upside down by the tail using adhesive laboratory tape (VWR International), to a flat metal bar connected to a strain gauge within a tail suspension chamber (Med Associates). The time spent immobile during a 6-minute test session is automatically recorded. 8 mice are simultaneously tested within separate chambers. Data collected are expressed as a mean of immobility time and statistical analysis is performed using a one-way ANOVA with least significant difference (LSD) post-hoc test.

The entire disclosure of each patent, patent application, and publication cited or described in this document is hereby incorporated by reference.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

We claim:

1. A compound of formula I: 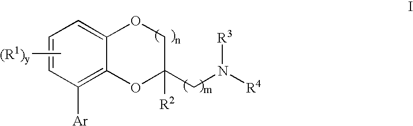
or a pharmaceutically acceptable salt thereof, wherein:
m is 1 or 2;
n is 0 or 1;
Ar is phenyl, an 8-10 membered bicyclic partially unsaturated or aryl carbocyclic ring, a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur, wherein Ar is optionally substituted with one or more R^{x} groups;
each R^{x} is independently selected from —R, -Ph —CN, halogen, —OR, —C(O)NH₂, —C(O)OR, —NHC(O)R, —SO₂R, or —NHSO₂R;
y is 0-3;
each R¹ is independently —R, —CN, halogen, —OR, —C(O)NH₂, —C(O)OR, —NHC(O)R, —SO₂R, or —NHSO₂R;
each R is independently hydrogen or C₁₋₆ aliphatic or fluoro-substituted C₁₋₆ aliphatic;
R² is hydrogen, C₁₋₃ alkyl, or —O(C₁₋₃ alkyl); and
each of R³ and R⁴ is independently hydrogen or C₁₋₆ aliphatic.

2. The compound according to claim 1, wherein said compound is of formula Ia: 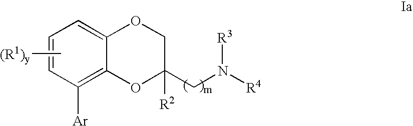
or a pharmaceutically acceptable salt thereof.

3. The compound according to claim 2, wherein each R¹ is independently —R, —CN, halogen or —OR.

4. The compound according to claim 3, wherein said compound has the formula IIa or IIb: 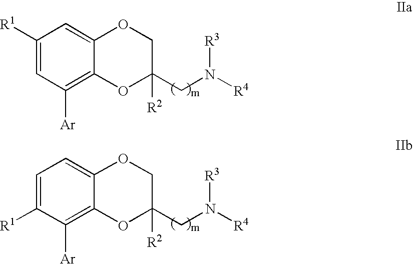
or a pharmaceutically acceptable salt thereof.

5. The compound according to claim 4, wherein Ar is phenyl, an 8-10 membered bicyclic partially unsaturated or aryl carbocyclic ring, or a 5-6 membered monocyclic heteroaryl having 1-4 heteroatoms independently selected from nitrogen, oxygen, or sulfur.

6. The compound according to claim 5, wherein Ar is pyridyl, pyrimidinyl, thienyl, or furanyl.

7. The compound according to claim 5, wherein said compound is of formula IIIa or IIIc: 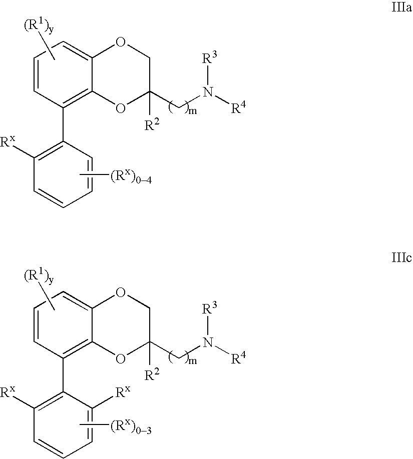
or a pharmaceutically acceptable salt thereof.

8. The compound according to claim 7, wherein each R^{x} is independently selected from —R -Ph, —CN, halogen, or —OR.

9. The compound according to claim 2, wherein:
each R¹ is independently —R, —CN, halogen, or —OR;
R² is hydrogen, methyl, or methoxy;
Ar is pyridyl, pyrimidinyl, thienyl, furanyl, or phenyl optionally substituted with one or more R^{x} groups;
each R^{x} is independently selected from —R, -Ph, —CN, halogen, or —OR; and
each of R³ and R⁴ is independently hydrogen, methyl, ethyl, cyclopropyl, cyclopropylmethyl, n-propyl, allyl, or cyclobutyl.

10. The compound according to claim 1, wherein said compound is of formula Ib: 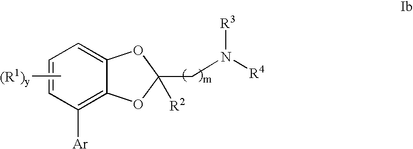
or a pharmaceutically acceptable salt thereof.

11. The compound according to claim 10, wherein each R¹ is independently —R, —CN, halogen, or —OR.

12. The compound according to claim 11, wherein said compound is of formula IIc or IId: 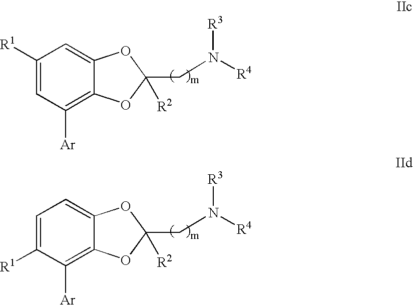
or a pharmaceutically acceptable salt thereof.

13. The compound according to claim 12, wherein Ar is phenyl, an 8-10 membered bicyclic partially unsaturated or aryl carbocyclic ring, or a 5-6 membered monocyclic heteroaryl having 1-4 heteroatoms independently selected from nitrogen, oxygen, or sulfur.

14. The compound according to claim 13, wherein Ar is pyridyl, pyrimidinyl, thienyl, or furanyl.

15. The compound according to claim 13, wherein said compound is of formula IIIb or IIId: 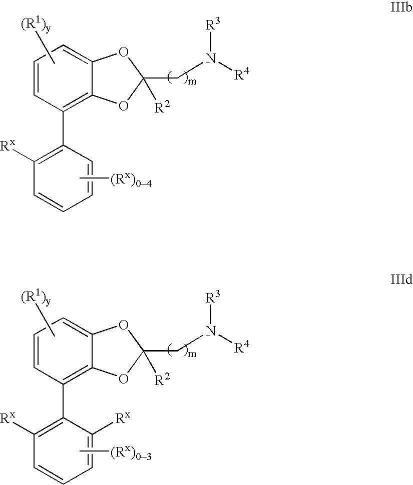
or a pharmaceutically acceptable salt thereof.

16. The compound according to claim 15, wherein each R^{x} is independently selected from R, Ph, CN, halogen, or OR.

17. The compound according to claim 10, wherein:
each R¹ is independently —R, —CN, halogen or —OR;
R² is hydrogen, methyl, or methoxy;
Ar is pyridyl, pyrimidinyl, thienyl, furanyl, or phenyl optionally substituted with one or more R^{x} groups;
each R^{x} is independently selected from —R, -Ph, —CN, halogen or —OR; and
each of R³ and R⁴ is independently hydrogen, methyl, ethyl, cyclopropyl, cyclopropylmethyl, n-propyl, allyl, or cyclobutyl.

18. The compound according to claim 1, wherein Ar is selected from:

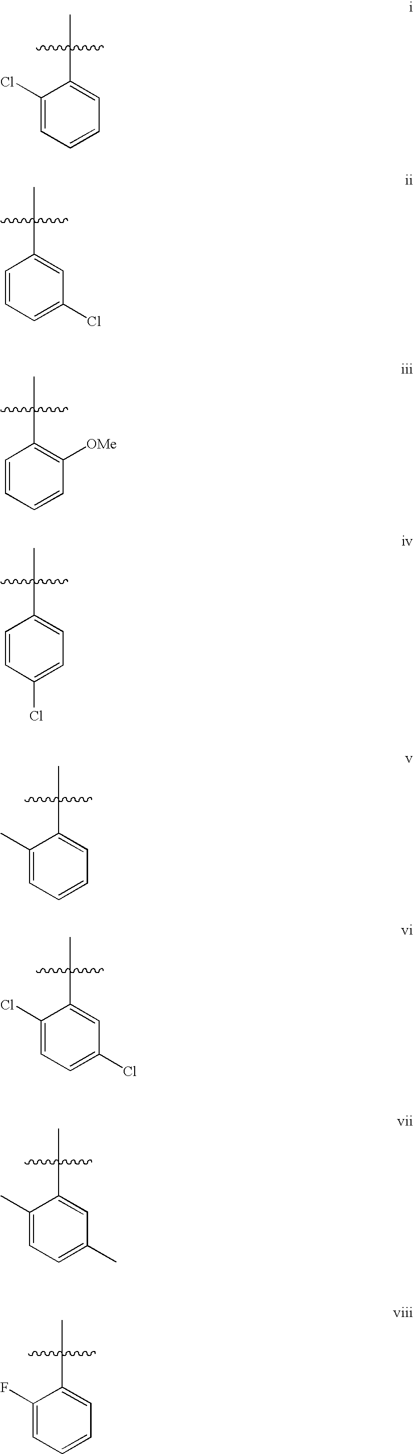

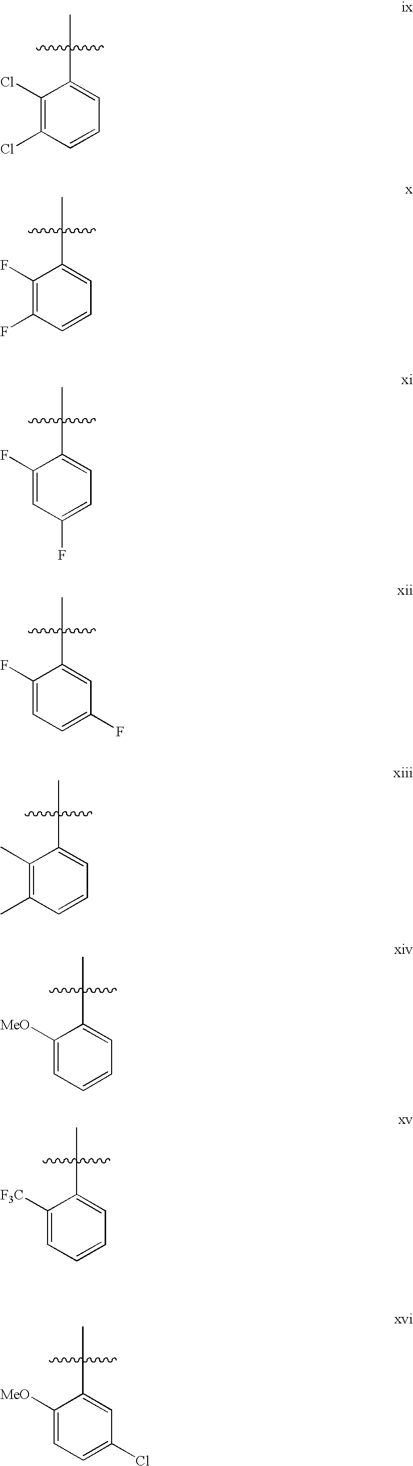

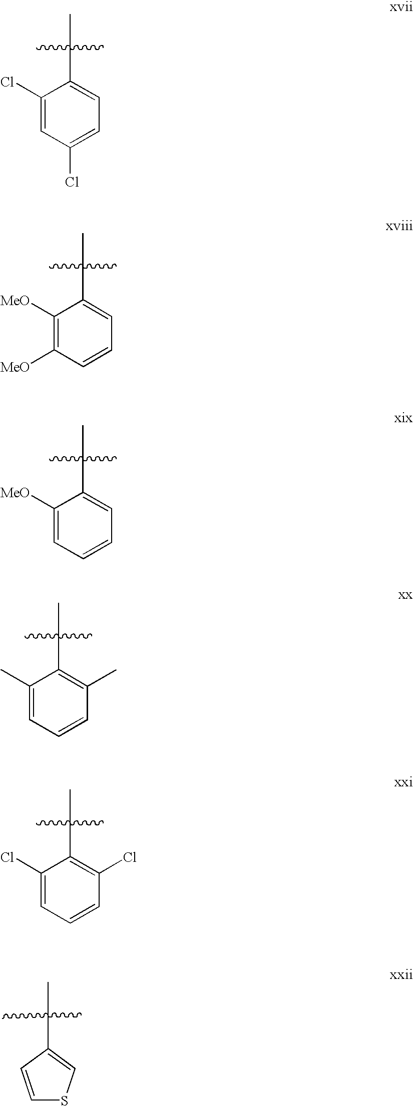

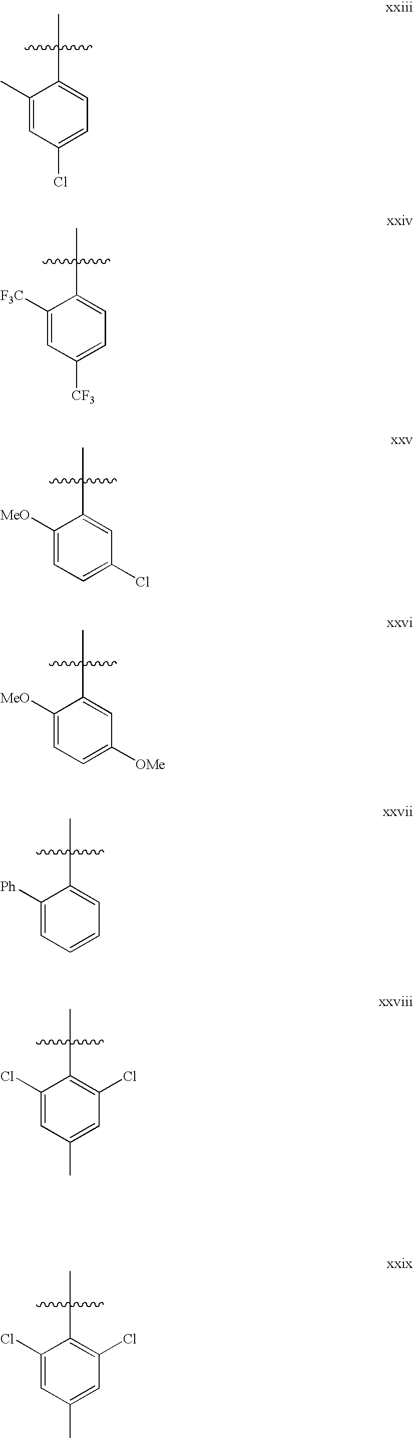

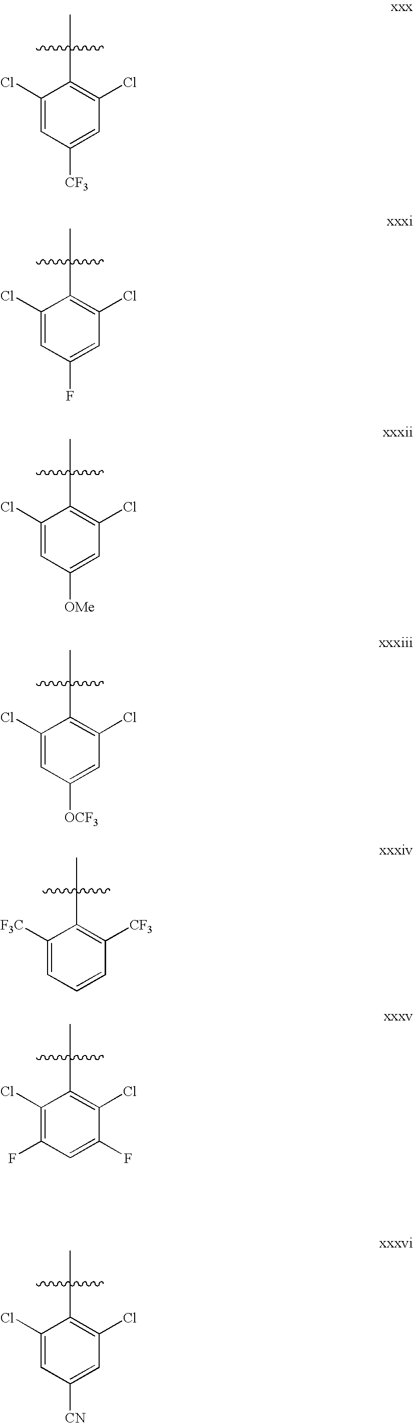

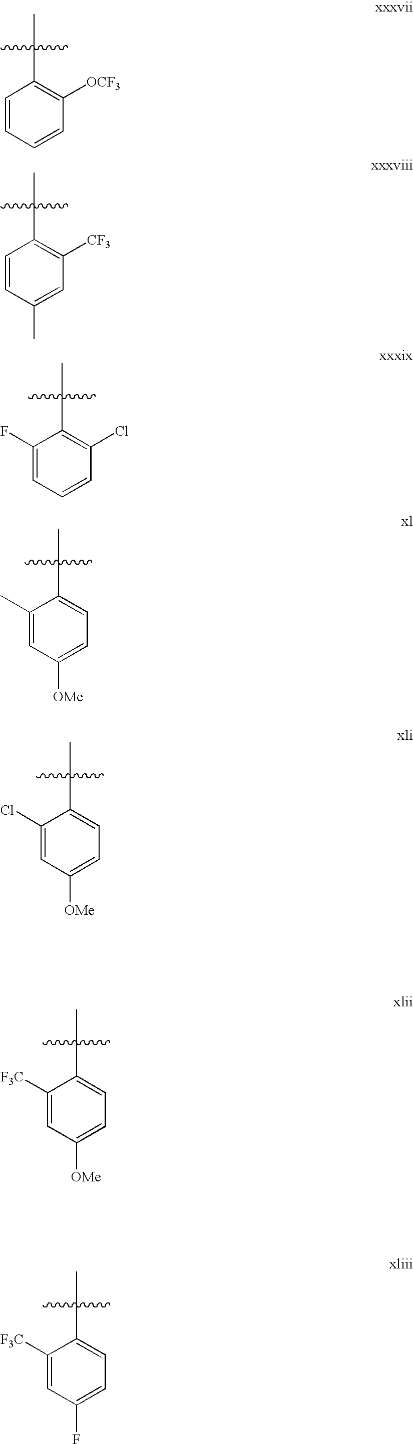

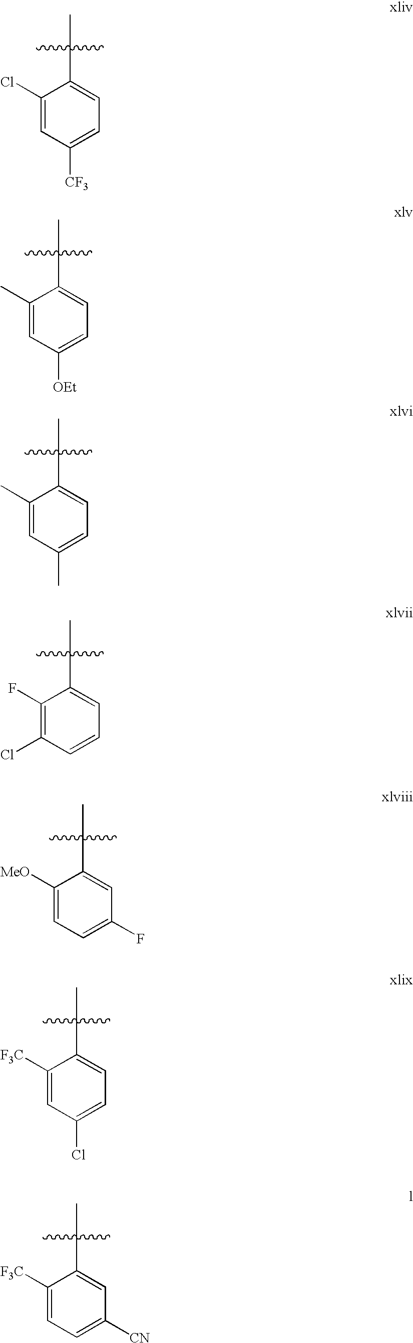

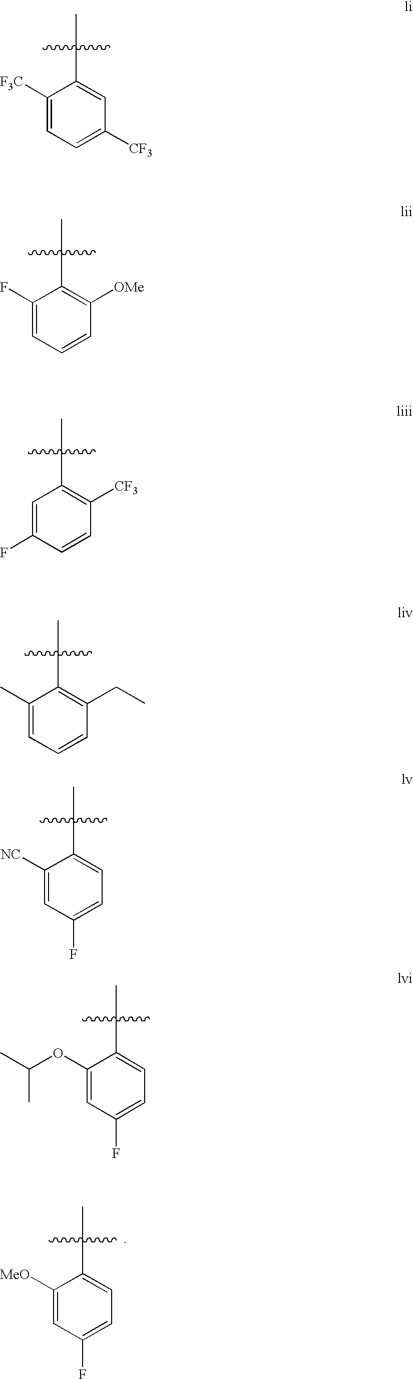

19. The compound according to claim 1, wherein said compound is selected from: 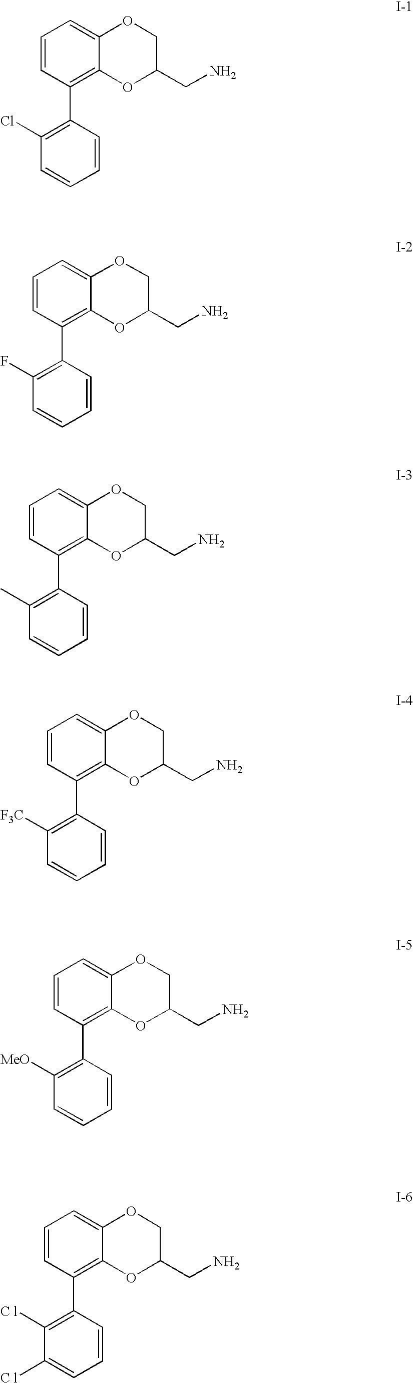 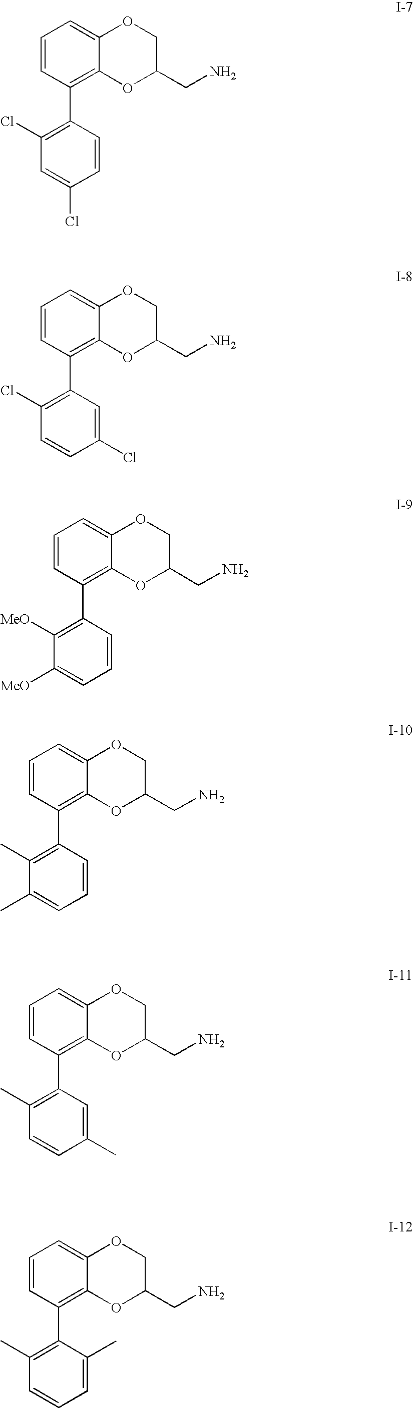 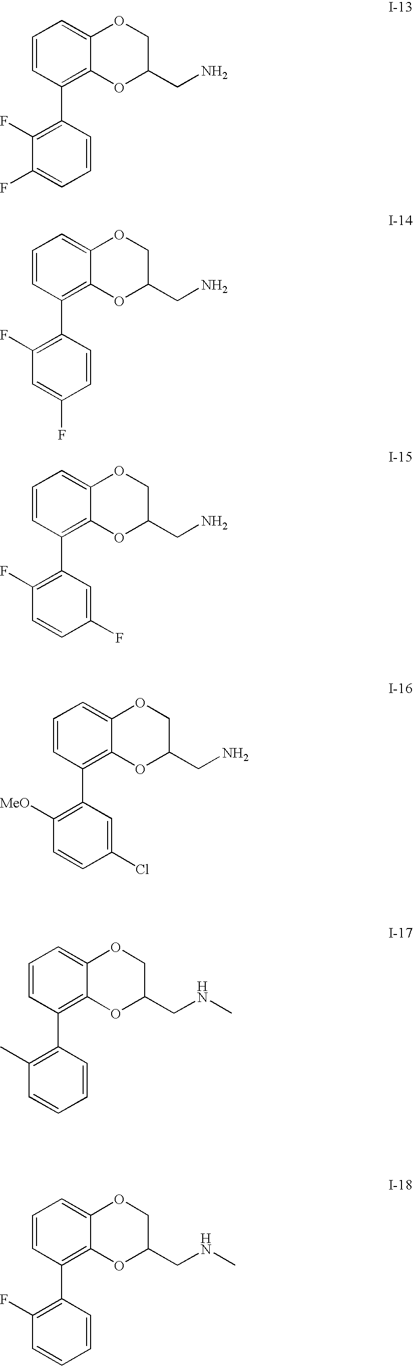 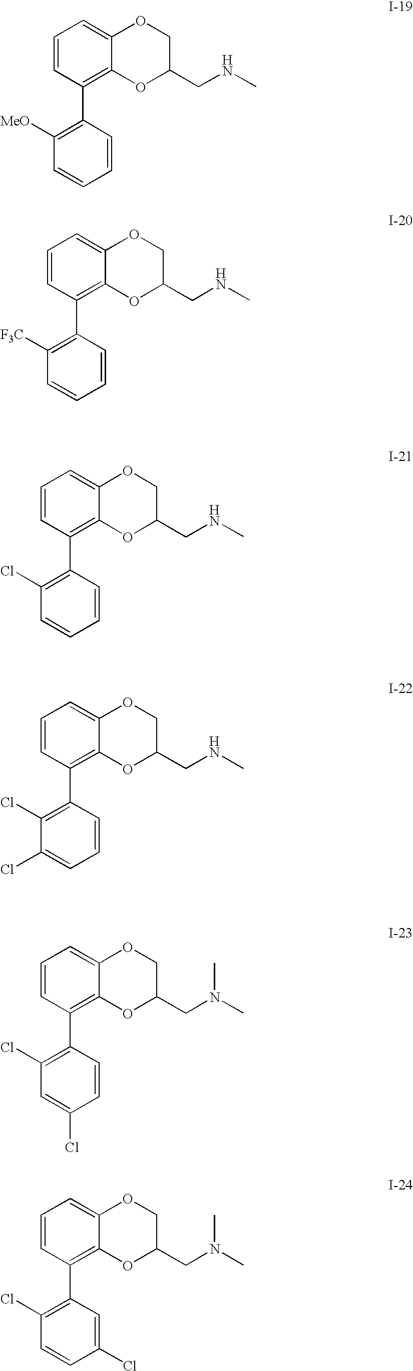 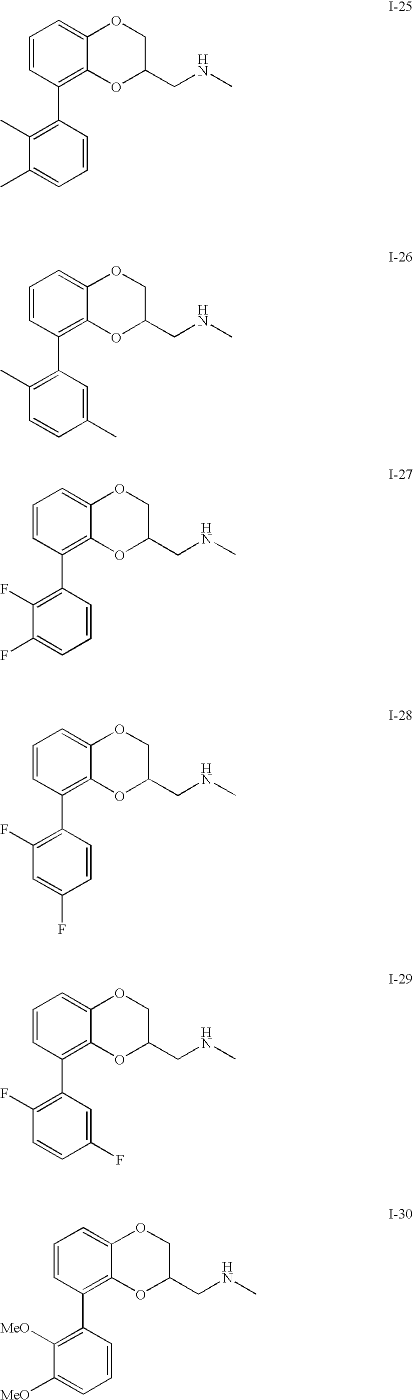 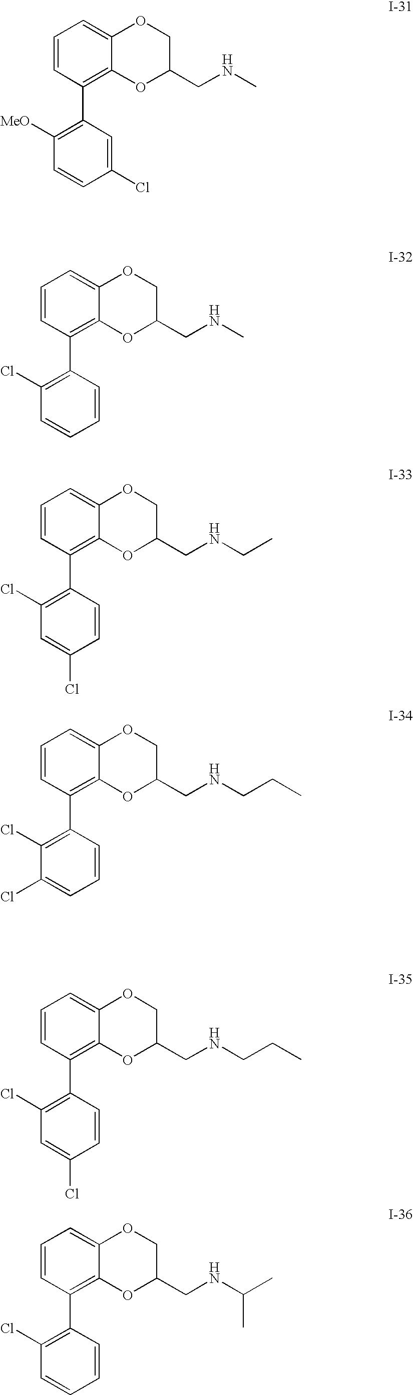 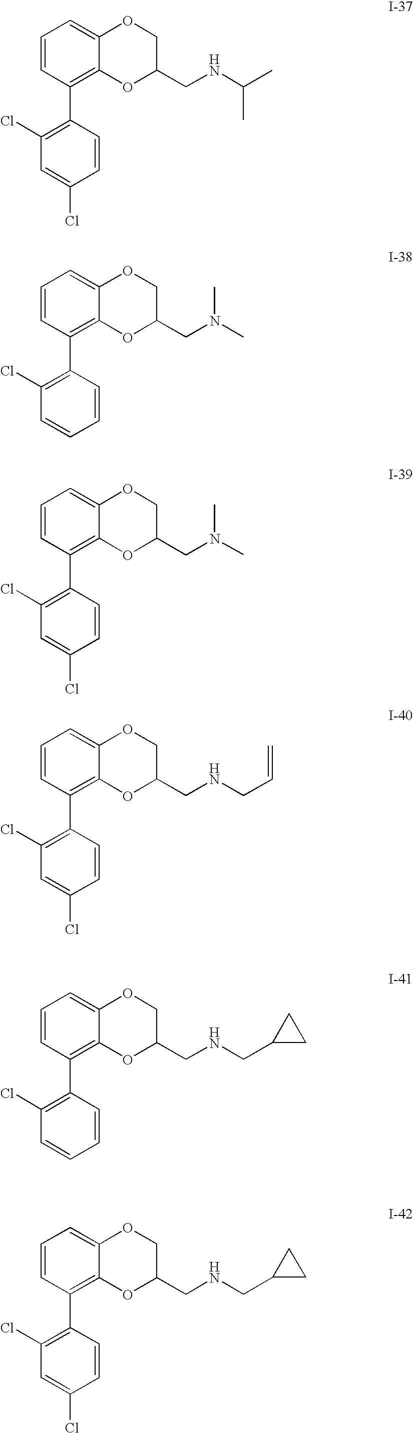 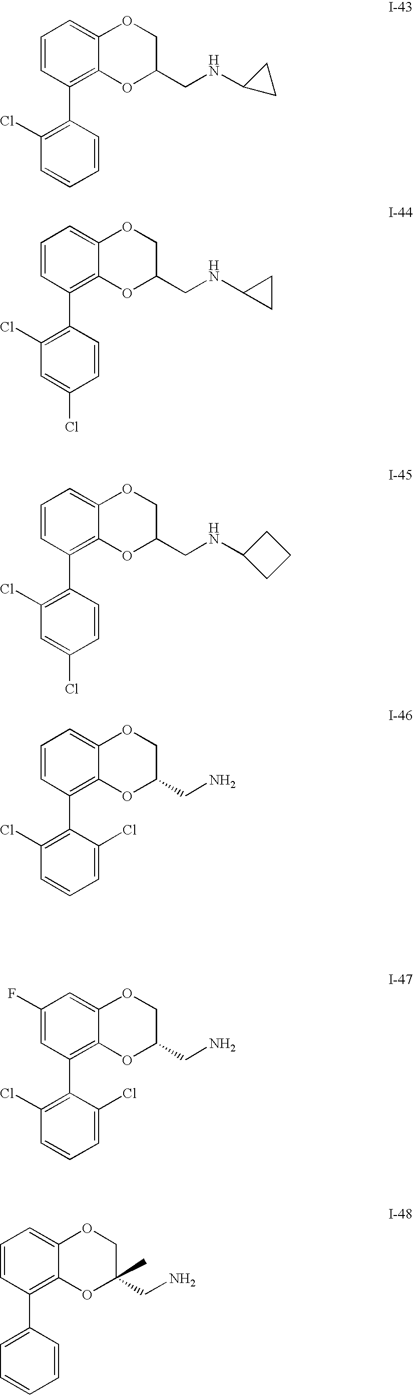 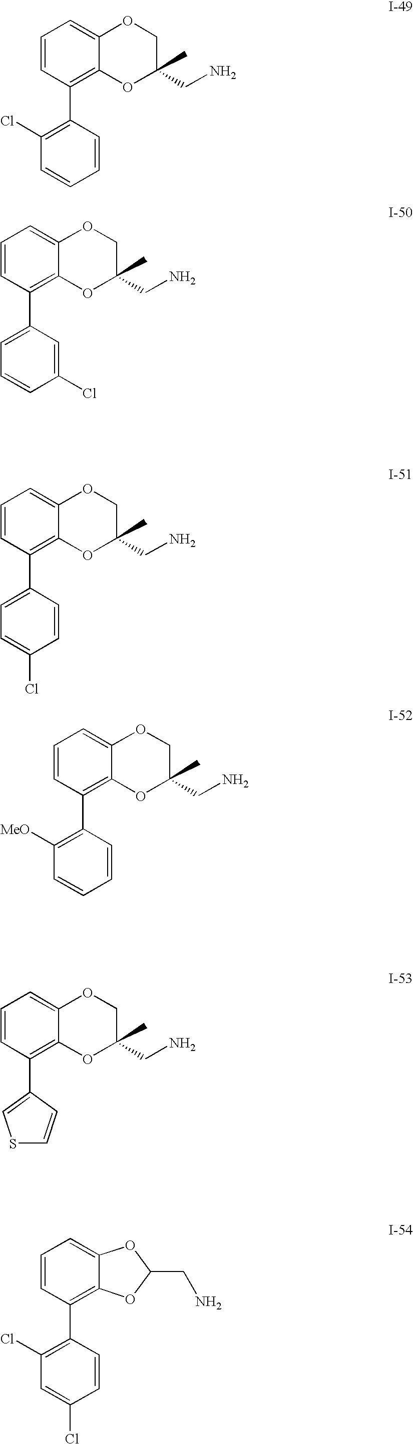 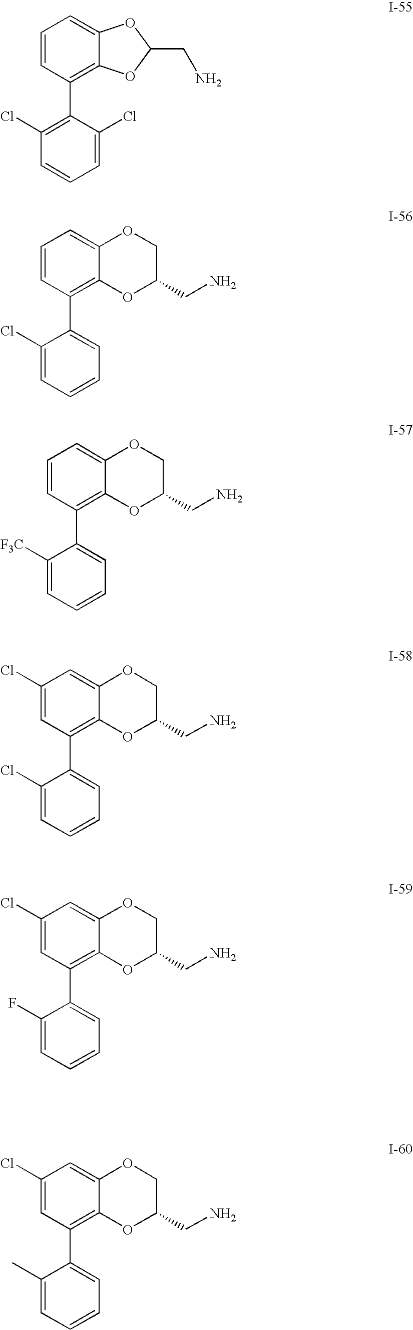 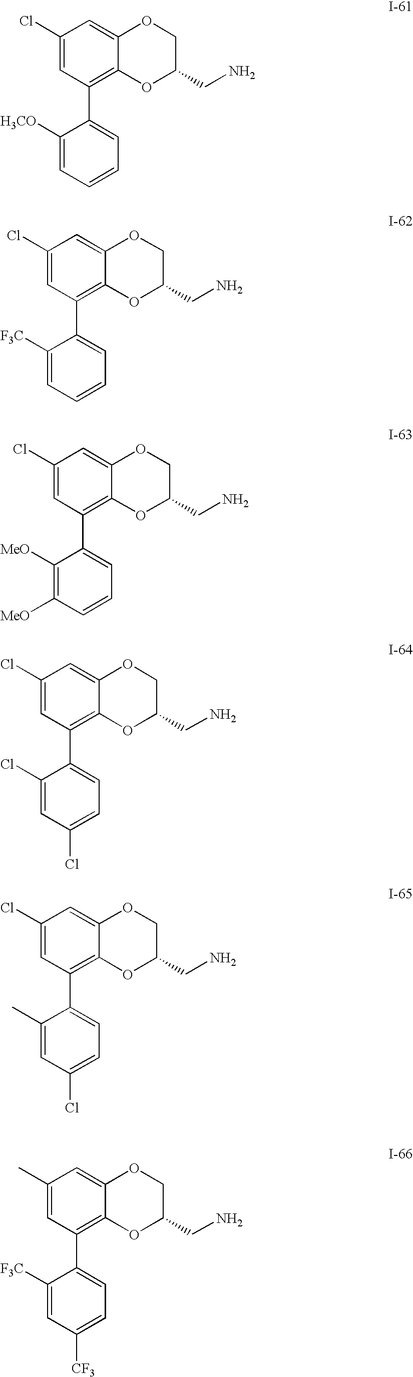 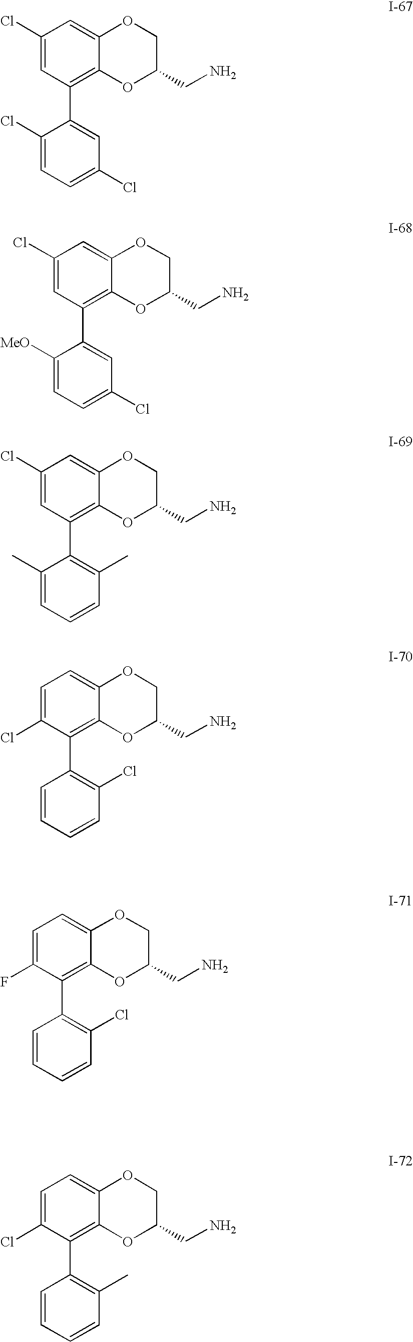 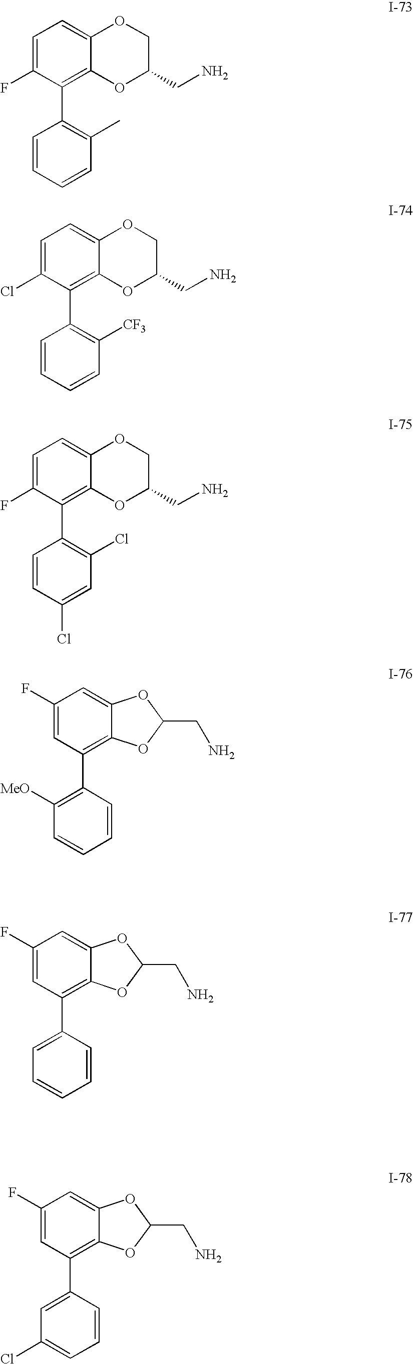 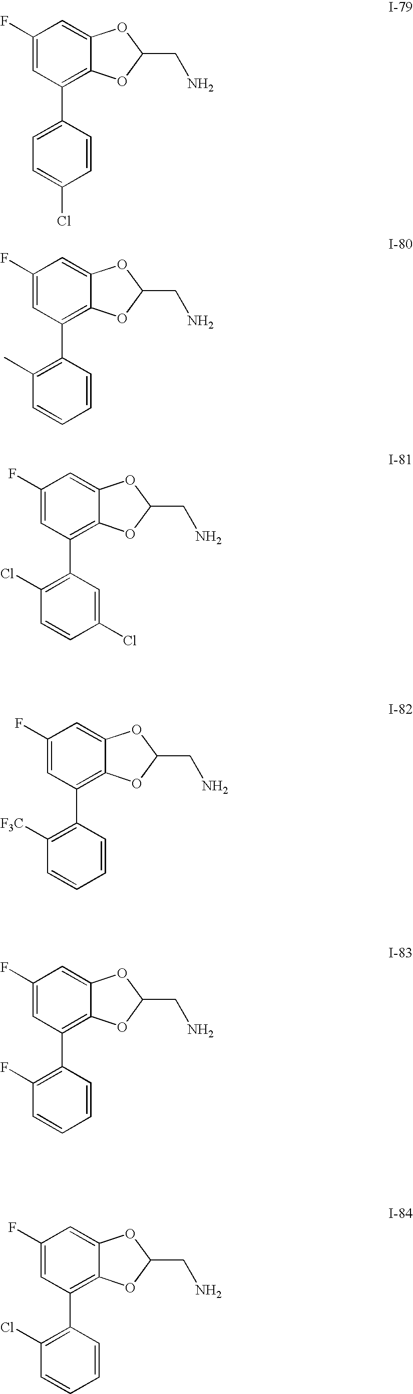 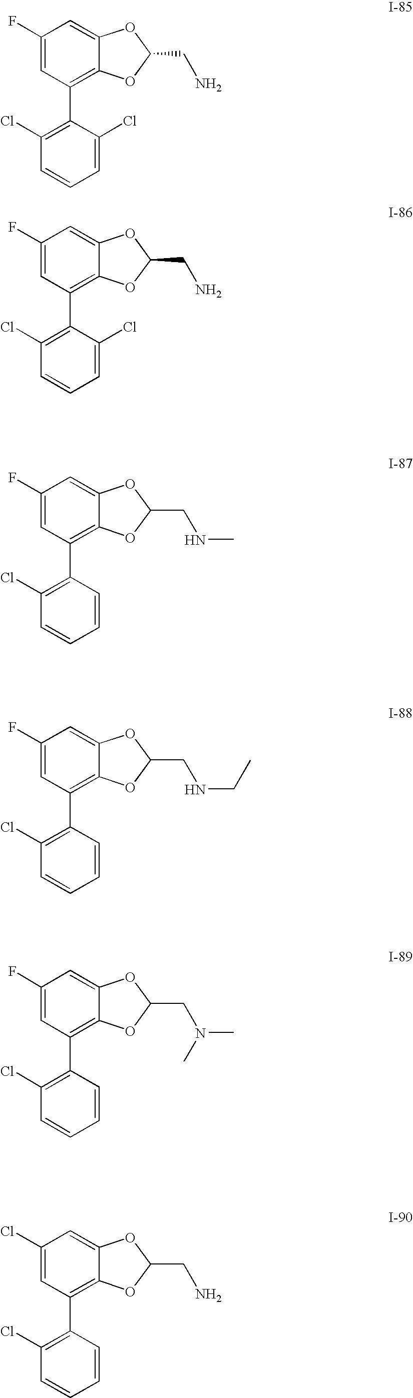 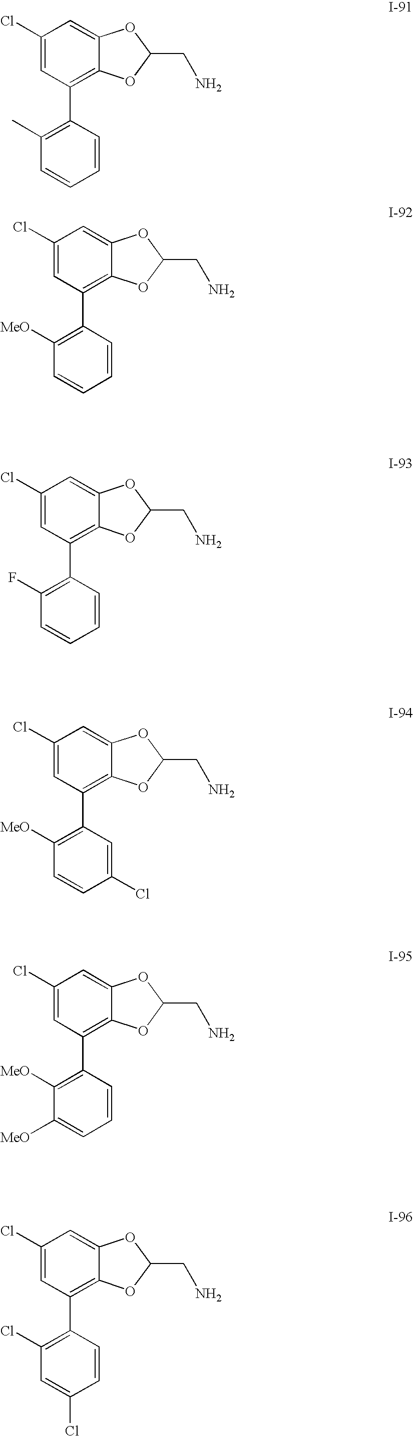 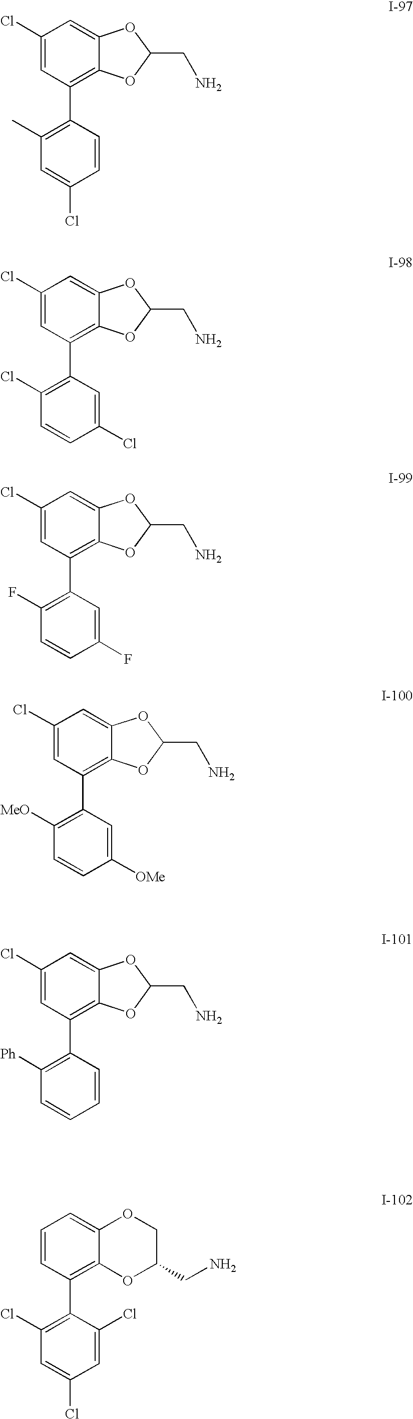 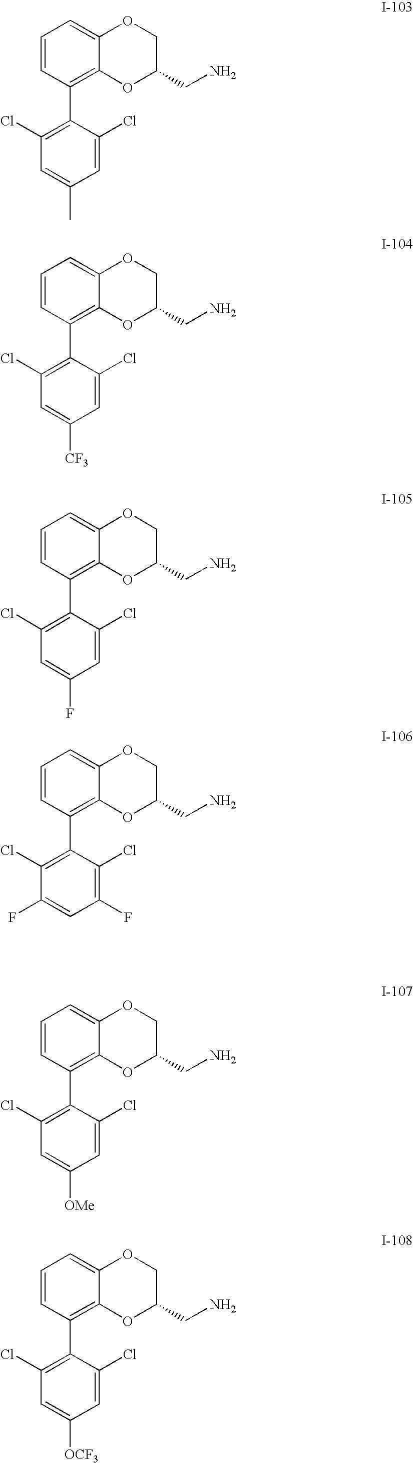 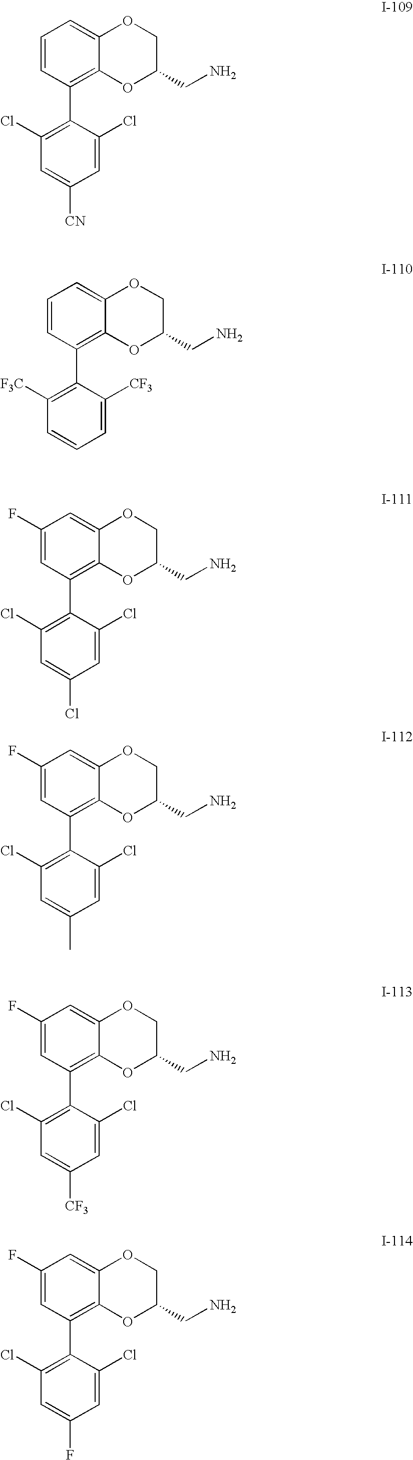 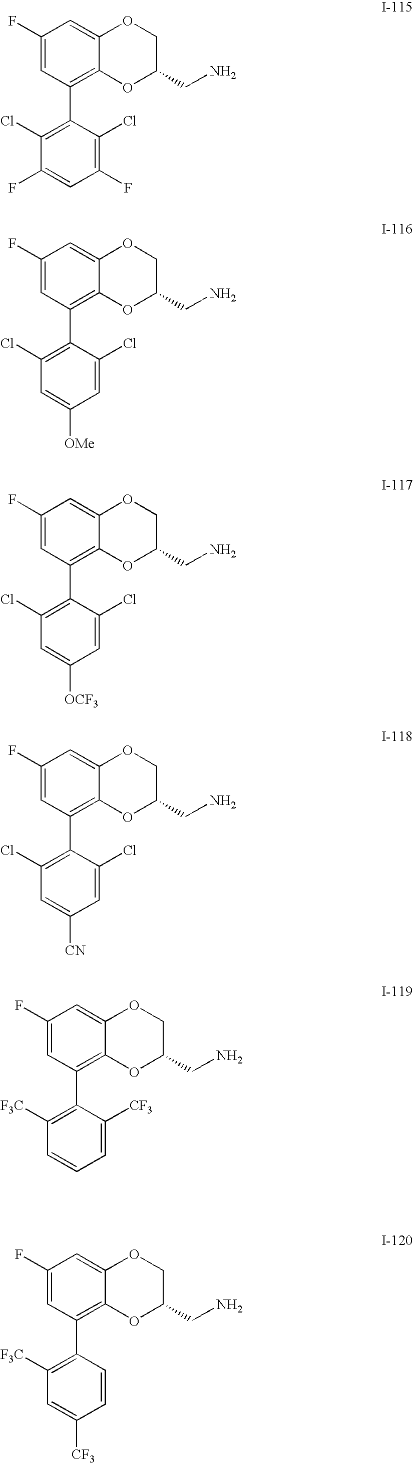 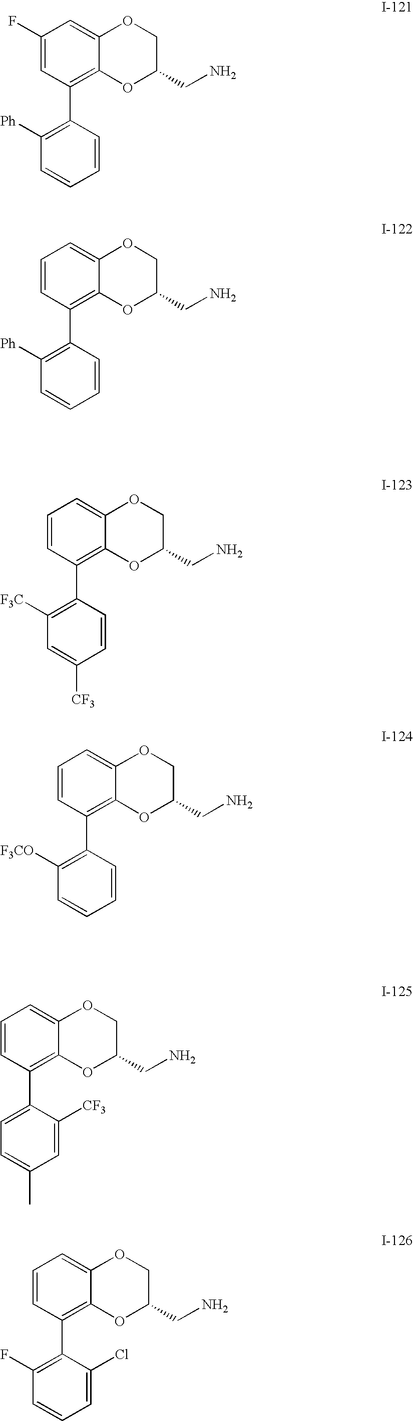 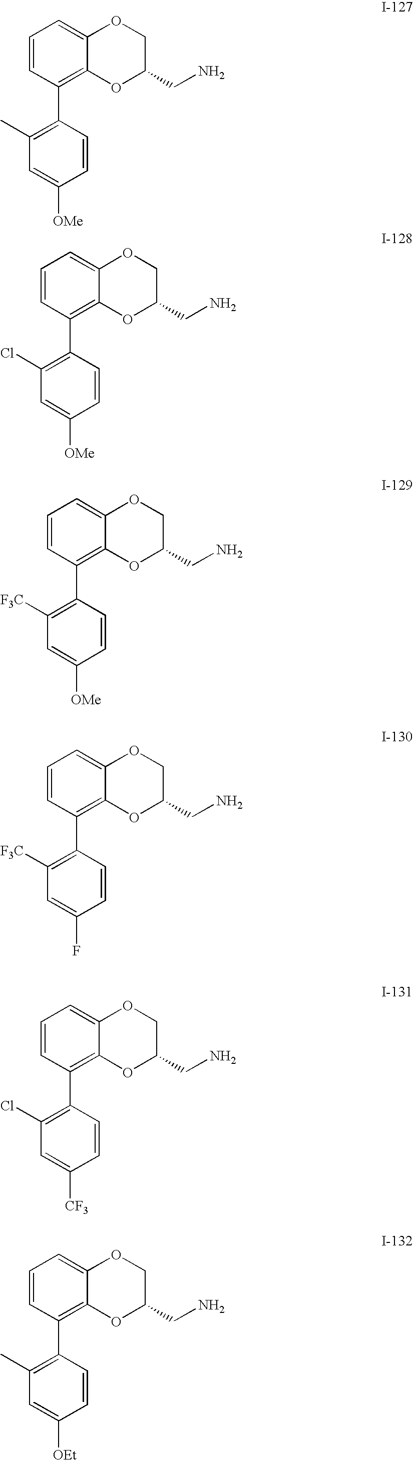 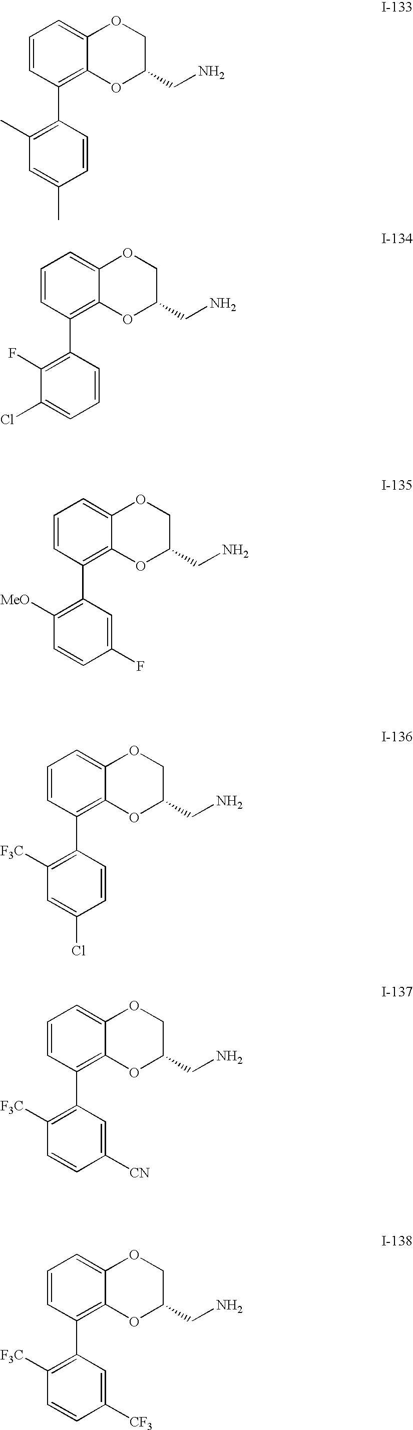 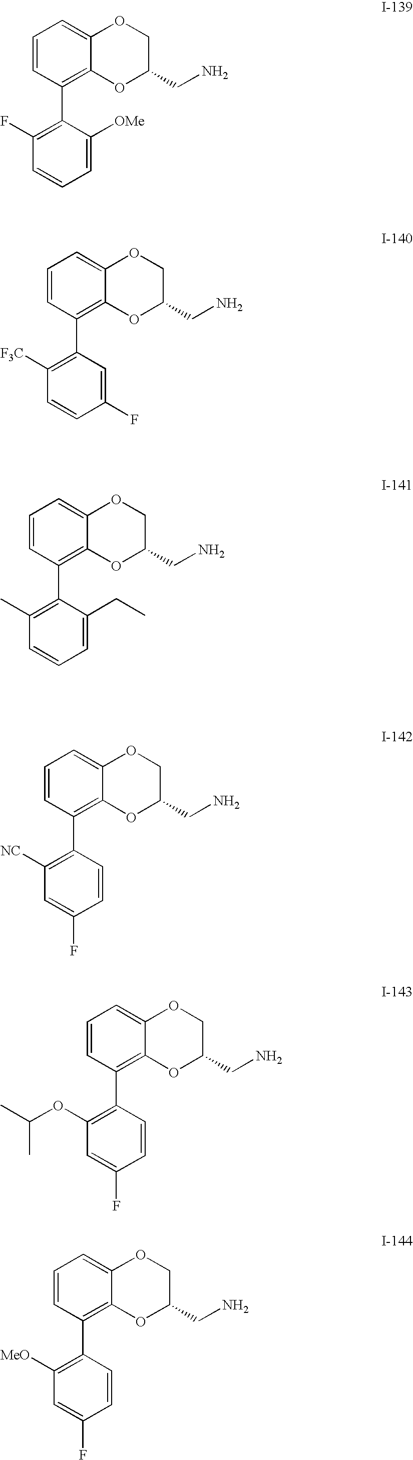 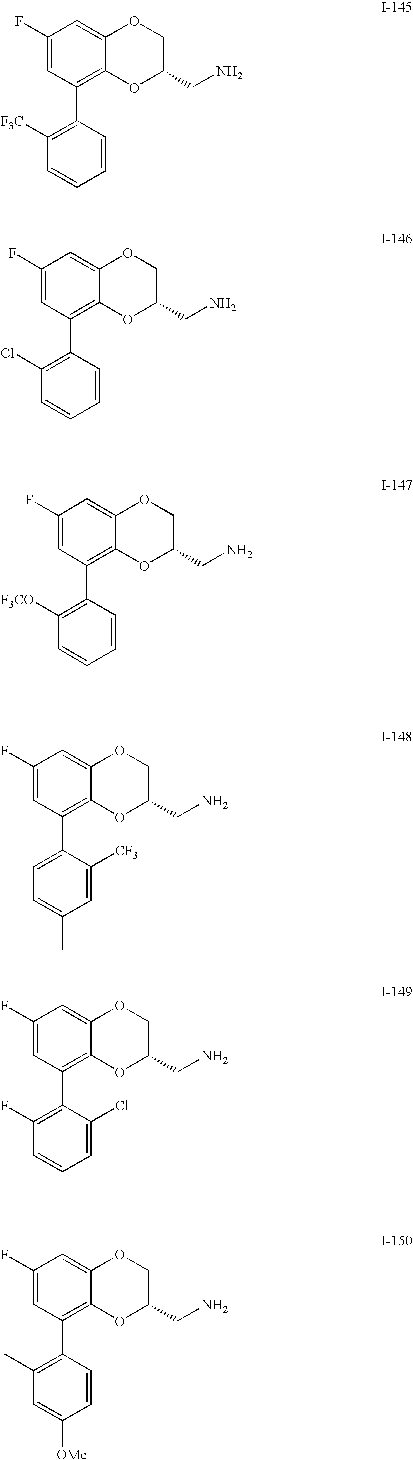 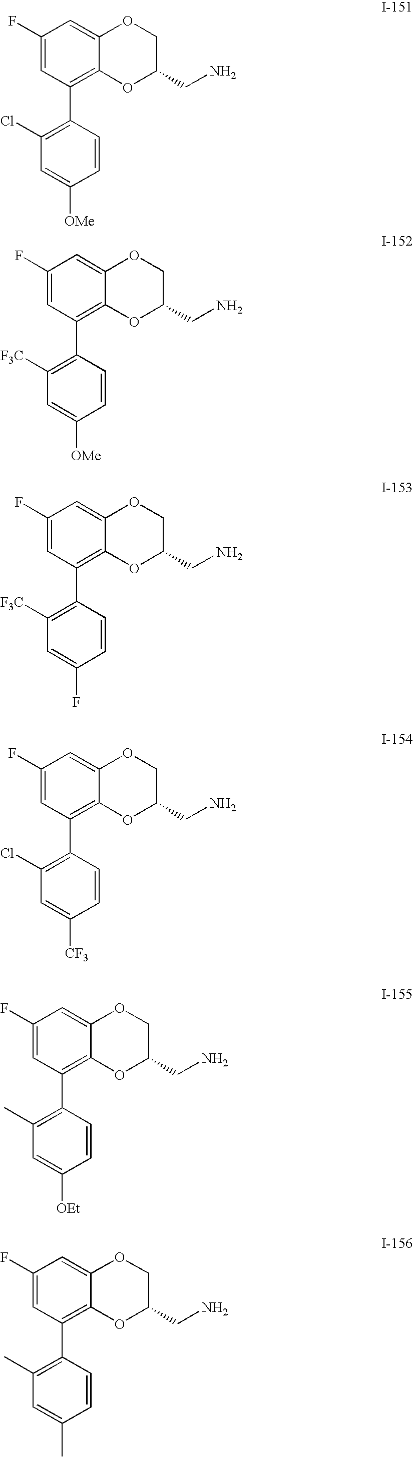 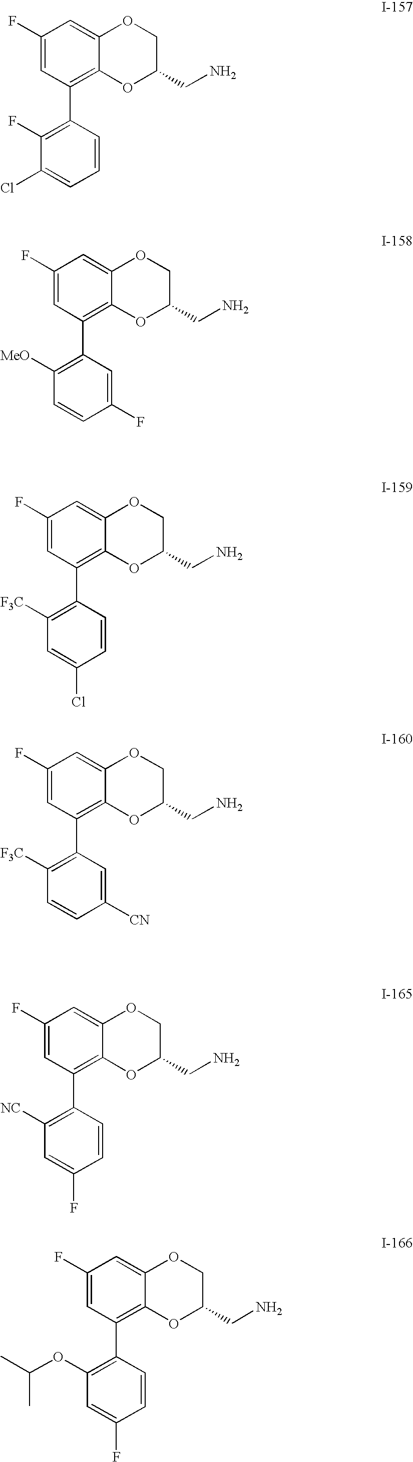 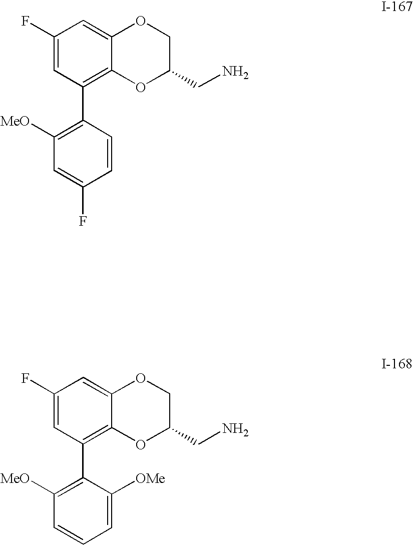
or an enantiomer or racemate thereof.

20. A composition comprising a compound according to claim 1, and one or more pharmaceutically acceptable carriers, diluents, or excipients.

21. The composition of claim 20, further comprising an additional pharmaceutical agent selected from an anti-psychotic agent, an antidepressive agent, an anti-obesity agent, an agent useful in the modulation of bladder activity, an opioid antagonist, an agent for treating ADD or ADHD, a cognitive improvement agent, an agent for treating sexual dysfunction, or a pain relieving agent.

22. A method for treating a condition selected from at least one of psychotic disorder, an anxiety disorder, a bipolar disorder, a depressive disorder, premenstrual syndrome (PMS), premenstrual dysphoric disorder (PMDD), an eating disorder, a bladder control disorder, substance abuse or substance dependence, a cognition disorder, ADD or ADHD, an impulsivity disorder, an addictive disorder, male or female sexual dysfunction, pain, a motion or motor disorder, Parkinson's disease epilepsy, migraine, chronic fatigue syndrome, anorexia nervosa, a sleep disorder, mutism, or one or more central nervous system deficiencies in a patient, comprising administering to the patient a therapeutically effective amount of a compound according to claim 1 or a composition comprising a compound according to claim 1.

23. The method of claim 22 wherein the psychotic disorder is schizophrenia, paranoid type schizophrenia, disorganized type schizophrenia, catatonic type schizophrenia, undifferentiated type schizophrenia, a schizophreniform disorder, a schizoaffective disorder, a delusional disorder, substance-induced psychotic disorder, a psychotic disorder not otherwise specified; L-DOPA-induced psychosis; psychosis associated with Alzheimer's dementia; psychosis associated with Parkinson's disease; or psychosis associated with Lewy body disease

24. The method of claim 22, wherein the condition is bipolar disorder and is selected from bipolar I disorder, bipolar II disorder, cyclothymic disorder; bipolar mania, dementia, depression with psychotic features, or cycling between bipolar depression and bipolar mania.

25. The method of claim 22, wherein the depressive disorder is major depressive disorder, seasonal affective disorder, dysthymic disorder, substance-induced mood disorder, depressive disorder not otherwise specified, treatment resistant depression, major depressive episode.

26. The method of claim 25, further comprising administering to the patient an antidepressive agent selected from serotonin reuptake inhibitors (SRIs), norepinephrine reuptake inhibitors (NRIs), combined serotonin-norepinephrine reuptake inhibitors (SNRIs), monoamine oxidase inhibitors (MAOIs), reversible inhibitors of monoamine oxidase (RIMAs), phosphodiesterase-4 (PDE4) inhibitors, corticotropin releasing factor (CRF) antagonists, alpha.-adrenoreceptor antagonists, triple uptake inhibitors, melatonin agonists, super neurotransmitter uptake blockers (SNUBs), noradrenergic and specific serotonergic antidepressants (NaSSAs), or substance P/neurokinin receptor antagonists.

27. The method of claim 22, wherein the cognitive disorder is a learning disorder.

28. The method of claim 22, wherein the patient is treated for obesity.

29. The method of claim 22, wherein the patient is treated for ADD or ADHD.

30. The method of claim 22, wherein the substance abuse substance dependence is of a recreational substance, a pharmacologic agent, a tranquilizer, a stimulant, sedative, or illicit drug.

31. The method of claim 22, further comprising administering to the patient an additional pharmaceutical agent selected from an anti-psychotic agent, an antidepressive agent, an anti-obesity agent, an agent useful in the modulation of bladder activity, an opioid antagonist, an agent for treating ADD or ADHD, a cognitive improvement agent, an agent for treating sexual dysfunction, or a pain relieving agent.

32. A method for treating schizophrenia in a patient, comprising administering to the patient a therapeutically effective amount of a composition according to claim 20.

33. A method for treating obesity in a patient, comprising administering to the patient a therapeutically effective amount of a composition according to claim 20.

34. A method for treating bipolar disorder in a patient, comprising administering to the patient a therapeutically effective amount of a composition according to claim 20.

35. A method for treating depression in a patient, comprising administering to the patient a therapeutically effective amount of a composition according to claim 20.
